(19) United States
(12) Patent Application Publication
(10) Pub. No.: US 2012/0027771 A1
(43) Pub. Date: Feb. 2, 2012

(54) AFFINITY PURIFIED HUMAN POLYCLONAL ANTIBODIES AGAINST VIRAL, BACTERIAL AND/OR FUNGAL INFECTIONS AND METHODS OF MAKING AND USING THE SAME

(76) Inventor: THOMAS L. CANTOR, EL CAJON, CA (US)

(21) Appl. No.: 13/085,347

(22) Filed: Apr. 12, 2011

Related U.S. Application Data
(60) Provisional application No. 61/323,335, filed on Apr. 12, 2010.
(60) Provisional application No. 61/345,543, filed on May 17, 2010.
(60) Provisional application No. 61/350,900, filed on Jun. 2, 2010.
(60) Provisional application No. 61/352,336, filed on Jun. 7, 2010.

(51) Int. Cl. A61K 39/395; A61K 39/145; A61K 39/42; A61K 39/116; C07K 16/12; C07K 16/10; A61P 31/04; A61P 31/14; A61P 31/16; A61P 31/22; A61P 33/02; A61P 31/10; A61P 33/06; A61P 37/04; A61P 11/00; A61K 39/40

(52) U.S. Cl. 424/158.1; 424/164.1; 424/163.1; 424/186.1; 424/159.1; 424/165.1; 424/170.1; 424/168.1; 424/172.1; 424/203.1; 530/389.5; 530/389.4

(57) ABSTRACT

The present invention discloses compositions and methods for treating, preventing and/or monitoring viral, bacterial, eukaryotic protist and/or fungal infections. In some embodiments, these compositions and methods involve human polyclonal antibodies affinity purified from human blood using certain viral, bacterial, eukaryotic protist and/or fungal antigens as described herein. Methods of making the antigenic preparations and the affinity-purified human polyclonal antibodies for passive immunization are also provided.

### RELATED APPLICATIONS

This application claims benefit of priority to U.S. Provisional Application Ser. Nos. 61/323,335, filed Apr. 12, 2010, 61/345,543, filed May 17, 2010, 61/350,900, filed Jun. 2, 2010, and 61/352,336, filed Jun. 7, 2010, the contents of which are incorporated herein by reference in their entireties.

### FIELD OF THE INVENTION

This invention generally relates to the field of viral, bacterial, eukaryotic protist and/or fungal infections, particularly to immunological compositions and therapeutic uses thereof, i.e., methods for treating and preventing viral, bacterial and/or fungal infections, and more specifically to the use of affinity purified human polyclonal antibodies for the prevention, treatment and/or monitoring of viral, bacterial and/or fungal infections.

### BACKGROUND OF THE INVENTION

Despite great advances in the treatment and prevention of viral infections, they remain a significant cause of illness and death in both clinical and non-clinical settings. Influenza is one of the most common viral infections that spreads around the world in seasonal epidemics. The most common symptoms of the disease are chills, fever, sore throat, muscle pains, severe headache, coughing, fatigue and general discomfort. In more serious cases, influenza causes pneumonia, which can be fatal, particularly in the elderly and the very young. Every year, influenza results in about 250,000 to 500,000 deaths worldwide, up to millions in pandemic years. In the U.S. alone, influenza killed approximately 41,000 people per year between 1979 and 2001.

Influenza is caused by three genera of RNA viruses of the family Orthomyxoviridiae: Influenzavirus A, Influenzavirus B and Influenzavirus C. Each genus includes a single species, or type: Influenza A virus, Influenza B virus, and Influenza C virus, respectively. Influenza A and C infect multiple species, while influenza B almost exclusively infects humans. Influenza A viruses are further classified, based on the viral surface proteins hemagglutinin (HA or H) and neuraminidase (NA or N). Sixteen H subtypes (or serotypes) and nine N subtypes of Influenza A virus have been identified. The type A viruses are the most virulent human pathogens among the three influenza types and causes the most severe disease. The serotypes that have been confirmed in humans, ordered by the number of known human pandemic deaths, are: H1N1 (cause of the “Spanish Flu” in 1918-20 and the “swine flu” pandemic in 2009-10), H2N2 (cause of the “Russian Flu” in 1889-90 and the “Asian Flu” in 1957-58), H3N2 (cause of the “Hong Kong Flu” in 1968-69), H5N1 (cause of the “avian flu” pandemic threat), H7N7, H1N2, H9N2, H7N2, H7N3, H10N7, H3N2 and H5N2.

Vaccinations against influenza are usually given to people in developed countries and to farmed poultry. The most common human vaccine is the trivalent influenza vaccine (TIV) that contains purified and inactivated material from three viral strains. Typically, this vaccine includes material from two influenza A virus subtypes and one influenza B virus strain. The TIV carries no risk of transmitting the disease, and it has very low reactivity. A vaccine formulated for one year may be ineffective in the following year, since the influenza virus evolves rapidly, and new strains quickly replace the older ones. Antiviral drugs such as neuraminidase and M2 inhibitors are used to treat influenza, with varying degrees of success.

Influenza infections are sometimes accompanied by acute bacterial infections, which tend to exacerbate the effects of the primary viral infection, particularly in patients with a weakened immune system. One of the most troublesome aspects of bacterial infections, e.g., S. aureus infection is the recent proliferation of bacterial strains that are resistant to a broad spectrum of antibiotics. For example, a 2007 report by the U.S. Centers for Disease Control and Prevention (CDC) estimated that the number of methicillin-resistant S. aureus (MRSA) infections treated in hospitals doubled nationwide, from approximately 127,000 in 1999 to 278,000 in 2005, while the number of deaths increased from 11,000 to more than 17,000 at the same time. See Klein et al., Emerg. Infect. Dis. 2007, 13:1840-1846. Another recent CDC study estimated that MRSA was responsible for 94,360 serious infections and was associated with 18,650 hospital stay-related deaths in the United States in 2005. See Klevens et al., J.A.M.A. 2007, 298:1763-1771; CDC Features, “MRSA: Methicillin-resistant Staphylococcus aureus in Healthcare Settings,” Oct. 17, 2007.

Much like vaccinations against influenza, active anti-bacterial vaccinations are designed to be effective against the particular strain(s) selected by the vaccine maker. Therefore, active vaccinations are often ineffective due to the perpetual evolution of new bacterial strains that do not express the antigens used to elicit immune response in a vaccinated individual. Moreover, active immunization takes time to achieve its full effect, whereas many acute bacterial infections require immediate intervention.

Antibody-based therapeutics have a number of advantages over other immune-modulating strategies such as vaccines because antibodies function immediately upon administration, irrespective of whether the patient has a fully functional immune system. Since their first administration in the form of antisera in the 1890s, they have come a long way with the development of monoclonal antibodies (mAbs), antibody fragments, domain antibodies and polyclonal antibodies today. The original infusion of immunoglobulins extracted from human plasma had the advantage of reflecting the natural immune response, relating to the breadth of its repertoire and its diversity. However, several limitations including scarcity of suitable immune plasma, batch-to-batch variation, cost and safety issues have prevented the widespread use of immunoglobulin therapy in its original form.

The development of the hybridoma technique revolutionized the antibody field. This technique allows virtually unlimited production of pure, highly specific monoclonal antibodies in vitro. mAbs have a number of disadvantages, however, which are related to their narrow specificity. Their effects do not cover the full spectrum of effector mechanisms of a natural immune response and mAbs are, therefore, less effective in the treatment of diseases that have complex target antigens. In cases of antigen mutation, or when facing a disease caused by a pathogen with multiple strains, mAbs can also become ineffective. In addition, in spite of efforts to humanize the monoclonal antibodies, there is still a problem with induction of human antibodies against the therapeutic monoclonal antibodies leading to inactivation of the therapeutic monoclonal antibodies and risk of anaphylaxis.

The so-called multi-hit theory teaches that neutralization of a given pathogen depends primarily on achieving a sufficient antibody density on the pathogen's surface and less on the specific epitopes utilized. Since mAbs inherently target a single epitope, pathogen-specific mAbs may, even at high concentrations, be unable to provide a sufficient antibody coating density to mediate bacterial neutralization or elimination, including neutralization or elimination of bacterial toxins and virulence factors. Under normal conditions, the diversity of the human antibody repertoire comprises antibodies against multiple epitopes on the pathogen's surface, thereby securing sufficient antibody coverage to neutralize and eliminate the pathogen. Additionally, the polyclonal nature of the human antibody response reduces the likelihood of immune escape, since a bacterial cell would need to simultaneously acquire escape mutations in several, if not all of the targeted epitopes.

Early beginnings of passive antibody therapy involved the purification of the immunoglobulin fraction of human donor plasma and its infusion into patients. Plasma-derived immunoglobulin from normal healthy donors offers the advantage of mimicking the polyclonal natural immune response with a diverse and specific repertoire, and remains a preferred choice in the treatment of selected conditions. Plasma-derived immunoglobulins reflect the breadth of the human antibody repertoire and, yet, the specificity of the antibody response, with the presence of several antibodies against the pathogen's multiple epitopes increasing the chance of triggering effector mechanisms.

Deriving immunoglobulin from whole human plasma, reflecting the multitude of binding specificities in the natural antibody, implies that only a small fraction of all the immunoglobulin injected is targeting the antigen of interest. This can be partially overcome by the injection of hyperimmune immunoglobulin—derived from individuals who have developed a high titre of antibodies against certain disease-related antigens following (for instance) recovery from infection. Today, hyperimmune immunoglobulin is used for prophylaxis or therapy against infections with hepatitis B virus, respiratory syncytial virus (RSV), cytomegalovirus (CMV) and rabies virus, as well as tetanus, botulinum intoxication and Rhesus D (RhD) alloimmunization.

A more widespread use of immunoglobulin products has been prevented by the fact that the products are highly dependent on donor blood availability, both in terms of quantity and suitability, resulting in considerable variation between batches. Additionally, since only a small fraction of immunoglobulins are specific to the bacterial or viral pathogens of interest, e.g., bacterial toxins, a relatively large amount of immunoglobulins must be administered to a patient in order to achieve the desired bacterial or viral neutralization. Given the advantages of polyclonal antibodies in the immunity to bacteria, bacterial toxins, viruses and virulence factors and the challenges associated with developing effective mAb-based drugs to most bacterial and viral infections, technologies to identify and produce more complex antibody compositions have been developed. Thus, the combination of two or more mAb into cocktails has been attempted, and this approach may in some cases circumvent limitations associated with anti-viral mAb products. However, the cost associated with production and characterization of separate batches of individual mAb components may limit the number of antibodies feasibly included in such cocktails and thereby possibly their efficacy and applicability. Alternative strategies to overcome these challenges rely on using animals such as cows transgenic for human antibody genes for production of plasma-derived polyclonal antibodies after immunization with a given pathogen. Although these technologies appear promising, they suffer from the reduced specific activity due to the presence of a predominance of irrelevant antibody molecules, the need for knocking-out the animal's endogenous antibody genes, and the risk of transferring zoonosis or prions to the recipient.

Thus, no fully effective solution has been found for the prevention, treatment and monitoring of Influenza A virus infections and accompanying bacterial infections. Thus, there is a need to develop new therapeutic and prophylactic, prognostic, diagnostic and treatment monitoring compositions and methods to address these problems. The present invention addressed this and other related needs.

### SUMMARY OF THE INVENTION

In one aspect, the present invention provides an antigenic composition comprising at least one, preferably two or more, Influenza A virus polypeptides, wherein each of the polypeptides comprises an amino acid sequence selected from the following amino acid sequences:

a) polymerase B1 (PB1) sequence, from N-terminus to C-terminus, DAVATTHSWIPKRNRSIL (SEQ ID NO:1),

b) PB1 sequence, from N-terminus to C-terminus, FLKDVMESM (SEQ ID NO:2),

c) PB1 sequence, from N-terminus to C-terminus, FNMLSTVLGV (SEQ ID NO:3),

d) PB1 sequence, from N-terminus to C-terminus, FSMELPSFGV (SEQ ID NO:4),

e) PB1 sequence, from N-terminus to C-terminus, GPATAQMAL (SEQ ID NO:5),

f) PB1 sequence, from N-terminus to C-terminus, DTVNRTHQY (SEQ ID NO:6),

g) polymerase B2 (PB2) sequence, from N-terminus to C-terminus, YMLERELVRKTRFLPVA (SEQ ID NO:7),

h) PB2 sequence, from N-terminus to C-terminus, NFVNRANQRLNPMHQLLR (SEQ ID NO:8),

i) polymerase A (PA) sequence, from N-terminus to C-terminus, FMYSDFHFI (SEQ ID NO:9),

j) PA sequence, from N-terminus to C-terminus, RSKFLLMDALKLSIE (SEQ ID NO:10),

k) PA sequence, from N-terminus to C-terminus, SVKEKDMTK (SEQ ID NO:11),

l) PA sequence, from N-terminus to C-terminus, MRRNYFTAEVSHCRATEY (SEQ ID NO:12),

m) PA sequence, from N-terminus to C-terminus, AESRKLLLI (SEQ ID NO:13),

n) hemagglutinin (HA) sequence, from N-terminus to C-terminus, GLFGAIAGFC (SEQ ID NO:14),

o) HA sequence, from N-terminus to C-terminus, GLFGAIAGFI (SEQ ID NO:15),

p) HA sequence, from N-terminus to C-terminus, TGMVDGWYGYHHQNEQGS (SEQ ID NO:16),

q) HA sequence, from N-terminus to C-terminus, WTYNAELLVLLENERTLD (SEQ ID NO:17),

r) HA sequence, from N-terminus to C-terminus, NKVNSVIEKMNTQFTAVG (SEQ ID NO:18),

s) HA sequence, from N-terminus to C-terminus, GLFGAIAGFIE (SEQ ID NO:19),

t) HA sequence, from N-terminus to C-terminus, YPYDVPDYA (SEQ ID NO:20),

u) HA sequence, from N-terminus to C-terminus, VTGLRNIPSIQCR (SEQ ID NO:21),

v) HA sequence, from N-terminus to C-terminus, SVSSFERFEIFPK (SEQ ID NO:22),

w) nucleoprotein (NP) sequence, from N-terminus to C-terminus, RRSGAAGAAVK (SEQ ID NO:23),

x) NP sequence, from N-terminus to C-terminus, QLVWMACHSAA (SEQ ID NO:24),

y) NP sequence, from N-terminus to C-terminus, YERMCNILKG (SEQ ID NO:25),

z) NP sequence, from N-terminus to C-terminus, TYQRTRALV (SEQ ID NO:26),

aa) NP sequence, from N-terminus to C-terminus, RMVLSAFDER (SEQ ID NO:27),

bb) NP sequence, from N-terminus to C-terminus, LELRSRYWAI (SEQ ID NO:28),

cc) NP sequence, from N-terminus to C-terminus, KLSTRGVQIASNEN (SEQ ID NO:29),

dd) neuraminidase (NA) sequence, from N-terminus to C-terminus, SWPDGAELPF (SEQ ID NO:30),

ee) NA sequence, from N-terminus to C-terminus, PIRGWAI (SEQ ID NO: 31),

ff) NA sequence, from N-terminus to C-terminus, SGSFVQHPELTGL (SEQ ID NO:32),

gg) NA sequence, from N-terminus to C-terminus, VGLISLILQI (SEQ ID NO:33),

hh) matrix protein 1 (M1) sequence, from N-terminus to C-terminus, KTRPILSPLTK (SEQ ID NO:34),

ii) M1 sequence, from N-terminus to C-terminus, QKRMGVQMQRFK (SEQ ID NO:35),

jj) M1 sequence, from N-terminus to C-terminus, AGKNTDLEALMEWLKTR (SEQ ID NO:36),

kk) M1 sequence, from N-terminus to C-terminus, IRHENRMVL (SEQ ID NO:37),

ll) M1 sequence, from N-terminus to C-terminus, GILGFVFTL (SEQ ID NO:38),

mm) M1 sequence, from N-terminus to C-terminus, SLLTEVETYVL (SEQ ID NO:39),

nn) M1 sequence, from N-terminus to C-terminus, KGILGFVFTLTVPSE (SEQ ID NO:40),

oo) M1 sequence, from N-terminus to C-terminus, ILSPLTKGIL (SEQ ID NO:41),

pp) M1 sequence, from N-terminus to C-terminus, RMVLASTTAKAMEQM (SEQ ID NO:42),

qq) matrix protein 2 (M2) sequence, from N-terminus to C-terminus, SLLTEVET (SEQ ID NO:43),

rr) M2 sequence, from N-terminus to C-terminus, EVETPIRN (SEQ ID NO:44),

ss) non-structural protein 1 (NS1) sequence, from N-terminus to C-terminus, GEISPLPSL (SEQ ID NO:45),

tt) NS1 sequence, from N-terminus to C-terminus, DRLRRDQKS (SEQ ID NO:46),

uu) NS1 sequence, from N-terminus to C-terminus, AIMDKNIIL (SEQ ID NO:47),

vv) non-structural protein 2 (NS2) sequence, from N-terminus to C-terminus, ITFMQALQLL (SEQ ID NO:48), and

ww) NS2 sequence, from N-terminus to C-terminus, RTFSFQLI (SEQ ID NO:49).

While in some embodiments the polypeptides may include one or more amino acid residues in addition to the amino acid sequences recited in SEQ ID NO:1 to SEQ ID NO:49 (e.g., a cysteine linker for conjugating the polypeptides to a solid substrate), the polypeptides do not include any additional amino acid sequence(s) of a naturally occurring Influenza A virus protein besides the amino acid sequences recited in SEQ ID NO:1 to SEQ ID NO:49. None of the polypeptides of the present disclosure comprises an amino acid sequence of a naturally occurring, full-length Influenza A virus protein.

In some embodiments, the composition includes 2-49, e.g., at least 5, 10, 15, 20, 25, 30, 35, 40, 45, or all 49 of the Influenza A virus polypeptides, each comprising the amino acid sequences recited in SEQ ID NO:1 to SEQ ID NO:49 and not including any additional amino acid sequences of a naturally occurring Influenza A virus protein. In some embodiments, at least one of the Influenza A virus polypeptides in the composition consists essentially of an amino acid sequence selected from SEQ ID NO:1 to SEQ ID NO:49. In some embodiments, 2-49, e.g., at least 5, 10, 15, 20, 25, 30, 35, 40, 45, or all 49 of the Influenza A virus polypeptides in the composition consist essentially of amino acid sequences selected from SEQ ID NO:1 to SEQ ID NO:49. In some embodiments, at least one of the Influenza A virus polypeptides in the composition consists of an amino acid sequence selected from SEQ ID NO:1 to SEQ ID NO:49. In some embodiments, 2-49, e.g., at least 5, 10, 15, 20, 25, 30, 35, 40, 45, or all 49 of the Influenza A virus polypeptides in the composition consist of amino acid sequences selected from SEQ ID NO:1 to SEQ ID NO:49.

In a related aspect, the invention provides an antigenic composition comprising at least one, preferably two, Influenza A virus polypeptides, wherein each of the polypeptides comprises an amino acid sequence selected from the following amino acid sequences:

a) polymerase B1 (PB1) sequence, from N-terminus to C-terminus, FLKDVMESM (SEQ ID NO:2),

b) PB1 sequence, from N-terminus to C-terminus, FNMLSTVLGV (SEQ ID NO:3),

c) PB1 sequence, from N-terminus to C-terminus, FSMELPSFGV (SEQ ID NO:4),

d) polymerase B2 (PB2) sequence, from N-terminus to C-terminus, YMLERELVRKTRFLPVA (SEQ ID NO:7),

e) PB2 sequence, from N-terminus to C-terminus, NFVNRANQRLNPMHQLLR (SEQ ID NO:8),

f) polymerase A (PA) sequence, from N-terminus to C-terminus, MRRNYFTAEVSHCRATEY (SEQ ID NO:12),

g) PA sequence, from N-terminus to C-terminus, AESRKLLLI (SEQ ID NO:13),

h) hemagglutinin (HA) sequence, from N-terminus to C-terminus, GLFGAIAGFC (SEQ ID NO:14),

i) HA sequence, from N-terminus to C-terminus, TGMVDGWYGYHHQNEQGS (SEQ ID NO:16),

j) HA sequence, from N-terminus to C-terminus, WTYNAELLVLLENERTLD (SEQ ID NO:17),

k) HA sequence, from N-terminus to C-terminus, NKVNSVIEKMNTQFTAVG (SEQ ID NO:18),

l) HA sequence, from N-terminus to C-terminus, VTGLRNIPSIQCR (SEQ ID NO:21),

m) nucleoprotein (NP) sequence, from N-terminus to C-terminus, RRSGAAGAAVK (SEQ ID NO:23),

n) NP sequence, from N-terminus to C-terminus, QLVWMACHSAA (SEQ ID NO:24),

o) NP sequence, from N-terminus to C-terminus, YERMCNILKG (SEQ ID NO:25),

p) NP sequence, from N-terminus to C-terminus, KLSTRGVQIASNEN (SEQ ID NO:29),

q) neuraminidase (NA) sequence, from N-terminus to C-terminus, SWPDGAELPF (SEQ ID NO:30),

r) NA sequence, from N-terminus to C-terminus, PIRGWAI (SEQ ID NO: 31),

s) NA sequence, from N-terminus to C-terminus, SGSFVQHPELTGL (SEQ ID NO:32),

t) NA sequence, from N-terminus to C-terminus, VGLISLILQI (SEQ ID NO:33),

u) matrix protein 1 (M1 sequence, from N-terminus to C-terminus, KTRPILSPLTK (SEQ ID NO:34),

v) M1 sequence, from N-terminus to C-terminus, QKRMGVQMQRFK (SEQ ID NO:35),

w) M1 sequence, from N-terminus to C-terminus, AGKNTDLEALMEWLKTR (SEQ ID NO:36), and

x) non-structural protein 2 (NS2) sequence, from N-terminus to C-terminus, ITFMQALQLL (SEQ ID NO:48).

While in some embodiments the polypeptides may include one or more amino acid residues in addition to the amino acid sequences recited in parts a) to x) (e.g., a cysteine linker for conjugating the polypeptides to a solid substrate), the polypeptides do not include any additional amino acid sequences of a naturally occurring Influenza A virus protein besides the amino acid sequences recited in parts a) to x).

In some embodiments, the composition includes 2-24, e.g., at least 5, 10, 15, 20, or all 24 of the Influenza A virus polypeptides, each comprising the amino acid sequences recited in parts a) to x) and not including any additional amino acid sequences of a naturally occurring Influenza A virus protein. In some embodiments, at least one of the Influenza A virus polypeptides in the composition consists essentially of an amino acid sequence selected from parts a) to x). In some embodiments, 2-24, e.g., at least 5, 10, 15, 20, or all 24 of the Influenza A virus polypeptides in the composition consist essentially of amino acid sequences selected from parts a) to x). In some embodiments, at least one of the Influenza A virus polypeptides in the composition consists of an amino acid sequence selected from parts a) to x). In some embodiments, 2-24, e.g., at least 5, 10, 15, 20, or all 24 of the Influenza A virus polypeptides in the composition consist of amino acid sequences selected from parts a) to x).

In a further aspect, the invention provides a pharmaceutical composition for treating or preventing an Influenza A virus infection, comprising an effective amount of human polyclonal antibodies affinity purified from a human blood sample with one of the Influenza A virus antigenic compositions according to the present invention.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Influenza A virus polypeptides used in the affinity purification. In some embodiments, the affinity purified human polyclonal antibodies are substantially free of human antibodies that specifically bind to non-Influenza A virus antigens in the human blood sample. In some embodiments, the human blood sample is collected from one or more normal human(s). Alternatively, the human blood sample may be collected from one or more human(s) infected with an Influenza A virus. In some embodiments, the human blood sample may be collected from one or more human(s) infected with the Influenza A virus having one the following subtypes: H1N1, H2N2, H3N2, H5N1, H7N7, H1N2, H9N2, H7N2, H3N2, H7N3, H5N2, or H10N7.

In another aspect, the invention provides a method for purifying human polyclonal antibodies to an Influenza A virus, comprising the steps of a) binding human polyclonal antibodies to the Influenza A virus in a human blood sample to an Influenza A virus antigenic composition according to the present invention; and b) recovering the human polyclonal antibodies to the Influenza A virus bound to the antigenic composition to produce affinity purified human polyclonal antibodies to the Influenza A virus.

In a related aspect, the invention provides affinity purified human polyclonal antibodies to an Influenza A virus produced by the present purification process, as well as a pharmaceutical composition for treating or preventing an Influenza A virus infection, comprising an effective amount of such affinity purified human polyclonal antibodies to the Influenza A virus.

In yet another aspect, the invention provides a method for treating or preventing an Influenza A virus infection, comprising administering to a human patient suffering, suspected of suffering or at risk of suffering from an Influenza A virus infection, an effective amount of one of the pharmaceutical compositions for treating or preventing an Influenza A virus infection according to the present invention.

The present methods can be used to treat any suitable human patient. In some embodiments, the human patient is a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an Influenza A virus infection. The present methods can be used to treat a human patient with any suitable Influenza A virus infection. In some embodiments, the Influenza A virus has a subtype selected from H1N1, H2N2, H3N2, H5N1, H7N7, H1N2, H9N2, H7N2, H3N2, H7N3, H5N2, and H10N7. In some embodiments, the Influenza A virus is a strain that caused the “Spanish Flu” and the 2009 swine flu outbreak (H1N1), caused the “Asian Flu” in the late 1950s (H2N2), or caused the “Hong Kong Flu” in the late 1960s (H3N2). In some embodiments, the Influenza A virus is resistant to an antiviral drug, such as amantadine, rimantadine, oseltamivir, or zanamivir.

In some embodiments, the therapeutic or preventive treatment further comprises, prior to administering the affinity purified human polyclonal antibodies to the human patient, conducting an immunotest to determine the presence, absence and/or amount of Influenza A viral antigens in a blood sample of the patient using the same affinity purified human polyclonal antibodies, to assess the suitability of the patient for the therapeutic or preventive treatment. In these embodiments, a positive immunotest result indicates that the patient is suitable for therapy or prevention of Influenza A viral infection using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of Influenza A viral antigens in a blood sample of the patient, preferably using the same affinity purified human polyclonal antibodies, to monitor the efficacy of the therapeutic or preventive treatment. In these embodiments, the absence or reduction in the Influenza A viral antigens after administering the affinity purified human polyclonal antibodies to the patient relative to the amount of Influenza A viral antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of Influenza A viral antigens in a blood sample of the patient preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the Influenza A viral antigens remaining after administering the affinity purified human polyclonal antibodies to the patient and the extent of reduction in the Influenza A viral antigens after administering the affinity purified human polyclonal antibodies to the patient relative to the amount of Influenza A viral antigens before the administration.

In one aspect, the invention provides a pharmaceutical composition for treating or preventing a Klebsiella pneumoniae (K. pneumoniae) infection, comprising an effective amount of human polyclonal antibodies affinity purified from a human blood sample with an antigenic preparation comprising at least one, preferably two or more, cellular and/or secreted antigens of K. pneumoniae cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the K. pneumoniae antigens used in the affinity purification. In some embodiments, the affinity purified human polyclonal antibodies are substantially free of human antibodies that specifically bind to non-K. pneumoniae antigens in the human blood sample. In some embodiments, the human blood sample is collected from one or more normal human(s). Alternatively, the human blood sample may be collected from one or more human(s) infected with K. pneumoniae.

In some embodiments, the antigenic preparation used for affinity purification of the human polyclonal antibodies against K. pneumoniae comprises K. pneumoniae ◯ antigen, K antigen, a K. pneumoniae toxin, and/or a K. pneumoniae antigen that confers antibiotic resistance. In some embodiments, the antigenic preparation comprises a whole cell extract and/or a secreted antigen of K. pneumoniae.

In some embodiments, the antigenic preparation used for affinity purification of the human polyclonal antibodies against K. pneumoniae is prepared by the following process: a) growing K. pneumoniae cells in a first protein containing culture medium; b) collecting and resuspending the K. pneumoniae cells in a second non-protein containing culture medium; c) growing the K. pneumoniae cells in the second non-protein containing culture medium; and d) disrupting the K. pneumoniae cells and collecting a whole cell extract from the disrupted K. pneumoniae cells. In some embodiments, the process of making the antigenic preparation further comprises a step of removing an exotoxin from the whole cell extract and/or a step of collecting a secreted antigen from the second non-protein containing culture medium in which the K. pneumoniae cells were grown.

In another aspect, the invention provides a method for treating or preventing a K. pneumoniae infection, comprising administering to a human patient suffering, suspected of suffering or at risk of suffering from a K. pneumoniae infection, an effective amount of one of the pharmaceutical compositions for treating or preventing a K. pneumoniae infection according to the present invention.

The present methods can be used to treat any suitable human patient. In some embodiments, the human patient is selected from a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from a K. pneumoniae infection. In some embodiments, the patient has a weakened immune system, pneumonia or urinary tract infection. In some embodiments, the K. pneumoniae infection is caused by a K. pneumoniae strain that is resistant to an anti-bacterial drug or treatment.

In some embodiments, the therapeutic or preventive treatment further comprises, prior to administering the affinity purified human polyclonal antibodies to the human patient, conducting an immunotest to determine the presence, absence and/or amount of K. pneumoniae antigens in a blood sample of the patient, preferably using the same affinity purified human polyclonal antibodies, to assess the suitability of the patient for the therapeutic or preventive treatment. In these embodiments, a positive immunotest result indicates that the patient is suitable for therapy or prevention of K. pneumoniae infection using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of K. pneumoniae antigens in a blood sample of the patient, preferably using the same affinity purified human polyclonal antibodies, to monitor the efficacy of the therapeutic or preventive treatment. In these embodiments, the absence or reduction in the K. pneumoniae antigens after administering the affinity purified human polyclonal antibodies to the patient relative to the amount of K. pneumoniae antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of K. pneumoniae antigens in a blood sample of the patient, preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the K. pneumoniae antigens remaining after administering the affinity purified human polyclonal antibodies to the patient and the extent of reduction in the K. pneumoniae antigens after administering the affinity purified human polyclonal antibodies to the patient relative to the amount of K. pneumoniae antigens before the administration.

In another aspect, the invention provides a pharmaceutical composition for treating or preventing an Enterococcus faecalis (E. faecalis) infection, comprising an effective amount of human polyclonal antibodies affinity purified from a human blood sample with an antigenic preparation comprising at least one, preferably two or more, cellular and/or secreted antigens of E. faecalis cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the E. faecalis antigens used in the affinity purification. In some embodiments, the affinity purified human polyclonal antibodies are substantially free of human antibodies that specifically bind to non-E. faecalis antigens in the human blood sample. In some embodiments, the human blood sample is collected from one or more normal human(s). Alternatively, the human blood sample may be collected from one or more human(s) infected with E. faecalis.

In some embodiments, the antigenic preparation used for affinity purification of the human polyclonal antibodies against E. faecalis comprises E. faecalis gelatinase, enterococcal surface protein, aggregation substance, serine protease, capsular polysaccharide, cell wall polysaccharide, hemagglutinin, hemolysin/cytolysin, an E. faecalis toxin, and/or an E. faecalis antigen that confers antibiotic resistance. In some embodiments, the antigenic preparation comprises a whole cell extract and/or a secreted antigen of E. faecalis.

In some embodiments, the antigenic preparation used for affinity purification of the human polyclonal antibodies against E. faecalis is prepared by the following process: a) growing E. faecalis cells in a first protein containing culture medium; b) collecting and resuspending the E. faecalis cells in a second non-protein containing culture medium; c) growing the E. faecalis cells in the second non-protein containing culture medium; and d) disrupting the E. faecalis cells and collecting a whole cell extract from the disrupted E. faecalis cells. In some embodiments, the process of making the antigenic preparation further comprises a step of removing an exotoxin from the whole cell extract and/or a step of collecting a secreted antigen from the second non-protein containing culture medium in which the E. faecalis cells were grown.

In another aspect, the invention provides a method for treating or preventing an E. faecalis infection, comprising administering to a human patient suffering, suspected of suffering or at risk of suffering from an E. faecalis infection, an effective amount of one of the pharmaceutical compositions for treating or preventing an E. faecalis infection according to the present invention.

The present methods can be used to treat any suitable human patient. In some embodiments, the human patient is selected from a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. faecalis infection. In some embodiments, the patient has a weakened immune system, pneumonia or urinary tract infection. In some embodiments, the E. faecalis infection is caused by an E. faecalis strain that is resistant to an anti-bacterial drug or treatment.

In some embodiments, the therapeutic or preventive treatment further comprises, prior to administering the affinity purified human polyclonal antibodies to the human patient, conducting an immunotest to determine the presence, absence and/or amount of E. faecalis antigens in a blood sample of the patient, preferably using the same affinity purified human polyclonal antibodies, to assess the suitability of the patient for the therapeutic or preventive treatment. In these embodiments, a positive immunotest result indicates that the patient is suitable for therapy or prevention of an E. faecalis infection using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of E. faecalis antigens in a blood sample of the patient, preferably using the same affinity purified human polyclonal antibodies, to monitor the efficacy of the therapeutic or preventive treatment. In these embodiments, the absence or reduction in the E. faecalis antigens after administering the affinity purified human polyclonal antibodies to the patient relative to the amount of E. faecalis antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of E. faecalis antigens in a blood sample of the patient, preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the E. faecalis antigens remaining after administering the affinity purified human polyclonal antibodies to the patient and the extent of reduction in the E. faecalis antigens after administering the affinity purified human polyclonal antibodies to the patient relative to the amount of E. faecalis antigens before the administration.

In yet another aspect, the invention provides a pharmaceutical composition for treating or preventing an Enterobacter aerogenes (E. aerogenes) infection, comprising an effective amount of human polyclonal antibodies affinity purified from a human blood sample with an antigenic preparation comprising at least one, preferably two or more, cellular and/or secreted antigens of E. aerogenes cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the E. aerogenes antigens used in the affinity purification. In some embodiments, the affinity purified human polyclonal antibodies are substantially free of human antibodies that specifically bind to non-E. aerogenes antigens in the human blood sample. In some embodiments, the human blood sample is collected from one or more normal human(s). Alternatively, the human blood sample may be collected from one or more human(s) infected with E. aerogenes.

In some embodiments, the antigenic preparation used for affinity purification of the human polyclonal antibodies against E. aerogenes comprises E. aerogenes 0 antigen, K antigen, an E. aerogenes toxin, and/or an E. aerogenes antigen that confers antibiotic resistance. In some embodiments, the antigenic preparation comprises a whole cell extract and/or a secreted antigen of E. aerogenes.

In some embodiments, the antigenic preparation used for affinity purification of the human polyclonal antibodies against E. aerogenes is prepared by the following process: a) growing E. aerogenes cells in a first protein containing culture medium; b) collecting and resuspending the E. aerogenes cells in a second non-protein containing culture medium; c) growing the E. aerogenes cells in the second non-protein containing culture medium; and d) disrupting the E. aerogenes cells and collecting a whole cell extract from the disrupted E. aerogenes cells. In some embodiments, the process of making the antigenic preparation further comprises a step of removing an exotoxin from the whole cell extract and/or a step of collecting a secreted antigen from the second non-protein containing culture medium in which the E. aerogenes cells were grown.

In another aspect, the invention provides a method for treating or preventing an E. aerogenes infection, comprising administering to a human patient suffering, suspected of suffering or at risk of suffering from an E. aerogenes infection, an effective amount of one of the pharmaceutical compositions for treating or preventing an E. aerogenes e infection according to the present invention.

The present methods can be used to treat any suitable human patient. In some embodiments, the human patient is selected from a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. aerogenes infection. In some embodiments, the patient has a weakened immune system, pneumonia or urinary tract infection. In some embodiments, the E. aerogenes infection is caused by an E. aerogenes strain that is resistant to an anti-bacterial drug or treatment.

In some embodiments, the therapeutic or preventive treatment further comprises, prior to administering the affinity purified human polyclonal antibodies to the human patient, conducting an immunotest to determine the presence, absence and/or amount of E. aerogenes antigens in a blood sample of the patient, preferably using the same affinity purified human polyclonal antibodies, to assess the suitability of the patient for the therapeutic or preventive treatment. In these embodiments, a positive immunotest result indicates that the patient is suitable for therapy or prevention of an E. aerogenes infection using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of E. aerogenes antigens in a blood sample of the patient, preferably using the same affinity purified human polyclonal antibodies, to monitor the efficacy of the therapeutic or preventive treatment. In these embodiments, the absence or reduction in the E. aerogenes antigens after administering the affinity purified human polyclonal antibodies to the patient relative to the amount of E. aerogenes antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of E. aerogenes antigens in a blood sample of the patient, preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the E. aerogenes antigens remaining after administering the affinity purified human polyclonal antibodies to the patient and the extent of reduction in the E. aerogenes antigens after administering the affinity purified human polyclonal antibodies to the patient relative to the amount of E. aerogenes antigens before the administration.

In a further aspect, the invention provides a pharmaceutical composition for treating or preventing an Enterobacter cloacae (E. cloacae) infection, comprising an effective amount of human polyclonal antibodies affinity purified from a human blood sample with an antigenic preparation comprising at least one, preferably two or more, cellular and/or secreted antigens of E. cloacae cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the E. cloacae antigens used in the affinity purification. In some embodiments, the affinity purified human polyclonal antibodies are substantially free of human antibodies that specifically bind to non-E. cloacae antigens in the human blood sample. In some embodiments, the human blood sample is collected from one or more normal human(s). Alternatively, the human blood sample may be collected from one or more human(s) infected with E. cloacae.

In some embodiments, the antigenic preparation used for affinity purification of the human polyclonal antibodies against E. cloacae comprises E. cloacae 0 antigen, K antigen, an E. cloacae toxin, and/or an E. cloacae antigen that confers antibiotic resistance. In some embodiments, the antigenic preparation comprises a whole cell extract and/or a secreted antigen of E. cloacae.

In some embodiments, the antigenic preparation used for affinity purification of the human polyclonal antibodies against E. cloacae is prepared by the following process: a) growing E. cloacae cells in a first protein containing culture medium; b) collecting and resuspending the E. cloacae cells in a second non-protein containing culture medium; c) growing the E. cloacae cells in the second non-protein containing culture medium; and d) disrupting the E. cloacae cells and collecting a whole cell extract from the disrupted E. cloacae cells. In some embodiments, the process of making the antigenic preparation further comprises a step of removing an exotoxin from the whole cell extract and/or a step of collecting a secreted antigen from the second non-protein containing culture medium in which the E. cloacae cells were grown.

In another aspect, the invention provides a method for treating or preventing an E. cloacae infection, comprising administering to a human patient suffering, suspected of suffering or at risk of suffering from an E. cloacae infection, an effective amount of one of the pharmaceutical compositions for treating or preventing an E. cloacae infection according to the present invention.

The present methods can be used to treat any suitable human patient. In some embodiments, the human patient is selected from a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. cloacae infection. In some embodiments, the patient has a weakened immune system, pneumonia or urinary tract infection. In some embodiments, the E. cloacae infection is caused by an E. cloacae strain that is resistant to an anti-bacterial drug or treatment.

In some embodiments, the therapeutic or preventive treatment further comprises, prior to administering the affinity purified human polyclonal antibodies to the human patient, conducting an immunotest to determine the presence, absence and/or amount of E. cloacae antigens in a blood sample of the patient, preferably using the same affinity purified human polyclonal antibodies, to assess the suitability of the patient for the therapeutic or preventive treatment. In these embodiments, a positive immunotest result indicates that the patient is suitable for therapy or prevention of an E. cloacae infection using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of E. cloacae antigens in a blood sample of the patient, preferably using the same affinity purified human polyclonal antibodies, to monitor the efficacy of the therapeutic or preventive treatment. In these embodiments, the absence or reduction in the E. cloacae antigens after administering the affinity purified human polyclonal antibodies to the patient relative to the amount of E. cloacae antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of E. cloacae antigens in a blood sample of the patient, preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the E. cloacae antigens remaining after administering the affinity purified human polyclonal antibodies to the patient and the extent of reduction in the E. cloacae antigens after administering the affinity purified human polyclonal antibodies to the patient relative to the amount of E. cloacae antigens before the administration.

In one aspect, the invention provides a pharmaceutical composition for treating or preventing an Influenza A virus infection and an accompanying bacterial infection, comprising an effective amount of human polyclonal antibodies affinity purified from a human blood sample with one of the Influenza A virus antigenic compositions according to the present invention; and an effective amount of human polyclonal antibodies affinity purified from a human blood sample with an antigenic preparation comprising cellular and/or secreted antigens from bacterial cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae) and/or a combination thereof.

In some embodiments, the affinity purified human polyclonal antibodies to the Influenza A virus and the bacterial cells are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Influenza A virus and/or the bacterial antigens used in the affinity purification. In some embodiments, the affinity purified human polyclonal antibodies are substantially free of human antibodies that specifically bind to non-Influenza A virus antigens and/or non-bacterial antigens in the human blood sample. In some embodiments, the human blood sample is collected from one or more normal human(s). Alternatively, the human blood sample may be collected from one or more human(s) infected with the Influenza A virus and/or a bacterium selected from S. aureus, a Streptococcus, E. coli, P. aeruginosa, A. baumannii, E. faecium, E. faecalis, E. aerogenes, E. cloacae, C. difficile, K. pneumoniae and/or a combination thereof.

In some embodiments, the affinity purification of human polyclonal antibodies comprises a step of substantially inactivating and/or removing a virus. Any virus that may contaminate or compromise the therapeutic or preventive use of the affinity purified human polyclonal antibodies may be substantially inactivated and/or removed. In some embodiments, the virus to be substantially inactivated and/or removed is a lipid-enveloped or non-enveloped virus.

In some embodiments, the bacterial antigenic preparation comprises cellular and/or secreted antigens from a combination of: a) any two bacterial species selected from S. aureus, a Streptococcus, E. coli, P. aeruginosa, A. baumannii, E. faecium, E. faecalis, E. aerogenes, E. cloacae, C. difficile and K. pneumoniae; or b) any three bacterial species selected from S. aureus, a Streptococcus, E. coli, P. aeruginosa, A. baumannii, E. faecium, E. faecalis, E. aerogenes, E. cloacae, C. difficile and K. pneumoniae; or c) any four bacterial species selected from S. aureus, a Streptococcus, E. coli, P. aeruginosa, A. baumannii, E. faecium, E. faecalis, E. aerogenes, E. cloacae, C. difficile and K. pneumoniae; or d) any five bacterial species selected from S. aureus, a Streptococcus, E. coli, P. aeruginosa, A. baumannii, E. faecium, E. faecalis, E. aerogenes, E. cloacae, C. difficile and K. pneumoniae; or e) any six bacterial species selected from S. aureus, a Streptococcus, E. coli, P. aeruginosa, A. baumannii, E. faecium, E. faecalis, E. aerogenes, E. cloacae, C. difficile and K. pneumoniae; or f) any seven bacterial species selected from S. aureus, a Streptococcus, E. coli, P. aeruginosa, A. baumannii, E. faecium, E. faecalis, E. aerogenes, E. cloacae, C. difficile and K. pneumoniae; or g) any eight bacterial species selected from S. aureus, a Streptococcus, E. coli, P. aeruginosa, A. baumannii, E. faecium, E. faecalis, E. aerogenes, E. cloacae, C. difficile and K. pneumoniae; or h) any nine bacterial species selected from S. aureus, a Streptococcus, E. coli, P. aeruginosa, A. baumannii, E. faecium, E. faecalis, E. aerogenes, E. cloacae, C. difficile and K. pneumoniae; or i) any ten bacterial species selected from S. aureus, a Streptococcus, E. coli, P. aeruginosa, A. baumannii, E. faecium, E. faecalis, E. aerogenes, E. cloacae, C. difficile and K. pneumoniae; or j) each of S. aureus, a Streptococcus, E. coli, P. aeruginosa, A. baumannii, E. faecium, E. faecalis, E. aerogenes, E. cloacae, C. difficile and K. pneumoniae; or k) each of S. aureus, S. pneumoniae, E. coli, P. aeruginosa, A. baumannii, E. faecalis and K. pneumoniae; or l) each of S. aureus, S. pneumoniae, E. coli, P. aeruginosa, A. baumannii, E. faecium and K. pneumoniae .

In some embodiments, the bacterial antigenic preparations comprise cellular and/or secreted antigens from each of S. aureus, S. pneumoniae, E. coli, P. aeruginosa, A. baumannii, E. faecalis and K. pneumoniae. In some embodiments, the bacterial antigenic preparations comprise cellular and/or secreted antigens from each of S. aureus, S. pneumoniae, E. coli, P. aeruginosa, A. baumannii, E. faecium and K. pneumoniae .

In some embodiments, the bacterial antigenic preparation is prepared by the following process: a) growing bacterial cells in a first protein containing culture medium; b) collecting and resuspending the bacterial cells in a second non-protein containing culture medium; c) growing the bacterial cells in the second non-protein containing culture medium; and d) disrupting the bacterial cells and collecting a whole cell extract from the disrupted bacterial cells. In some embodiments, the process of making the bacterial antigenic preparation further comprises a step of removing an exotoxin from the whole cell extract and/or a step of collecting a secreted antigen from the second non-protein containing culture medium in which the bacterial cells were grown.

In another aspect, the invention provides a pharmaceutical composition for treating or preventing an Influenza A virus infection and an accompanying bacterial infection, comprising an effective amount of human polyclonal antibodies affinity purified from a human blood sample, the antibodies having specificity for an Influenza A viral antigen, a bacterial antigen and an antigen from human tumor necrosis factor alpha (TNF-α). In some embodiments, the human polyclonal antibodies having specificity for the Influenza A viral antigen are affinity purified from the human blood sample with one of the Influenza A virus antigenic compositions according to the present invention. In some embodiments, the human polyclonal antibodies having specificity for the bacterial antigen are affinity purified from the human blood sample with an antigenic preparation comprising cellular and/or secreted antigens from bacterial cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae) and/or a combination thereof.

In some embodiments, the affinity purified human polyclonal antibodies to the Influenza A viral antigen, the bacterial antigen and the TNF-α antigen are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Influenza A viral antigen, the bacterial antigen and the TNF-α antigen used in the affinity purification. In some embodiments, the affinity purified human polyclonal antibodies are substantially free of human antibodies that specifically bind to non-Influenza A virus, non-bacterial and non-TNF-α antigens in the human blood sample. In some embodiments, the human blood sample is collected from one or more normal human(s). Alternatively, the human blood sample may be collected from one or more human(s) infected with the Influenza A virus and/or a bacterium selected from S. aureus, a Streptococcus, E. coli, P. aeruginosa, A. baumannii, E. faecium, E. faecalis, E. aerogenes, E. cloacae, C. difficile, K. pneumoniae and/or a combination thereof.

In some embodiments, the human polyclonal antibodies specific for the bacterial antigen are affinity purified from the human blood sample with an antigenic preparation comprising cellular and/or secreted antigens from: a) any two bacterial species selected from S. aureus, a Streptococcus, E. coli, P. aeruginosa, A. baumannii, E. faecium, E. faecalis, E. aerogenes, E. cloacae, C. difficile and K. pneumoniae; or b) any three bacterial species selected from S. aureus, a Streptococcus, E. coli, P. aeruginosa, A. baumannii, E. faecium, E. faecalis, E. aerogenes, E. cloacae, C. difficile and K. pneumoniae; or c) any four bacterial species selected from S. aureus, a Streptococcus, E. coli, P. aeruginosa, A. baumannii, E. faecium, E. faecalis, E. aerogenes, E. cloacae, C. difficile and K. pneumoniae; or d) any five bacterial species selected from S. aureus, a Streptococcus, E. coli, P. aeruginosa, A. baumannii, E. faecium, E. faecalis, E. aerogenes, E. cloacae, C. difficile and K. pneumoniae; or e) any six bacterial species selected from S. aureus, a Streptococcus, E. coli, P. aeruginosa, A. baumannii, E. faecium, E. faecalis, E. aerogenes, E. cloacae, C. difficile and K. pneumoniae; or f) any seven bacterial species selected from S. aureus, a Streptococcus, E. coli, P. aeruginosa, A. baumannii, E. faecium, E. faecalis, E. aerogenes, E. cloacae, C. difficile and K. pneumoniae; or g) any eight bacterial species selected from S. aureus, a Streptococcus, E. coli, P. aeruginosa, A. baumannii, E. faecium, E. faecalis, E. aerogenes, E. cloacae, C. difficile and K. pneumoniae; or h) any nine bacterial species selected from S. aureus, a Streptococcus, E. coli, P. aeruginosa, A. baumannii, E. faecium, E. faecalis, E. aerogenes, E. cloacae, C. difficile and K. pneumoniae; or i) any ten bacterial species selected from S. aureus, a Streptococcus, E. coli, P. aeruginosa, A. baumannii, E. faecium, E. faecalis, E. aerogenes, E. cloacae, C. difficile and K. pneumoniae; or j) each of S. aureus, a Streptococcus, E. coli, P. aeruginosa, A. baumannii, E. faecium, E. faecalis, E. aerogenes, E. cloacae, C. difficile and K. pneumoniae; or k) each of S. aureus, S. pneumoniae, E. coli, P. aeruginosa, A. baumannii, E. faecalis and K. pneumoniae; or 1) each of S. aureus, S. pneumoniae, E. coli, P. aeruginosa, A. baumannii, E. faecium and K. pneumoniae .

In some embodiments, the human polyclonal antibodies specific for the bacterial antigen are affinity purified from the human blood sample with an antigenic preparation comprising cellular and/or secreted antigens from the bacterial cells of each of S. aureus, S. pneumoniae, E. coli, P. aeruginosa, A. baumannii, E. faecalis and K. pneumoniae. In some embodiments, the human polyclonal antibodies specific for the bacterial antigen are affinity purified from the human blood sample with an antigenic preparation comprising cellular and/or secreted antigens from the bacterial cells of each of S. aureus, S. pneumoniae, E. coli, P. aeruginosa, A. baumannii, E. faecium and K. pneumoniae .

In some embodiments, the bacterial antigen is prepared by the following process: a) growing bacterial cells in a first protein containing culture medium; b) collecting and resuspending the bacterial cells in a second non-protein containing culture medium; c) growing the bacterial cells in the second non-protein containing culture medium; and d) disrupting the bacterial cells and collecting a whole cell extract from the disrupted bacterial cells. In some embodiments, the process of making the bacterial antigen further comprises a step of removing an exotoxin from the whole cell extract and/or a step of collecting a secreted antigen from the second non-protein containing culture medium in which the bacterial cells were grown.

In yet another aspect, the invention provides a method for treating or preventing an Influenza A virus infection and a bacterial infection, comprising administering to a human patient suffering, suspected of suffering or at risk of suffering from an Influenza A virus infection, an S. aureus infection, a Streptococcus infection, an E. coli infection, a P. aeruginosa infection, an A. baumannii infection, an E. faecium infection, an E. faecalis infection, an E. aerogenes infection, an E. cloacae infection, a C. difficile infection, and/or a K. pneumoniae infection, an effective amount of one of the pharmaceutical compositions for treating or preventing an Influenza A virus infection and a bacterial infection according to the present invention.

The present methods can be used to treat any suitable human patient. In some embodiments, the human patient is selected from a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an Influenza A virus infection, an S. aureus infection, a Streptococcus infection, an E. coli infection, a P. aeruginosa infection, an A. baumannii infection, an E. faecium infection, an E. faecalis infection, an E. aerogenes infection, an E. cloacae infection, a C. difficile infection, and/or a K. pneumoniae infection. In some embodiments, the patient suffers, is suspected of suffering, or is at risk of suffering from bacteremia, septicemia, bacterial pneumonia, bacterial meningitis, otitis media, streptococcal pharyngitis (strep throat), scarlet fever, acute rheumatic fever, endocarditis, streptococcal toxic shock syndrome, perinatal Group B streptococcal disease, gastroenteritis, urinary tract infection, hemolytic-uremic syndrome (HUS), peritonitis, or mastitis.

The present methods can be used to treat a human patient with any suitable Influenza A virus infection. In some embodiments, the Influenza A virus has a subtype selected from H1N1, H2N2, H3N2, H5N1, H7N7, H1N2, H9N2, H7N2, H3N2, H7N3, H5N2, and/or H10N7. In some embodiments, the Influenza A virus is a strain that caused the “Spanish Flu” and the 2009 swine flu outbreak (H1N1), caused the “Asian Flu” in the late 1950s (H2N2), or caused the “Hong Kong Flu” in the late 1960s (H3N2). In some embodiments, the Influenza A virus is resistant to an antiviral drug, such as amantadine, rimantadine, oseltamivir, or zanamivir. In some embodiments, the bacterial infection is caused by a bacterial strain that is resistant to an anti-bacterial drug or treatment.

In some embodiments, the therapeutic or preventive treatment further comprises, prior to administering the affinity purified human polyclonal antibodies to the human patient, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the patient, preferably using the same affinity purified human polyclonal antibodies, to assess the suitability of the patient for the therapeutic or preventive treatment. In these embodiments, a positive immunotest result indicates that the patient is suitable for therapy or prevention of an Influenza A virus, S. aureus, a Streptococcus, E. coli, P. aeruginosa, A. baumannii, E. faecium, E. faecalis, E. aerogenes, E. cloacae, C. difficile, and/or K. pneumoniae infection using the affinity purified human polyclonal antibodies.

In some embodiments, the immunotest is conducted to determine the presence, absence and/or amount of an Influenza A virus, S. aureus, S. pneumoniae, E. coli, P. aeruginosa, A. baumannii, E. faecalis, K. pneumoniae and TNF-α antigens, and a positive immunotest result indicates that the human is suitable for therapy or prevention of Influenza A virus, S. aureus, S. pneumoniae, E. coli, P. aeruginosa, A. baumannii, E. faecalis and K. pneumoniae infections using the affinity purified human polyclonal antibodies.

In some embodiments, the immunotest is conducted to determine the presence, absence and/or amount of an Influenza A virus, S. aureus, S. pneumoniae, E. coli, P. aeruginosa, A. baumannii, E. faecium, K. pneumoniae and TNF-α antigens, and a positive immunotest result indicates that the human is suitable for therapy or prevention of Influenza A virus, S. aureus, S. pneumoniae, E. coli, P. aeruginosa, A. baumannii, E. faecium and K. pneumoniae infections using the affinity purified human polyclonal antibodies.

In some embodiments, the therapeutic or preventive treatment further comprises, before and after administering the affinity purified human polyclonal antibodies to the human patient, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the patient, preferably using the same affinity purified human polyclonal antibodies, to monitor the efficacy of the therapeutic or preventive treatment. In these embodiments, the absence or reduction in the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens after administering the affinity purified human polyclonal antibodies to the patient relative to the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens before the administration indicates efficacy of the therapeutic or preventive treatment.

In some embodiments, the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, and the absence or reduction in the Influenza A virus, S. aureus, S. pneumoniae, E. coli, P. aeruginosa, A. baumannii, E. faecalis, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecalis, K. pneumoniae and TNF-α antigens before the administration indicates efficacy of the therapeutic or preventive treatment.

In some embodiments, the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, and the absence or reduction in the Influenza A virus, S. aureus, S. pneumoniae, E. coli, P. aeruginosa, A. baumannii, E. faecium, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecium, K. pneumoniae and TNF-α antigens before the administration indicates efficacy of the therapeutic or preventive treatment.

In some embodiments, the therapeutic or preventive treatment further comprises, before and after administering the affinity purified human polyclonal antibodies to the human patient, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the patient, preferably using the same affinity purified human polyclonal antibodies, to determine an optimal therapeutic or preventive dose of the affinity purified human polyclonal antibodies. In these embodiments, the optimal therapeutic or preventive dose is determined based on the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens remaining after administering the affinity purified human polyclonal antibodies to the patient and the extent of reduction in the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens after administering the affinity purified human polyclonal antibodies to the patient relative to the amount of the Influenza A virus, S. aureus, a Streptococcus, E. coli, P. aeruginosa, A. baumannii, E. faecium, E. faecalis, E. aerogenes, E. cloacae, C. difficile, K pneumoniae and/or TNF-α antigens before the administration.

In some embodiments, the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecalis, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecalis, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecalis, K. pneumoniae and TNF-α antigens before the administration.

In some embodiments, the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecium, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecium, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecium, K. pneumoniae and TNF-α antigens before the administration.

In one aspect, the invention provides a pharmaceutical composition for treating or preventing a Salmonella infection, which composition comprises an effective amount of human polyclonal antibodies affinity purified from a human blood sample with an antigenic preparation comprising at least one, preferably two or more, cellular and/or secreted antigens of Salmonella cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Salmonella antigens used in the affinity purification. In some embodiments, the affinity purified human polyclonal antibodies are substantially free of human antibodies that specifically bind to non-Salmonella antigens in said human blood sample.

The affinity purified human polyclonal antibodies can have any suitable concentrations. In some embodiments, the affinity purified human polyclonal antibodies specific to the Salmonella antigens have a concentration ranging from about 10 μg/ml to about 10 mg/ml.

In some embodiments, the human blood sample is collected from one or more normal human(s). Alternatively, the human blood sample may be collected from one or more human(s) infected with Salmonella, e.g., collected from 2, 10, 50, 100, 500, 1,000, 5,000, 10,000 or more humans.

The affinity purified human polyclonal antibodies can be purified from any suitable human blood samples. In some embodiments, the affinity purified human polyclonal antibodies is purified from a normal human sample. In other embodiments, the affinity purified human polyclonal antibodies is purified from a blood sample of a human infected with Salmonella.

The antigenic preparation can comprise an antigen(s) from any suitable Salmonella species or serovars. In some embodiments, the antigenic preparation comprises an antigen(s) from Salmonella bongori (S. bongori) and/or Salmonella enterica (S. enterica). In other embodiments, the antigenic preparation comprises an antigen(s) from Salmonella enterica enterica, Salmonella enterica salamae, Salmonella enterica arizonae, Salmonella enterica diarizonae, Salmonella enterica houtenae, and/or Salmonella enterica indica. In other embodiments, the Salmonella enterica enterica has serovars selected from the group consisting of Salmonella Choleraesuis, Salmonella Dublin, Salmonella Enteritidis, Salmonella Gallinarum, Salmonella Hadar, Salmonella Heidelberg, Salmonella Infantis, Salmonella Paratyphi, Salmonella Typhi and Salmonella Typhimurium.

In some embodiments, the antigenic preparation comprises a Salmonella antigen(s) that confers antibiotic resistance. In other embodiments, the antibiotic resistant strain is Salmonella Typhimurium DT104 (DT104), multidrug-resistant typhoid (MDR typhoid), or MDR-AmpC.

The antigenic preparation can comprise any suitable Salmonella antigen(s). In other embodiments, the antigenic preparation comprises a Salmonella toxin, O-somatic antigen and/or H-flagellar antigen.

The antigenic preparation can comprise a single, but often multiple Salmonella antigens. In some embodiments, the antigenic preparation comprises a whole cell extract and a secreted antigen of Salmonella.

The antigenic preparation can be prepared by any suitable methods. In some embodiments, the antigenic preparation is prepared by the following steps: a) growing Salmonella cells in a first protein containing culture medium; b) collecting and resuspending the Salmonella cells in a second non-protein containing culture medium; c) growing the Salmonella cells in the second non-protein containing culture medium; and d) disrupting the bacterial cells and collecting a whole cell extract from the disrupted Salmonella cells. In other embodiments, the antigen preparation can further comprise a step of removing toxin from the whole cell extract. In other embodiments, the antigen preparation can further comprises a step of collecting a secreted antigen from the second non-protein containing culture medium in which the Salmonella cells have grown.

In another aspect, the invention provides a method for treating or preventing Salmonella infection, which method comprises administering to a human suffering, suspected of suffering or at risk of suffering from Salmonella infection, an effective amount of the pharmaceutical composition for treating or preventing Salmonella infection according to the present application.

Any suitable human can be treated by the present methods. In some embodiments, the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Salmonella infection. In other embodiments, the human for treatment or prevention has a weakened immune system, typhoid fever (also known as Salmonella typhi or commonly just typhoid), paratyphoid fevers (or enteric fevers), foodborne illness (also foodborne disease and colloquially referred to as food poisoning) or Salmonellosis.

The present methods can be used to treat or prevent infection any suitable Salmonella species, strain or serovars. In some embodiments, the present methods can be used to treat or prevent infection caused by a Salmonella strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Salmonella antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Salmonella infection using the affinity purified human polyclonal antibodies.

In some embodiments, the present methods can further comprise, before and after administering the affinity purified human polyclonal antibodies to the human, conducting an immunotest to determine the presence, absence and/or amount of Salmonella antigens in a blood sample of the human using the same affinity purified human polyclonal antibodies, to monitor the efficacy of the therapeutic, removal or preventive treatment, wherein the absence or reduction in the Salmonella antigens after administering the affinity purified human polyclonal antibodies to the human relative to the amount of Salmonella antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Salmonella antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Salmonella antigens remaining after administering the affinity purified human polyclonal antibodies to the human and the extent of reduction in the Salmonella antigens after administering the affinity purified human polyclonal antibodies to the human relative to the amount of Salmonella antigens before the administration.

In one aspect, the invention provides a pharmaceutical composition for treating or preventing tuberculosis (TB), which composition comprises an effective amount of human polyclonal antibodies affinity purified from a human blood sample with an antigenic preparation comprising at least one, preferably two or more, cellular and/or secreted antigens of TB-causing Mycobacterium cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Mycobacterium antigens used in the affinity purification. In some embodiments, the affinity purified human polyclonal antibodies are substantially free of human antibodies that specifically bind to non-Mycobacterium antigens in said human blood sample.

The affinity purified human polyclonal antibodies can have any suitable concentrations. In some embodiments, the affinity purified human polyclonal antibodies specific to the Mycobacterium antigens have a concentration ranging from about 10 μg/ml to about 10 mg/ml.

In some embodiments, the human blood sample is collected from one or more normal human(s). Alternatively, the human blood sample may be collected from one or more human(s) infected with the Mycobacterium, e.g., collected from 2, 10, 50, 100, 500, 1,000, 5,000, 10,000 or more humans.

The antigenic preparation can comprise an antigen(s) from any suitable Mycobacterium species or serovars. In some embodiments, the antigenic preparation comprises antigens from Mycobacterium tuberculosis (MTB), M. bovis, M. africanum, M. canetti, and/or M. microti. In other embodiments, the antigenic preparation comprises antigens from a hypervirulent strain of M. tuberculosis. In other embodiments, the antigenic preparation comprises a Mycobacterium antigen that confers antibiotic resistance. The exemplary antibiotic resistant strains include multi-drug-resistant tuberculosis (MDR-TB) or extensively drug-resistant TB (XDR-TB).

The antigenic preparation can comprise any suitable Mycobacterium antigen(s). In some embodiments, the antigenic preparation comprises a Mycobacterium toxin.

The antigenic preparation can comprise a single, but often multiple Mycobacterium antigens. In some embodiments, the antigenic preparation comprises a whole cell extract and a secreted antigen of Mycobacterium.

The antigenic preparation can be prepared by any suitable methods. In some embodiments, the antigenic preparation is prepared by the following steps: a) growing Mycobacterium cells in a first protein containing culture medium; b) collecting and resuspending the Mycobacterium cells in a second non-protein containing culture medium; c) growing the Mycobacterium a cells in the second non-protein containing culture medium; and d) disrupting the bacterial cells and collecting a whole cell extract from the disrupted Mycobacterium cells. In other embodiments, the antigen preparation can further comprise a step of removing toxin from the whole cell extract. In other embodiments, the antigen preparation can further comprise a step of collecting a secreted antigen from the second non-protein containing culture medium in which the Mycobacterium cells have grown.

In another aspect, the invention provides a method for treating or preventing tuberculosis (TB), which method comprises administering to a human suffering, suspected of suffering or at risk of suffering from Mycobacterium infection, an effective amount of the pharmaceutical composition for treating or preventing Mycobacterium infection according to the present application.

Any suitable human can be treated by the present methods. In some embodiments, the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Mycobacterium infection. In other embodiments, the human for treatment has a weakened immune system, is a women of reproductive age or a person with HIV/AIDS.

The present methods can be used to treat or prevent infection any suitable Mycobacterium species, strain or serovars. In some embodiments, the present methods can be used to treat or prevent infection caused by a Mycobacterium strain that is resistant to an anti-bacterial drug or treatment.

In some embodiments, the present methods further comprises, prior to administering the affinity purified human polyclonal antibodies to the human, conducting an immunotest to determine the presence, absence and/or amount of Mycobacterium antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Mycobacterium infection using the affinity purified human polyclonal antibodies.

In some embodiments, the present methods further comprises, before and after administering the affinity purified human polyclonal antibodies to the human, conducting an immunotest to determine the presence, absence and/or amount of Mycobacterium antigens in a blood sample of the human using the same affinity purified human polyclonal antibodies, to monitor the efficacy of the therapeutic, removal or preventive treatment, wherein the absence or reduction in the Mycobacterium antigens after administering the affinity purified human polyclonal antibodies to the human relative to the amount of Mycobacterium antigens before the administration indicates efficacy of the therapeutic, removal or preventive treatment.

In some embodiments, the present methods further comprises, before and after administering the affinity purified human polyclonal antibodies to the human, conducting an immunotest to determine the presence, absence and/or amount of Mycobacterium antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycobacterium antigens remaining after administering the affinity purified human polyclonal antibodies to the human and the extent of reduction in the Mycobacterium antigens after administering the affinity purified human polyclonal antibodies to the human relative to the amount of Mycobacterium antigens before the administration.

In one aspect, the invention provides a pharmaceutical composition for treating or preventing anthrax, which composition comprises an effective amount of human polyclonal antibodies affinity purified from a human blood sample with an antigenic preparation comprising at least one, preferably two or more, cellular and/or secreted antigens of Bacillus anthracis cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Bacillus anthracis antigens used in the affinity purification. In some embodiments, the affinity purified human polyclonal antibodies are substantially free of human antibodies that specifically bind to non-Bacillus anthracis antigens in said human blood sample.

The affinity purified human polyclonal antibodies can have any suitable concentrations. In some embodiments, the affinity purified human polyclonal antibodies specific to the Bacillus anthracis antigens have a concentration ranging from about 10 μg/ml to about 10 mg/ml.

In some embodiments, the human blood sample is collected from one or more normal human(s). Alternatively, the human blood sample may be collected from one or more human(s) infected with the Bacillus anthracis, e.g., collected from 2, 10, 50, 100, 500, 1,000, 5,000, 10,000 or more humans.

The antigenic preparation can comprise an antigen(s) from any suitable Bacillus anthracis strains or serovars. In some embodiments, the antigenic preparation comprises antigens from the Ames strain, the Vollum strain (also incorrectly referred to as Vellum) strain and/or the Vollum 1B strain.

The antigenic preparation can comprise any suitable Bacillus anthracis antigen(s). In some embodiments, the antigenic preparation comprises an exotoxin, e.g., edema toxin or lethal toxin. In other embodiments, the antigenic preparation comprises the poly-D-glutamic acid capsule, the protective antigen (PA), the edema factor (EF), and/or the lethal factor (LF). In other embodiments, the antigenic preparation comprises a Bacillus anthracis antigen that confers antibiotic resistance.

The antigenic preparation can comprise a single, but often multiple Bacillus anthracis antigens. In some embodiments, the antigenic preparation comprises a whole cell extract and a secreted antigen of Bacillus anthracis.

The antigenic preparation can be prepared by any suitable methods. In some embodiments, the antigenic preparation is prepared by the following steps: a) growing Bacillus anthracis cells in a first protein containing culture medium; b) collecting and resuspending the Bacillus anthracis cells in a second non-protein containing culture medium; c) growing the Bacillus anthracis cells in the second non-protein containing culture medium; and d) disrupting the bacterial cells and collecting a whole cell extract from the disrupted Bacillus anthracis cells. In other embodiments, the antigen preparation can further comprise a step of removing toxin from the whole cell extract. In other embodiments, the antigen preparation can further comprise a step of collecting a secreted antigen from the second non-protein containing culture medium in which the Bacillus anthracis cells have grown.

In another aspect, the invention provides a method for treating or preventing anthrax, which method comprises administering to a human suffering, suspected of suffering or at risk of suffering from anthrax, an effective amount of the pharmaceutical composition for treating or preventing Bacillus anthracis infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Bacillus anthracis infection. In other embodiments, the human for treatment or prevention has a weakened immune system, pulmonary infection, gastrointestinal infection, or cutaneous infection.

The present methods can be used to treat or prevent infection any suitable Bacillus anthracis strain or serovars. In some embodiments, the present methods can be used to treat or prevent infection caused by a Bacillus anthracis strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Bacillus anthracis antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Bacillus anthracis infection using the affinity purified human polyclonal antibodies.

In some embodiments, the present methods can further comprise, before and after administering the affinity purified human polyclonal antibodies to the human, conducting an immunotest to determine the presence, absence and/or amount of Bacillus anthracis antigens in a blood sample of the human using the same affinity purified human polyclonal antibodies, to monitor the efficacy of the therapeutic, removal or preventive treatment, wherein the absence or reduction in the Bacillus anthracis antigens after administering the affinity purified human polyclonal antibodies to the human relative to the amount of Bacillus anthracis antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Bacillus anthracis antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Bacillus anthracis antigens remaining after administering the affinity purified human polyclonal antibodies to the human and the extent of reduction in the Bacillus anthracis antigens after administering the affinity purified human polyclonal antibodies to the human relative to the amount of Bacillus anthracis antigens before the administration.

In one aspect, the invention provides a pharmaceutical composition for treating or preventing listeriosis, which composition comprises an effective amount of human polyclonal antibodies affinity purified from a human blood sample with an antigenic preparation comprising at least one, preferably two or more, cellular and/or secreted antigens of Listeria monocytogenes cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Listeria monocytogenes antigens used in the affinity purification. In some embodiments, the affinity purified human polyclonal antibodies are substantially free of human antibodies that specifically bind to non-Listeria monocytogenes antigens in said human blood sample.

The affinity purified human polyclonal antibodies can have any suitable concentrations. In some embodiments, the affinity purified human polyclonal antibodies specific to the Listeria monocytogenes antigens have a concentration ranging from about 10 μg/ml to about 10 mg/ml.

In some embodiments, the human blood sample is collected from one or more normal human(s). Alternatively, the human blood sample may be collected from one or more human(s) infected with the Listeria monocytogenes, e.g., collected from 2, 10, 50, 100, 500, 1,000, 5,000, 10,000 or more humans.

The antigenic preparation can comprise an antigen(s) from any suitable Listeria monocytogenes strains or serovars. In some embodiments, the Listeria monocytogenes has serotypes: 1/2a, 1/2b, and/or 4b.

In some embodiments, the antigenic preparation comprises a Listeria monocytogenes antigen that confers antibiotic resistance. Exemplary antibiotic resistant strains include Listeria monocytogenes BM4210 or BM4293.

The antigenic preparation can comprise any suitable Listeria monocytogenes antigen(s). In some embodiments, the antigenic preparation can comprise a Listeria monocytogenes toxin, listerial internalins (Inl), listeriolysin O (LLO—encoded by hly), phospholipase A (encoded by plcA) and/or phospholipase B (plcB).

The antigenic preparation can comprise a single, but often multiple Listeria monocytogenes antigens. In some embodiments, the antigenic preparation comprises a whole cell extract and a secreted antigen of Listeria monocytogenes.

The antigenic preparation can be prepared by any suitable methods. In some embodiments, the antigenic preparation is prepared by the following steps: a) growing Listeria monocytogenes cells in a first protein containing culture medium; b) collecting and resuspending the Listeria monocytogenes cells in a second non-protein containing culture medium; c) growing the Listeria monocytogenes cells in the second non-protein containing culture medium; and d) disrupting the bacterial cells and collecting a whole cell extract from the disrupted Listeria monocytogenes cells. In other embodiments, the antigen preparation can further comprise a step of removing toxin from the whole cell extract. In other embodiments, the antigen preparation can further comprise a step of collecting a secreted antigen from the second non-protein containing culture medium in which the Listeria monocytogenes cells have grown.

In another aspect, the invention provides a method for treating or preventing listeriosis, which method comprises administering to a human suffering, suspected of suffering or at risk of suffering from listeriosis, an effective amount of the pharmaceutical composition for treating or preventing Listeria monocytogenes infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Listeria monocytogenes infection. In other embodiments, the human for treatment or prevention has a weakened immune system, septicemia, meningitis (or meningoencephalitis), encephalitis, corneal ulcer, pneumonia, or intrauterine or cervical infectious in pregnant women.

The present methods can be used to treat or prevent infection any suitable Listeria monocytogenes strain or serovars. In some embodiments, the present methods can be used to treat or prevent infection caused by a Listeria monocytogenes strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Listeria monocytogenes antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Listeria monocytogenes infection using the affinity purified human polyclonal antibodies.

In some embodiments, the present methods can further comprise, before and after administering the affinity purified human polyclonal antibodies to the human, conducting an immunotest to determine the presence, absence and/or amount of Listeria monocytogenes antigens in a blood sample of the human using the same affinity purified human polyclonal antibodies, to monitor the efficacy of the therapeutic, removal or preventive treatment, wherein the absence or reduction in the Listeria monocytogenes antigens after administering the affinity purified human polyclonal antibodies to the human relative to the amount of Listeria monocytogenes antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Listeria monocytogenes antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Listeria monocytogenes antigens remaining after administering the affinity purified human polyclonal antibodies to the human and the extent of reduction in the Listeria monocytogenes antigens after administering the affinity purified human polyclonal antibodies to the human relative to the amount of Listeria monocytogenes antigens before the administration.

In one aspect, the invention provides a pharmaceutical composition for treating or preventing a Chlamydophila pneumoniae infection, which composition comprises an effective amount of human polyclonal antibodies affinity purified from a human blood sample with an antigenic preparation comprising cellular and/or secreted at least one, preferably two or more, antigens of Chlamydophila pneumoniae cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Chlamydophila pneumoniae antigens used in the affinity purification. In some embodiments, the affinity purified human polyclonal antibodies are substantially free of human antibodies that specifically bind to non-Chlamydophila pneumoniae antigens in said human blood sample.

The affinity purified human polyclonal antibodies can have any suitable concentrations. In some embodiments, the affinity purified human polyclonal antibodies specific to the Chlamydophila pneumoniae antigens have a concentration ranging from about 10 μg/ml to about 10 mg/ml.

In some embodiments, the human blood sample is collected from one or more normal human(s). Alternatively, the human blood sample may be collected from one or more human(s) infected with the Chlamydophila pneumoniae, e.g., collected from 2, 10, 50, 100, 500, 1,000, 5,000, 10,000 or more humans.

The antigenic preparation can comprise an antigen(s) from any suitable Chlamydophila pneumoniae strains or serovars. In some embodiments, the Chlamydophila pneumoniae is strain TWAR, A-03, BAL-6, TW-183, T-2634 or AR-39. In some embodiments, the antigenic preparation comprises a Chlamydophila pneumoniae antigen that confers antibiotic resistance.

The antigenic preparation can comprise any suitable Chlamydophila pneumoniae antigen(s). In some embodiments, the antigenic preparation can comprise a Chlamydophila pneumoniae toxin, Omp11, type III secretion system ATPase, PmpG and/or IncA.

The antigenic preparation can comprise a single, but often multiple Chlamydophila pneumoniae antigens. In some embodiments, the antigenic preparation comprises a whole cell extract and a secreted antigen of Chlamydophila pneumoniae.

The antigenic preparation can be prepared by any suitable methods. In some embodiments, the antigenic preparation is prepared by the following steps: a) growing Chlamydophila pneumoniae cells in a first protein containing culture medium; b) collecting and resuspending the Chlamydophila pneumoniae cells in a second non-protein containing culture medium; c) growing the Chlamydophila pneumoniae cells in the second non-protein containing culture medium; and d) disrupting the bacterial cells and collecting a whole cell extract from the disrupted Chlamydophila pneumoniae cells. In other embodiments, the antigen preparation can further comprise a step of removing toxin from the whole cell extract. In other embodiments, the antigen preparation can further comprise a step of collecting a secreted antigen from the second non-protein containing culture medium in which the Chlamydophila pneumoniae cells have grown.

In another aspect, the invention provides a method for treating or preventing Chlamydophila pneumoniae infection, which method comprises administering to a human suffering, suspected of suffering or at risk of suffering from Chlamydophila pneumoniae infection, an effective amount of the pharmaceutical composition for treating or preventing Chlamydophila pneumoniae infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Chlamydophila pneumoniae infection. In other embodiments, the human for treatment or prevention has a weakened immune system, pneumoniae (also known as Chlamydia pneumonia), atherosclerosis, Alzheimer's disease and/or asthma.

The present methods can be used to treat or prevent infection any suitable Chlamydophila pneumoniae strain or serovars. In some embodiments, the present methods can be used to treat or prevent infection caused by a Chlamydophila pneumoniae strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Chlamydophila pneumoniae antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Chlamydophila pneumoniae infection using the affinity purified human polyclonal antibodies.

In some embodiments, the present methods can further comprise, before and after administering the affinity purified human polyclonal antibodies to the human, conducting an immunotest to determine the presence, absence and/or amount of Chlamydophila pneumoniae antigens in a blood sample of the human using the same affinity purified human polyclonal antibodies, to monitor the efficacy of the therapeutic, removal or preventive treatment, wherein the absence or reduction in the Chlamydophila pneumoniae antigens after administering the affinity purified human polyclonal antibodies to the human relative to the amount of Chlamydophila pneumoniae antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Chlamydophila pneumoniae antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Chlamydophila pneumoniae antigens remaining after administering the affinity purified human polyclonal antibodies to the human and the extent of reduction in the Chlamydophila pneumoniae antigens after administering the affinity purified human polyclonal antibodies to the human relative to the amount of Chlamydophila pneumoniae antigens before the administration.

In one aspect, the invention provides a pharmaceutical composition for treating or preventing an Ureaplasma urealyticum infection, which composition comprises an effective amount of human polyclonal antibodies affinity purified from a human blood sample with an antigenic preparation comprising at least one, preferably two or more, cellular and/or secreted antigens of Ureaplasma urealyticum cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Ureaplasma urealyticum antigens used in the affinity purification. In some embodiments, the affinity purified human polyclonal antibodies are substantially free of human antibodies that specifically bind to non-Ureaplasma urealyticum antigens in said human blood sample.

The affinity purified human polyclonal antibodies can have any suitable concentrations. In some embodiments, the affinity purified human polyclonal antibodies specific to the Ureaplasma urealyticum antigens have a concentration ranging from about 10 μg/ml to about 10 mg/ml.

In some embodiments, the human blood sample is collected from one or more normal human(s). Alternatively, the human blood sample may be collected from one or more human(s) infected with the Ureaplasma urealyticum, e.g., collected from 2, 10, 50, 100, 500, 1,000, 5,000, 10,000 or more humans.

The antigenic preparation can comprise an antigen(s) from any suitable Ureaplasma urealyticum strains or serovars. In some embodiments, the Ureaplasma urealyticum has serovars 1-14.

The antigenic preparation can comprise any suitable antigen(s). In some embodiments. In other embodiments, the Ureaplasma urealyticum antigenic preparation comprises multiple banded (MB) antigen. In other embodiments, the antigenic preparation comprises an Ureaplasma urealyticum antigen that confers antibiotic resistance.

The antigenic preparation can comprise a single, but often multiple Ureaplasma urealyticum antigens. In some embodiments, the antigenic preparation comprises a whole cell extract and a secreted antigen of Ureaplasma urealyticum.

The antigenic preparation can be prepared by any suitable methods. In some embodiments, the antigenic preparation is prepared by the following steps: a) growing Ureaplasma urealyticum cells in a first protein containing culture medium; b) collecting and resuspending the Ureaplasma urealyticum cells in a second non-protein containing culture medium; c) growing the Ureaplasma urealyticum cells in the second non-protein containing culture medium; and d) disrupting the bacterial cells and collecting a whole cell extract from the disrupted Ureaplasma urealyticum cells. In other embodiments, the antigen preparation can further comprise a step of removing toxin from the whole cell extract. In other embodiments, the antigen preparation can further comprise a step of collecting a secreted antigen from the second non-protein containing culture medium in which the Ureaplasma urealyticum cells have grown.

In another aspect, the present invention provides a method for treating or preventing Ureaplasma urealyticum infection, which method comprises administering to a human suffering, suspected of suffering or at risk of suffering from Ureaplasma urealyticum infection, an effective amount of the pharmaceutical composition for treating or preventing Ureaplasma urealyticum infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Ureaplasma urealyticum infection. In other embodiments, the human for treatment or prevention has a weakened immune system, non-specific urethritis (NSU), infertility, chorioamnionitis, stillbirth, premature birth, pneumonia, bronchopulmonary dysplasia and meningitis.

The present methods can be used to treat or prevent infection any suitable Ureaplasma urealyticum strain or serovars. In some embodiments, the present methods can be used to treat or prevent infection caused by a Ureaplasma urealyticum strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Ureaplasma urealyticum antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Ureaplasma urealyticum infection using the affinity purified human polyclonal antibodies.

In some embodiments, the present methods can further comprise, before and after administering the affinity purified human polyclonal antibodies to the human, conducting an immunotest to determine the presence, absence and/or amount of Ureaplasma urealyticum antigens in a blood sample of the human using the same affinity purified human polyclonal antibodies, to monitor the efficacy of the therapeutic, removal or preventive treatment, wherein the absence or reduction in the Ureaplasma urealyticum antigens after administering the affinity purified human polyclonal antibodies to the human relative to the amount of Ureaplasma urealyticum antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Ureaplasma urealyticum antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Ureaplasma urealyticum antigens remaining after administering the affinity purified human polyclonal antibodies to the human and the extent of reduction in the Ureaplasma urealyticum antigens after administering the affinity purified human polyclonal antibodies to the human relative to the amount of Ureaplasma urealyticum antigens before the administration.

In one aspect, the invention provides a pharmaceutical composition for treating or preventing a Mycoplasma hominis infection, which composition comprises an effective amount of human polyclonal antibodies affinity purified from a human blood sample with an antigenic preparation comprising at least one, preferably two or more, cellular and/or secreted antigens of Mycoplasma hominis cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Mycoplasma hominis antigens used in the affinity purification. In some embodiments, the affinity purified human polyclonal antibodies are substantially free of human antibodies that specifically bind to non-Mycoplasma hominis antigens in said human blood sample.

The affinity purified human polyclonal antibodies can have any suitable concentrations. In some embodiments, the affinity purified human polyclonal antibodies specific to the Mycoplasma hominis antigens have a concentration ranging from about 10 μg/ml to about 10 mg/ml.

In some embodiments, the human blood sample is collected from one or more normal human(s). Alternatively, the human blood sample may be collected from one or more human(s) infected with the Mycoplasma hominis, e.g., collected from 2, 10, 50, 100, 500, 1,000, 5,000, 10,000 or more humans.

The antigenic preparation can comprise an antigen(s) from any suitable Mycoplasma hominis strains or serovars. In some embodiments, the Mycoplasma hominis is a strain selected from the group consisting of 1620, 2101, PG21, LBD4, r. Taub, W1458, 1611, F4238, M5039, H5488, 11085, 13428, 1184, 1888, 11932 and 13408.

The antigenic preparation can comprise any suitable antigen(s). In some embodiments, the Mycoplasma hominis antigenic preparation comprises a surface antigen. In other embodiments, the antigenic preparation comprises an Mycoplasma hominis antigen that confers antibiotic resistance.

The antigenic preparation can comprise a single, but often multiple Mycoplasma hominis antigens. In some embodiments, the antigenic preparation comprises a whole cell extract and a secreted antigen of Mycoplasma hominis.

The antigenic preparation can be prepared by any suitable methods. In some embodiments, the antigenic preparation is prepared by the following steps: a) growing Mycoplasma hominis cells in a first protein containing culture medium; b) collecting and resuspending the Mycoplasma hominis cells in a second non-protein containing culture medium; c) growing the Mycoplasma hominis cells in the second non-protein containing culture medium; and d) disrupting the bacterial cells and collecting a whole cell extract from the disrupted Mycoplasma hominis cells. In other embodiments, the antigen preparation can further comprise a step of removing toxin from the whole cell extract. In other embodiments, the antigen preparation can further comprise a step of collecting a secreted antigen from the second non-protein containing culture medium in which the Mycoplasma hominis cells have grown.

In another aspect, the present invention provides a method for treating or preventing Mycoplasma hominis infection, which method comprises administering to a human suffering, suspected of suffering or at risk of suffering from Mycoplasma hominis infection, an effective amount of the pharmaceutical composition for treating or preventing Mycoplasma hominis infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Mycoplasma hominis infection. In other embodiments, the human for treatment or prevention has a weakened immune system, pelvic inflammatory disease, post-abortal fever, post-partum fever, septic arthritis and nongonococcal urethritis.

The present methods can be used to treat or prevent infection any suitable Mycoplasma hominis strain or serovars. In some embodiments, the present methods can be used to treat or prevent infection caused by a Mycoplasma hominis strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Mycoplasma hominis antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Mycoplasma hominis infection using the affinity purified human polyclonal antibodies.

In some embodiments, the present methods can further comprise, before and after administering the affinity purified human polyclonal antibodies to the human, conducting an immunotest to determine the presence, absence and/or amount of Mycoplasma hominis antigens in a blood sample of the human using the same affinity purified human polyclonal antibodies, to monitor the efficacy of the therapeutic, removal or preventive treatment, wherein the absence or reduction in the Mycoplasma hominis antigens after administering the affinity purified human polyclonal antibodies to the human relative to the amount of Mycoplasma hominis antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Mycoplasma hominis antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycoplasma hominis antigens remaining after administering the affinity purified human polyclonal antibodies to the human and the extent of reduction in the Mycoplasma hominis antigens after administering the affinity purified human polyclonal antibodies to the human relative to the amount of Mycoplasma hominis antigens before the administration.

In one aspect, the invention provides a pharmaceutical composition for treating or preventing a Mycoplasma pneumoniae infection, which composition comprises an effective amount of human polyclonal antibodies affinity purified from a human blood sample with an antigenic preparation comprising at least one, preferably two or more, cellular and/or secreted antigens of Mycoplasma pneumoniae cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Mycoplasma pneumoniae antigens used in the affinity purification. In some embodiments, the affinity purified human polyclonal antibodies are substantially free of human antibodies that specifically bind to non-Mycoplasma pneumoniae antigens in said human blood sample.

The affinity purified human polyclonal antibodies can have any suitable concentrations. In some embodiments, the affinity purified human polyclonal antibodies specific to the Mycoplasma pneumoniae antigens have a concentration ranging from about 10 μg/ml to about 10 mg/ml.

In some embodiments, the human blood sample is collected from one or more normal human(s). Alternatively, the human blood sample may be collected from one or more human(s) infected with the Mycoplasma pneumoniae, e.g., collected from 2, 10, 50, 100, 500, 1,000, 5,000, 10,000 or more humans.

The antigenic preparation can comprise an antigen(s) from any suitable Mycoplasma pneumoniae strains or serovars. In some embodiments, the Mycoplasma pneumoniae is a strain M129 (ATCC 29342), strain FH or strain MPN372.

The antigenic preparation can comprise any suitable antigen(s). In some embodiments, the Mycoplasma pneumoniae antigenic preparation comprises a surface antigen, adhesin P1, the 30 Kda adhesin-related protein on the tip structure of Mycoplasma pneumoniae cells or CARDS toxin. In other embodiments, the antigenic preparation comprises a Mycoplasma pneumoniae antigen that confers antibiotic resistance.

The antigenic preparation can comprise a single, but often multiple Mycoplasma pneumoniae antigens. In some embodiments, the antigenic preparation comprises a whole cell extract and a secreted antigen of Mycoplasma pneumoniae.

The antigenic preparation can be prepared by any suitable methods. In some embodiments, the antigenic preparation is prepared by the following steps: a) growing Mycoplasma pneumoniae cells in a first protein containing culture medium; b) collecting and resuspending the Mycoplasma pneumoniae cells in a second non-protein containing culture medium; c) growing the Mycoplasma pneumoniae cells in the second non-protein containing culture medium; and d) disrupting the bacterial cells and collecting a whole cell extract from the disrupted Mycoplasma pneumoniae cells. In other embodiments, the antigen preparation can further comprise a step of removing toxin from the whole cell extract. In other embodiments, the antigen preparation can further comprise a step of collecting a secreted antigen from the second non-protein containing culture medium in which the Mycoplasma pneumoniae cells have grown.

In another aspect, the present invention provides a method for treating or preventing Mycoplasma pneumoniae infection, which method comprises administering to a human suffering, suspected of suffering or at risk of suffering from Mycoplasma pneumoniae infection, an effective amount of the pharmaceutical composition for treating or preventing Mycoplasma pneumoniae infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Mycoplasma pneumoniae infection. In other embodiments, the human for treatment or prevention has a weakened immune system, acute or chronic respiratory infection, asthma or fulminant disease. The exemplary acute or chronic respiratory infection includes human primary atypical pneumonia (PAP) (walking pneumonia), tracheobronchitis, pharyngitis or community acquired pneumonia.

The present methods can be used to treat or prevent infection any suitable Mycoplasma pneumoniae strain or serovars. In some embodiments, the present methods can be used to treat or prevent infection caused by a Mycoplasma pneumoniae strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Mycoplasma pneumoniae antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Mycoplasma pneumoniae infection using the affinity purified human polyclonal antibodies.

In some embodiments, the present methods can further comprise, before and after administering the affinity purified human polyclonal antibodies to the human, conducting an immunotest to determine the presence, absence and/or amount of Mycoplasma pneumoniae antigens in a blood sample of the human using the same affinity purified human polyclonal antibodies, to monitor the efficacy of the therapeutic, removal or preventive treatment, wherein the absence or reduction in the Mycoplasma pneumoniae antigens after administering the affinity purified human polyclonal antibodies to the human relative to the amount of Mycoplasma pneumoniae antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Mycoplasma pneumoniae antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycoplasma pneumoniae antigens remaining after administering the affinity purified human polyclonal antibodies to the human and the extent of reduction in the Mycoplasma pneumoniae antigens after administering the affinity purified human polyclonal antibodies to the human relative to the amount of Mycoplasma pneumoniae antigens before the administration.

In one aspect, the invention provides a pharmaceutical composition for treating or preventing Haemophilus influenzae (formerly called Pfeiffer's bacillus or Bacillus influenzae) infection, which composition comprises an effective amount of human polyclonal antibodies affinity purified from a human blood sample with an antigenic preparation comprising at least one, preferably two or more, cellular and/or secreted antigens of Haemophilus influenzae cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Haemophilus influenzae antigen used in the affinity purification. In some embodiments, the affinity purified human polyclonal antibodies are substantially free of human antibodies that specifically bind to non-Haemophilus influenzae antigen in said human blood sample.

The affinity purified human polyclonal antibodies can have any suitable concentrations. In some embodiments, the affinity purified human polyclonal antibodies specific to the Haemophilus influenzae antigen have a concentration ranging from about 10 μg/ml to about 10 mg/ml.

In some embodiments, the human blood sample is collected from one or more normal human(s). Alternatively, the human blood sample may be collected from one or more human(s) infected with the Haemophilus influenzae, e.g., collected from 2, 10, 50, 100, 500, 1,000, 5,000, 10,000 or more humans.

The antigenic preparation can comprise an antigen(s) from any suitable Haemophilus influenzae strains or serovars. In some embodiments, the Haemophilus influenzae is an unencapsulated strain or an encapsulated strain. Exemplary encapsulated strains include the serotype a, b, c, d, e, or f.

The antigenic preparation can comprise any suitable antigen(s). In some embodiments, the antigenic preparation comprises a Haemophilus influenzae antigen that confers antibiotic resistance.

The antigenic preparation can comprise a single, but often multiple Haemophilus influenzae antigens. In some embodiments, the antigenic preparation comprises a whole cell extract and a secreted antigen of Haemophilus influenzae.

The antigenic preparation can be prepared by any suitable methods. In some embodiments, the antigenic preparation is prepared by the following steps: a) growing Haemophilus influenzae cells in a first protein containing culture medium; b) collecting and resuspending the Haemophilus influenzae cells in a second non-protein containing culture medium; c) growing the Haemophilus influenzae cells in the second non-protein containing culture medium; and d) disrupting the bacterial cells and collecting a whole cell extract from the disrupted Haemophilus influenzae cells. In other embodiments, the antigen preparation can further comprise a step of removing toxin from the whole cell extract. In other embodiments, the antigen preparation can further comprise a step of collecting a secreted antigen from the second non-protein containing culture medium in which the Haemophilus influenzae cells have grown.

In another aspect, the present invention provides a method for treating or preventing Haemophilus influenzae infection, which method comprises administering to a human suffering, suspected of suffering or at risk of suffering from Haemophilus influenzae infection, an effective amount of the pharmaceutical composition for treating or preventing Haemophilus influenzae infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Haemophilus influenzae infection. In other embodiments, the human for treatment or prevention has a weakened immune system, bacteremia, pneumonia, acute bacterial meningitis, cellulitis, osteomyelitis, epiglottitis, infectious arthritis, ear infections (otitis media), eye infections (conjunctivitis), and/or sinusitis.

The present methods can be used to treat or prevent infection any suitable Haemophilus influenzae strain or serovars. In some embodiments, the present methods can be used to treat or prevent infection caused by a Haemophilus influenzae strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Haemophilus influenzae antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Haemophilus influenzae infection using the affinity purified human polyclonal antibodies.

In some embodiments, the present methods can further comprise, before and after administering the affinity purified human polyclonal antibodies to the human, conducting an immunotest to determine the presence, absence and/or amount of Haemophilus influenzae antigen in a blood sample of the human using the same affinity purified human polyclonal antibodies, to monitor the efficacy of the therapeutic, removal or preventive treatment, wherein the absence or reduction in the Haemophilus influenzae antigen after administering the affinity purified human polyclonal antibodies to the human relative to the amount of Haemophilus influenzae antigen before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Haemophilus influenzae antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Haemophilus influenzae antigen remaining after administering the affinity purified human polyclonal antibodies to the human and the extent of reduction in the Haemophilus influenzae antigen after administering the affinity purified human polyclonal antibodies to the human relative to the amount of Haemophilus influenzae antigen before the administration.

In one aspect, the invention provides a pharmaceutical composition for treating or preventing Campylobacter jejuni infection, which composition comprises an effective amount of human polyclonal antibodies affinity purified from a human blood sample with an antigenic preparation comprising at least one, preferably two or more, cellular and/or secreted antigens of Campylobacter jejuni cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Campylobacter jejuni antigen used in the affinity purification. In some embodiments, the affinity purified human polyclonal antibodies are substantially free of human antibodies that specifically bind to non-Campylobacter jejuni antigen in said human blood sample.

The affinity purified human polyclonal antibodies can have any suitable concentrations. In some embodiments, the affinity purified human polyclonal antibodies specific to the Campylobacter jejuni antigen have a concentration ranging from about 10 μg/ml to about 10 mg/ml.

In some embodiments, the human blood sample is collected from one or more normal human(s). Alternatively, the human blood sample may be collected from one or more human(s) infected with the Campylobacter jejuni, e.g., collected from 2, 10, 50, 100, 500, 1,000, 5,000, 10,000 or more humans.

The antigenic preparation can comprise an antigen(s) from any suitable Campylobacter jejuni strains or serovars. In some embodiments, the Campylobacter jejuni is strain NCTC11168.

The antigenic preparation can comprise any suitable antigen(s). In some embodiments, the antigenic preparation comprises a Campylobacter jejuni antigen that confers antibiotic resistance.

The antigenic preparation can comprise a single, but often multiple Campylobacter jejuni antigens. In some embodiments, the antigenic preparation comprises a whole cell extract and a secreted antigen of Campylobacter jejuni.

The antigenic preparation can be prepared by any suitable methods. In some embodiments, the antigenic preparation is prepared by the following steps: a) growing Campylobacter jejuni cells in a first protein containing culture medium; b) collecting and resuspending the Campylobacter jejuni cells in a second non-protein containing culture medium; c) growing the Campylobacter jejuni cells in the second non-protein containing culture medium; and d) disrupting the bacterial cells and collecting a whole cell extract from the disrupted Campylobacter jejuni cells. In other embodiments, the antigen preparation can further comprise a step of removing toxin from the whole cell extract. In other embodiments, the antigen preparation can further comprise a step of collecting a secreted antigen from the second non-protein containing culture medium in which the Campylobacter jejuni cells have grown.

In another aspect, the present invention provides a method for treating or preventing Campylobacter jejuni infection, which method comprises administering to a human suffering, suspected of suffering or at risk of suffering from Campylobacter jejuni infection, an effective amount of the pharmaceutical composition for treating or preventing Campylobacter jejuni infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Campylobacter jejuni infection. In some embodiments, the human for treatment or prevention has a weakened immune system, enteritis, food poisoning and/or Guillain-Barré syndrome (GBS). Exemplary enteritis includes human gastroenteritis.

The present methods can be used to treat or prevent infection any suitable Campylobacter jejuni strain or serovars. In some embodiments, the present methods can be used to treat or prevent infection caused by a Campylobacter jejuni strain that is resistant to an anti-bacterial drug or treatment.

In some embodiments, the present methods can further comprise, prior to administering the affinity purified human polyclonal antibodies to the human, conducting an immunotest to determine the presence, absence and/or amount of Campylobacter jejuni antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Campylobacter jejuni infection using the affinity purified human polyclonal antibodies.

In some embodiments, the present methods can further comprise, before and after administering the affinity purified human polyclonal antibodies to the human, conducting an immunotest to determine the presence, absence and/or amount of Campylobacter jejuni antigen in a blood sample of the human using the same affinity purified human polyclonal antibodies, to monitor the efficacy of the therapeutic, removal or preventive treatment, wherein the absence or reduction in the Campylobacter jejuni antigen after administering the affinity purified human polyclonal antibodies to the human relative to the amount of Campylobacter jejuni antigen before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Campylobacter jejuni antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Campylobacter jejuni antigen remaining after administering the affinity purified human polyclonal antibodies to the human and the extent of reduction in the Campylobacter jejuni antigen after administering the affinity purified human polyclonal antibodies to the human relative to the amount of Campylobacter jejuni antigen before the administration.

In one aspect, the invention provides a pharmaceutical composition for treating or preventing malaria, which composition comprises an effective amount of human polyclonal antibodies affinity purified from a human blood sample with an antigenic preparation comprising at least one, preferably two or more, cellular and/or secreted antigens of malaria-causing Plasmodium cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Plasmodium antigens used in the affinity purification. In some embodiments, the affinity purified human polyclonal antibodies are substantially free of human antibodies that specifically bind to non-Plasmodium antigens in said human blood sample.

The affinity purified human polyclonal antibodies can have any suitable concentrations. In some embodiments, the affinity purified human polyclonal antibodies specific to the Plasmodium antigens have a concentration ranging from about 10 μg/ml to about 10 mg/ml.

In some embodiments, the human blood sample is collected from one or more normal human(s). Alternatively, the human blood sample may be collected from one or more human(s) infected with the Plasmodium, e.g., collected from 2, 10, 50, 100, 500, 1,000, 5,000, 10,000 or more humans.

The antigenic preparation can comprise an antigen(s) from any suitable Plasmodium species, strains or serovars. In some embodiments, the antigenic preparation comprises antigens from P. falciparum, P. malariae, P. ovale, P. vivax and P. knowlesi, P. inui, P. cynomolgi, P. simiovale, P. brazilianum, P. schwetzi and P. simium.

The antigenic preparation can comprise any suitable antigen(s). In some embodiments, the antigenic preparation comprises a surface antigen from a Plasmodium cell, Plasmodium glutamate dehydrogenase or Plasmodium lactate dehydrogenase. In other embodiments, the antigenic preparation comprises a Plasmodium strain or antigen that confers resistance to an anti-malaria drug. In other embodiments, the antigenic preparation comprises a Plasmodium toxin.

The antigenic preparation can comprise a single, but often multiple Plasmodium antigens. In some embodiments, the antigenic preparation comprises a whole cell extract and a secreted antigen of Plasmodium.

The antigenic preparation can be prepared by any suitable methods. In some embodiments, the antigenic preparation is prepared by the following steps: a) growing Plasmodium cells in a first protein containing culture medium; b) collecting and resuspending the Plasmodium cells in a second non-protein containing culture medium; c) growing the Plasmodium cells in the second non-protein containing culture medium; and d) disrupting the bacterial cells and collecting a whole cell extract from the disrupted Plasmodium cells. In other embodiments, the antigen preparation can further comprise a step of removing toxin from the whole cell extract. In other embodiments, the antigen preparation can further comprise a step of collecting a secreted antigen from the second non-protein containing culture medium in which the Plasmodium cells have grown.

In another aspect, the present invention provides a method for treating or preventing malaria, which method comprises administering to a human suffering, suspected of suffering or at risk of suffering from malaria, an effective amount of the pharmaceutical composition for treating or preventing Plasmodium infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Plasmodium infection. In other embodiments, the human for treatment or prevention has a weakened immune system, fever, shivering, arthralgia (joint pain), vomiting, anemia (caused by hemolysis), hemoglobinuria, retinal damage, and/or convulsions.

The present methods can be used to treat or prevent infection any suitable malaria-causing Plasmodium species, strain or serovars. In some embodiments, the present methods can be used to treat or prevent infection caused by a malaria-causing Plasmodium strain that is resistant to an anti-malaria drug or treatment.

In some embodiments, the present methods can further comprise, prior to administering the affinity purified human polyclonal antibodies to the human, conducting an immunotest to determine the presence, absence and/or amount of Plasmodium antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Plasmodium infection using the affinity purified human polyclonal antibodies.

In some embodiments, the present methods can further comprise, before and after administering the affinity purified human polyclonal antibodies to the human, conducting an immunotest to determine the presence, absence and/or amount of Plasmodium antigens in a blood sample of the human using the same affinity purified human polyclonal antibodies, to monitor the efficacy of the therapeutic, removal or preventive treatment, wherein the absence or reduction in the Plasmodium antigens after administering the affinity purified human polyclonal antibodies to the human relative to the amount of Plasmodium antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Plasmodium antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Plasmodium antigens remaining after administering the affinity purified human polyclonal antibodies to the human and the extent of reduction in the Plasmodium antigens after administering the affinity purified human polyclonal antibodies to the human relative to the amount of Plasmodium antigens before the administration.

In one aspect, the invention provides a pharmaceutical composition for treating or preventing a Pneumocystis jirovecii infection, which composition comprises an effective amount of human polyclonal antibodies affinity purified from a human blood sample with an antigenic preparation comprising at least one, preferably two or more, cellular and/or secreted antigens of Pneumocystis jirovecii cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Pneumocystis jirovecii antigens used in the affinity purification. In some embodiments, the affinity purified human polyclonal antibodies are substantially free of human antibodies that specifically bind to non-Pneumocystis jirovecii antigens in said human blood sample.

The affinity purified human polyclonal antibodies can have any suitable concentrations. In some embodiments, the affinity purified human polyclonal antibodies specific to the Pneumocystis jirovecii antigens have a concentration ranging from about 10 μg/ml to about 10 mg/ml.

In some embodiments, the human blood sample is collected from one or more normal human(s). Alternatively, the human blood sample may be collected from one or more human(s) infected with the Pneumocystis jirovecii, e.g., collected from 2, 10, 50, 100, 500, 1,000, 5,000, 10,000 or more humans.

The antigenic preparation can comprise an antigen(s) from any suitable Pneumocystis jirovecii strains or serovars.

The antigenic preparation can comprise any suitable antigen(s). In some embodiments, the Pneumocystis jirovecii antigenic preparation comprises a major surface glycoprotein (MSG), e.g., MSG-14 of Pneumocystis jirovecii. In other embodiments, the antigenic preparation comprises a Pneumocystis jirovecii antigen that confers resistance to an anti-fungal drug, e.g., an antigen from dihydropteroate synthase (DHPS) and/or dihydrofolate reductase (DHFR). Exemplary anti-fungal drugs include a sulfa drug.

The antigenic preparation can comprise a single, but often multiple Pneumocystis jirovecii antigens. In some embodiments, the antigenic preparation comprises a whole cell extract and a secreted antigen of Pneumocystis jirovecii.

The antigenic preparation can be prepared by any suitable methods. In some embodiments, the antigenic preparation is prepared by the following steps: a) growing Pneumocystis jirovecii cells in a first protein containing culture medium; b) collecting and resuspending the Pneumocystis jirovecii cells in a second non-protein containing culture medium; c) growing the Pneumocystis jirovecii cells in the second non-protein containing culture medium; and d) disrupting the bacterial cells and collecting a whole cell extract from the disrupted Pneumocystis jirovecii cells. In other embodiments, the antigen preparation can further comprise a step of removing toxin from the whole cell extract. In other embodiments, the antigen preparation can further comprise a step of collecting a secreted antigen from the second non-protein containing culture medium in which the Pneumocystis jirovecii cells have grown.

In another aspect, the present invention provides a method for treating or preventing Pneumocystis jirovecii infection, which method comprises administering to a human suffering, suspected of suffering or at risk of suffering from Pneumocystis jirovecii infection, an effective amount of the pharmaceutical composition for treating or preventing Pneumocystis jirovecii infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Pneumocystis jirovecii infection. In other embodiments, the human for treatment or prevention has a weakened immune system, pneumocystis pneumonia (PCP or pneumocystosis), cancer, and/or HIV/AIDS.

The present methods can be used to treat or prevent infection any suitable malaria-causing Pneumocystis jirovecii, strain or serovars. In some embodiments, the present methods can be used to treat or prevent infection caused by a malaria-causing Pneumocystis jirovecii strain that is resistant to an anti-fungal drug or treatment, e.g., a sulfa drug.

In some embodiments, the present methods can further comprise, prior to administering the affinity purified human polyclonal antibodies to the human, conducting an immunotest to determine the presence, absence and/or amount of Pneumocystis jirovecii antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Pneumocystis jirovecii infection using the affinity purified human polyclonal antibodies.

In some embodiments, the present methods can further comprise, before and after administering the affinity purified human polyclonal antibodies to the human, conducting an immunotest to determine the presence, absence and/or amount of Pneumocystis jirovecii antigens in a blood sample of the human using the same affinity purified human polyclonal antibodies, to monitor the efficacy of the therapeutic, removal or preventive treatment, wherein the absence or reduction in the Pneumocystis jirovecii antigens after administering the affinity purified human polyclonal antibodies to the human relative to the amount of Pneumocystis jirovecii antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Pneumocystis jirovecii antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Pneumocystis jirovecii antigens remaining after administering the affinity purified human polyclonal antibodies to the human and the extent of reduction in the Pneumocystis jirovecii antigens after administering the affinity purified human polyclonal antibodies to the human relative to the amount of Pneumocystis jirovecii antigens before the administration.

In one aspect, the invention provides a pharmaceutical composition for treating or preventing a Histoplasma capsulatum infection, which composition comprises an effective amount of human polyclonal antibodies affinity purified from a human blood sample with an antigenic preparation comprising at least one, preferably two or more, cellular and/or secreted antigens of Histoplasma capsulatum cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Histoplasma capsulatum antigens used in the affinity purification. In some embodiments, the affinity purified human polyclonal antibodies are substantially free of human antibodies that specifically bind to non-Histoplasma capsulatum antigens in said human blood sample.

The affinity purified human polyclonal antibodies can have any suitable concentrations. In some embodiments, the affinity purified human polyclonal antibodies specific to the Histoplasma capsulatum antigens have a concentration ranging from about 10 μg/ml to about 10 mg/ml.

In some embodiments, the human blood sample is collected from one or more normal human(s). Alternatively, the human blood sample may be collected from one or more human(s) infected with the Histoplasma capsulatum e.g., collected from 2, 10, 50, 100, 500, 1,000, 5,000, 10,000 or more humans.

The antigenic preparation can comprise an antigen(s) from any suitable Histoplasma capsulatum strains or serovars. In some embodiments, the antigenic preparation comprises antigen(s) from Histoplasma capsulatum var. capsulatum,

The antigenic preparation can comprise any suitable antigen(s). In some embodiments, the Histoplasma capsulatum antigenic preparation comprises a 69- to 70-kDa H. capsulatum var. capsulatum-specific antigen. In other embodiments, the antigenic preparation comprises a Histoplasma capsulatum antigen that confers resistance to an anti-fungal drug, e.g., fluconazole.

The antigenic preparation can comprise a single, but often multiple Histoplasma capsulatum antigens. In some embodiments, the antigenic preparation comprises a whole cell extract and a secreted antigen of Histoplasma capsulatum.

The antigenic preparation can be prepared by any suitable methods. In some embodiments, the antigenic preparation is prepared by the following steps: a) growing Histoplasma capsulatum cells in a first protein containing culture medium; b) collecting and resuspending the Histoplasma capsulatum cells in a second non-protein containing culture medium; c) growing the Histoplasma capsulatum cells in the second non-protein containing culture medium; and d) disrupting the bacterial cells and collecting a whole cell extract from the disrupted Histoplasma capsulatum cells. In other embodiments, the antigen preparation can further comprise a step of removing toxin from the whole cell extract. In other embodiments, the antigen preparation can further comprise a step of collecting a secreted antigen from the second non-protein containing culture medium in which the Histoplasma capsulatum cells have grown.

In another aspect, the present invention provides a method for treating or preventing Histoplasma capsulatum infection, which method comprises administering to a human suffering, suspected of suffering or at risk of suffering from Histoplasma capsulatum infection, an effective amount of the pharmaceutical composition for treating or preventing Histoplasma capsulatum infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Histoplasma capsulatum infection. In other embodiments, the human for treatment or prevention has a weakened immune system, histoplasmosis (also known as Cave disease, Darling's disease, Ohio valley disease, and “Reticuloendotheliosis) and/or HIV/AIDS.

The present methods can be used to treat or prevent infection any suitable Histoplasma capsulatum strain or serovars. In some embodiments, the present methods can be used to treat or prevent infection caused by a Histoplasma capsulatum strain that is resistant to an anti-fungal drug or treatment, e.g., fluconazole.

In some embodiments, the present methods can further comprise, prior to administering the affinity purified human polyclonal antibodies to the human, conducting an immunotest to determine the presence, absence and/or amount of Histoplasma capsulatum antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Histoplasma capsulatum infection using the affinity purified human polyclonal antibodies.

In some embodiments, the present methods can further comprise, before and after administering the affinity purified human polyclonal antibodies to the human, conducting an immunotest to determine the presence, absence and/or amount of Histoplasma capsulatum antigens in a blood sample of the human using the same affinity purified human polyclonal antibodies, to monitor the efficacy of the therapeutic, removal or preventive treatment, wherein the absence or reduction in the Histoplasma capsulatum antigens after administering the affinity purified human polyclonal antibodies to the human relative to the amount of Histoplasma capsulatum antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Histoplasma capsulatum antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Histoplasma capsulatum antigens remaining after administering the affinity purified human polyclonal antibodies to the human and the extent of reduction in the Histoplasma capsulatum antigens after administering the affinity purified human polyclonal antibodies to the human relative to the amount of Histoplasma capsulatum antigens before the administration.

In one aspect, the invention provides a pharmaceutical composition for treating or preventing a Blastomyces dermatitidis infection, which composition comprises an effective amount of human polyclonal antibodies affinity purified from a human blood sample with an antigenic preparation comprising at least one, preferably two or more, cellular and/or secreted antigens of Blastomyces dermatitidis cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Blastomyces dermatitidis antigens used in the affinity purification. In some embodiments, the affinity purified human polyclonal antibodies are substantially free of human antibodies that specifically bind to non-Blastomyces dermatitidis antigens in said human blood sample.

The affinity purified human polyclonal antibodies can have any suitable concentrations. In some embodiments, the affinity purified human polyclonal antibodies specific to the Blastomyces dermatitidis antigens have a concentration ranging from about 10 μg/ml to about 10 mg/ml.

In some embodiments, the human blood sample is collected from one or more normal human(s). Alternatively, the human blood sample may be collected from one or more human(s) infected with the Blastomyces dermatitidis e.g., collected from 2, 10, 50, 100, 500, 1,000, 5,000, 10,000 or more humans.

The antigenic preparation can comprise an antigen(s) from any suitable Blastomyces dermatitidis strains or serovars. In some embodiments, the antigenic preparation comprises antigen(s) from Blastomyces dermatitidis strain SLH-14081, ER-3, ATCC18188 or ATCC26199.

The antigenic preparation can comprise any suitable antigen(s). In some embodiments, the Blastomyces dermatitidis antigenic preparation comprises an immunodominant cell wall antigen. In other embodiments, the immunodominant cell wall antigen is WI-1. (See e.g., Newman et al., The Journal of Immunology, 154(2):753-761, (1995)). In other embodiments, the antigenic preparation comprises a Blastomyces dermatitidis antigen that confers resistance to an anti-fungal drug.

The antigenic preparation can comprise a single, but often multiple Blastomyces dermatitidis antigens. In some embodiments, the antigenic preparation comprises a whole cell extract and a secreted antigen of Blastomyces dermatitidis.

The antigenic preparation can be prepared by any suitable methods. In some embodiments, the antigenic preparation is prepared by the following steps: a) growing Blastomyces dermatitidis cells in a first protein containing culture medium; b) collecting and resuspending the Blastomyces dermatitidis cells in a second non-protein containing culture medium; c) growing the Blastomyces dermatitidis cells in the second non-protein containing culture medium; and d) disrupting the bacterial cells and collecting a whole cell extract from the disrupted Blastomyces dermatitidis cells. In other embodiments, the antigen preparation can further comprise a step of removing toxin from the whole cell extract. In other embodiments, the antigen preparation can further comprise a step of collecting a secreted antigen from the second non-protein containing culture medium in which the Blastomyces dermatitidis cells have grown. Exemplary secreted antigens include WI-1. (See e.g., Audet et al. Protein Expr. Purif., 11:219-26 (1997)).

In another aspect, the present invention provides a method for treating or preventing Blastomyces dermatitidis infection, which method comprises administering to a human suffering, suspected of suffering or at risk of suffering from Blastomyces dermatitidis infection, an effective amount of the pharmaceutical composition for treating or preventing Blastomyces dermatitidis infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Blastomyces dermatitidis infection. In other embodiments, the human for treatment or prevention has a weakened immune system, blastomycosis (also known as “North American blastomycosis,” “blastomycetic dermatitis,” and “Gilchrist's disease), and/or HIV/AIDS.

The present methods can be used to treat or prevent infection any suitable Blastomyces dermatitidis strain or serovars. In some embodiments, the present methods can be used to treat or prevent infection caused by a Blastomyces dermatitidis strain that is resistant to an anti-fungal drug or treatment.

In some embodiments, the present methods can further comprise, prior to administering the affinity purified human polyclonal antibodies to the human, conducting an immunotest to determine the presence, absence and/or amount of Blastomyces dermatitidis antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Blastomyces dermatitidis infection using the affinity purified human polyclonal antibodies.

In some embodiments, the present methods can further comprise, before and after administering the affinity purified human polyclonal antibodies to the human, conducting an immunotest to determine the presence, absence and/or amount of Blastomyces dermatitidis antigens in a blood sample of the human using the same affinity purified human polyclonal antibodies, to monitor the efficacy of the therapeutic, removal or preventive treatment, wherein the absence or reduction in the Blastomyces dermatitidis antigens after administering the affinity purified human polyclonal antibodies to the human relative to the amount of Blastomyces dermatitidis antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Blastomyces dermatitidis antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Blastomyces dermatitidis antigens remaining after administering the affinity purified human polyclonal antibodies to the human and the extent of reduction in the Blastomyces dermatitidis antigens after administering the affinity purified human polyclonal antibodies to the human relative to the amount of Blastomyces dermatitidis antigens before the administration.

In one aspect, the invention provides a pharmaceutical composition for treating or preventing a Coccidioides infection, which composition comprises an effective amount of human polyclonal antibodies affinity purified from a human blood sample with an antigenic preparation comprising at least one, preferably two or more, cellular and/or secreted antigens of Coccidioides cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Coccidioides antigens used in the affinity purification. In some embodiments, the affinity purified human polyclonal antibodies are substantially free of human antibodies that specifically bind to non-Coccidioides antigens in said human blood sample.

The affinity purified human polyclonal antibodies can have any suitable concentrations. In some embodiments, the affinity purified human polyclonal antibodies specific to the Coccidioides antigens have a concentration ranging from about 10 μg/ml to about 10 mg/ml.

In some embodiments, the human blood sample is collected from one or more normal human(s). Alternatively, the human blood sample may be collected from one or more human(s) infected with the Coccidioides e.g., collected from 2, 10, 50, 100, 500, 1,000, 5,000, 10,000 or more humans.

The antigenic preparation can comprise an antigen(s) from any suitable Coccidioides species, strains or serovars. In some embodiments, the antigenic preparation comprises antigen(s) from C. immitis or C. posadasii.

The antigenic preparation can comprise any suitable antigen(s). In some embodiments, the Coccidioides antigenic preparation comprises galactomannan (See e.g., Durkin et al., Clin. Infect. Dis., 47(8):e69-73 (2008), the tube precipitin (TP) antigen (Gade, et al., J. Clin. Microbiol., 30(8): 1907-1912 (1992)) or a Coccidioides-specific antigen (CS-Ag) (See e.g., Pan and Cole, Infect. Immun., 63:3994-4002, (1995)). In other embodiments, the antigenic preparation comprises a Coccidioides antigen that confers resistance to an anti-fungal drug.

The antigenic preparation can comprise a single, but often multiple Coccidioides antigens. In some embodiments, the antigenic preparation comprises a whole cell extract and a secreted antigen of Coccidioides.

The antigenic preparation can be prepared by any suitable methods. In some embodiments, the antigenic preparation is prepared by the following steps: a) growing Coccidioides cells in a first protein containing culture medium; b) collecting and resuspending the Coccidioides cells in a second non-protein containing culture medium; c) growing the Coccidioides cells in the second non-protein containing culture medium; and d) disrupting the bacterial cells and collecting a whole cell extract from the disrupted Coccidioides cells. In other embodiments, the antigen preparation can further comprise a step of removing toxin from the whole cell extract. In other embodiments, the antigen preparation can further comprise a step of collecting a secreted antigen from the second non-protein containing culture medium in which the Coccidioides cells have grown. Exemplary secreted antigens include a Coccidioides-specific antigen (CS-Ag) (See e.g., Pan and Cole, Infect. Immun., 63:3994-4002. (1995))).

In another aspect, the present invention provides a method for treating or preventing Coccidioides infection, which method comprises administering to a human suffering, suspected of suffering or at risk of suffering from Coccidioides infection, an effective amount of the pharmaceutical composition for treating or preventing Coccidioides infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Coccidioides infection. In other embodiments, the human for treatment or prevention has a weakened immune system, coccidioidomycosis (also known as “California disease,” “Desert rheumatism,” “San Joaquin valley fever,” and “Valley fever”), and/or HIV/AIDS. Exemplary coccidioidomycoses include primary pulmonary coccidioidomycosis, disseminated coccidioidomycosis or primary cutaneous coccidioidomycosis.

The present methods can be used to treat or prevent infection any suitable Coccidioides species, strain or serovars. In some embodiments, the present methods can be used to treat or prevent infection caused by a Coccidioides strain that is resistant to an anti-fungal drug or treatment.

In some embodiments, the present methods can further comprise, prior to administering the affinity purified human polyclonal antibodies to the human, conducting an immunotest to determine the presence, absence and/or amount of Coccidioides antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Coccidioides infection using the affinity purified human polyclonal antibodies.

In some embodiments, the present methods can further comprise, before and after administering the affinity purified human polyclonal antibodies to the human, conducting an immunotest to determine the presence, absence and/or amount of Coccidioides antigens in a blood sample of the human using the same affinity purified human polyclonal antibodies, to monitor the efficacy of the therapeutic, removal or preventive treatment, wherein the absence or reduction in the Coccidioides antigens after administering the affinity purified human polyclonal antibodies to the human relative to the amount of Coccidioides antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Coccidioides antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Coccidioides antigens remaining after administering the affinity purified human polyclonal antibodies to the human and the extent of reduction in the Blastomyces Coccidioides antigens after administering the affinity purified human polyclonal antibodies to the human relative to the amount of Coccidioides antigens before the administration.

In one aspect, the invention provides a pharmaceutical composition for treating or preventing an Aspergillus infection, which composition comprises an effective amount of human polyclonal antibodies affinity purified from a human blood sample with an antigenic preparation comprising at least one, preferably two or more, cellular and/or secreted antigens of Aspergillus cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Aspergillus antigens used in the affinity purification. In some embodiments, the affinity purified human polyclonal antibodies are substantially free of human antibodies that specifically bind to non-Aspergillus antigens in said human blood sample.

The affinity purified human polyclonal antibodies can have any suitable concentrations. In some embodiments, the affinity purified human polyclonal antibodies specific to the Aspergillus antigens have a concentration ranging from about 10 μg/ml to about 10 mg/ml.

In some embodiments, the human blood sample is collected from one or more normal human(s). Alternatively, the human blood sample may be collected from one or more human(s) infected with the Aspergillus e.g., collected from 2, 10, 50, 100, 500, 1,000, 5,000, 10,000 or more humans.

The antigenic preparation can comprise an antigen(s) from any suitable Aspergillus species, strains or serovars. In some embodiments, the antigenic preparation comprises antigen(s) from Aspergillus fumigatus, Aspergillus flavus. or Aspergillus clavatus.

The antigenic preparation can comprise any suitable antigen(s). In some embodiments, the Aspergillus antigenic preparation comprises Aspergillus toxin, e.g., aflatoxin or galactomannan (See e.g., Klont et al., Clinical Infection Diseases, 39:1467-74 (2004)). In other embodiments, the antigenic preparation comprises an Aspergillus antigen that confers resistance to an anti-fungal drug.

The antigenic preparation can comprise a single, but often multiple Aspergillus antigens. In some embodiments, the antigenic preparation comprises a whole cell extract and a secreted antigen of Aspergillus, e.g., the secreted antigens disclosed in Medina et al., Proteomics, 5(12):3153-61 (2005).

The antigenic preparation can be prepared by any suitable methods. In some embodiments, the antigenic preparation is prepared by the following steps: a) growing Aspergillus cells in a first protein containing culture medium; b) collecting and resuspending the Aspergillus cells in a second non-protein containing culture medium; c) growing the Aspergillus cells in the second non-protein containing culture medium; and d) disrupting the bacterial cells and collecting a whole cell extract from the disrupted Aspergillus cells. In other embodiments, the antigen preparation can further comprise a step of removing toxin from the whole cell extract. In other embodiments, the antigen preparation can further comprise a step of collecting a secreted antigen from the second non-protein containing culture medium in which the Aspergillus cells have grown.

In another aspect, the present invention provides a method for treating or preventing Aspergillus infection, which method comprises administering to a human suffering, suspected of suffering or at risk of suffering from Aspergillus infection, an effective amount of the pharmaceutical composition for treating or preventing Aspergillus infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Aspergillus infection. In other embodiments, the human for treatment or prevention has a weakened immune system, aspergillosis, and/or HIV/AIDS. Exemplary aspergilloses include allergic bronchopulmonary aspergillosis (or ABPA), acute invasive aspergillosis, disseminated invasive aspergillosis, or Aspergilloma.

The present methods can be used to treat or prevent infection any suitable Aspergillus species, strain or serovars. In some embodiments, the present methods can be used to treat or prevent infection caused by an Aspergillus strain that is resistant to an anti-fungal drug or treatment.

In some embodiments, the present methods can further comprise, prior to administering the affinity purified human polyclonal antibodies to the human, conducting an immunotest to determine the presence, absence and/or amount of Aspergillus antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Aspergillus infection using the affinity purified human polyclonal antibodies.

In some embodiments, the present methods can further comprise, before and after administering the affinity purified human polyclonal antibodies to the human, conducting an immunotest to determine the presence, absence and/or amount of Aspergillus antigens in a blood sample of the human using the same affinity purified human polyclonal antibodies, to monitor the efficacy of the therapeutic, removal or preventive treatment, wherein the absence or reduction in the Aspergillus antigens after administering the affinity purified human polyclonal antibodies to the human relative to the amount of Aspergillus antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Aspergillus antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Aspergillus antigens remaining after administering the affinity purified human polyclonal antibodies to the human and the extent of reduction in the Aspergillus antigens after administering the affinity purified human polyclonal antibodies to the human relative to the amount of Aspergillus antigens before the administration.

In one aspect, the invention provides a pharmaceutical composition for treating or preventing a Variola virus infection, which composition comprises an effective amount of human polyclonal antibodies affinity purified from a human blood sample with an antigenic preparation comprising at least one, preferably two or more, antigens of Variola virus.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the Variola virus antigens used in the affinity purification. In some embodiments, the affinity purified human polyclonal antibodies are substantially free of human antibodies that specifically bind to non-Variola virus antigens in said human blood sample.

The affinity purified human polyclonal antibodies can have any suitable concentrations. In some embodiments, the affinity purified human polyclonal antibodies specific to the Variola virus antigens have a concentration ranging from about 10 μg/ml to about 10 mg/ml.

In some embodiments, the human blood sample is collected from one or more normal human(s). Alternatively, the human blood sample may be collected from one or more human(s) infected with the Variola virus e.g., collected from 2, 10, 50, 100, 500, 1,000, 5,000, 10,000 or more humans.

The antigenic preparation can comprise an antigen(s) from any suitable Variola virus species, strains or serovars. In some embodiments, the antigenic preparation comprises antigen(s) from antigen(s) from Variola major and/or Variola minor.

The antigenic preparation can comprise any suitable antigen(s). In some embodiments, the Variola antigen(s) comprise A30, B7, F8 (See e.g., Sakhatskyy et al., Virology, 371(1): 98-107 (2008)), an antigen from Variola-associated DNA-dependent RNA polymerase and/or a viral surface protein, e.g., hemagglutinin. In other embodiments, the antigenic preparation comprises a Variola antigen that confers resistance to anti-viral drug or treatment.

In another aspect, the present invention provides a method for treating or preventing Variola virus infection, which method comprises administering to a human suffering, suspected of suffering or at risk of suffering from Variola virus infection, an effective amount of the pharmaceutical composition for treating or preventing Variola virus infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from Variola virus infection. In other embodiments, the human for treatment has a weakened immune system or smallpox, e.g., the smallpox caused by Variola major infection, ordinary, modified, flat, and hemorrhagic smallpox.

The present methods can be used to treat or prevent infection any suitable Variola virus species, strain or serovars. In some embodiments, the present methods can be used to treat or prevent infection caused by an Variola virus strain that is resistant to an anti-fungal drug or treatment.

In some embodiments, the present methods can further comprise, prior to administering the affinity purified human polyclonal antibodies to the human, conducting an immunotest to determine the presence, absence and/or amount of Variola antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of Variola virus infection using the affinity purified human polyclonal antibodies.

In some embodiments, the present methods can further comprise, before and after administering the affinity purified human polyclonal antibodies to the human, conducting an immunotest to determine the presence, absence and/or amount of Variola antigen(s) in a blood sample of the human using the same affinity purified human polyclonal antibodies, to monitor the efficacy of the therapeutic, removal or preventive treatment, wherein the absence or reduction in the Variola antigen(s) after administering the affinity purified human polyclonal antibodies to the human relative to the amount of Variola antigen(s) before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of Variola antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Variola antigen(s) remaining after administering the affinity purified human polyclonal antibodies to the human and the extent of reduction in the Variola antigen(s) after administering the affinity purified human polyclonal antibodies to the human relative to the amount of Variola antigen(s) before the administration.

In one aspect, the invention provides a pharmaceutical composition for treating or preventing a respiratory syncytial virus (RSV) infection, which composition comprises an effective amount of human polyclonal antibodies affinity purified from a human blood sample with an antigenic preparation comprising at least one, preferably two or more, antigens of RSV.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the RSV antigens used in the affinity purification. In some embodiments, the affinity purified human polyclonal antibodies are substantially free of human antibodies that specifically bind to non-RSV antigens in said human blood sample.

The affinity purified human polyclonal antibodies can have any suitable concentrations. In some embodiments, the affinity purified human polyclonal antibodies specific to the RSV antigens have a concentration ranging from about 10 μg/ml to about 10 mg/ml.

In some embodiments, the human blood sample is collected from one or more normal human(s). Alternatively, the human blood sample may be collected from one or more human(s) infected with the RSV e.g., collected from 2, 10, 50, 100, 500, 1,000, 5,000, 10,000 or more humans.

The antigenic preparation can comprise an antigen(s) from any suitable RSV strains or serovars. In some embodiments, the antigenic preparation comprises antigen(s) from antigen(s) from the RSV is subgroup A and B virus.

The antigenic preparation can comprise any suitable antigen(s). In some embodiments, the RSV antigen(s) comprise an antigen from a RSV protein selected from the group consisting of NS1, NS2, N (nucleocapsid protein), M (matrix protein), SH (viral coat), G (viral coat), F (viral coat), M2 (the second matrix protein), L (the RNA polymerase) and the P (phosphoprotein). In other embodiments, the antigenic preparation comprises a RSV antigen that confers resistance to anti-viral drug or treatment.

In another aspect, the present invention provides a method for treating or preventing RSV infection, which method comprises administering to a human suffering, suspected of suffering or at risk of suffering from RSV infection, an effective amount of the pharmaceutical composition for treating or preventing RSV infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from RSV infection. In other embodiments, the human for treatment or prevention has a weakened immune system or bronchiolitis. In some embodiments, the human for treatment or prevention has RSV infection caused by a RSV strain that is resistant to anti-viral drug or treatment.

In some embodiments, the present methods can further comprise, prior to administering the affinity purified human polyclonal antibodies to the human, conducting an immunotest to determine the presence, absence and/or amount of RSV antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of RSV infection using the affinity purified human polyclonal antibodies.

In some embodiments, the present methods can further comprise, before and after administering the affinity purified human polyclonal antibodies to the human, conducting an immunotest to determine the presence, absence and/or amount of RSV antigen(s) in a blood sample of the human using the same affinity purified human polyclonal antibodies, to monitor the efficacy of the therapeutic, removal or preventive treatment, wherein the absence or reduction in the RSV antigen(s) after administering the affinity purified human polyclonal antibodies to the human relative to the amount of RSV antigen(s) before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of RSV antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the RSV antigen(s) remaining after administering the affinity purified human polyclonal antibodies to the human and the extent of reduction in the RSV antigen(s) after administering the affinity purified human polyclonal antibodies to the human relative to the amount of RSV antigen(s) before the administration.

In one aspect, the invention provides a pharmaceutical composition for treating or preventing a Cytomegalovirus (CMV) infection, which composition comprises an effective amount of human polyclonal antibodies affinity purified from a human blood sample with an antigenic preparation comprising at least one, preferably two or more, antigens of CMV.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the CMV antigens used in the affinity purification. In some embodiments, the affinity purified human polyclonal antibodies are substantially free of human antibodies that specifically bind to non-CMV antigens in said human blood sample.

The affinity purified human polyclonal antibodies can have any suitable concentrations. In some embodiments, the affinity purified human polyclonal antibodies specific to the CMV antigens have a concentration ranging from about 10 μg/ml to about 10 mg/ml.

In some embodiments, the human blood sample is collected from one or more normal human(s). Alternatively, the human blood sample may be collected from one or more human(s) infected with the CMV e.g., collected from 2, 10, 50, 100, 500, 1,000, 5,000, 10,000 or more humans.

The antigenic preparation can comprise an antigen(s) from any suitable CMV strains or serovars, e.g., strains resistant to anti-viral drug or treatment. In some embodiments, the antigenic preparation comprises antigen(s) from HCMV (or Human Herpesvirus 5 or HHV-5).

The antigenic preparation can comprise any suitable antigen(s). In some embodiments, the antigenic preparation comprises an antigen from CMV glycoprotein I, glycoprotein III, a capsid protein, a coat protein or pp 65 protein. In other embodiments, the antigenic preparation comprises a CMV antigen that confers resistance to anti-viral drug or treatment.

In another aspect, the present invention provides a method for treating or preventing CMV infection, which method comprises administering to a human suffering, suspected of suffering or at risk of suffering from CMV infection, an effective amount of the pharmaceutical composition for treating or preventing CMV infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from CMV infection. In other embodiments, the human for treatment or prevention has a weakened immune system, leukemia, HIV and/or is an organ transplant recipient.

The present methods can be used to treat or prevent infection by various CMV species, e.g., the CMV infection caused by HCMV, or infection by various CMV strain, e.g., infection caused by strain AD169.

In some embodiments, the human for treatment is a fetus or infant having congenital CMV infection or perinatal CMV infection. In other embodiments, the human for treatment is an immunocompetent patient having CMV mononucleosis or post-transfusion CMV infection. In some embodiments, the human for treatment is an immunocompromised patient having CMV pneumonitis, CMV GI disease, CMV retinitis, polyradiculopathy, transverse myelitis, and/or subacute encephalitis. In some embodiments, the human for treatment has atherosclerosis. In some embodiments, the CMV infection is caused by a CMV strain that is resistant to anti-viral drug or treatment.

In some embodiments, the present methods can further comprise, prior to administering the affinity purified human polyclonal antibodies to the human, conducting an immunotest to determine the presence, absence and/or amount of CMV antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of CMV infection using the affinity purified human polyclonal antibodies.

In some embodiments, the present methods can further comprise, before and after administering the affinity purified human polyclonal antibodies to the human, conducting an immunotest to determine the presence, absence and/or amount of CMV antigen(s) in a blood sample of the human using the same affinity purified human polyclonal antibodies, to monitor the efficacy of the therapeutic, removal or preventive treatment, wherein the absence or reduction in the CMV antigen(s) after administering the affinity purified human polyclonal antibodies to the human relative to the amount of CMV antigen(s) before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of CMV antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the CMV antigen(s) remaining after administering the affinity purified human polyclonal antibodies to the human and the extent of reduction in the CMV antigen(s) after administering the affinity purified human polyclonal antibodies to the human relative to the amount of CMV antigen(s) before the administration.

In one aspect, the invention provides a pharmaceutical composition for treating or preventing a viral, bacterial, eukaryotic protist and/or fungal infection, which composition comprises at least two of the followings: 1) an effective amount of human polyclonal antibodies affinity purified from a human blood sample with an antigenic preparation comprising at least one, preferably two or more, antigens of an Influenza A virus; 2) an effective amount of human polyclonal antibodies affinity purified from a human blood sample with an antigenic preparation comprising at least one, preferably two or more, antigens of a Variola virus; 3) an effective amount of human polyclonal antibodies affinity purified from a human blood sample with an antigenic preparation comprising at least one, preferably two or more, antigens of a respiratory syncytial virus (RSV); 4) an effective amount of human polyclonal antibodies affinity purified from a human blood sample with an antigenic preparation comprising at least one, preferably two or more, antigens of a Cytomegalovirus (CMV); 5) an effective amount of human polyclonal antibodies affinity purified from a human blood sample with an antigenic preparation comprising at least one, preferably two or more, cellular and/or secreted antigens of Staphylococcus aureus (S. aureus); 6) an effective amount of human polyclonal antibodies affinity purified from a human blood sample with an antigenic preparation comprising at least one, preferably two or more, cellular and/or secreted antigens of a Streptococcus; 7) an effective amount of human polyclonal antibodies affinity purified from a human blood sample with an antigenic preparation comprising at least one, preferably two or more, cellular and/or secreted antigens of Escherichia coli (E. coli); 8) an effective amount of human polyclonal antibodies affinity purified from a human blood sample with an antigenic preparation comprising at least one, preferably two or more, cellular and/or secreted antigens of Pseudomonas aeruginosa (P. aeruginosa); 9) an effective amount of human polyclonal antibodies affinity purified from a human blood sample with an antigenic preparation comprising at least one, preferably two or more, cellular and/or secreted antigens of Acinetobacter baumannii (A. baumannii); 10) an effective amount of human polyclonal antibodies affinity purified from a human blood sample with an antigenic preparation comprising at least one, preferably two or more, cellular and/or secreted antigens of Enterococcus faecium (E. faecium); 11) an effective amount of human polyclonal antibodies affinity purified from a human blood sample with an antigenic preparation comprising at least one, preferably two or more, cellular and/or secreted antigens of Enterococcus faecalis (E. faecalis); 12) an effective amount of human polyclonal antibodies affinity purified from a human blood sample with an antigenic preparation comprising at least one, preferably two or more, cellular and/or secreted antigens of Enterobacter aerogenes (E. aerogenes); 13) an effective amount of human polyclonal antibodies affinity purified from a human blood sample with an antigenic preparation comprising at least one, preferably two or more, cellular and/or secreted antigens of Enterobacter cloacae (E. cloacae); 14) an effective amount of human polyclonal antibodies affinity purified from a human blood sample with an antigenic preparation comprising at least one, preferably two or more, cellular and/or secreted antigens of Clostridium difficile (C. difficile); 15) an effective amount of human polyclonal antibodies affinity purified from a human blood sample with an antigenic preparation comprising at least one, preferably two or more, cellular and/or secreted antigens of Klebsiella pneumoniae (K. pneumoniae); 16) an effective amount of human polyclonal antibodies affinity purified from a human blood sample with an antigenic preparation comprising at least one, preferably two or more, cellular and/or secreted antigens of a Salmonella; 17) an effective amount of human polyclonal antibodies affinity purified from a human blood sample with an antigenic preparation comprising at least one, preferably two or more, cellular and/or secreted antigens of a TB-causing Mycobacterium; 18) an effective amount of human polyclonal antibodies affinity purified from a human blood sample with an antigenic preparation comprising at least one, preferably two or more, cellular and/or secreted antigens of Bacillus anthracis; 19) an effective amount of human polyclonal antibodies affinity purified from a human blood sample with an antigenic preparation comprising at least one, preferably two or more, cellular and/or secreted antigens of Listeria monocytogenes; 20) an effective amount of human polyclonal antibodies affinity purified from a human blood sample with an antigenic preparation comprising at least one, preferably two or more, cellular and/or secreted antigens of Chlamydophila pneumoniae; 21) an effective amount of human polyclonal antibodies affinity purified from a human blood sample with an antigenic preparation comprising at least one, preferably two or more, cellular and/or secreted antigens of Ureaplasma urealyticum; 22) an effective amount of human polyclonal antibodies affinity purified from a human blood sample with an antigenic preparation comprising at least one, preferably two or more, cellular and/or secreted antigens of Mycoplasma hominis; 23) an effective amount of human polyclonal antibodies affinity purified from a human blood sample with an antigenic preparation comprising at least one, preferably two or more, cellular and/or secreted antigens of Mycoplasma pneumoniae; 24) an effective amount of human polyclonal antibodies affinity purified from a human blood sample with an antigenic preparation comprising at least one, preferably two or more, cellular and/or secreted antigens of a Plasmodium; 25) an effective amount of human polyclonal antibodies affinity purified from a human blood sample with an antigenic preparation comprising at least one, preferably two or more, cellular and/or secreted antigens of Pneumocystis jirovecii; 26) an effective amount of human polyclonal antibodies affinity purified from a human blood sample with an antigenic preparation comprising at least one, preferably two or more, cellular and/or secreted antigens of Histoplasma capsulatum; 27) an effective amount of human polyclonal antibodies affinity purified from a human blood sample with an antigenic preparation comprising at least one, preferably two or more, cellular and/or secreted antigens of Blastomyces dermatitidis; 28) an effective amount of human polyclonal antibodies affinity purified from a human blood sample with an antigenic preparation comprising at least one, preferably two or more, cellular and/or secreted antigens of a Coccidioides; 29) an effective amount of human polyclonal antibodies affinity purified from a human blood sample with an antigenic preparation comprising at least one, preferably two or more, cellular and/or secreted antigens of an Aspergillus; 30) an effective amount of human polyclonal antibodies affinity purified from a human blood sample with an antigenic preparation comprising at least one, preferably two or more, antigens cellular and/or secreted antigens of Haemophilus influenzae cells; and 31) an effective amount of human polyclonal antibodies affinity purified from a human blood sample with an antigenic preparation comprising at least one, preferably two or more, cellular and/or secreted antigens of Campylobacter jejuni cells.

In some embodiments, the affinity purified human polyclonal antibodies are purified, e.g., from about 2 fold to about 50,000 fold, e.g., about 5, 10, 50, 100, 500, 1,000, 5,000, 10,000, 20,000, 30,000, 40,000 or 50,000 fold, relative to the same human polyclonal antibodies in the unpurified or non-affinity-purified human blood sample. In some embodiments, the affinity purified human polyclonal antibodies are specific for the viral, bacterial, eukaryotic protist and/or fungal antigens used in the affinity purification. In some embodiments, the affinity purified human polyclonal antibodies are substantially free of human antibodies that specifically bind to non-viral, non-bacterial, non-eukaryotic protist and/or non-fungal antigens in said human blood sample.

The affinity purified human polyclonal antibodies can have any suitable concentrations. In some embodiments, the affinity purified human polyclonal antibodies specific to the viral, bacterial, eukaryotic protist and/or fungal antigens have a concentration ranging from about 10 μg/ml to about 10 mg/ml.

In some embodiments, the human blood sample is collected from one or more normal human(s). Alternatively, the human blood sample may be collected from one or more human(s) having the viral, bacterial, eukaryotic protist and/or fungal infection, e.g., collected from 2, 10, 50, 100, 500, 1,000, 5,000, 10,000 or more humans.

The antigenic preparation can comprise an antigen(s) from any suitable virus, bacterium, eukaryotic protist and/or fungus. In some embodiments, the antigenic preparation comprises an antigen from a virus selected from the group consisting of an influenza A virus, a Variola virus, a respiratory syncytial virus (RSV) and a Cytomegalovirus (CMV). In other embodiments, the virus is an influenza A virus. In other embodiments, the antigenic preparation comprises a viral antigen that confers resistance to an anti-viral drug or treatment.

In some embodiments, the antigenic preparation comprises an antigen from a bacterium selected from the group consisting of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae; Haemophilus influenzae, and Campylobacter jejuni. In other embodiments, the antigenic preparation comprises a bacterial antigen that confers resistance to an antibiotic or anti-bacterial drug or treatment.

The antigenic preparation can comprise a single, but often multiple bacterial antigens. In some embodiments, the antigenic preparation comprises a whole cell extract and a secreted antigen of a bacterium.

The antigenic preparation can be prepared by any suitable methods. In some embodiments, the antigenic preparation is prepared by the following steps: a) growing bacterial cells in a first protein containing culture medium; b) collecting and resuspending the bacterial cells in a second non-protein containing culture medium; c) growing the bacterial cells in the second non-protein containing culture medium; and d) disrupting the bacterial cells and collecting a whole cell extract from the disrupted bacterial cells. In other embodiments, the antigen preparation can further comprise a step of removing toxin from the whole cell extract. In other embodiments, the antigen preparation can further comprise a step of collecting a secreted antigen from the second non-protein containing culture medium in which the bacterial cells have grown.

In some embodiments, the antigenic preparation comprises an antigen from an eukaryotic protist or a fungus selected from the group consisting of a Plasmodium, Pneumocystis jirovecii, Histoplasma capsulatum, Blastomyces dermatitidis, a Coccidioides and an Aspergillus. In other embodiments, the antigenic preparation comprises an eukaryotic protist or a fungal antigen that confers resistance to an anti-protist or anti-fungal drug or treatment.

The antigenic preparation can comprise a single, but often multiple eukaryotic protist or fungal antigens. In some embodiments, the antigenic preparation comprises a whole cell extract and a secreted antigen of a an eukaryotic protist or a fungus.

The antigenic preparation can be prepared by any suitable methods. In some embodiments, the antigenic preparation is prepared by the following steps: a) growing eukaryotic protist or fungal cells in a first protein containing culture medium; b) collecting and resuspending the eukaryotic protist or fungal cells in a second non-protein containing culture medium; c) growing the eukaryotic protist or fungal cells in the second non-protein containing culture medium; and d) disrupting the eukaryotic protist or fungal cells and collecting a whole cell extract from the disrupted eukaryotic protist or fungal cells. In other embodiments, the antigen preparation can further comprise a step of removing toxin from the whole cell extract. In other embodiments, the antigen preparation can further comprise a step of collecting a secreted antigen from the second non-protein containing culture medium in which the eukaryotic protist or fungal cells have grown.

In some embodiments, the antigenic preparation comprises at least two antigens from a virus, a bacterium and/or an eukaryotic protest or a fungus. In other embodiments, the antigenic preparation comprises viral, bacterial, eukaryotic protist and fungal antigens.

In some embodiments, the pharmaceutical composition comprises at least 3, 4, 5, 6, 7, 8, 9, 10, 11, 12, 13, 14, 15, 16, 17, 18, 19, 20, 21, 22, 23, 24, 25, 26, 27, 28, 29, 30, or all 31 of the followings: 1) an effective amount of human polyclonal antibodies affinity purified from a human blood sample with an antigenic preparation comprising at least one, preferably two or more, antigens of an Influenza A virus; 2) an effective amount of human polyclonal antibodies affinity purified from a human blood sample with an antigenic preparation comprising at least one, preferably two or more, antigens of a Variola virus; 3) an effective amount of human polyclonal antibodies affinity purified from a human blood sample with an antigenic preparation comprising at least one, preferably two or more, antigens of a respiratory syncytial virus (RSV); 4) an effective amount of human polyclonal antibodies affinity purified from a human blood sample with an antigenic preparation comprising at least one, preferably two or more, antigens of a Cytomegalovirus (CMV); 5) an effective amount of human polyclonal antibodies affinity purified from a human blood sample with an antigenic preparation comprising at least one, preferably two or more, cellular and/or secreted antigens of Staphylococcus aureus (S. aureus); 6) an effective amount of human polyclonal antibodies affinity purified from a human blood sample with an antigenic preparation comprising at least one, preferably two or more, cellular and/or secreted antigens of a Streptococcus; 7) an effective amount of human polyclonal antibodies affinity purified from a human blood sample with an antigenic preparation comprising at least one, preferably two or more, cellular and/or secreted antigens of Escherichia coli (E. coli); 8) an effective amount of human polyclonal antibodies affinity purified from a human blood sample with an antigenic preparation comprising at least one, preferably two or more, cellular and/or secreted antigens of Pseudomonas aeruginosa (P. aeruginosa); 9) an effective amount of human polyclonal antibodies affinity purified from a human blood sample with an antigenic preparation comprising at least one, preferably two or more, cellular and/or secreted antigens of Acinetobacter baumannii (A. baumannii); 10) an effective amount of human polyclonal antibodies affinity purified from a human blood sample with an antigenic preparation comprising at least one, preferably two or more, cellular and/or secreted antigens of Enterococcus faecium (E. faecium); 11) an effective amount of human polyclonal antibodies affinity purified from a human blood sample with an antigenic preparation comprising at least one, preferably two or more, cellular and/or secreted antigens of Enterococcus faecalis (E. faecalis); 12) an effective amount of human polyclonal antibodies affinity purified from a human blood sample with an antigenic preparation comprising at least one, preferably two or more, cellular and/or secreted antigens of Enterobacter aerogenes (E. aerogenes); 13) an effective amount of human polyclonal antibodies affinity purified from a human blood sample with an antigenic preparation comprising at least one, preferably two or more, cellular and/or secreted antigens of Enterobacter cloacae (E. cloacae); 14) an effective amount of human polyclonal antibodies affinity purified from a human blood sample with an antigenic preparation comprising at least one, preferably two or more, cellular and/or secreted antigens of Clostridium difficile (C. difficile); 15) an effective amount of human polyclonal antibodies affinity purified from a human blood sample with an antigenic preparation comprising at least one, preferably two or more, cellular and/or secreted antigens of Klebsiella pneumoniae (K. pneumoniae); 16) an effective amount of human polyclonal antibodies affinity purified from a human blood sample with an antigenic preparation comprising at least one, preferably two or more, cellular and/or secreted antigens of a Salmonella; 17) an effective amount of human polyclonal antibodies affinity purified from a human blood sample with an antigenic preparation comprising at least one, preferably two or more, cellular and/or secreted antigens of a TB-causing Mycobacterium; 18) an effective amount of human polyclonal antibodies affinity purified from a human blood sample with an antigenic preparation comprising at least one, preferably two or more, cellular and/or secreted antigens of Bacillus anthracis; 19) an effective amount of human polyclonal antibodies affinity purified from a human blood sample with an antigenic preparation comprising at least one, preferably two or more, cellular and/or secreted antigens of Listeria monocytogenes; 20) an effective amount of human polyclonal antibodies affinity purified from a human blood sample with an antigenic preparation comprising at least one, preferably two or more, cellular and/or secreted antigens of Chlamydophila pneumoniae; 21) an effective amount of human polyclonal antibodies affinity purified from a human blood sample with an antigenic preparation comprising at least one, preferably two or more, cellular and/or secreted antigens of Ureaplasma urealyticum; 22) an effective amount of human polyclonal antibodies affinity purified from a human blood sample with an antigenic preparation comprising at least one, preferably two or more, cellular and/or secreted antigens of Mycoplasma hominis; 23) an effective amount of human polyclonal antibodies affinity purified from a human blood sample with an antigenic preparation comprising at least one, preferably two or more, cellular and/or secreted antigens of Mycoplasma pneumoniae; 24) an effective amount of human polyclonal antibodies affinity purified from a human blood sample with an antigenic preparation comprising at least one, preferably two or more, cellular and/or secreted antigens of a Plasmodium; 25) an effective amount of human polyclonal antibodies affinity purified from a human blood sample with an antigenic preparation comprising at least one, preferably two or more, cellular and/or secreted antigens of Pneumocystis jirovecii; 26) an effective amount of human polyclonal antibodies affinity purified from a human blood sample with an antigenic preparation comprising at least one, preferably two or more, cellular and/or secreted antigens of Histoplasma capsulatum; 27) an effective amount of human polyclonal antibodies affinity purified from a human blood sample with an antigenic preparation comprising at least one, preferably two or more, cellular and/or secreted antigens of Blastomyces dermatitidis; 28) an effective amount of human polyclonal antibodies affinity purified from a human blood sample with an antigenic preparation comprising at least one, preferably two or more, cellular and/or secreted antigens of a Coccidioides; 29) an effective amount of human polyclonal antibodies affinity purified from a human blood sample with an antigenic preparation comprising at least one, preferably two or more, cellular and/or secreted antigens of an Aspergillus; 30) an effective amount of human polyclonal antibodies affinity purified from a human blood sample with an antigenic preparation comprising at least one, preferably two or more, antigens cellular and/or secreted antigens of Haemophilus influenzae cells; and 31) an effective amount of human polyclonal antibodies affinity purified from a human blood sample with an antigenic preparation comprising at least one, preferably two or more, cellular and/or secreted antigens of Campylobacter jejuni cells.

The present pharmaceutical composition can comprise additional substances. In some embodiments, the present pharmaceutical composition further comprises an effective amount of affinity purified human polyclonal antibodies specific to an TNF-α antigen. In other embodiments, the present pharmaceutical composition further comprises a pharmaceutically acceptable carrier or excipient. In other embodiments, the present pharmaceutical composition further comprises additional anti-viral, anti-bacterial, anti-eukaryotic protist and/or anti-fungal substance(s) or drug(s).

In another aspect, the present invention provides a method for treating or preventing a viral, bacterial, eukaryotic protist and/or fungal infection, which method comprises administering to a human suffering, suspected of suffering or at risk of suffering from a viral, bacterial, eukaryotic protist and/or fungal infection, an effective amount of the pharmaceutical composition for treating or preventing viral, bacterial, eukaryotic protist and/or fungal infection according to the present application.

Any suitable human can be treated by the present methods. In some embodiments, the human for treatment or prevention is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the viral, bacterial, eukaryotic protist and/or fungal infection. In other embodiments, the human for treatment or prevention is infected by an viral infection caused by a viral strain that is resistant to an anti-viral drug or treatment. In other embodiments, the human for treatment or prevention is infected a bacterial infection caused by a bacterial strain that is resistant to an anti-bacterial drug or treatment. In other embodiments, the human for treatment or prevention is infected by an eukaryotic protist infection caused by an eukaryotic protist strain that is resistant to an anti-eukaryotic protist drug or treatment. In other embodiments, the human for treatment or prevention is infected a fungal infection caused by a bacterial strain that is resistant to an anti-fungal drug or treatment.

In some embodiments, the present methods can further comprise, prior to administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of viral, bacterial, eukaryotic protist and/or fungal infection using the affinity purified human polyclonal antibodies.

In some embodiments, the present methods can further comprise, before and after administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to monitor the efficacy of the therapeutic, removal or preventive treatment, wherein the absence or reduction in the viral, bacterial, eukaryotic protist and/or fungal antigens after administering the affinity purified human polyclonal antibodies to the human relative to the amount of the viral, bacterial, eukaryotic protist and/or fungal antigens before the administration indicates efficacy of the therapeutic, removal or preventive treatment.

In some embodiments, the present methods can further comprise, before and after administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the viral, bacterial, eukaryotic protist and/or fungal antigens remaining after administering the affinity purified human polyclonal antibodies to the human and the extent of reduction in the viral, bacterial, eukaryotic protist and/or fungal antigens after administering the affinity purified human polyclonal antibodies to the human relative to the amount of the viral, bacterial, eukaryotic protist and/or fungal antigens before the administration.

In some embodiments, the present methods can further comprise administering another anti-viral, anti-bacterial, anti-eukaryotic protist and/or anti-fungal drug and/or treatment.

Any suitable immunotest formats can be used in the present methods. In some embodiments, the immunotest is conducted in a format selected from an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescence assay (IFA), nephelometry, flow cytometry assay, plasmon resonance assay, chemiluminescence assay, lateral flow immunoassay, μ-capture assay, inhibition assay and avidity assay.

In one aspect, the present invention provides an immunological composition for treating or preventing bacterial infections. The immunological composition comprises an effective amount of one or more of the antigenic preparations according to the invention.

In some embodiments, the immunological composition comprises an effective amount of an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus, e.g., Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

In some embodiments, the immunological composition comprises an antigenic preparation comprising cellular and secreted antigens from at least 3, 4, 5, 6, 7, 8, 9, 10, 11, 12, 13, 14, 15, 16, 17, 18, 19, 20, or all 21 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

In some embodiments, the immunological composition comprises an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising cellular and secreted antigens from at least 3, 4, 5, 6, 7, or all 8 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising cellular and secreted antigens from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising multiple bacterial antigens. In some embodiments, the antigenic preparation comprises a whole cell extract and a secreted antigen of a bacterium. In some embodiments, the antigenic preparation comprises an endotoxin and/or an exotoxin.

In some embodiments, the immunological composition comprises an antigenic preparation comprising cellular and secreted antigens from S. aureus. In some embodiments, the immunological composition comprises an antigenic preparation comprising cellular and secreted antigens from a Streptococcus, such as S. pneumoniae. In some embodiments, the immunological composition comprises an antigenic preparation comprising cellular and secreted antigens from E. coli. In some embodiments, the immunological composition comprises an antigenic preparation comprising cellular and secreted antigens from K. pneumoniae. In some embodiments, the immunological composition comprises an antigenic preparation comprising cellular and secreted antigens from an Enterococcus, such as E. faecium or E. faecalis. In some embodiments, the immunological composition comprises an antigenic preparation comprising cellular and secreted antigens from H. influenzae. In some embodiments, the immunological composition comprises an antigenic preparation comprising cellular and secreted antigens from P. aeruginosa. In some embodiments, the immunological composition comprises an antigenic preparation comprising cellular and secreted antigens from A. baumannii.

In some embodiments, the immunological composition comprises an antigenic preparation comprising a whole cell extract and a secreted antigen from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae, Pseudomonas aeruginosa (P. aeruginosa), and Acinetobacter baumannii (A. baumannii).

In one aspect, the invention provides an immunological composition for treating cystic fibrosis, which immunological composition comprises an antigenic composition comprising cellular and secreted antigens from Pseudomonas aeruginosa (P. aeruginosa). In some embodiments, the antigenic preparation comprises a whole cell extract and a secreted antigen of P. aeruginosa.

In another aspect, the invention provides an immunological composition for treating cystic fibrosis, which immunological composition comprises an antigenic composition comprising cellular and secreted antigens from Burkholderia cepacia complex (BCC). the antigenic composition comprises cellular and secreted antigens from B. cepacia, B. multivorans, B. cenocepacia, B. vietnamiensis, B. stabilis, B. ambifaria, B. dolosa, B. anthina, and B. pyrrocinia. In some embodiments, the antigenic preparation comprises a whole cell extract and a secreted antigen of BCC. In some embodiments, the antigenic composition comprises a whole cell extract and a secreted antigen of B. cepacia, B. multivorans, B. cenocepacia, B. vietnamiensis, B. stabilis, B. ambifaria, B. dolosa, B. anthina, and B. pyrrocinia.

The antigenic preparation for the immunological composition can be prepared by any suitable methods. In some embodiments, the immunological composition comprises an antigenic preparation prepared by the following steps: a) growing bacterial cells in a first protein containing culture medium; b) collecting and resuspending the bacterial cells in a second non-protein containing culture medium; c) growing the bacterial cells in the second non-protein containing culture medium; and d) disrupting the bacterial cells and collecting a whole cell extract from the disrupted bacterial cells. In other embodiments, the antigen preparation can further comprise a step of collecting a secreted antigen from the second non-protein containing culture medium in which the bacterial cells have grown.

In one aspect, the present invention provides a vaccine for immunizing or treating a subject, preferably a human subject. The vaccine comprises an effective amount of one or more of the immunological compositions according to the invention.

In some embodiments, the vaccine further comprises an adjuvant (e.g., an aluminum salt or gel), an excipient (e.g., an antibiotic, an egg protein, a stabilizer or a preservative), or an immune response potentiator (e.g., Bacille Calmette-Guerin (BCG), Corynebacterium parvum, Brucella abortus extract, glucan, levamisole, tilorone, an enzyme, or a non-virulent virus). In some embodiments, the stabilizer is monosodium glutamate (MSG) or 2-phenoxyethanol. In some embodiments, the preservative is formaldehyde, phenoxyethanol, Thimerosal or a mercury-containing preservative.

In some embodiments, the vaccine is formulated for intravenous, intraperitoneal, intracorporeal, intra-articular, intraventricular, intrathecal, intramuscular, subcutaneous, intranasal, intravaginal, topical or oral administration. In some embodiments, the vaccine is formulated as a solid (e.g., a tablet), a semi-solid, a gel, a liquid, a semi-liquid, a skin patch, or an aerosol. In some embodiments, the vaccine is formulated for administration with a liposome, an immune stimulating complex (ISCOM), or a micro-needle.

In one aspect, the present invention provides a method for immunizing or treating a subject, which method comprises administering to a subject, preferably a human, for whom such immunization or treatment is needed or desirable, an effective amount of a vaccine according to the present invention.

In some embodiments, the method is used to immunize or treat the human from a bacterial infection, especially an antibiotic-resistant bacterial infection, a tumor, or a cancer. In some embodiments, the human is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, a patient in an emergency room, a dialysis patient, a surgery patient, e.g., a patient with elective surgery, especially orthopedic surgery patient for hip, knee, shoulder, or other body part replacement, an athlete, a healthcare worker, or a tumor or cancer patient. In some embodiments, the human suffers, is suspected of suffering, or is at risk of suffering from bacteremia.

In some embodiments, the vaccine comprises an antigenic preparation comprising cellular and secreted antigens from S. aureus, and the human suffers, is suspected of suffering, or is at risk of suffering from toxic shock syndrome (TSS), Staphylococcal scalded skin syndrome (SSSS, also known as pemphigus neonatorum or Ritter's disease, or localized bullous impetigo), pyaemia (or pyemia), a boil (or furuncle), a carbuncle, staphylococcal endocarditis, staphylococcal pneumonia or atopic dermatitis.

In some embodiments, the vaccine comprises an antigenic preparation comprising cellular and secreted antigens from a Streptococcus, especially S. pneumoniae, and the human suffers, is suspected of suffering, or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease.

In some embodiments, the vaccine comprises an antigenic preparation comprising cellular and secreted antigens from E. coli, and the human suffers, is suspected of suffering, or is at risk of suffering from gastroenteritis, a urinary tract infection, neonatal meningitis, hemolytic-uremic syndrome (HUS), peritonitis, mastitis, septicemia or Gram-negative pneumonia.

In some embodiments, the vaccine comprises an antigenic preparation comprising cellular and secreted antigens from K. pneumoniae, and the human suffers, is suspected of suffering, or is at risk of suffering from Klebsiella pneumonia, ankylosing spondylitis (AS, previously known as Bekhterev's disease, Bekhterev syndrome, and Marie-Strümpell disease, a form of Spondyloarthritis), a urinary tract infection, a patient with chronic pulmonary disease, enteric pathogenicity, nasal mucosa atrophy, and rhinoscleroma.

In some embodiments, the vaccine comprises an antigenic preparation comprising cellular and secreted antigens from E. faecium, and the human suffers, is suspected of suffering, or is at risk of suffering from neonatal meningitis.

In some embodiments, the vaccine comprises an antigenic preparation comprising cellular and secreted antigens from H. influenzae, and the human suffers, is suspected of suffering, or is at risk of suffering from bacteremia, pneumonia, acute bacterial meningitis, cellulitis, osteomyelitis, epiglottitis, infectious arthritis, ear infection (otitis media), eye infection (conjunctivitis), sinusitis or pneumonia.

In some embodiments, the vaccine comprises an antigenic preparation comprising cellular and secreted antigens from P. aeruginosa, and the human suffers, is suspected of suffering, or is at risk of suffering from pneumonia, septic shock, urinary tract infection, gastrointestinal infection, skin and soft tissue infection, infection of a burn injury, infection of an external ear (otitis externa), hot-tub rash (dermatitis), post-operative infection in a radial keratotomy surgery patient, ecthyma gangrenosum, osteomyelitis involving puncture wound of the foot. In some embodiments, the vaccine comprises an antigenic preparation comprising cellular and secreted antigens from P. aeruginosa, and the human is a cystic fibrosis patient, a neutropenic patient, a premature infant, a neutropaenic cancer patient, a burns victim or a patient with wound infection. In some embodiments, the vaccine comprises an antigenic preparation comprising cellular and secreted antigens from BCC (i.e., a combination of B. cepacia, B. multivorans, B. cenocepacia, B. vietnamiensis, B. stabilis, B. ambifaria, B. dolosa, B. anthina, and B. pyrrocinia), and the human is a cystic fibrosis patient.

In some embodiments, the vaccine comprises an antigenic preparation comprising cellular and secreted antigens from A. baumannii, and the human suffers, is suspected of suffering, or is at risk of suffering from pneumonia, infection of the urinary tract, bloodstream or other part of the body, wound, necrotizing fasciitis, or nosocomial A. baumannii bacteremia.

In some embodiments, the bacterial infection is caused by at least two different bacteria selected from the group consisting of S. aureus, E. coli, a Streptococcus, e.g., S. pneumoniae, K. pneumoniae, an Enterococcus, e.g., E. faecium, H. influenzae, P. aeruginosa, and A. baumannii. In some embodiments, the vaccine comprises an antigenic preparation comprising cellular and secreted antigens from at least two different bacteria selected from the group consisting of S. aureus, E. coli, a Streptococcus, e.g., S. pneumoniae, K. pneumoniae, an Enterococcus, e.g., E. faecium, H. influenzae, P. aeruginosa, and A. baumannii, and the bacterial infection is caused by at least two different bacteria selected from the group consisting of S. aureus, E. coli, a Streptococcus, e.g., S. pneumoniae, K pneumoniae, an Enterococcus, e.g., E. faecium, H. influenzae, P. aeruginosa, and A. baumannii.

In some embodiments, the method comprises administering the vaccine to the subject via intravenous, intraperitoneal, intracorporeal, intra-articular, intraventricular, intrathecal, intramuscular, subcutaneous, intranasal, intravaginal, topical or oral route. In some embodiments, the method comprises administering the vaccine to a tumor or cancer site (e.g., bladder or colorectal cancer, particularly a superficial form of bladder cancer). In some embodiments, the method comprises administering the vaccine to the subject as a solid (e.g., a tablet), a semi-solid, a gel, a liquid, a semi-liquid, a skin patch or an aerosol. In some embodiments, the method comprises administering the vaccine to the subject with a liposome, an immune stimulating complex (ISCOM), or a micro-needle. In some embodiments, the method comprises administering the vaccine to the subject with a pharmaceutically acceptable carrier or excipient.

In some embodiments, the method further comprises administering to the subject an additional therapeutic or preventive agent, such as an antibiotic (e.g., penicillin, a penicillinase-resistant penicillin, a glycopeptide, or an aminoglycoside), an antimicrobial agent (e.g., lysostaphin), a bactericidal agent, a bacteriostatic agent, or an immunostimulatory compound (e.g., a beta-glucan or GM-CSF). Examples of penicillinase-resistant penicillins include methicillin, oxacillin, cloxacillin, dicloxacillin and flucloxacillin Examples of aminoglycosides include kanamycin, gentamicin and streptomycin. In some embodiments, the glycopeptide is vancomycin.

In some embodiments, the method further comprises assaying S. aureus infection, a Streptococcus infection, E. coli infection, K. pneumoniae infection, E. faecium infection, H. influenzae infection, P. aeruginosa infection, and/or A. baumannii infection in the human. Any suitable assay formats can be used in the present methods. In some embodiments, the assay is conducted in a format selected from an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescence assay (IFA), nephelometry, flow cytometry assay, plasmon resonance assay, chemiluminescence assay, lateral flow immunoassay, μ-capture assay, inhibition assay and avidity assay.

### BRIEF DESCRIPTION OF THE DRAWINGS

FIGS. 1A-C show the results of an animal protection experiment, wherein BALB/C mice were infected with H1N1 virus and subsequently treated with control buffer (FIG. 1B) or with human polyclonal antibodies that were affinity purified from human blood using a mixture of forty-nine influenza A peptides having amino acid sequences of SEQ ID NOS: 1-49 (FIG. 1C). FIG. 1A shows data for untreated control animals.

### DETAILED DESCRIPTION OF THE INVENTION

### I. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are incorporated herein by reference, the definition set forth in this section prevails over the definition that is incorporated herein by reference.

As used herein, “a” or “an” means “at least one” or “one or more.”

As used herein, the term “treating or preventing” refers to any and all uses which remedy or prevent a diseased or infected state or symptoms, or otherwise deter, hinder, retard, or reverse the progression of a disease/infection or other undesirable symptoms. As used herein, the terms “treating” and “therapeutic” refer to any improvement or amelioration of any consequence of disease; full eradication of disease is not required. Amelioration of symptoms of a particular disorder refers to any lessening of symptoms, whether permanent or temporary, that can be attributed to or associated with administration of a therapeutic composition of the present invention.

As used herein, the terms “administration” or “administering” refers to any suitable method of providing a composition of the present invention of the invention to a subject. It is not intended that the present invention be limited to particular modes of administration. The affinity purified polyclonal human antibodies and pharmaceutical compositions of the present invention may be administered by oral, parenteral (e.g., intramuscular, intraperitoneal, intravenous, intracisternal injection or infusion, subcutaneous injection, or implant), inhalation spray, nasal, vaginal, rectal, sublingual, or topical routes of administration. The pharmaceutical compositions may be formulated in suitable dosage unit formulations appropriate for each route of administration.

As used herein, the term “effective amount” or “therapeutically effective amount” of an active agent refers to a nontoxic but sufficient amount of the agent to provide the desired therapeutic or prophylactic effect to most patients or individuals. It is commonly recognized that the effective amount of a pharmacologically active agent may vary depending on the route of administration, as well as the age, weight, and sex and medical conditions of the individual to which the drug or pharmacologically active agent is administered. It is also commonly recognized that one of skill in the art can determine appropriate effective amounts by taking into account such factors as metabolism, bioavailability, and other factors that affect plasma levels of an active agent following administration within the unit dose ranges disclosed further herein for different routes of administration.

As used herein, the term “immunological composition” refers to compositions capable of producing any effect on the immune system, including, without limitation, an immuno-prophylactic, immuno-therapeutic, immuno-potentiating, or immuno-suppressive effect.

As used herein, the term “vaccine” refers to refers to an antigenic suspension or solution usually comprising an immunological composition, administered into the body to produce active immunity against an infectious disease or a form of cancer mediated by an infectious organism. The antigenic portion that constitutes a vaccine can be a single macromolecular product purified from an infectious microorganism (e.g., protein, peptide, polysaccharide, etc.) or a combination of such products, including, but not limited to, a whole cell extract and secreted toxins. Accordingly, a vaccine may contain one or more antigens from one or more infectious microorganisms.

As used herein, the term “antibody” refers to monoclonal and polyclonal antibodies, whole antibodies, antibody fragments, and antibody sub-fragments that exhibit specific binding to a specific antigen of interest. Thus, “antibodies” can be whole immunoglobulin of any class, e.g., IgG, IgM, IgA, IgD and IgE. The ability of a given molecule, including an antibody fragment or sub-fragment, to act like an antibody and specifically bind to a specific antigen can be determined by binding assays known in the art, for example, using the antigen of interest as the binding partner.

As used herein, the term “specific binding” refers to the specificity of an antibody such that it preferentially binds to a defined target, such as a viral polypeptide or a cellular and/or secreted bacterial antigen. Recognition by an antibody of a particular target in the presence of other potential targets is one characteristic of such binding. Preferably, antibodies or antibody fragments that are specific for or bind specifically to a viral or bacterial antigen bind to the target viral or bacterial antigen with higher affinity than binding to other non-target antigens. Also preferably, antibodies or antibody fragments that are specific for or bind specifically to a viral or bacterial antigen avoid binding to a significant percentage of non-target, non-viral and/or non-bacterial antigens, e.g., substances used in the preparation of the viral and/or bacterial antigens. In some embodiments, antibodies or antibody fragments of the present disclosure avoid binding greater than about 90% of non-target, non-viral and/or non-bacterial antigens, although higher percentages are clearly contemplated and preferred. For example, antibodies or antibody fragments of the present disclosure avoid binding about 91%, about 92%, about 93%, about 94%, about 95%, about 96%, about 97%, about 98%, about 99%, and about 99% or more of non-target, non-viral and/or non-bacterial antigens. In other embodiments, antibodies or antibody fragments of the present disclosure avoid binding greater than about 10%, 20%, 30%, 40%, 50%, 60%, or 70%, or greater than about 75%, or greater than about 80%, or greater than about 85% of non-target, non-viral and/or non-bacterial antigens.

As used herein, the term “polyclonal antibodies” refers to a heterogeneous population of antibody molecules that bind to different antigens and/or different epitopes of the same antigen. In some embodiments, the polyclonal antibodies of the present invention bind to different antigens, different epitopes and/or different polypeptides of an influenza A virus, a Variola virus, a respiratory syncytial virus (RSV) and/or a Cytomegalovirus (CMV), and/or cellular and/or secreted antigens of a bacterium including Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or secreted antigens of an eukaryotic protist or a fungus including a Plasmodium, Pneumocystis jirovecii, Histoplasma capsulatum, Blastomyces dermatitidis, a Coccidioides and/or an Aspergillus. As used herein, the “polyclonal antibodies” specific for an Influenza A virus do not include the VH germline VH1-69.

The mixture of polyclonal antibodies includes polyclonal antibodies from a plurality of different subjects. In some contexts, the terms “individual,” “host,” “subject,” and “patient” are used interchangeably to refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the polyclonal antibodies are derived from the blood, plasma or sera of human subjects.

In some embodiments, the mixture of polyclonal antibodies can be obtained from 2, 3, 4, 5, 6, 7, 8, 9, 10, 11, 12, 13, 14, 15, 16, 17, 28, 19, 20, 21, 22, 23, 24, 25, 26, 27, 28, 29, 30, 35, 40, 45, 50, 55, 60, 65, 70, 75, 80, 85, 90, 95, 100, 500, 1,000, 5,000, 10,000 or more, individual subjects, or any number in between. In some embodiments, all of the individual subjects from whom the pool of polyclonal antibodies is obtained are infected with the target pathogenic organism. In other embodiments, some, but not all of the subjects from whom the pool of polyclonal antibodies are obtained are infected with the target pathogen. In some embodiments, none of the individuals show symptoms or clinical indications of being infected with the target pathogen. In some embodiments, some or all of the individuals have been exposed to the target pathogenic organism, but do not show the symptoms or clinical indications of being infected with the target pathogenic organism. As used herein, an individual “infected with” a target pathogen refers to individuals in which the target pathogen is present. As used herein, an individual that has been “exposed to” a target pathogen refers to an individual that was at one point in time infected with a target pathogen, but in whom the target pathogen is not necessarily still present. As discussed further below, routine diagnostic tests can be used to determine whether an individual is infected with, or has been exposed to, a target pathogen. Preferably, all or almost all of the individuals from whom the polyclonal antibodies are obtained have mounted an immune response against the target pathogen, and, as such, have plasma that contains a detectable concentration of target-specific antibodies.

As used herein, the term “antigen” refers to a target molecule that is specifically bound by an antibody through its antigen recognition site. The antigen may be monovalent or polyvalent, i.e. it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a peptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a peptide having the exact sequence of the portion of the parent protein from which it is derived, but also encompasses sequences identical to the native sequence, as well as modifications to the native sequence, such as deletions, additions and substitutions (conservative in nature).

As used herein, the term “non-viral antigen,” e.g., “non-Influenza A virus antigen,” “non-bacterial antigen,” “non-eukaryotic protist antigen,” or “non-fungal antigen” refers to a target molecule of non-viral, non-bacterial, non-eukaryotic protest or non-fungal origin. More specifically, the term “non-viral antigen,” e.g., “non-Influenza A virus antigen,” “non-bacterial antigen,” “non-eukaryotic protist antigen,” or “non-fungal antigen” refers to a substance such as protein, peptide, oligosaccharide, glycoprotein, polynucleotide or lipid that is not derived from a virus such as an influenza A virus, a Variola virus, a respiratory syncytial virus (RSV) and/or a Cytomegalovirus (CMV), a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus.

As used herein the phrase “polypeptide does not comprise any additional amino acid sequence of a naturally occurring Influenza A virus protein besides the amino acid sequences recited in SEQ ID NO:1 to SEQ ID NO:49” means that although the polypeptides may include additional amino acid residues (e.g., a cysteine linker for conjugating the polypeptides to a solid substrate), they do not include any additional amino acid sequences, i.e., contiguous strings of at least 2, 3, 4, 5, 6, 7, 8, 9, 10, 20, 30, 40, 50, 60, 70, 80, 90, 100 or more amino acid residues, of a naturally occurring Influenza A virus protein. The term “naturally occurring” as used herein refers to the fact that an object can be found in nature. For example, a polypeptide sequence that is present in an organism that can be isolated from a source in nature and which has not been intentionally modified by man in the laboratory is naturally occurring.

As used herein, the term “Influenza A virus” refers to any serotype of Influenza A virus capable of infecting a human host. The serotypes of Influenza A virus that have been confirmed in humans are: H1N1 (cause of the “Spanish Flu” in 1918-20 and the “swine flu” pandemic in 2009-10), H2N2 (cause of the “Russian Flu” in 1889-90 and the “Asian Flu” in 1957-58), H3N2 (cause of the “Hong Kong Flu” in 1968-69), H5N1 (cause of the “avian flu” pandemic threat), H7N7, H1N2, H9N2, H7N2, H7N3, H10N7, H3N2 and H5N2.

As used herein, the term “S. aureus” refers to a pathogenic strain of Staphylococcus aureus, including antibiotic resistant strains, such as methicillin resistant strains (MRSA) and vancomycin resistant strains (VISA and VRSA). In some embodiments, “S. aureus” refers to a strain that is resistant to more than one antibiotic. In some embodiments, the term “S. aureus” refers to the methicillin resistant strains USA300 (also known as FPR 3757; ATCC # BAA-1556) and NYBK2464 (ATCC # BAA-51).

As used herein, the term “Streptococcus” refers to a pathogenic strain of Streptococcus pneumoniae, Group A Streptococcus (GAS; e.g., Streptococcus pyogenes) and Group B Streptococcus (GBS; e.g., Streptococcus agalactiae), including antibiotic-resistant strains, such as S. pneumoniae strains resistant to penicillin, tetracycline, clindamycin, a cephalosporin, a macrolide or a quinolone. In some embodiments, “Streptococcus” refers to the GAS strain ATCC # 19615 and the GBS strain ATCC # 25663.

As used herein, the term “E. coli” refers to a pathogenic strain of Escherichia coli, including antibiotic resistant strains, such as E. coli strains resistant to penicillin, streptomycin, chloramphenicol, ampicillin, cephalosporin or tetracycline. As used herein, “E. coli” encompasses enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC). In some embodiments, “E. coli” refers to a Shiga toxin-producing E. coli (STEC), such as the strain O157:H7 (ATCC # 43895).

As used herein, the term “P. aeruginosa” refers to a pathogenic strain of Pseudomonas aeruginosa, including antibiotic-resistant strains, such as P. aeruginosa strains resistant to β-lactam antibiotics (e.g., penicillin), piperacillin, imipenem, tobramycin or ciprofloxacin. In some embodiments, “P. aeruginosa” may refer to the strains identified as ATCC # 9027, ATCC # 10145 or ATCC # 15442. In some embodiments, the term “P. aeruginosa” refers to a pathogenic strain that infects cystic fibrosis patients.

As used herein, the term “A. baumannii” refers to a pathogenic strain of Acinetobacter baumannii, including any antibiotic-resistant strains, such as A. baumannii strains resistant to ceftazidime, gentamicin, ticarcillin, piperacillin, aztreonam, cefepime, ciprofloxacin, imipenem or meropenem. In some embodiments, “A. baumannii” may refer to the strain identified as ATCC # BAA-1605.

As used herein, the term “E. faecium” refers to a pathogenic strain of Enterococcus faecium, including antibiotic-resistant strains, such as E. faecium strains resistant to β-lactam antibiotics (e.g., penicillins and cephalosporins) or aminoglycosides. In some embodiments, “E. faecium” may refer to the strain identified as ATCC # 51559.

As used herein, the term “E. faecalis” refers to a pathogenic strain of Enterococcus faecalis, including antibiotic-resistant strains, such as E. faecalis strains resistant to aminoglycosides, penicillins, cephalosporins, aztreonam, clindamycin or vancomycin. In some embodiments, “E. faecalis” may refer to the strains identified as ATCC # 29212 or ATCC # 51299.

As used herein, the term “E. aerogenes” refers to a pathogenic strain of Enterobacter aerogenes, including antibiotic-resistant strains, such as E. aerogenes strains resistant to penicillins, cephalosporins, carbapenems, aminoglycosides, ciprofloxacin, trimethoprim-sufamethoxazole or quinolones. In some embodiments, “E. aerogenes” may refer to the strain identified as ATCC # 13048.

As used herein, the term “E. cloacae” refers to a pathogenic strain of Enterobacter cloacae, including antibiotic-resistant strains, such as E. cloacae strains resistant to penicillins, cephalosporins, carbapenems, aminoglycosides, ciprofloxacin, trimethoprim-sufamethoxazole or quinolones. In some embodiments, “E. cloacae” may refer to the strain identified as ATCC # 13047.

As used herein, the term “C. difficile” refers to a pathogenic strain of Clostridium difficile, including any antibiotic-resistant strains. In some embodiments, “C. difficile” may refer to the strains identified as ATCC # 9689 or ATCC # BAA-1382.

As used herein, the term “K. pneumoniae” refers to a pathogenic strain of Klebsiella pneumoniae, including any antibiotic-resistant strains, such as extended-spectrum β-lactamase (ESBL)-producing K. pneumoniae strains that are resistant to aminoglycosides, penicillins or cephalosporins (e.g., ceftazidime or carbapenem). In some embodiments, “K. pneumoniae” may refer to the strains identified as ATCC # 13883, ATCC # BAA-1705 or ATCC # 700603.

As used herein, the term “Salmonella” refers to a pathogenic strain of Salmonella, including any antibiotic-resistant strains. In some embodiments, “Salmonella” refers to Salmonella bongori (S. bongori) and/or Salmonella enterica (S. enterica). In some embodiments, S. enterica refers to Salmonella enterica enterica, Salmonella enterica salamae, Salmonella enterica arizonae, Salmonella enterica diarizonae, Salmonella enterica houtenae, and/or Salmonella enterica indica. In some embodiments, the Salmonella enterica enterica has serovars selected from the group consisting of Salmonella Choleraesuis, Salmonella Dublin, Salmonella Enteritidis, Salmonella Gallinarum, Salmonella Hadar, Salmonella Heidelberg, Salmonella Infantis, Salmonella Paratyphi, Salmonella Typhi and Salmonella Typhimurium. In some embodiments, “Salmonella” refers to an antibiotic resistant strain, such as Salmonella Typhimurium DT104 (DT104), multidrug-resistant typhoid (MDR typhoid), or MDR-AmpC.

As used herein, the term “Mycobacterium” refers to a pathogenic strain of Mycobacterium, including any antibiotic-resistant strains. In some embodiments, “Mycobacterium” refers to Mycobacterium tuberculosis (MTB), M. bovis, M. africanum, M. canetti, and/or M. microti. In some embodiments, “Mycobacterium” refers to a hypervirulent strain of M. tuberculosis. In some embodiments, “Mycobacterium” refers to antibiotic resistant strains, such as multidrug-resistant tuberculosis (MDR-TB) or extensively drug-resistant TB (XDR-TB).

As used herein, the term “Bacillus anthracis” refers to a pathogenic strain of Bacillus anthracis, including any antibiotic-resistant strains. In some embodiments, “Bacillus anthracis” refers to the Ames strain, the Vollum strain (also incorrectly referred to as Vellum) strain and/or the Vollum 1B strain.

As used herein, the term “Haemophilus influenzae” refers to a pathogenic strain of Haemophilus influenzae, including any antibiotic-resistant strains. In some embodiments, “Haemophilus influenzae” refers to an unencapsulated strain or an encapsulated strain, such as the serotype a, b, c, d, e, or f.

As used herein, the term “Campylobacter jejuni” refers to a pathogenic strain of Campylobacter jejuni, including any antibiotic-resistant strains. In some embodiments, “Campylobacter jejuni” refers to strain NCTC11168.

As used herein, the term “Listeria monocytogenes” refers to a pathogenic strain of Listeria monocytogenes, including any antibiotic-resistant strains. In some embodiments, “Listeria monocytogenes” refers to Listeria monocytogenes having one of the following serotypes: 1/2a, 1/2b, and/or 4b. In some embodiments, “Listeria monocytogenes” refers to antibiotic resistant strains, such as BM4210 or BM4293.

As used herein, the term “Chlamydophila pneumoniae” refers to a pathogenic strain of Chlamydophila pneumoniae, including any antibiotic-resistant strains. In some embodiments, “Chlamydophila pneumoniae” refers to strain TWAR, A-03, BAL-16, TW-183,T-2634 or AR-39.

As used herein, the term “Ureaplasma urealyticum” refers to a pathogenic strain of Ureaplasma urealyticum, including any antibiotic-resistant strains. In some embodiments, “Ureaplasma urealyticum” refers to Ureaplasma urealyticum having serovars (serotypes) 1 to 14.

As used herein, the term “Mycoplasma hominis” refers to a pathogenic strain of Mycoplasma hominis, including any antibiotic-resistant strains. In some embodiments, “Mycoplasma hominis” refers to strain 1620, 2101, PG21, LBD4, r. Taub, W1458, 1611, F4238, M5039, H5488, 11085, 13428, 1184, 1888, 11932 or 13408.

As used herein, the term “Mycoplasma pneumoniae” refers to a pathogenic strain of Mycoplasma pneumoniae, including any antibiotic-resistant strains. In some embodiments, “Mycoplasma pneumoniae” refers to strain M129 (ATCC # 29342), FH or MPN371

As used herein, the term “Plasmodium” refers to a pathogenic strain of Plasmodium, including any antibiotic-resistant strains. In some embodiments, “Plasmodium” refers to P. falciparum, P. malariae, P. ovale, P. vivax and P. knowlesi, P. inui, P. cynomolgi, P. simiovale, P. brazilianum, P. schwetzi and P. simium.

As used herein, the term “Pneumocystis jirovecii” refers to a pathogenic strain of Pneumocystis jirovecii, including any antibiotic-resistant strains, particularly Pneumocystis jirovecii strains that are resistant to sulfa drugs.

As used herein, the term “Histoplasma capsulatum” refers to a pathogenic strain of Histoplasma capsulatum, including any antibiotic-resistant strains. In some embodiments, “Histoplasma capsulatum” refers to Histoplasma capsulatum var. capsulatum. In some embodiments, “Histoplasma capsulatum” refers to Histoplasma capsulatum strains that are resistant to an anti-fungal drug or treatment, e.g., fluconazole.

As used herein, the term “Blastomyces dermatitidis” refers to a pathogenic strain of Blastomyces dermatitidis, including any antibiotic-resistant strains. In some embodiments, “Blastomyces dermatitidis” refers to strain SLH-14081, ER-3, ATCC # 18188 or ATCC # 26199.

As used herein, the term “Coccidioides” refers to a pathogenic strain of Coccidioides, including any antibiotic-resistant strains. In some embodiments, “Coccidioides” refers to C. immitis or C. posadasii.

As used herein, the term “Aspergillus” refers to a pathogenic strain of Aspergillus, including any antibiotic-resistant strains. In some embodiments, “Aspergillus” refers to A. fumigatus, A. flavus or A. clavatus.

As used herein, the term “Variola” refers to a pathogenic strain of Variola, including any antiviral-resistant strains. In some embodiments, “Variola” refers to Variola major and/or Variola minor.

As used herein, the term “RSV” refers to a pathogenic strain of respiratory syncytial virus, including any antiviral-resistant strains. In some embodiments, “RSV” refers to subgroup A and B RSV.

As used herein, the term “CMV” refers to a pathogenic strain of cytomegalovirus, including any antiviral-resistant strains. In some embodiments, “CMV” refers to HCMV (also referred to as Human Herpesvirus 5 or HHV-5).

As used herein, the term “antigenic preparation comprising cellular and/or secreted antigen(s)” refers to a preparation comprising any antigen(s) secreted by cells of a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus, and/or any cellular, e.g., soluble, antigens produced by the disruption of such cells using any physical and/or chemical means. Thus, the term encompasses soluble bacterial cell extracts, including whole cell extracts or cell surface or membrane extracts. In some embodiments, the “antigenic preparation” does not include intact bacterial, eukaryotic protist or fungal cells or insoluble particulate matter, such as walls or nuclei of bacterial, eukaryotic protist or fungal cells. In some embodiments, the antigenic preparation may comprise secreted bacterial, eukaryotic protist or fungal toxin(s), oligosaccharide(s), protein(s), peptide(s), lipid(s), and other soluble cellular component(s). In some embodiments, the antigenic preparation may comprise secreted toxin(s), oligosaccharide(s), protein(s), peptide(s) and glycoprotein(s) from a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus. In some embodiments, the “antigenic preparation” comprises a single cellular antigen and/or a single secreted antigen. In other embodiments, the “antigenic preparation” comprises a single cellular antigen and/or multiple secreted antigens. In still other embodiments, the “antigenic preparation” comprises multiple cellular antigens and/or a single secreted antigen. In yet other embodiments, the “antigenic preparation” comprises multiple cellular antigens and/or multiple secreted antigens.

As used herein, the term “whole cell extract” refers to any cellular components of cells of a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus, that remain in extraction solution, e.g., an aqueous solution or a non-aqueous solution, following a physical or chemical disruption of the bacterial cells. “Whole cell extract” is not meant to encompass intact bacterial, eukaryotic protist or fungal cells and insoluble components, such as bacterial, eukaryotic protist or fungal walls and nuclei that can be removed from the extraction solution by any suitable methods, such as filtration or centrifugation. In some embodiments, the whole cell extract may contain soluble proteins, glycoproteins, peptides, oligosaccharides, lipids, polynucleotides from S. aureus, a Streptococcus, E. coli, P. aeruginosa, A. baumannii, E. faecium, E. faecalis, E. aerogenes, E. cloacae, C. difficile and/or K. pneumoniae .

As used herein, the term “human blood sample” refers to whole blood, plasma or serum obtained from one or more human subjects. “Whole blood” refers to the fluid and cellular portion of the plasma in circulating blood. “Plasma” refers to the fluid, non-cellular portion of the blood, distinguished from the serum obtained after coagulation. “Serum” refers to the fluid portion of the blood obtained after removal of the fibrin clot and blood cells, distinguished from the plasma in circulating blood. In some embodiments, “human blood sample” refers to a serum sample obtained from a normal human subject. In some embodiments, serum samples from multiple human subjects, preferably normal humans, are pooled in order to generate greater diversity of polyclonal antibodies.

As used herein, the term “normal human” or “healthy individual” refers to a human subject that is not hyperimmune to a virus, such as an influenza A virus, a Variola virus, a respiratory syncytial virus (RSV) and a Cytomegalovirus (CMV), a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus, as a result of vaccination, especially recent vaccination, against these microorganisms, or recent exposure, especially an infection that led to bacteremia, to these microorganisms.

As used herein, the term “substantially free of human antibodies that specifically bind to” non-viral, non-bacterial, non-eukaryotic protist and/or non-fungal antigens refers to a composition of affinity purified polyclonal human antibodies that contains no more than about 90%, 80%, 70%, 60%, 50%, 40%, or 30%, preferably no more than about 20%, more preferably no more than about 10% and most preferably no more than about 5% of antibodies that specifically bind to non-viral, non-bacterial, non-eukaryotic protist and/or non-fungal antigens. As explained above, the terms non-viral, non-bacterial, non-eukaryotic protist and/or non-fungal antigens as used herein usually refer to polypeptides, oligosaccharides, glycoproteins, polynucleotides or lipids derived from sources other than a virus, such as an influenza A virus, a Variola virus, a respiratory syncytial virus (RSV) and a Cytomegalovirus (CMV), a bacterium such as Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, and/or an eukaryotic protist or a fungus such as a Plasmodium, Pneumocystis jirovecii, Histoplasma capsulatum, Blastomyces dermatitidis, a Coccidioides and/or an Aspergillus.

As used herein, “bacteremia” refers to the presence of viable bacteria and/or bacterial toxin(s) in the bloodstream of a human subject. “Bacteremia caused by S. aureus” or “S. aureus bacteremia” refers to bacteremia in which at least some of the bacteria in the blood are S. aureus. Other bacterial species, such as a Streptococcus, E. coli, P. aeruginosa, A. baumannii, E. faecium, E. faecalis, E. aerogenes, E. cloacae, C. difficile and/or K. pneumoniae, also may be present in the bloodstream.

As used herein, the term “substantially removed in the antigenic preparation” generally refers to an antigenic preparation in which more than about 10%, 20%, 30%, 40%, 50%, 60%, 70%, preferably more than about 80%, more preferably more than about 90% and most preferably more than about 95% of a recited component has been removed. For example, the phrase “S. aureus Protein A is substantially removed in the antigenic preparation” means that more than about 70%, preferably more than about 80%, more preferably more than about 90% and most preferably more than about 95% of Protein A has been removed. Because S. aureus Protein A is a gamma globulin (IgG) binding protein which binds to the non-variable Fc region of an antibody, its effective removal is important to ensure that the antigenic preparation is substantially free of human antibodies that specifically bind to non-bacterial antigens.

As used herein, the phrase “substantially inactivating and/or removing a virus” generally refers to an antigenic preparation in which more than about 10%, 20%, 30%, 40%, 50%, 60%, 70%, preferably more than about 80%, more preferably more than about 90% and most preferably more than about 95% of a recited or target virus has been removed.

As used herein, the term “capsular polysaccharide” refers to a layer of polysaccharide external to but contiguous with the cell wall of a microorganism. Capsular polysaccharides are distinct from lipopolysaccharides (LPS) and the polysaccharides derived therefrom. The term “lipopolysaccharide” is commonly used to refer to the endotoxic component of the outer membrane in Gram negative bacteria.

As used herein, the term “toxin” refers to any cytotoxic molecule secreted from bacterial cells or associated with the bacterial cell wall. The secreted toxins are commonly referred to as “exotoxins,” and the cell-associated toxins are referred to as “endotoxins.” Most endotoxins are located in the cell envelope. As used herein, endotoxins refer specifically to the lipopolysaccharide (LPS) or lipooligosaccharide (LOS) located in the outer membrane of Gram-negative bacteria. Although they are structural components of bacterial cells, soluble endotoxins may be released from growing bacteria or from cells that are lysed as a result of host defense mechanisms or by the activities of certain antibiotics. Endotoxins generally act in the vicinity of bacterial growth or presence. In contrast, exotoxins are usually secreted by bacteria and act at a site removed from bacterial growth. However, in some cases, exotoxins are only released by lysis of the bacterial cell. Exotoxins are usually proteins or polypeptides that act enzymatically or through direct action with host cells and stimulate a variety of responses.

As used herein, the term “protein containing culture medium” refers to any suitable bacterial growth medium that contains a protein, peptide and/or amino acid nutrient, such as a yeast extract, tryptone, casein peptone, and the like. As used herein, a protein containing culture medium is used to grow bacterial cells to a desired density, after which it is substituted with a protein-free culture medium in order to avoid the binding of human antibodies to non-bacterial antigens associated with the protein-containing culture medium. In some embodiments, the term “protein containing culture medium” refers to Acumedia™ TSB, containing 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Neogen Cat No. 7729, Lansing, Mich.). In some embodiments, the term “protein containing culture medium” refers to Acumedia™ THB, containing 3.1 g/L heart infusion (dehydrated), 20.0 g/L yeast enriched peptone, 2.0 g/L dextrose, 2.0 g/L sodium chloride, 0.4 g/L disodium phosphate, and 2.5 g/L sodium carbonate, pH 7.8±0.2. (Neogen Cat No. 7110, Lansing Mich.). In some embodiments, the term “protein containing culture medium” refers to Bacto™ Tryptic Soy Broth containing 17.0 g/L pancreatic digest of casein; 3.0 g/L enzymatic digest of soybean meal, 5.0 g/L NaCl, 2.5 g/L K₂HPO₄ and 2.5 g/L dextrose (VWR Cat. No. 90000-378; Becton Dickinson Cat. No. 211825; 30% w/v in de-ionized H₂O). In some embodiments, the term “protein containing culture medium” refers to Difco™ Reinforced Clostridial Media containing 5.0 g/L pancreatic digest of casein, 5.0 g/L proteose peptone #3, 10.0 g/L beef extract, 3.0 g/L yeast extract, 5.0 g/L NaCl, 1.0 g/L soluble starch, 5.0 g/L dextrose, 0.5 g/L cysteine HCl and 3.0 g/L sodium acetate (Becton Dickinson Cat. No. 218081; 38% w/v in de-ionized H₂O).

As used herein, the term “non-protein containing culture medium” refers to any suitable minimal bacterial growth medium that does not contain a biologically significant amount of proteins, peptides and/or amino acids. Such a minimal bacterial growth medium usually contains water, a source of carbon (e.g., a sugar such as glucose, or a less energy-rich source such as succinate) and various salts (e.g., sodium chloride, sodium phosphate). In is understood that the composition of a non-protein containing culture medium may vary depending on the bacterial species. In some embodiments, “non-protein containing culture medium” refers to a phosphate-buffered 0.9% NaCl solution (Baxter Cat. No. 2F7125) supplemented with 2 g/L D-(+)-glucose (dextrose) (Sigma Cat. No. G5146).

As used herein, the term “insoluble cellular debris” refers to those bacterial cellular components that are insoluble in an extraction solution, e.g., an aqueous solution or a non-aqueous solution, following a physical or chemical disruption of bacterial cells. Although the term typically encompasses bacterial cell wall and bacterial nuclei, it also refers to any other bacterial components that can be filtered out or precipitated from an extraction solution following a bacterial cell disruption.

As used herein, the term “precipitation or agglutination assay” refers to an immunotest format wherein the interaction between an antibody and a particular antigen results in visible precipitation or clumping. Precipitation reactions are similar in principle to agglutination reactions; they depend on the cross linking of polyvalent antigens. When the antigen is soluble, antibody and antigen form a lattice that eventually develops into a visible precipitate. When the antigen is particulate, the reaction of an antibody with the antigen can be detected by agglutination (clumping) of the antigen. It is commonly understood that both precipitation and agglutination assays can be qualitative or quantitative.

As used herein, the term “pharmaceutical excipient” refers to a material such as an adjuvant, a carrier, pH-adjusting and a buffering agent, a tonicity adjusting agent, a wetting agent, a preservative, and the like.

As used herein, the term “pharmaceutically acceptable” refers to a non-toxic, inert composition that is physiologically compatible with humans or other mammals.

As used herein, the term “pharmaceutically acceptable formulation” or “pharmaceutical composition” refers to a composition or formulation that allows for the effective distribution of a moiety or a compound, e.g., an antibody, of the invention in that physical location most suitable for their desired activity.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### II. Antigenic Compositions

The influenza A genome contains 11 genes on eight pieces of RNA, encoding for 11 proteins: hemagglutinin (HA), neuraminidase (NA), nucleoprotein (NP), matrix protein 1 (M1), matrix protein 2 (M2), non-structural protein 1 (NS1), non-structural protein 2 (NS2 or NEP), polymerase A (PA), polymerase B1 (PB1 and PB1-F2) and polymerase B2 (PB2). Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. These proteins are both targets for antiviral drugs and antigens to which antibodies are most commonly raised. As noted above, Influenza A viruses are classified into subtypes based on antibody responses to HA and NA. These different types of HA and NA form the basis of the H and N distinctions in, for example, the H1N1 or H5N1 serotypes.

As discussed above, the present invention is concerned with antigenic compositions for affinity purification of human polyclonal antibodies that are specific for Influenza A virus. Although HA and NA are the most common antigens used for active immunization against Influenza A virus, human blood contains numerous polyclonal antibodies against all the structural and non-structural proteins of the Influenza A virus. These antibodies can be used in combination for passive immunization against influenza.

In one aspect, the present invention provides an antigenic composition comprising at least one, preferably two, Influenza A virus polypeptides, wherein each of the polypeptides comprises an amino acid sequence selected from the following amino acid sequences:

a) polymerase B1 (PB 1) sequence, from N-terminus to C-terminus, DAVATTHSWIPKRNRSIL (SEQ ID NO:1),

b) PB1 sequence, from N-terminus to C-terminus, FLKDVMESM (SEQ ID NO:2),

c) PB1 sequence, from N-terminus to C-terminus, FNMLSTVLGV (SEQ ID NO:3),

d) PB1 sequence, from N-terminus to C-terminus, FSMELPSFGV (SEQ ID NO:4),

e) PB1 sequence, from N-terminus to C-terminus, GPATAQMAL (SEQ ID NO:5),

f) PB1 sequence, from N-terminus to C-terminus, DTVNRTHQY (SEQ ID NO:6),

g) polymerase B2 (PB2) sequence, from N-terminus to C-terminus, YMLERELVRKTRFLPVA (SEQ ID NO:7),

h) PB2 sequence, from N-terminus to C-terminus, NFVNRANQRLNPMHQLLR (SEQ ID NO:8),

i) polymerase A (PA) sequence, from N-terminus to C-terminus, FMYSDFHFI (SEQ ID NO:9),

j) PA sequence, from N-terminus to C-terminus, RSKFLLMDALKLSIE (SEQ ID NO:10),

k) PA sequence, from N-terminus to C-terminus, SVKEKDMTK (SEQ ID NO:11),

l) PA sequence, from N-terminus to C-terminus, MRRNYFTAEVSHCRATEY (SEQ ID NO:12),

m) PA sequence, from N-terminus to C-terminus, AESRKLLLI (SEQ ID NO:13),

n) hemagglutinin (HA) sequence, from N-terminus to C-terminus, GLFGAIAGFC (SEQ ID NO:14),

o) HA sequence, from N-terminus to C-terminus, GLFGAIAGFI (SEQ ID NO:15),

p) HA sequence, from N-terminus to C-terminus, TGMVDGWYGYHHQNEQGS (SEQ ID NO:16),

q) HA sequence, from N-terminus to C-terminus, WTYNAELLVLLENERTLD (SEQ ID NO:17),

r) HA sequence, from N-terminus to C-terminus, NKVNSVIEKMNTQFTAVG (SEQ ID NO:18),

s) HA sequence, from N-terminus to C-terminus, GLFGAIAGFIE (SEQ ID NO:19),

t) HA sequence, from N-terminus to C-terminus, YPYDVPDYA (SEQ ID NO:20),

u) HA sequence, from N-terminus to C-terminus, VTGLRNIPSIQCR (SEQ ID NO:21),

v) HA sequence, from N-terminus to C-terminus, SVSSFERFEIFPK (SEQ ID NO:22),

w) nucleoprotein (NP) sequence, from N-terminus to C-terminus, RRSGAAGAAVK (SEQ ID NO:23),

x) NP sequence, from N-terminus to C-terminus, QLVWMACHSAA (SEQ ID NO:24),

y) NP sequence, from N-terminus to C-terminus, YERMCNILKG (SEQ ID NO:25),

z) NP sequence, from N-terminus to C-terminus, TYQRTRALV (SEQ ID NO:26),

aa) NP sequence, from N-terminus to C-terminus, RMVLSAFDER (SEQ ID NO:27),

bb) NP sequence, from N-terminus to C-terminus, LELRSRYWAI (SEQ ID NO:28),

cc) NP sequence, from N-terminus to C-terminus, KLSTRGVQIASNEN (SEQ ID NO:29),

dd) neuraminidase (NA) sequence, from N-terminus to C-terminus, SWPDGAELPF (SEQ ID NO:30),

ee) NA sequence, from N-terminus to C-terminus, PIRGWAI (SEQ ID NO: 31),

ff) NA sequence, from N-terminus to C-terminus, SGSFVQHPELTGL (SEQ ID NO:32),

gg) NA sequence, from N-terminus to C-terminus, VGLISLILQI (SEQ ID NO:33),

hh) matrix protein 1 (M1) sequence, from N-terminus to C-terminus, KTRPILSPLTK (SEQ ID NO:34),

ii) M1 sequence, from N-terminus to C-terminus, QKRMGVQMQRFK (SEQ ID NO:35),

jj) M1 sequence, from N-terminus to C-terminus, AGKNTDLEALMEWLKTR (SEQ ID NO:36),

kk) M1 sequence, from N-terminus to C-terminus, IRHENRMVL (SEQ ID NO:37),

ll) M1 sequence, from N-terminus to C-terminus, GILGFVFTL (SEQ ID NO:38),

mm) M1 sequence, from N-terminus to C-terminus, SLLTEVETYVL (SEQ ID NO:39),

nn) M1 sequence, from N-terminus to C-terminus, KGILGFVFTLTVPSE (SEQ ID NO:40),

oo) M1 sequence, from N-terminus to C-terminus, ILSPLTKGIL (SEQ ID NO:41),

pp) M1 sequence, from N-terminus to C-terminus, RMVLASTTAKAMEQM (SEQ ID NO:42),

qq) matrix protein 2 (M2) sequence, from N-terminus to C-terminus, SLLTEVET (SEQ ID NO:43),

rr) M2 sequence, from N-terminus to C-terminus, EVETPIRN (SEQ ID NO:44),

ss) non-structural protein 1 (NS1) sequence, from N-terminus to C-terminus, GEISPLPSL (SEQ ID NO:45),

tt) NS1 sequence, from N-terminus to C-terminus, DRLRRDQKS (SEQ ID NO:46),

uu) NS1 sequence, from N-terminus to C-terminus, AIMDKNIIL (SEQ ID NO:47),

vv) non-structural protein 2 (NS2) sequence, from N-terminus to C-terminus, ITFMQALQLL (SEQ ID NO:48), and

ww) NS2 sequence, from N-terminus to C-terminus, RTFSFQLI (SEQ ID NO:49).

In some embodiments, these polypeptides may include one or more amino acid residues in addition to the amino acid sequences recited in SEQ ID NO:1 to SEQ ID NO:49. However, the polypeptides do not include any additional amino acid sequences of a naturally occurring Influenza A virus protein besides the amino acid sequences recited in SEQ ID NO:1 to SEQ ID NO:49.

In some embodiments, the composition includes at least 5, 10, 15, 20, 25, 30, 35, 40, 45, or all 49 of the Influenza A virus polypeptides, each comprising the amino acid sequences recited in SEQ ID NO:1 to SEQ ID NO:49 and not including any additional amino acid sequences of a naturally occurring Influenza A virus protein. In some embodiments, at least one of the Influenza A virus polypeptides in the composition consists essentially of an amino acid sequence selected from SEQ ID NO:1 to SEQ ID NO:49. In some embodiments, at least 5, 10, 15, 20, 25, 30, 35, 40, 45, or all 49 of the Influenza A virus polypeptides in the composition consist essentially of amino acid sequences selected from SEQ ID NO:1 to SEQ ID NO:49. In some embodiments, at least one of the Influenza A virus polypeptides in the composition consists of an amino acid sequence selected from SEQ ID NO:1 to SEQ ID NO:49. In some embodiments, at least 5, 10, 15, 20, 25, 30, 35, 40, 45, or all 49 of the Influenza A virus polypeptides in the composition consist of amino acid sequences selected from SEQ ID NO:1 to SEQ ID NO:49.

In a related aspect, the invention provides an antigenic composition comprising at least one, preferably two, Influenza A virus polypeptides, wherein each of the polypeptides comprises an amino acid sequence selected from the following amino acid sequences:

a) polymerase B1 (PB1) sequence, from N-terminus to C-terminus, FLKDVMESM (SEQ ID NO:2),

b) PB1 sequence, from N-terminus to C-terminus, FNMLSTVLGV (SEQ ID NO:3),

c) PB1 sequence, from N-terminus to C-terminus, FSMELPSFGV (SEQ ID NO:4),

d) polymerase B2 (PB2) sequence, from N-terminus to C-terminus, YMLERELVRKTRFLPVA (SEQ ID NO:7),

e) PB2 sequence, from N-terminus to C-terminus, NFVNRANQRLNPMHQLLR (SEQ ID NO:8),

f) polymerase A (PA) sequence, from N-terminus to C-terminus, MRRNYFTAEVSHCRATEY (SEQ ID NO:12),

g) PA sequence, from N-terminus to C-terminus, AESRKLLLI (SEQ ID NO:13),

h) hemagglutinin (HA) sequence, from N-terminus to C-terminus, GLFGAIAGFC (SEQ ID NO:14),

i) HA sequence, from N-terminus to C-terminus, TGMVDGWYGYHHQNEQGS (SEQ ID NO:16),

j) HA sequence, from N-terminus to C-terminus, WTYNAELLVLLENERTLD (SEQ ID NO:17),

k) HA sequence, from N-terminus to C-terminus, NKVNSVIEKMNTQFTAVG (SEQ ID NO:18),

l) HA sequence, from N-terminus to C-terminus, VTGLRNIPSIQCR (SEQ ID NO:21),

m) nucleoprotein (NP) sequence, from N-terminus to C-terminus, RRSGAAGAAVK (SEQ ID NO:23),

n) NP sequence, from N-terminus to C-terminus, QLVWMACHSAA (SEQ ID NO:24),

o) NP sequence, from N-terminus to C-terminus, YERMCNILKG (SEQ ID NO:25),

p) NP sequence, from N-terminus to C-terminus, KLSTRGVQIASNEN (SEQ ID NO:29),

q) neuraminidase (NA) sequence, from N-terminus to C-terminus, SWPDGAELPF (SEQ ID NO:30),

r) NA sequence, from N-terminus to C-terminus, PIRGWAI (SEQ ID NO: 31),

s) NA sequence, from N-terminus to C-terminus, SGSFVQHPELTGL (SEQ ID NO:32),

t) NA sequence, from N-terminus to C-terminus, VGLISLILQI (SEQ ID NO:33),

u) matrix protein 1 (M1) sequence, from N-terminus to C-terminus, KTRPILSPLTK (SEQ ID NO:34),

v) M1 sequence, from N-terminus to C-terminus, QKRMGVQMQRFK (SEQ ID NO:35),

w) M1 sequence, from N-terminus to C-terminus, AGKNTDLEALMEWLKTR (SEQ ID NO:36), and

x) non-structural protein 2 (NS2) sequence, from N-terminus to C-terminus, ITFMQALQLL (SEQ ID NO:48).

In some embodiments, these polypeptides may include one or more amino acid residues in addition to the amino acid sequences recited in parts a) to x). However, the polypeptides do not include any additional amino acid sequences of a naturally occurring Influenza A virus protein besides the amino acid sequences recited in parts a) to x).

In some embodiments, the composition includes at least 5, 10, 15, 20, or all 24 of the Influenza A virus polypeptides, each comprising the amino acid sequences recited in parts a) to x) and not including any additional amino acid sequences of a naturally occurring Influenza A virus protein. In some embodiments, at least one of the Influenza A virus polypeptides in the composition consists essentially of an amino acid sequence selected from parts a) to x). In some embodiments, at least 5, 10, 15, 20, or all 24 of the Influenza A virus polypeptides in the composition consist essentially of amino acid sequences selected from parts a) to x). In some embodiments, at least one of the Influenza A virus polypeptides in the composition consists of an amino acid sequence selected from parts a) to x). In some embodiments, at least 5, 10, 15, 20, or all 24 of the Influenza A virus polypeptides in the composition consist of amino acid sequences selected from parts a) to x).

In some embodiments, at least one of the Influenza A virus polypeptides used for affinity purification of the human polyclonal antibodies is conjugated to a solid surface. In some embodiments, all of the Influenza A virus polypeptides used for affinity purification of the human polyclonal antibodies are conjugated to a solid surface. In some embodiments, at least one Influenza A virus polypeptide is conjugated to the solid surface via a linker. The linker can be an additional amino acid residue such as cysteine, which can be attached to either the N-terminus or C-terminus of the conjugated Influenza A virus polypeptide. In some embodiments, the solid surface is suitable to be used in chromatography, such as column chromatography or batch chromatography. In some embodiments, the solid surface is suitable to be used in polypeptide analysis. For example, in some embodiments, the solid surface may be a part of a test tube or a microtiter plate.

As discussed above, viral infections, e.g., infections by an influenza A virus, a Variola virus, a respiratory syncytial virus (RSV) and/or a Cytomegalovirus (CMV), are often accompanied by bacterial, eukaryotic protist and/or fungal infections, which tend to exacerbate the effects of the primary viral infection, particularly in patients with a weakened immune system. Examples of such bacterial infections include infections by Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni, which account for a significant portion of nosocomial bacterial infections worldwide. Examples of such eukaryotic protist and/or fungal infections include infections by a Plasmodium, Pneumocystis jirovecii, Histoplasma capsulatum, Blastomyces dermatitidis, a Coccidioides and/or an Aspergillus.

For this reason, in addition to the compositions comprising viral antigens, e.g., antigen(s) from an influenza A virus, a Variola virus, a respiratory syncytial virus (RSV) and/or a Cytomegalovirus (CMV), the present invention further provides bacterial, eukaryotic protist and/or fungal antigenic preparations comprising secreted and/or cellular antigen(s) of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus.

In some embodiments, the antigenic preparations may comprise Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus antigen(s) comprising a peptide, a protein, a polynucleotide, a nucleic acid, a vitamin, a polysaccharide, a carbohydrate, a lipid and/or a complex thereof. In other embodiments, the lipid or the lipid component in the complex may be substantially removed in the antigenic preparation. In some embodiments, the polysaccharide, carbohydrate, or the polysaccharide or carbohydrate component in the complex may be substantially removed in the antigenic preparation.

In other embodiments, S. aureus Protein A is substantially removed in the antigenic preparation in order to eliminate or substantially reduce the recovery of human antibodies that specifically bind to non-bacterial antigens. S. aureus Protein A may be substantially removed from the antigenic preparation by any suitable methods, e.g., by running the preparation through a chromatography column packed with cyanogen bromide (CNBR)-activated Sepharose 4B coupled to purified or highly purified human gamma globulin or human gamma globulin Fc fragments and collecting the eluate.

In some embodiments, the bacterial antigenic preparations may comprise a S. aureus capsular polysaccharide antigen, such as Type 5 antigen and Type 8 antigen. In other embodiments, the bacterial antigenic preparations may also comprise the S. aureus 336 antigen. In some embodiments, the bacterial antigenic preparations may comprise S. aureus toxins, such as pyrogenic toxin superantigens, exfoliative toxins and/or Staphylococcal toxins. Pyrogenic toxin superantigens (PTSAgs) have superantigen activities that induce toxic shock syndrome (TSS). This group includes the toxin TSST-1, which causes TSS associated with tampon use, and staphylococcal enterotoxins, such as S. aureus enterotoxin A (SEA) and S. aureus enterotoxin B (SEB), which cause food poisoning. Exfoliative toxins are implicated in the disease staphylococcal scalded-skin syndrome (SSSS), which occurs most commonly in infants and young children. Staphylococcal toxins that act on cell membranes include α-toxin, β-toxin, δ-toxin, and several bicomponent toxins. The bicomponent toxin Panton-Valentine leukocidin (PVL) is associated with severe necrotizing pneumonia in children. The genes encoding the components of PVL are encoded on a bacteriophage found in community-associated MRSA strains. In some embodiments, the bacterial antigenic preparations may further comprise staphyloxanthin, a carotenoid pigment that has an antioxidant action that helps S. aureus cells evade killing with reactive oxygen radicals used by the host immune system.

In some embodiments, the bacterial antigenic preparations may also comprise a S. aureus antigen that confers resistance to antibiotics, such as penicillin, methicillin, aminoglycosides and/or vancomycin. Staphylococcal resistance to penicillin is mediated by penicillinase (a form of β-lactamase) production: an enzyme which breaks down the β-lactam ring of the penicillin molecule. Penicillinase-resistant penicillins such as methicillin, oxacillin, cloxacillin, dicloxacillin and flucloxacillin are able to resist degradation by staphylococcal penicillinase. Resistance to methicillin is mediated via the mec operon, part of the staphylococcal cassette chromosome mec (SCCmec). Resistance is conferred by the mecA gene, which codes for an altered penicillin-binding protein (PBP2a or PBP2′) that has a lower affinity for binding β-lactams (penicillins, cephalosporins and carbapenems). Resistance to aminoglycosides, such as kanamycin, gentamicin and streptomycin, is mediated by aminoglycoside modifying enzymes, ribosomal mutations and active efflux of the drug out of the bacteria. Aminoglycoside modifying enzymes inactivate the aminoglycoside by covalently attaching a phosphate, nucleotide or acetyl moiety to either the amine and/or alcohol functionality of the antibiotic, thereby rendering the antibiotic ineffective. The best characterized S. aureus aminoglycoside modifying enzyme is aminoglycoside 4′-O-nucleotidyltransferase, encoded by the ant(4′)-Ia gene. Vancomycin resistance is mediated by acquisition of the vanA gene, which codes for an enzyme that produces an alternative peptidoglycan to which vancomycin will not bind.

In some embodiments, the bacterial antigenic preparations may comprise a combination of two or more different antigens selected from a S. aureus capsular polysaccharide antigen, a S. aureus toxin, staphyloxanthin, and a S. aureus antigen that confers antibiotic resistance. Alternatively, the bacterial antigenic preparations may comprise a combination of two or more different antigens selected from a S. aureus toxin, staphyloxanthin, and a S. aureus antigen that confers antibiotic resistance.

In some embodiments, the Streptococcus infection may be caused by Streptococcus pneumoniae (S. pneumoniae), a Group A Streptococcus (GAS), such as Streptococcus pyogenes (S. pyogenes) or a Group B Streptococcus (GBS), such as Streptococcus agalactiae (S. agalactiae). In another embodiment, the Streptococcus may be selected from S. pneumoniae, S. pyogenes and S. agalactiae. In a further embodiment, the Streptococcus may be selected from GAS strain ATCC # 19615 and GBS strain ATCC # 25663.

S. pneumoniae expresses a number of different virulence factors on its cell surface and inside the organism. These virulence factors contribute to some of the clinical manifestations during infection with S. pneumoniae. S. pneumoniae polysaccharide capsule prevents phagocytosis by host immune cells by inhibiting C3b opsonization of the bacterial cells. Pneumolysin (Ply) is a toxin that causes lysis of host cells and activates complement. Activation of autolysin (LytA) leads to bacterial lysis releasing its internal contents, e.g., pneumolysin. Choline binding protein A/Pneumococcal surface protein A (CbpA/PspA) is an adhesin protein that can interact with carbohydrates on the cell surface of pulmonary epithelial cells and can inhibit complement-mediated opsonization of pneumococci.

In some embodiments, the bacterial antigenic preparations of the present invention may comprise two or more S. pneumoniae virulence factors selected from S. pneumoniae capsular polysaccharide antigens, autolysin (LytA), choline binding protein A/pneumococcal surface protein A (CbpA/PspA) and S. pneumoniae toxins, such as pneumolysin.

S. pyogenes has several virulence factors that enable it to attach to host tissues, evade the immune response, and spread by penetrating host tissue layers. A polysaccharide capsule composed of hyaluronic acid surrounds the bacterium, protecting it from phagocytosis by neutrophils. In addition, the capsule and several factors embedded in the cell wall, including M protein, lipoteichoic acid, and fibronectin-binding protein (protein F) facilitate attachment to various host cells. The M protein also inhibits opsonization by the alternative complement pathway by binding to host complement regulators.

S. pyogenes also secretes a number of virulence factors into its host, such as streptolysins O and S, streptococcal pyrogenic exotoxins (Spe) A, B and C, streptokinase, hyaluronidase, streptodornase, C5a peptidase and streptococcal chemokine protease. Streptolysins O and S are toxins which provide the basis of the organism's hemolytic property. Streptolysin O is a potent toxin affecting many cell types including neutrophils, platelets, and sub-cellular organelles. It causes an immune response and detection of antibodies to it, antistreptolysin O (ASO), can be clinically used to confirm a recent infection. Streptococcal pyrogenic exotoxins (Spe) A, B and C are superantigens secreted by many strains of S. pyogenes. These pyrogenic exotoxins are responsible for the rash of scarlet fever and many of the symptoms of streptococcal toxic shock syndrome. Streptokinase enzymatically activates plasminogen, a proteolytic enzyme, into plasmin, which in turn digests fibrin and other proteins. Hyaluronidase is currently presumed to facilitate the spread of S. pyogenes through infected tissues by breaking down hyaluronic acid, an important component of connective tissue. S. pyogenes streptodornases (DNAses) A-D protect the bacteria from being trapped in neutrophil extracellular traps (NETs) by destroying the NET's DNA, which serves as a scaffold for neutrophil serine proteases. C5a peptidase cleaves the potent neutrophil chemotaxin C5a, which reduces the influx of neutrophils early in infection as the bacteria start colonizing the host's tissue. Streptococcal chemokine protease (ScpC) also prevents the migration of neutrophils by degrading the chemokine IL-8, which normally attracts neutrophils to the site of infection.

In some embodiments, the bacterial antigenic preparations of the present invention may comprise two or more S. pyogenes virulence factors selected from S. pyogenes capsular polysaccharide antigens, M protein, lipoteichoic acid (LTA), fibronectin-binding protein (protein F), streptokinase, hyaluronidase, streptodornases A-D, C5a peptidase and streptococcal chemokine protease (ScpC), S. pyogenes toxins, such as streptolysins O and S, and streptococcal pyrogenic exotoxins (Spe), such as SpeA, SpeB and SpeC.

S. agalactiae's antiphagocytic polysaccharide capsule is this bacterium's main virulence factor. However, S. agalactiae also utilizes a number of accessory virulence factors, such as hyaluronidase, C5a peptidase, alpha C protein, glyceraldehyde 3-phosphate dehydrogenase (GAPDH) and S. agalactiae toxins, such as β-hemolysin (cytolysin) and the CAMP factor (protein B). Thus, in some embodiments, the bacterial antigenic preparations of the present invention may comprise two or more S. agalactiae virulence factors selected from S. agalactiae capsular polysaccharide antigens, hyaluronidase, C5a peptidase, alpha C protein, GAPDH and S. agalactiae toxins, such as β-hemolysin (cytolysin) and the CAMP factor (protein B).

In some embodiments, the E. coli infection may be caused by E. coli selected from enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC).

Enterotoxigenic E. coli (ETEC) is a causative agent of feverless diarrhea in humans. ETEC uses fimbrial adhesins, projections from the bacterial cell surface, to bind enterocytes in the small intestine. ETEC can produce two proteinaceous enterotoxins: the larger of the two proteins, the heat-labile LT enterotoxin, is similar to cholera toxin in structure and function, while the smaller protein, the heat-stable ST enterotoxin, causes cyclic guanosine monophosphate (cGMP) accumulation in the target cells and a subsequent secretion of fluid and electrolytes into the intestinal lumen.

Enteropathogenic E. coli (EPEC) is another causative agent of diarrhea in humans. EPEC lacks ST and LT toxins and fimbriae, but utilizes another adhesin known as intimin to bind host intestinal cells. This virotype has an array of virulence factors that are similar to those found in Shigella, and may possess a Shiga-like toxin.

Enteroinvasive E. coli (EIEC) is found exclusively in humans and causes a syndrome that is identical to Shigellosis, with profuse diarrhea and high fever. EIEC is highly invasive, and utilizes adhesin proteins to bind to and enter intestinal cells. It does not secrete toxins, but severely damages the intestinal wall through mechanical cell destruction.

Enterohemorrhagic E. coli (EHEC) typically causes bloody diarrhea and no fever, but can also cause hemolytic-uremic syndrome and sudden kidney failure. The best known member of this virotype is Shiga toxin-producing E. coli (STEC) strain O157:H7 (ATCC # 43895). It uses bacterial fimbriae for attachment, is moderately-invasive and possesses a phage-encoded Shiga-like toxin that can elicit an intense inflammatory response.

Enteroaggregative E. coli (EAggEC) is found exclusively in humans and cause watery diarrhea without fever. EAggEC is non-invasive and uses fimbriae to binds to the intestinal mucosa. It produces a hemolysin and an ST enterotoxin similar to that of ETEC.

Uropathogenic E. coli (UPEC) is responsible for the bulk of human urinary tract infections (UTI). UPEC utilizes P fimbriae (pyelonephritis-associated pili) to bind urinary tract endothelial cells and colonize the bladder. These adhesins specifically bind D-galactose-D-galactose moieties on the P blood group antigen of erythrocytes and uroepithelial cells. UPEC also produces α- and β-hemolysins, which cause lysis of urinary tract cells. It also has the ability to form K antigen, a capsular polysaccharide that contributes to biofilm formation.

In some embodiments, the bacterial antigenic compositions of the present invention may comprise two or more E. coli virulence factors selected from E. coli capsular polysaccharide antigens, such as K antigen, enterotoxins, such as heat-labile LT enterotoxins and heat-stable ST enterotoxins, adhesins, such as fimbrial adhesins and intimin, hemolysins, such as α-hemolysin and β-hemolysin, and Shiga toxins.

P. aeruginosa features a number of virulence factors involved in colonization, invasion, and toxicogenesis. Virulence factors involved in colonization include adhesins, such as fimbriae (N-methyl-phenylalanine pili), capsule polysaccharides (glycocalyx) and mucoid exopolysaccharides (alginate). Virulence factors involved in invasion include invasins, such as proteases (elastase and alkaline protease), hemolysins (phospholipase and lecithinase), cytotoxin (leukocidin), and diffusible pigments (pyochelin and pyocyanin). Finally, virulence factors involved in toxicogenesis include lipopolysaccharide endotoxin and extracellular toxins, such as exoenzyme S and exotoxin A. Exoenzyme S has the characteristic subunit structure of the A-component of a bacterial toxin, and it has ADP-ribosylating activity for a variety of eukaryotic proteins that is characteristic of many bacterial exotoxins. Exotoxin A causes the ADP ribosylation of eukaryotic elongation factor 2 resulting in inhibition of protein synthesis in the affected cell.

In some embodiments, the bacterial antigenic preparations may comprise two or more P. aeruginosa virulence factors selected from adhesins, such as fimbrial adhesins, capsule polysaccharides and mucoid exopolysaccharides, invasins, such as an elastase, an alkaline protease, hemolysins, such as a phospholipase and a lecithinase, leukocidin, a diffusible pigment, such as pyochelin and pyocyanin, lipopolysaccharide endotoxin, and extracellular toxins, such as exoenzyme S and exotoxin A.

Relatively, little is known about the virulence, antibiotic resistance, or persistence strategies of A. baumannii. The pathogenic determinants that have been reported thus far for A. baumannii include lipopolysaccharide 0, capsular exopolysaccharide, a novel pilus assembly system involved in biofilm formation, an outer membrane protein (Omp38) that causes apoptosis in human epithelial cells, and a polycistronic siderophore-mediated iron-acquisition system conserved between A. baumannii and Vibrio anguillarum. These factors presumably constitute a small fraction of elements involved in A. baumannii pathogenesis.

In some embodiments, the bacterial antigenic preparations may comprise two or more A. baumannii virulence factors selected from lipopolysaccharide 0, capsular exopolysaccharide, pilus assembly system, membrane protein Omp38, and proteins of the polycistronic siderophore-mediated iron-acquisition system.

A number of enterococcal virulence factors have been described, including gelatinase, enterococcal surface protein (ESP), aggregation substance (AS), serine protease, capsular polysaccharide, cell wall polysaccharide, hemagglutinin, hemolysin/cytolysin. Among them, gelatinase, ESP, AS, hemagglutinin and cytolysin have been studied most intensively. Gelatinase is a secreted extracellular zinc metalloendopeptidase secreted that shares homologies with gelatinase of Bacillus species and P. aeruginosa elastase. Gelatinase can hydrolyze gelatin, casein, hemoglobin, and other bioactive peptides, which suggests its potential role as a virulence factor in enterococci. ESP is an enterococcal cell wall-associated protein that has been shown to enhance the persistence of E. faecalis in urinary bladed during experimental urinary tract infections. It is believed that ESP facilitates the adhesion of Enterococcus cells to bladder epithelium through specific components of the bladder wall such as mucin or uroplakin. AS is a pheromone inducible surface protein which promotes mating aggregate formation during bacterial conjugation and mediates efficient enterococcal donor-recipient contact to facilitate plasmid transfer. It has been demonstrated to mediate adhesion to cultured renal cells, suggesting that it may be important in the pathogenesis of infection. In addition to AS, hemagglutinin also contributes to the attachment to host cells. Hemolysin/cytolysin is a bacterial toxin that is encoded by an operon consisting of 8 genes localized on a pheromone-responsive plasmid or chromosome. Hemolysin/cytolysin shows hemolytic and bactericidal activity against other Gram-positive bacteria.

In some embodiments, the bacterial antigenic preparations may comprise two or more E. faecium and/or E. faecalis virulence factors selected from gelatinase, enterococcal surface protein (ESP), aggregation substance (AS), serine protease, capsular polysaccharide, cell wall polysaccharide, hemagglutinin and/or hemolysin/cytolysin. Pathogenic C. difficile strains produce a number of virulence factors. The best characterized are enterotoxin (toxin A) and cytotoxin (toxin B), both of which are responsible for the diarrhea and inflammation seen in infected patients. Another toxin, referred to as “binary toxin,” has also been described in the scientific literature, but its role in Clostridium pathogenesis is not yet understood.

In some embodiments, the bacterial antigenic preparations may comprise two or more C. difficile virulence factors selected from an enterotoxin, such as toxin A, a cytotoxin, such as toxin B, and a binary toxin.

K. pneumoniae has multiple known pathogenicity factors, including capsular polysaccharides (CPS), lipopolysaccharides (LPS), adhesins (e.g., type 1 and type 3 pili, KPF-28 fimbriae, CF29K. and aggregative adhesins), serum resistance factors (e.g., TraT lipoprotein and porins) and siderophores (e.g., aerobactin and enterobactin/enterochelin). The capsular polysaccharides have been classified into over 80 serological subtypes and are commonly referred to as K antigens. At present, strains expressing capsule types K1 and K2 are considered especially likely to be virulent. The lipopolysaccharides have been classified into 9 different serotypes, which are commonly referred to as O antigens. O1 is the most common O antigen found among clinical isolates. The K and O antigens form the basis for K. pneumoniae subtyping and are the primary antigens used for active vaccination.

In some embodiments, the bacterial antigenic preparations may comprise one or more K. pneumoniae virulence factors selected from a CPS, an LPS, an adhesin, a serum resistance factor and a siderophore. In some embodiments, the K. pneumoniae virulence factor is selected from an O antigen, a K antigen and a combination thereof. In some embodiments, the bacterial antigenic preparations may comprise a K. pneumoniae toxin or an antigen that confers antibiotic resistance, such as the extended spectrum β-lactamase (ESBL) resistance.

The pathogenic mechanisms expressed by species of Enterobacter bacteria are relatively poorly studied. Similar to K. pneumonia, they express adhesins (e.g., type 1 or type 3 fimbriae and type 1 or type 3 mannose-sensitive hemagglutinins (MSHA)). Most Enterobacter strains also express an aerobactin-mediated iron uptake system, commonly associated with extra-intestinal human bacterial pathogens. Additionally, an outer membrane protein, OmpX, appears to be a virulence factor for E. cloacae by reducing the production of porins, leading to decreased sensitivity to β-lactam antibiotics, and therefore might play a role in cell invasion of the host. Enterobacter species also produce a variety of siderophores, e.g., aerobactin, which is commonly associated with bacterial invasion. Several toxins have been found to be produced by Enterobacter species, such as the E. cloacae Shiga-like toxin II-related cytotoxin and a hemolysin that resembles the E. coli α-hemolysin. Much like K pneumonia, Enterobacter species also produce capsular polysaccharides (K-antigens) and lipopolysaccharides (O-antigens), which are used commonly for typing and immunization.

In some embodiments, the bacterial antigenic preparations may comprise one or more E. aerogenes and/or E. cloacae virulence factors selected from a CPS, an LPS, an adhesin, an outer membrane protein and a siderophore. In some embodiments, the E. aerogenes and/or E. cloacae virulence factor is selected from an O antigen, a K antigen and a combination thereof. In some embodiments, the bacterial antigenic preparations may comprise an E. aerogenes and/or E. cloacae toxin, e.g., a Shiga-like toxin II-related cytotoxin or a hemolysin, or an antigen that confers antibiotic resistance, such as ESBL resistance.

The bacterial, eukaryotic protist and/or fungal antigenic preparations of the present invention may comprise a whole cell extract and/or secreted antigens of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus. In some embodiments, the bacterial, eukaryotic protist and/or fungal antigenic preparations may comprise cellular and/or secreted antigens from a combination of any 2, 3, 4, 5, 6, 7, 8, 9, 10, 11, 12, 13, 14, 15, 16, 17, 18, 19, 20, 21, 22, 23, 24, 25, 26, or all 27 of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus.

In some embodiments, the bacterial antigenic preparations comprise cellular and/or secreted antigens from a combination of any 2, 3, 4, 5, 6, 7, 8, 9, 10, 11, 12, 13, 14, 15, 16, 17, 18, 19, 20, or all 21 of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and Campylobacter jejuni. In other embodiments, the eukaryotic protist and/or fungal antigenic preparations comprise cellular and/or secreted antigens from a combination of any 2, 3, 4, 5, all 6 of a Plasmodium, Pneumocystis jirovecii, Histoplasma capsulatum, Blastomyces dermatitidis, a Coccidioides and an Aspergillus.

In some embodiments, the bacterial antigenic preparations comprise cellular and/or secreted antigens from a combination of any two bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any three bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any four bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any five bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any six bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any seven bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any eight bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any nine bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from a combination of any ten bacterial species selected from S. aureus, a Streptococcus, E. coli, P. aeruginosa, A. baumannii, E. faecium, E. faecalis, E. aerogenes, E. cloacae, C. difficile and K. pneumoniae.

Alternatively, the present bacterial antigenic preparations may comprise cellular and/or secreted antigens from each of S. aureus, a Streptococcus, E. coli, P. aeruginosa, A. baumannii, E. faecium, E. faecalis, E. aerogenes, E. cloacae, C. difficile and K. pneumoniae. In some embodiments, the bacterial antigenic preparations comprise cellular and/or secreted antigens from each of S. aureus, S. pneumoniae, E. coli, P. aeruginosa, A. baumannii, E. faecalis and K. pneumoniae. In some embodiments, the bacterial antigenic preparations comprise cellular and/or secreted antigens from each of S. aureus, S. pneumoniae, E. coli, P. aeruginosa, A. baumannii, E. faecium and K. pneumoniae.

In some embodiments, the method comprising growing bacterial cells in a protein containing culture medium, transferring them to a protein-free culture medium, and then growing the bacterial cells to secrete the toxins. Then without separating the cells from the toxins, the bacterial cells are disrupted in the same medium that contain the bacterial toxins to obtain the antigenic preparations comprising a whole cell extract and secreted toxins. In some embodiments, the whole cell extract is separated from the exotoxins. The bacterial cells are collected, and disrupted to obtain a whole cell extract. The exotoxins are separately collected from the protein-free culture medium in which the bacterial cells were grown. The whole cell extract and the collected toxins may optionally be combined to form the desired antigenic preparations.

In one embodiment, first, bacterial cells are grown in a protein containing culture medium for a specified period of time to reach a desired density. Second, the bacterial cells are collected (e.g., by filtration or centrifugation at 3,000 rpm for 15 minutes at 2-8° C.), resuspended in a non-protein containing culture medium and grown for another specified period of time in order to give the cells enough time to produce and secrete antigens (e.g., exotoxins) into the non-protein containing culture medium. Since different bacterial cells, e.g., cells of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, typically have different nutritional requirements and different growth rates, these growth steps are usually performed separately for each bacterial strain used. In some embodiments, different bacterial strains may be grown together in the same culture media if their nutritional requirements and growth conditions are sufficiently similar to permit joint culture.

Any suitable protein containing culture medium may be used to grow bacterial cells, e.g., cells of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni. In some embodiments, the protein containing culture medium for growing the bacterial cells may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, the bacterial cells are grown in the protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the non-protein containing culture medium for growing the bacterial cells may comprise an aqueous solution comprising sodium chloride, sodium phosphate, and optionally comprising a source of carbon, such as glucose or succinate. Typically, the bacterial cells are grown in the non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow S. aureus cells. In some embodiments, the S. aureus protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, S. aureus cells are grown in a protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the S. aureus non-protein containing culture medium may comprise an aqueous solution comprising sodium chloride, sodium phosphate, and optionally comprising a source of carbon, such as glucose or succinate. Typically, S. aureus cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow Streptococcus cells. In some embodiments, the Streptococcus protein containing culture medium may comprise the following ingredients: 3.1 g/L heart infusion (dehydrated), 20.0 g/L yeast enriched peptone, 2.0 g/L dextrose, 2.0 g/L sodium chloride, 0.4 g/L disodium phosphate, and 2.5 g/L sodium carbonate, pH 7.8±0.2. (Neogen Acumedia™ THB 7110, Lansing Mich.). The medium is autoclaved at 121° C. for 15 minutes after reconstitution in de-ionized H₂O. Typically, Streptococcus cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the Streptococcus non-protein containing culture medium may comprise an aqueous solution comprising sodium chloride, sodium phosphate, and optionally comprising a source of carbon, such as glucose or succinate. Typically, Streptococcus cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow E. coli cells. In some embodiments, the E. coli protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, E. coli cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the E. coli non-protein containing culture medium may comprise an aqueous solution comprising sodium chloride, sodium phosphate, and optionally comprising a source of carbon, such as glucose or succinate. Typically, E. coli cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow P. aeruginosa cells. In some embodiments, the P. aeruginosa protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, P. aeruginosa cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the P. aeruginosa non-protein containing culture medium may comprise an aqueous solution comprising sodium chloride, sodium phosphate, and optionally comprising a source of carbon, such as glucose or succinate. Typically, P. aeruginosa cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow A. baumannii cells. In some embodiments, the A. baumannii protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, A. baumannii cells are grown in a protein containing culture medium for about 10 hours to about 72 hours, preferably for about 48 hours, at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the A. baumannii non-protein containing culture medium may comprise an aqueous solution comprising sodium chloride, sodium phosphate, and optionally comprising a source of carbon, such as glucose or succinate. Typically, A. baumannii cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow E. faecium or E. faecalis cells. In some embodiments, the E. faecium or E. faecalis protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, E. faecium or E. faecalis cells are grown in a protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the E. faecium or E. faecalis non-protein containing culture medium may comprise an aqueous solution comprising sodium chloride, sodium phosphate, and optionally comprising a source of carbon, such as glucose or succinate. Typically, E. faecium or E. faecalis cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow E. aerogenes or E. cloacae cells. In some embodiments, the E. aerogenes or E. cloacae protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, E. aerogenes or E. cloacae cells are grown in a protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the E. aerogenes or E. cloacae non-protein containing culture medium may comprise an aqueous solution comprising sodium chloride, sodium phosphate, and optionally comprising a source of carbon, such as glucose or succinate. Typically, E. aerogenes or E. cloacae cells are grown in a non-protein containing culture medium for about 10 hours to about 48 hours at an appropriate temperature, e.g., 37° C. under conditions (for example mixing) familiar to those of ordinary skill in the art.

Any suitable protein containing culture medium may be used to grow C. difficile cells. In some embodiments, the C. difficile protein containing culture medium may comprise the following ingredients: 5.0 g/L pancreatic digest of casein, 5.0 g/L proteose peptone #3, 10.0 g/L beef extract, 3.0 g/L yeast extract, 5.0 g/L NaCl, 1.0 g/L soluble starch, 5.0 g/L dextrose, 0.5 g/L cysteine HCl and 3.0 g/L sodium acetate (Difco™ Reinforced Clostridial Media, 38% w/v in de-ionized H₂O; Becton Dickinson Cat. No. 218081). Typically, C. difficile cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the C. difficile non-protein containing culture medium may comprise an aqueous solution comprising sodium chloride, sodium phosphate, and optionally comprising a source of carbon, such as glucose or succinate. Typically, C. difficile cells are grown in a non-protein containing culture medium for about 10 hours to about 48 hours at an appropriate temperature, e.g., 37° C. under conditions (for example mixing) familiar to those of skill in the art.

Any suitable protein containing culture medium may be used to grow K. pneumoniae cells. In some embodiments, the K. pneumoniae protein containing culture medium may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, K. pneumoniae cells are grown in a protein containing culture medium for about 10 hours to about 72 hours at an appropriate temperature, e.g., 37° C. under conditions (for example mixing) familiar to those of skill in the art to reach a density from 1×10⁹ to about 2×10⁹ before the cell collecting, e.g., pelleting, step. In some embodiments, the K. pneumoniae non-protein containing culture medium may comprise an aqueous solution comprising sodium chloride, sodium phosphate, and optionally comprising a source of carbon, such as glucose or succinate. Typically, K. pneumoniae cells are grown in a non-protein containing culture medium for about 10 hours to about 48 hours at an appropriate temperature, e.g., 37° C. under conditions (for example mixing) familiar to those of skill in the art.

In the next step, the bacterial cells are harvested by centrifugation at 20,000 rpm for 15-30 minutes at 2-8° C., resuspended in 10 volumes of sterile phosphate buffered saline (PBS), pH 7.5, and pelleted by another centrifugation at 20,000 rpm for 15-30 minutes at 2-8° C. The wash procedure is repeated two more times in order to completely remove the culture medium. The bacterial cells can be disrupted by any suitable methods. In some embodiments, the bacterial cells are disrupted with a Microfluidizer® high-shear fluid processor (Microfluidics Corp., Newton, Mass.) twice under 20,000 psi at 150 ml/min Disruption of the bacterial cells can also be accomplished by homogenization (e.g., by using the Potter-Elvehjem homogenizer, Dounce homogenizer, or French press), freeze thaw and/or sonication, after which insoluble cellular debris (e.g., bacterial walls and nuclei) are removed, e.g., filtered or pelleted (e.g., by centrifugation at 4,000 rpm for 30 minutes at 2-8° C.), and the supernatant containing cellular antigens is collected. In some embodiments, detergent cell lysis may be used alone or in conjunction with homogenization, freeze thaw and/or sonication to disrupt the bacterial cells. The choice of detergent depends on the cells to be disrupted, particularly on the presence or absence of a bacterial cell wall. In general, non-ionic (e.g., Triton-X®) and zwitterionic (e.g., CHAPS) detergents are milder and less denaturing than ionic detergents. In contrast, ionic detergents (e.g., SDS) are strong solubilizing agents and tend to denature proteins, thereby destroying protein activity and function. There are also ionic detergents that are only mildly denaturing (e.g., sodium cholate and sodium deoxycholate). In some embodiments, it may be preferable to use a dialyzable detergent to facilitate its removal from the lysis solution.

Antigens secreted by the bacterial cells into the non-protein containing culture medium are also collected. In some embodiments, the secreted antigens are collected separately from the whole cell extract by precipitating the bacterial cells prior to the bacterial cell disruption step and by collecting the supernatant. In other embodiments, the disruption step is carried out in the presence of the secreted antigens, so that the secreted antigens are combined with the cellular antigens immediately upon the disruption of the bacterial cells.

In some embodiments, the disruption and collection steps are performed separately for each bacterial strain. In other embodiments, two or more bacterial strains are combined prior to the disruption and collection of the cellular and secreted antigens. In some embodiments, the cultures of the bacterial cells, e.g., cells of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni, are pooled prior to the bacterial cell disruption and the collection of the secreted and cellular antigens.

In some embodiments, optimal bacterial cell lysis conditions are used to maximize the amount of extracted protein while minimizing protein oxidation, unwanted proteolysis and sample contamination with genomic DNA. See e.g., Protein production and purification, Nature Methods, 5(2):135-146 (2008). Mechanical lysis by high-pressure homogenization or sonication, or lysis by freeze-thaw procedures with lysozyme are equivalent in most cases. The lysis buffer may contain a strong buffer (e.g., 50-100 mM phosphate or HEPES) to overcome the contribution of the bacterial lysate, high ionic strength (e.g., equivalent to 300-500 mM NaCl) to enhance protein solubility and stability, protease inhibitors and a reducing agent such as dithiothreitol (DTT) or Tris(2-carboxyethyl) phosphine hydrochloride (TCEP) to prevent oxidation of the protein. Inclusion of glycerol (10%) during protein purification enhances the solubility and stability of many proteins. Loading large amounts of bacterial lysate (e.g., >1 L culture volume) on relatively small (e.g., <1 ml) affinity columns may require prior removal of any particulate or viscous material. This can be accomplished by using enzymes that degrade nucleic acid and cell-wall material, such as DNase or Benzonase (Merck/EMD) and lysozyme, respectively. Some of the enzymes used in lysis are less active in the presence of reducing agents or high salt concentration; optimal lysis may require sequential addition of the components. Clarified lysates can also be filtered before loading on the affinity columns

A wide variety of bacterial lysis solutions that are suitable for total protein extraction are currently available. By way of illustration and not limitation, suitable bacterial lysis compositions may include: 20 mM HEPES, pH 7.6, 500 mM NaCl, 1 mM EDTA, 10% (v/v) glycerol, 1 mM PMSF, 5 μg/ml leupeptine, 1% (v/v) aprotinin and 0.1% NP-40; 10 mM Tris-HCl, pH 7.4, 1 mM EDTA, 8 M Urea, 50 mM DTT, 10% (v/v) glycerol, 5% v/v NP-40 and 6% (w/v) ampholytes (i.e., amphoteric compounds containing both acidic and basic groups); CelLytic™ B, CelLytic™ B-II and CelLytic™ B Plus Protein Extraction Reagents (Sigma-Aldrich, Part Nos. B3553, B3678 and CB0500); B-PER® Bacterial Protein Extraction Reagent (Pierce Biotechnology, Part No. 78248); EasyLyse™ Bacterial Protein Extraction Solution (Epicentre Biotechnologies, Part No. RP03750); or Easy BacLysis Protein Extraction Solution (GenScript, Part Nos. L00230 and L00240).

The secreted antigens of the bacterial cells, e.g., cells of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni, may comprise well-characterized exotoxins that may be used as benchmarks for assessing the quality and/or concentration of the antigenic preparation.

In some embodiments, the secreted antigens of the bacterial cells may comprise exotoxin(s). The exotoxin(s) may be present in the secreted antigens at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL. Similarly, in some embodiments, the antigenic preparations may comprise a bacterial whole cell extract and exotoxin(s). The exotoxin(s) may be present in the antigenic preparations at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL.

In some embodiments, the secreted antigens of S. aureus may comprise staphylococcal enterotoxin A (SEA) and/or staphylococcal enterotoxin B (SEB). The SEA may be present in the secreted antigens at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL. Similarly, in some embodiments, the antigenic preparations may comprise a S. aureus whole cell extract and SEA and/or SEB. The SEA may be present in the antigenic preparations at a concentration of about 0.01 μg/mL to about 400 μg/mL, preferably about 0.01 μg/mL to about 5 μg/mL, whereas the SEB may be present at a concentration of about 0.01 μg/mL to about 400 μg/mL, preferably about 10 μg/mL to about 400 μg/mL.

In some embodiments, the secreted antigens of Streptococcus may comprise Streptococcal pyrogenic exotoxin A (SpeA) and/or Streptococcal pyrogenic exotoxin C (SpeC). The SpeA may be present in the secreted antigens at a concentration of about 0.01 μg/mL to about 400 μg/mL, preferably about 5 μg/mL to about 20 μg/mL, whereas the SpeC may be present at a concentration of about 0.01 μg/mL to about 400 μg/mL, preferably about 0.01 μg/mL to about 10 μg/mL. Similarly, in some embodiments, the antigenic preparations may comprise a Streptococcus whole cell extract and SpeA and/or SpeC. The SpeA may be present in the antigenic preparations at a concentration of about 0.01 μg/mL to about 400 μg/mL, preferably about 5 μg/mL to about 20 μg/mL, whereas the SpeC may be present at a concentration of about 0.01 μg/mL to about 400 μg/mL, preferably about 0.01 μg/mL to about 10 μg/mL.

In some embodiments, the secreted antigens of E. coli may comprise a Shiga-like toxin. The Shiga-like toxin may be present in the secreted antigens at a concentration of about 0.01 μg/mL to about 400 μg/mL, preferably about 0.25 μg/mL to about 4 μg/mL. Similarly, in some embodiments, the antigenic preparations may comprise an E. coli whole cell extract and a Shiga-like toxin. The Shiga-like toxin may be present in the antigenic preparations at a concentration of about 0.01 μg/mL to about 400 μg/mL, preferably about 0.25 μg/mL to about 4 μg/mL.

In some embodiments, the secreted antigens of P. aeruginosa may comprise exoenzyme S (PES) and/or exotoxin A (PEA). The PES may be present in the secreted antigens at a concentration of about 0.01 μg/mL to about 400 μg/mL, whereas the PEA may be present at a concentration of about 0.01 μg/mL to about 400 μg/mL. Similarly, in some embodiments, the antigenic preparations may comprise a P. aeruginosa whole cell extract and PES and/or PEA. The PES may be present in the antigenic preparations at a concentration of about 0.01 μg/mL to about 400 μg/mL, whereas the PEA may be present at a concentration of about 0.01 μg/mL to about 400 μg/mL.

In some embodiments, the secreted antigens of C. difficile may comprise toxin A (CTA) and/or toxin B (CTB). The CTA may be present in the secreted antigens at a concentration of about 0.01 μg/mL to about 400 μg/mL, whereas the CTB may be present at a concentration of about 0.01 μg/mL to about 400 μg/mL. Similarly, in some embodiments, the antigenic preparations may comprise a C. difficile whole cell extract and CTA and/or CTB. The CTA may be present in the antigenic preparations at a concentration of about 0.01 μg/mL to about 400 μg/mL, whereas the CTB may be present at a concentration of about 0.01 μg/mL to about 400 μg/mL.

Any suitable protein containing culture medium may be used to grow eukaryotic protist or fungal cells, e.g., cells of a Plasmodium, Pneumocystis jirovecii, Histoplasma capsulatum, Blastomyces dermatitidis, a Coccidioides and an Aspergillus. In some embodiments, the protein containing culture medium for growing the eukaryotic protist or fungal cells may comprise the following ingredients: 17.0 g/L vegetable infusion (dehydrated), 3.0 g/L enzymatic digest of soybean meal, 5.0 g/L sodium chloride, 2.5 g/L dipotassium phosphate, and 2.5 g/L dextrose, pH 7.3±0.2 (Acumedia™ TSB 7728, Neogen, Lansing, Mich.). The medium is autoclaved at 121° C. for 15 minutes after reconstitution in de-ionized H₂O. Typically, the eukaryotic protist or fungal cells are grown in the protein containing culture medium for about 10 hours to about 72 hours at an appropriate temperature, e.g., 37° C. under conditions (for example mixing) familiar to those of skill in the art, to reach a density from 1×10⁹ to about 2×10⁹ before the cell collecting, e.g., pelleting, step. In some embodiments, the non-protein containing culture medium for growing the eukaryotic protist or fungal cells may comprise an aqueous solution comprising sodium chloride, sodium phosphate, and optionally comprising a source of carbon, such as glucose or succinate. Typically, the eukaryotic protist or fungal cells are grown in the non-protein containing culture medium for about 10 hours to about 48 hours at an appropriate temperature, e.g., 37° C. under conditions (for example mixing) familiar to those of ordinary skill in the art.

In the next step, the eukaryotic protist or fungal cells are harvested by centrifugation at 20,000 rpm for 15-30 minutes at 2-8° C., resuspended in 10 volumes of sterile phosphate buffered saline (PBS), pH 7.5, and pelleted by another centrifugation at 20,000 rpm for 15-30 minutes at 2-8° C. The wash procedure is repeated two more times in order to completely remove the culture medium. The eukaryotic protist or fungal cells can be disrupted by any suitable methods. In some embodiments, the eukaryotic protist or fungal cells are disrupted with a Microfluidizer® high-shear fluid processor (Microfluidics Corp., Newton, Mass.) twice under 20,000 psi at 150 ml/min. Disruption of the eukaryotic protist or fungal cells can also be accomplished by homogenization (e.g., by using the Potter-Elvehjem homogenizer, Dounce homogenizer, or French press), freeze thaw and/or sonication, after which insoluble cellular debris (e.g., bacterial walls and nuclei) are removed, e.g., filtered or pelleted (e.g., by centrifugation at 4,000 rpm for 30 minutes at 2-8° C.), and the supernatant containing cellular antigens is collected. In some embodiments, detergent cell lysis may be used alone or in conjunction with homogenization, freeze thaw and/or sonication to disrupt the eukaryotic protist or fungal cells. The choice of detergent depends on the cells to be disrupted, particularly on the presence or absence of an eukaryotic protist or fungal cell wall. In general, non-ionic (e.g., Triton-X®) and zwitterionic (e.g., CHAPS) detergents are milder and less denaturing than ionic detergents. In contrast, ionic detergents (e.g., SDS) are strong solubilizing agents and tend to denature proteins, thereby destroying protein activity and function. There are also ionic detergents that are only mildly denaturing (e.g., sodium cholate and sodium deoxycholate). In some embodiments, it may be preferable to use a dialyzable detergent to facilitate its removal from the lysis solution.

Antigens secreted by the eukaryotic protist or fungal cells into the non-protein containing culture medium are also collected. In some embodiments, the secreted antigens are collected separately from the whole cell extract by precipitating the eukaryotic protist or fungal cells prior to the eukaryotic protist or fungal cell disruption step and by collecting the supernatant. In other embodiments, the disruption step is carried out in the presence of the secreted antigens, so that the secreted antigens are combined with the cellular antigens immediately upon the disruption of the eukaryotic protist or fungal cells.

In some embodiments, the disruption and collection steps are performed separately for each eukaryotic protist or fungal strain. In other embodiments, two or more eukaryotic protist or fungal strains are combined prior to the disruption and collection of the cellular and secreted antigens. In some embodiments, the cultures of the eukaryotic protist or fungal cells, e.g., cells of a Plasmodium, Pneumocystis jirovecii, Histoplasma capsulatum, Blastomyces dermatitidis, a Coccidioides and an Aspergillus, are pooled prior to the eukaryotic protist or fungal cell disruption and the collection of the secreted and cellular antigens.

In some embodiments, optimal eukaryotic protist or fungal cell lysis conditions are used to maximize the amount of extracted protein while minimizing protein oxidation, unwanted proteolysis and sample contamination with genomic DNA. See e.g., Protein production and purification, Nature Methods, 5(2):135-146 (2008). Mechanical lysis by high-pressure homogenization or sonication, or lysis by freeze-thaw procedures with lysozyme are equivalent in most cases. The lysis buffer may contain a strong buffer (e.g., 50-100 mM phosphate or HEPES) to overcome the contribution of the eukaryotic protist or fungal lysate, high ionic strength (e.g., equivalent to 300-500 mM NaCl) to enhance protein solubility and stability, protease inhibitors and a reducing agent such as dithiothreitol (DTT) or Tris(2-carboxyethyl)phosphine hydrochloride (TCEP) to prevent oxidation of the protein. Inclusion of glycerol (10%) during protein purification enhances the solubility and stability of many proteins. Loading large amounts of eukaryotic protist or fungal lysate (e.g., >1 L culture volume) on relatively small (e.g., <1 ml) affinity columns may require prior removal of any particulate or viscous material. This can be accomplished by using enzymes that degrade nucleic acid and cell-wall material, such as DNase or Benzonase (Merck/EMD) and lysozyme, respectively. Some of the enzymes used in lysis are less active in the presence of reducing agents or high salt concentration; optimal lysis may require sequential addition of the components. Clarified lysates can also be filtered before loading on the affinity columns.

A wide variety of eukaryotic protist or fungal lysis solutions that are suitable for total protein extraction are currently available. By way of illustration and not limitation, suitable eukaryotic protist or fungal lysis compositions may include: 20 mM HEPES, pH 7.6, 500 mM NaCl, 1 mM EDTA, 10% (v/v) glycerol, 1 mM PMSF, 5 μg/ml leupeptine, 1% (v/v) aprotinin and 0.1% NP-40; 10 mM Tris-HCl, pH 7.4, 1 mM EDTA, 8 M Urea, 50 mM DTT, 10% (v/v) glycerol, 5% v/v NP-40 and 6% (w/v) ampholytes (i.e., amphoteric compounds containing both acidic and basic groups); CelLytic™ B, CelLytic™ B-II and CelLytic™ B Plus Protein Extraction Reagents (Sigma-Aldrich, Part Nos. B3553, B3678 and CB0500).

The secreted antigens of the eukaryotic protist or fungal cells, e.g., cells of a Plasmodium, Pneumocystis jirovecii, Histoplasma capsulatum, Blastomyces dermatitidis, a Coccidioides and an Aspergillus, may comprise well-characterized exotoxins that may be used as benchmarks for assessing the quality and/or concentration of the antigenic preparation.

### III. Affinity-Purified Human Polyclonal Antibodies

As noted above, in one aspect, the present invention provides human polyclonal antibodies for treating or preventing viral, bacterial, eukaryotic protist or fungal infection(s). The antibodies are affinity purified from a human blood sample using one or more of the antigenic compositions according to the present invention.

In a related aspect, the invention provides a combination of human polyclonal antibodies for treating or preventing viral, bacterial, eukaryotic protist or fungal infection(s). The combination includes (a) human polyclonal antibodies affinity purified from a human blood sample using one of an influenza A virus, a Variola virus, a respiratory syncytial virus (RSV) and/or a Cytomegalovirus (CMV) antigenic compositions according to the present invention; (b) human polyclonal antibodies affinity purified from a human blood sample with an antigenic preparation comprising cellular and/or secreted antigens from bacterial cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni and a combination thereof; and/or c) human polyclonal antibodies affinity purified from a human blood sample with an antigenic preparation comprising cellular and/or secreted antigens from eukaryotic protist or fungal cells selected from a Plasmodium, Pneumocystis jirovecii, Histoplasma capsulatum, Blastomyces dermatitidis, a Coccidioides and an Aspergillus.

In a related aspect, the invention provides a combination of human polyclonal antibodies for treating or preventing an Influenza A virus infection and an accompanying bacterial infection. The combination includes (a) human polyclonal antibodies affinity purified from a human blood sample using one of the Influenza A virus antigenic compositions according to the present invention; and (b) human polyclonal antibodies affinity purified from a human blood sample with an antigenic preparation comprising cellular and/or secreted antigens from bacterial cells selected from S. aureus, a Streptococcus, E. coli, P. aeruginosa, A. baumannii, E. faecium, E. faecalis, E. aerogenes, E. cloacae, C. difficile, K. pneumoniae and a combination thereof.

In further related aspect, the invention provides a combination of human polyclonal antibodies for treating or preventing viral, bacterial, eukaryotic protist or fungal infection(s), comprising human polyclonal antibodies affinity purified from a human blood sample having specificity for a viral, bacterial, eukaryotic protist and/or fungal antigen(s) and an antigen from human tumor necrosis factor alpha (TNF-α). TNF-α, also referred to as TNF, cachexin or cachectin, is a cytokine involved in systemic inflammation and a member of a group of cytokines that stimulate the acute phase reaction. TNF-α is produced mainly by macrophages, as well as by a wide variety of other cell types such as lymphoid cells, mast cells, endothelial cells, cardiac myocytes, adipose tissue, fibroblasts and neuronal tissue. Large quantities of TNF-α are released in response to lipopolysaccharide, other bacterial products, and interleukin-1 (IL-1). A local increase in concentration of TNF-α causes heat, swelling, redness, and pain, whereas high concentrations of TNF-α are known to induce shock-like symptoms. By combining human polyclonal antibodies specific for an Influenza A viral antigen and a bacterial antigen with polyclonal antibodies against TNF-α, it is possible to neutralize the viral and bacterial infection and suppress the acute immune response mediated by TNF-α.

Any suitable TNF-α antigen(s) can be used to affinity purify the desired polyclonal antibodies having specificity for TNF-α from a human blood sample. In some embodiments, an antigen within the soluble homotrimeric cytokine (sTNF) or the entire sTNF is used to affinity purify the desired polyclonal antibodies having specificity for TNF-α. In other embodiments, an antigen within TNF-α that binds to a TNF-α receptor, e.g., TNF receptor type 1 (TNF-R1, CD120a; p55/60) or TNF receptor type 2 (TNF-R2, CD120b, p75/80), is used to affinity purify the desired polyclonal antibodies having specificity for TNF-α. In still other embodiments, an antigen within TNF-α that binds to a therapeutic monoclonal antibody such as infliximab (REMICADE®) or adalimumab (HUMIRA®) is used to affinity purify the desired polyclonal antibodies having specificity for TNF-α. In yet other embodiments, the entire TNF-α is used to affinity purify the desired polyclonal antibodies having specificity for TNF-α.

In some embodiments, the human polyclonal antibodies having specificity for the Influenza A viral antigen are affinity purified from the human blood sample with one of the Influenza A virus antigenic compositions according to the present invention. In some embodiments, the human polyclonal antibodies having specificity for the bacterial antigen are affinity purified from the human blood sample with an antigenic preparation comprising cellular and/or secreted antigens from bacterial cells selected from S. aureus, a Streptococcus, E. coli, P. aeruginosa, A. baumannii, E. faecium, E. faecalis, E. aerogenes, E. cloacae, C. difficile, K. pneumoniae and a combination thereof.

In some embodiments, the human polyclonal antibodies having specificity for the bacterial antigen are affinity purified from the human blood sample using an antigenic preparation comprising cellular and/or secreted antigens from bacterial cells selected from S. aureus, a Streptococcus, E. coli, P. aeruginosa, A. baumannii, E. faecalis and K. pneumoniae. In some embodiments, the human polyclonal antibodies having specificity for the bacterial antigen are affinity purified from the human blood sample using an antigenic preparation comprising cellular and/or secreted antigens from bacterial cells selected from S. aureus, a Streptococcus, E. coli, P. aeruginosa, A. baumannii, E. faecium and K. pneumoniae.

In some embodiments, the starting material for the polyclonal antibodies of the present invention is a serum, plasma or whole blood sample. If a whole blood sample is used, it may be subjected to some preliminary processing steps such as dilution or removing particulate materials from the blood sample. In some embodiments, the blood sample is obtained from a normal human who is not hyperimmune to an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus, or recent exposure to an acute infection, especially the infection that led to bacteremia, of an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus.

In other embodiments, the blood sample is obtained from a human who is hyperimmune to an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus as a result of recent vaccination against these microorganisms or recent exposure to an acute infection, especially the infection that led to bacteremia, from these microorganisms. In some embodiments, the human blood sample is obtained from a human infected with or vaccinated against Influenza A virus having one the following serotypes: H1N1, H2N2, H3N2, H5N1, H7N7, H1N2, H9N2, H7N2, H3N2, H7N3, H5N2 or H10N7. In some embodiments, the amino acid residue at position 627 of the polymerase B2 (PB2) protein of the Influenza A virus is lysine. In some embodiments, the Influenza A virus hemagglutinin (HA) protein binds to alpha 2-6 sialic acid receptors.

In some embodiments, the blood sample is human plasma from a normal human donor that has been lipid stripped with the use of fumed silica, dextran sulfate or other conventional processes such as using organic solvents capable of solubulizing lipids. In some embodiments, the blood sample is human gamma globulin (IgG) from a normal human donor prepared by known methods, such as cold alcohol Cohn fractionation, ammonium sulfate precipitation, caprylic acid precipitation and/or sodium sulfate precipitation.

For viruses and bacteria that are ubiquitous, the person who is not currently infected with the organism may be de facto hyperimmune as the person may have been exposed and is now protected from that organism. The advantage of using hyperimmune plasma is only one of quantity, i.e., there is a greater quantity of antibody in the plasma from the hyperimmune individual. Normal human plasma may have just as potent and therapeutically effective antibodies as the hyperimmune person; it is only present in lower concentrations. This disadvantage can be overcome with the use of much greater quantities of normal human plasma as compared to the quantity of hyperimmune plasma.

One important advantage of the present compositions and methods is that they can be adapted to infectious viral, bacterial, eukaryotic protist and/or fungal serotypes typical of a particular geographic region by using locally collected and current human blood samples. Thus, in some embodiments, the human blood sample may be collected from a geographic area in which the anti-viral, anti-bacterial, anti-eukaryotic protest, and/or anti-fungal treatment is administered. In some embodiments, the human blood sample may be collected from a geographic area in which a recipient of the anti-viral, anti-bacterial, anti-eukaryotic protest, and/or anti-fungal treatment resides. Alternatively, the human blood sample may be collected from a geographic area to which a recipient of the anti-viral, anti-bacterial, anti-eukaryotic protest, and/or anti-fungal treatment intends to travel.

In some embodiments, the blood sample is pooled from at least 2 humans, preferably from at least 10 humans, more preferably from at least 100, 500, 1,000, 5,000, 10,000 or more humans. In some embodiments, the blood sample is pooled from at least 2 normal humans, preferably from at least 10 normal humans, more preferably from at least 100, 500, 1,000, 5,000, 10,000 or more normal humans.

The human polyclonal antibodies of the present invention can be purified or affinity purified from a human blood sample by any suitable methods. In some embodiments, to purify the human blood sample for the desired human polyclonal antibodies, one first attaches one of the influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides, Aspergillus and/or TNF-α antigenic preparations described above to cross linked agarose beads (e.g., cyanogen bromide (CNBr) activated Sepharose 4B from Pharmacia, Uppsala, Sweden), according to manufacturer's instructions. Prior to loading the antigenic preparation onto a chromatography column, CNBr-activated Sepharose 4B may be sterilized with 70% ethyl alcohol (pH 3.0) for about 30 minutes.

Next, one uses these agarose beads with the antigenic preparation coupled to them to pack an affinity separation column. The column is then washed and equilibrated with a suitable wash buffer, e.g., 0.01 M phosphate buffered saline (PBS), pH 7.4. The human blood sample is loaded onto the column and washed with 0.01 M PBS in order to remove antibodies without binding specificity to the influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides, Aspergillus and/or TNF-α antigenic preparations. The bound human polyclonal antibodies specific to the influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides, Aspergillus and/or TNF-α antigen(s) are eluted from the solid phase antigenic preparation in the column by passing an elution solution, e.g., 0.1 M glycine hydrochloride buffer, pH 2.5-3.0 through the column. The eluted polyclonal antibodies are neutralized after they leave the column with either the addition of a neutralizing solution or buffer, e.g., 1 M phosphate buffer, pH 8 or by a buffer exchange with 0.01 M PBS, as is known to those of skill in the art.

The eluate containing human polyclonal antibodies specific to the influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides, Aspergillus and/or TNF-α antigen(s) can optionally be concentrated and buffer exchanged into a solution for administering to a human, e.g., a sterile aqueous solution containing 10% maltose and 0.03% Polysorbate 80, pH 5.5. The solution can also be filtered to remove any residual particulate and stored at a suitable temperature, e.g., 2-8° C. In some embodiments, the preparation is purified to remove antibody aggregates in order to produce a monomeric antibody preparation.

In some embodiments, the affinity purification of human polyclonal antibodies comprises a step of substantially inactivating and/or removing a virus. Any virus that may contaminate or compromise the therapeutic or preventive use of the affinity purified human polyclonal antibodies may be substantially inactivated and/or removed. In some embodiments, the virus to be substantially inactivated and/or removed is a lipid-enveloped or non-enveloped virus. Any suitable methods can be used to substantially inactivate and/or remove a virus. In some embodiments, a virus is substantially inactivated and/or removed by ammonium sulfate fractionation of the human plasma (e.g., 50% saturated ammonium sulfate). In some embodiments, a lipid-enveloped virus is substantially inactivated and/or removed by a filtration based on the virus size and a solvent/detergent treatment step, e.g., a solvent/detergent treatment step using tri-n-butyl phosphate (TNBP) and Triton X-100. For example, an immunoglobulin preparation is treated with 1% Triton X-100 and 0.3% TNBP at room temperature for 4 hours, after which the Triton X-100 and the TNBP are removed from the fractionated plasma by tangential flow ultrafiltration against PBS (pH 7.4). See, e.g., Horowitz, B., “Investigations Into the Application of Tri(n-Butyl) Phosphate/Detergent Mixtures to Blood Derivatives,” Curr. Stud. Hematol. Transfus. 1989, 56:83-96; U.S. Pat. Nos. 3,962,421 and 4,540,573, which are incorporated herein by reference. In some embodiments, a virus is substantially removed from the antibody preparation by affinity chromatography. In some embodiments, a virus is substantially inactivated by an acid treatment (pH˜2.5-3.0) during antibody elution from the affinity column. In some embodiments, a virus is substantially inactivated by ion exchange chromatography and/or multiple filtrations steps following the antibody elution from the affinity column. Preferably, a virus is substantially inactivated and/or removed by a combination of some or all of the purification techniques described herein.

It is noted that the same human blood sample may be subjected to multiple cycles of affinity purification using different antigenic preparations. For example, a human blood sample that has been depleted of anti-Influenza A virus polyclonal antibodies may be collected and subjected to a further round of affinity purification using a S. aureus antigenic preparation, and so forth. Thus, the present invention contemplates both “parallel” affinity purification, wherein human polyclonal antibodies against multiple bacterial species are isolated simultaneously, and “serial” affinity purification, wherein human polyclonal antibodies against multiple viral and bacterial species are isolated sequentially by reusing the same human blood sample in multiple cycles of affinity purification.

The affinity purified human polyclonal antibodies can have suitable concentrations for a desired purpose, e.g., storage or administration. In some embodiments, the affinity purified human polyclonal antibodies of the present invention have a concentration in the range between about 1 μg/ml and about 10 mg/ml, such as between about 10 μg/ml and about 5 mg/ml, between about 100 μg/ml and about 3 mg/ml, between about 10 μg/ml and about 1 mg/ml, or between about 100 μg/ml and about 1 mg/ml. In some embodiments, the affinity purified human polyclonal antibodies can have suitable concentrations at about 1 μg/ml, 2 μg/ml, 5 μg/ml, 10 μg/ml, 50 μg/ml, 100 μg/ml, 200 μg/ml, 500 μg/ml, 1 mg/ml, 5 mg/ml, 10 mg/ml, 20 mg/ml, 30 mg/ml, 40 mg/ml, or 50 mg/ml.

In other embodiments, the affinity purified human polyclonal antibodies are purified from about 2 fold to about 50,000 fold, preferably at least 10 fold, more preferably at least 100 fold and most preferably at least 1,000 fold relative to the same human polyclonal antibodies in the human blood sample. In some embodiments, the affinity purified human polyclonal antibodies have an in vivo or in vitro antibacterial or antigen binding activity per milligram of protein that is about 2 to 50,000 times higher, preferably at least 10 times higher, more preferably at least 100 times higher and most preferably at least 1,000 times higher than the corresponding in vivo or in vitro antibacterial or antigen binding activity per milligram of unpurified human immunoglobulin, or non-affinity-purified human immunoglobulin sample, e.g., intravenous immunoglobulin (IVIG) sample.

In some embodiments, the affinity purified human polyclonal antibodies are substantially free of at least 10%, 20%, 30%, 40%, 50%, 60%, 70%, preferably at least 80%, more preferably at least 90% and most preferably at least 95% of human antibodies that specifically bind to non-viral (i.e. non-Influenza A virus) and non-bacterial antigens in the human blood sample.

### IV. Pharmaceutical Compositions

In one aspect, the present invention provides pharmaceutical compositions for treating or preventing viral, bacterial, eukaryotic protist and/or fungal infection(s), which comprise an effective amount of human polyclonal antibodies affinity purified from a human blood sample with one of the viral, bacterial, eukaryotic protist and/or fungal antigenic compositions according to the present invention. The viral, bacterial, eukaryotic protist or fungal antigenic compositions include an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus antigenic composition(s) according to the present invention,

In some embodiments, an antigenic preparation comprises cellular and/or secreted antigens from bacterial cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni and a combination thereof; and/or an antigenic preparation comprising cellular and/or secreted antigens from eukaryotic protist or fungal cells selected from a Plasmodium, Pneumocystis jirovecii, Histoplasma capsulatum, Blastomyces dermatitidis, a Coccidioides and an Aspergillus.

In one aspect, the invention provides pharmaceutical compositions for treating or preventing an Influenza A virus infection, which comprise an effective amount of human polyclonal antibodies affinity purified from a human blood sample with one of the Influenza A virus antigenic compositions according to the present invention.

In another aspect, the invention provides pharmaceutical compositions for treating or preventing a K. pneumoniae infection, which comprise an effective amount of human polyclonal antibodies affinity purified from a human blood sample with an antigenic preparation comprising cellular and/or secreted antigens of K. pneumoniae cells.

In a further aspect, the invention provides pharmaceutical compositions for treating or preventing viral, bacterial, eukaryotic protist and/or fungal infection(s), which comprise: (a) an effective amount of human polyclonal antibodies affinity purified from a human blood sample with one of the influenza A virus, a Variola virus, a respiratory syncytial virus (RSV), a Cytomegalovirus (CMV) antigenic compositions according to the present invention; (b) an effective amount of human polyclonal antibodies affinity purified from a human blood sample with an antigenic preparation comprising cellular and/or secreted antigens from bacterial, eukaryotic protist or fungal cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni, a Plasmodium, Pneumocystis jirovecii, Histoplasma capsulatum, Blastomyces dermatitidis, a Coccidioides and an Aspergillus, and a combination thereof, and/or an effective amount of human polyclonal antibodies affinity purified from a human blood sample, the antibodies having specificity for an antigen from human tumor necrosis factor alpha (TNF-α), as described in detail above.

In some embodiments, the pharmaceutical composition for treating or preventing an Influenza A virus infection and an accompanying bacterial infection comprises an effective amount of human polyclonal antibodies that are specific for antigens of Influenza A virus, S. aureus, S. pneumoniae, E. coli, P. aeruginosa, A. baumannii, E. faecalis and K. pneumoniae. In some embodiments, the pharmaceutical composition for treating or preventing an Influenza A virus infection and an accompanying bacterial infection comprises an effective amount of human polyclonal antibodies that are specific for antigens of Influenza A virus, S. aureus, S. pneumoniae, E. coli, P. aeruginosa, A. baumannii, E. faecium and K. pneumoniae.

In a related aspect, the invention provides pharmaceutical compositions for treating or preventing an Influenza A virus infection and an accompanying bacterial infection, which comprise an effective amount of human polyclonal antibodies affinity purified from a human blood sample, the antibodies having specificity for an Influenza A viral antigen, a bacterial antigen and an antigen from human tumor necrosis factor alpha (TNF-α). In some embodiments, the human polyclonal antibodies having specificity for the Influenza A viral antigen are affinity purified from the human blood sample with one of the Influenza A virus antigenic compositions according to the present invention. In some embodiments, the human polyclonal antibodies having specificity for the bacterial antigen are affinity purified from the human blood sample with an antigenic preparation comprising cellular and/or secreted antigens from bacterial cells selected from S. aureus, a Streptococcus, E. coli, P. aeruginosa, A. baumannii, E. faecium, E. faecalis, E. aerogenes, E. cloacae, C. difficile, K. pneumoniae and a combination thereof. As discussed above, by combining human polyclonal antibodies specific for an Influenza A viral antigen and a bacterial antigen with polyclonal antibodies against TNF-α, one can neutralize the viral and bacterial infections and suppress the acute immune response mediated by TNF-α at the same time.

In some embodiments, the pharmaceutical composition for treating or preventing an Influenza A virus infection and an accompanying bacterial infection comprises an effective amount of human polyclonal antibodies that are specific for antigens of Influenza A virus, S. aureus, S. pneumoniae, E. coli, P. aeruginosa, A. baumannii, E. faecalis, K. pneumoniae and TNF-α. In some embodiments, the pharmaceutical composition for treating or preventing an Influenza A virus infection and an accompanying bacterial infection comprises an effective amount of human polyclonal antibodies that are specific for antigens of Influenza A virus, S. aureus, S. pneumoniae, E. coli, P. aeruginosa, A. baumannii, E. faecium, K. pneumoniae and TNF-α.

The pharmaceutical compositions of the present invention may further comprise an additional therapeutic or preventive agent. The additional therapeutic or preventive agent may be an antiviral, such as amantadine, rimantadine, oseltamivir or zanamivir, an antibiotic, such as penicillin, a penicillinase-resistant penicillin (e.g., methicillin, oxacillin, cloxacillin, dicloxacillin or flucloxacillin), a glycopeptide (e.g., vancomycin) or an aminoglycoside (e.g., kanamycin, gentamicin or streptomycin), an antimicrobial agent, a bactericidal agent (e.g., lysostaphin), a bacteriostatic agent, or an immunostimulatory compound, such as a β-glucan or granulocyte macrophage colony-stimulating factor (GM-CSF).

### V. Immunological Compositions and Vaccines

In one aspect, the present invention provides an immunological composition for treating or preventing bacterial infections. The immunological composition comprises an effective amount of one or more of the antigenic preparations according to the invention.

In some embodiments, the immunological composition comprises an effective amount of an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus, e.g., Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

In some embodiments, the immunological composition comprises an antigenic preparation comprising cellular and secreted antigens from at least 3, 4, 5, 6, 7, 8, 9, 10, 11, 12, 13, 14, 15, 16, 17, 18, 19, 20, or all 21 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

In some embodiments, the immunological composition comprises an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising cellular and secreted antigens from at least 3, 4, 5, 6, 7, or all 8 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising cellular and secreted antigens from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

In some embodiments, the immunological composition comprises an antigenic preparation comprising multiple bacterial antigens. In some embodiments, the antigenic preparation comprises a whole cell extract and a secreted antigen of a bacterium. In some embodiments, the antigenic preparation comprises an endotoxin and/or an exotoxin.

In some embodiments, the immunological composition comprises an antigenic preparation comprising cellular and secreted antigens from S. aureus. In some embodiments, the immunological composition comprises an antigenic preparation comprising cellular and secreted antigens from a Streptococcus, such as S. pneumoniae. In some embodiments, the immunological composition comprises an antigenic preparation comprising cellular and secreted antigens from E. coli. In some embodiments, the immunological composition comprises an antigenic preparation comprising cellular and secreted antigens from K. pneumoniae. In some embodiments, the immunological composition comprises an antigenic preparation comprising cellular and secreted antigens from an Enterococcus, such as E. faecium or E. faecalis. In some embodiments, the immunological composition comprises an antigenic preparation comprising cellular and secreted antigens from H. influenzae. In some embodiments, the immunological composition comprises an antigenic preparation comprising cellular and secreted antigens from P. aeruginosa. In some embodiments, the immunological composition comprises an antigenic preparation comprising cellular and secreted antigens from A. baumannii.

In some embodiments, the immunological composition comprises an antigenic preparation comprising a whole cell extract and a secreted antigen from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae, Pseudomonas aeruginosa (P. aeruginosa), and Acinetobacter baumannii (A. baumannii).

In one aspect, the invention provides an immunological composition for treating cystic fibrosis, which immunological composition comprises an antigenic composition comprising cellular and secreted antigens from Pseudomonas aeruginosa (P. aeruginosa). In some embodiments, the antigenic preparation comprises a whole cell extract and a secreted antigen of P. aeruginosa.

In another aspect, the invention provides an immunological composition for treating cystic fibrosis, which immunological composition comprises an antigenic composition comprising cellular and secreted antigens from Burkholderia cepacia complex (BCC). the antigenic composition comprises cellular and secreted antigens from B. cepacia, B. multivorans, B. cenocepacia, B. vietnamiensis, B. stabilis, B. ambifaria, B. dolosa, B. anthina, and B. pyrrocinia. In some embodiments, the antigenic preparation comprises a whole cell extract and a secreted antigen of BCC. In some embodiments, the antigenic composition comprises a whole cell extract and a secreted antigen of B. cepacia, B. multivorans, B. cenocepacia, B. vietnamiensis, B. stabilis, B. ambifaria, B. dolosa, B. anthina, and B. pyrrocinia.

The antigenic preparation for the immunological composition can be prepared by any suitable methods. In some embodiments, the immunological composition comprises an antigenic preparation prepared by the following steps: a) growing bacterial cells in a first protein containing culture medium; b) collecting and resuspending the bacterial cells in a second non-protein containing culture medium; c) growing the bacterial cells in the second non-protein containing culture medium; and d) disrupting the bacterial cells and collecting a whole cell extract from the disrupted bacterial cells. In other embodiments, the antigen preparation can further comprise a step of collecting a secreted antigen from the second non-protein containing culture medium in which the bacterial cells have grown.

In one aspect, the present invention provides a vaccine for immunizing or treating a subject, preferably a human subject. The vaccine comprises an effective amount of one or more of the immunological compositions according to the invention.

### VI. Formulations

The affinity purified human polyclonal antibodies can be incorporated into a wide variety of pharmaceutical compositions suitable for administration. Such compositions typically comprise the agent and a pharmaceutically acceptable carrier or excipient. Supplementary active compounds can also be incorporated into the compositions. Various pharmaceutical compositions and techniques for their preparation and use will be known to those of skill in the art in light of the present disclosure. For a detailed listing of suitable pharmacological compositions and associated administrative techniques one may refer to the detailed teachings herein, which may be further supplemented by texts such as REMINGTON: THE SCIENCE AND PRACTICE OF PHARMACY, 20th Ed. (Lippincott, Williams & Wilkins 2003).

Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. Further, those of ordinary skill in the art can readily deduce that suitable formulations involving these compositions and dosage forms, including those formulations as described elsewhere herein.

Pharmaceutically-acceptable materials, composition or vehicle, such as a liquid or solid filler, diluent, excipient, solvent or encapsulating material, involved in carrying or transporting the subject moiety or chemical, e.g., an antibody, from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Therapeutic formulations can be solubilized and administered via any route capable of delivering the therapeutic composition to the subject. One exemplary formulation for intravenous injection comprises the therapeutic antibody composition in an aqueous solution comprising bacteriostatic or sterile water, 10% maltose and 0.03% Polysorbate 80, pH 5.5. Another formulation for intravenous injection comprises the therapeutic antibody composition in an aqueous solution comprising bacteriostatic or sterile water and about 0.2 M glycine, pH 4.0-4.5. Therapeutic preparations can be lyophilized and stored as sterile powders, preferably under vacuum, and then reconstituted in bacteriostatic water or in sterile water prior to injection.

The vaccine of the present invention may be formulated with an adjuvant (e.g., an aluminum salt or gel), an excipient (e.g., an antibiotic, an egg protein, a stabilizer or a preservative), or an immune response potentiator (e.g., Bacille Calmette-Guerin (BCG), Corynebacterium parvum, Brucella abortus extract, glucan, levamisole, tilorone, an enzyme, or a non-virulent virus). In some embodiments, the stabilizer is monosodium glutamate (MSG) or 2-phenoxyethanol. In some embodiments, the preservative is formaldehyde, phenoxyethanol, Thimerosal or a mercury-containing preservative.

In some embodiments, the vaccine is formulated for intravenous, intraperitoneal, intracorporeal, intra-articular, intraventricular, intrathecal, intramuscular, subcutaneous, intranasal, intravaginal, topical or oral administration. In some embodiments, the vaccine is formulated as a solid (e.g., a tablet), a semi-solid, a gel, a liquid, a semi-liquid, a skin patch, or an aerosol. In some embodiments, the vaccine is formulated for administration with a liposome, an immune stimulating complex (ISCOM), or a micro-needle.

### VII. Methods of Treatment and/or Prevention

In one aspect, the present invention provides methods for treating or preventing viral, bacterial, eukaryotic protist and/or fungal infection(s), which comprise administering to a human patient suffering, suspected of suffering or at risk of suffering from the viral, bacterial, eukaryotic protist and/or fungal infection(s), an effective amount of human polyclonal antibodies affinity purified from a human blood sample with one of the viral, bacterial, eukaryotic protist and/or fungal antigenic compositions according to the present invention. The viral, bacterial, eukaryotic protist or fungal antigenic compositions include an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus antigenic composition(s) according to the present invention. The antigenic compositions can further include a TNF-α antigen.

In some embodiments, an antigenic preparation comprises cellular and/or secreted antigens from bacterial cells selected from Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, and/or Campylobacter jejuni and a combination thereof; and/or an antigenic preparation comprising cellular and/or secreted antigens from eukaryotic protist or fungal cells selected from a Plasmodium, Pneumocystis jirovecii, Histoplasma capsulatum, Blastomyces dermatitidis, a Coccidioides and an Aspergillus.

In one aspect, the present invention provides methods for treating or preventing an Influenza A virus infection, which comprise administering to a human patient suffering, suspected of suffering or at risk of suffering from an Influenza A virus infection, an effective amount of one of the pharmaceutical compositions for treating or preventing an Influenza A virus infection according to the present invention.

In a related aspect, the invention provides methods for treating or preventing an Influenza A virus infection and a bacterial infection, comprising administering to a human patient suffering, suspected of suffering or at risk of suffering from an Influenza A virus infection, S. aureus infection, a Streptococcus infection, E. coli infection, P. aeruginosa infection, A. baumannii infection, E. faecium infection, an E. faecalis infection, an E. aerogenes infection, an E. cloacae infection, C. difficile infection, and/or K. pneumoniae infection, an effective amount of one of the pharmaceutical compositions for treating or preventing an Influenza A virus infection and a bacterial infection according to the present invention.

In some embodiments, the method for treating or preventing an Influenza A virus infection and a bacterial infection comprises administering to a human suffering, suspected of suffering, or at risk of suffering from an Influenza A virus infection, a S. aureus infection, a S. pneumoniae infection, an E. coli infection, a P. aeruginosa infection, an A. baumannii infection, an E. faecalis infection and/or a K. pneumoniae infection, an effective amount of a pharmaceutical composition comprising affinity-purified human polyclonal antibodies that are specific for Influenza A virus, S. aureus, S. pneumoniae, E. coli, P. aeruginosa, A. baumannii, E. faecalis, K. pneumoniae and TNF-α antigens. In some embodiments, the method for treating or preventing an Influenza A virus infection and a bacterial infection comprises administering to a human suffering, suspected of suffering, or at risk of suffering from an Influenza A virus infection, a S. aureus infection, a S. pneumoniae infection, an E. coli infection, a P. aeruginosa infection, an A. baumannii infection, an E. faecium infection and/or a K. pneumoniae infection, an effective amount of a pharmaceutical composition comprising affinity-purified human polyclonal antibodies that are specific for Influenza A virus, S. aureus, S. pneumoniae, E. coli, P. aeruginosa, A. baumannii, E. faecium, K. pneumoniae and TNF-α antigens.

In a further aspect, the invention provides methods for treating or preventing a K. pneumoniae infection, which comprise administering to a human patient suffering, suspected of suffering or at risk of suffering from a K. pneumoniae infection, an effective amount of one of the pharmaceutical compositions for treating or preventing a K. pneumoniae infection according to the present invention.

In some embodiments, the affinity purified human polyclonal antibodies are purified (e.g., as made more concentrated as compared to the starting or unpurified material) relative to the same human polyclonal antibodies in the unpurified or non-affinity-purified human blood sample, e.g., intravenous immunoglobulin (IVIG) sample. Preferably, the affinity purified human polyclonal antibodies are specific for the viral, bacterial, eukaryotic protest, fungal and/or TNF-α antigens used in the affinity purification. Also preferably, the affinity purified human polyclonal antibodies are substantially free of human antibodies that specifically bind to non-viral, non-bacterial, non-eukaryotic protest, non-fungal and/or non-TNF-α antigens in the human blood sample.

Preferably, the affinity purified human polyclonal antibodies are concentrated, enriched or purified relative to the same human polyclonal antibodies in the unpurified or non-affinity-purified human blood sample, e.g., intravenous immunoglobulin (IVIG) sample at least 2 fold. In one example, the specific polyclonal antibodies in the unpurified or non-affinity-purified human blood sample have a concentration of 1 mg polyclonal antibodies per 1,000 mg total antibodies, wherein 999 mg are non specific antibodies. The affinity purified human polyclonal antibodies used in the present methods have a concentration of at least 2 mg polyclonal antibodies per 1,000 mg total antibodies, wherein 998 mg are non specific antibodies. In still other embodiments, the affinity purified human polyclonal antibodies are concentrated, enriched or purified relative to the same human polyclonal antibodies in the unpurified or non-affinity-purified human blood sample, e.g., intravenous immunoglobulin (IVIG) sample at least 5, 10, 100, 1,000, 10,000 or 50,000 fold.

In some embodiments, the present methods are effective for treating the majority of the listed infections so that the combination therapy avoids the time of waiting for a time consuming diagnosis, e.g., a bacterial culture test.

The methods of the present invention overcome the narrow specificity of monoclonal antibodies by providing a wide assortment of human polyclonal antibodies that are specific to both viral and bacterial antigens. At the same time, the present methods also address the lack of specificity of some existing immunoglobulin preparations by providing human polyclonal antibodies that have been affinity purified with viral and bacterial antigens to substantially exclude those antibodies that specifically bind to non-viral, non-bacterial, non-eukaryotic protest, and/or non-fungal targets, thereby lowering the amount of antibodies that are required to achieve the desired therapeutic or preventive effect and reducing the likelihood of adverse side effects.

In some embodiments, the methods of the present invention may utilize an antigenic preparation comprising cellular and/or secreted antigen(s) from S. aureus. In some embodiments, the method may utilize an antigenic preparation comprising cellular and/or secreted antigen(s) from a Streptococcus. In some embodiments, the methods may utilize an antigenic preparation comprising cellular and/or secreted antigen(s) from E. coli. In some embodiments, the methods may utilize an antigenic preparation comprising cellular and/or secreted antigen(s) from P. aeruginosa. In some embodiments, the methods may utilize an antigenic preparation comprising cellular and/or secreted antigen(s) from A. baumannii. In some embodiments, the methods may utilize an antigenic preparation comprising cellular and/or secreted antigen(s) from E. faecium. In some embodiments, the methods may utilize an antigenic preparation comprising cellular and/or secreted antigen(s) from E. faecalis. In some embodiments, the methods may utilize an antigenic preparation comprising cellular and/or secreted antigen(s) from E. aerogenes. In some embodiments, the methods may utilize an antigenic preparation comprising cellular and/or secreted antigen(s) from E. cloacae. In some embodiments, the methods may utilize an antigenic preparation comprising cellular and/or secreted antigen(s) from C. difficile. In some embodiments, the methods may utilize an antigenic preparation comprising cellular and/or secreted antigen(s) from K. pneumoniae .

In some embodiments, the methods may utilize an antigenic preparation comprising cellular and/or secreted antigens from a combination of any two bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any three bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any four bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any five bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any six bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any seven bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any eight bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any nine bacterial species selected from S. aureus, a Streptococcus, E. coli, P. aeruginosa, A. baumannii, E. faecium, E. faecalis, E. aerogenes, E. cloacae, C. difficile and K. pneumoniae. In some embodiments, the methods may utilize an antigenic preparation comprising cellular and/or secreted antigens from a combination of any ten bacterial species selected from S. aureus, a Streptococcus, E. coli, P. aeruginosa, A. baumannii, E. faecium, E. faecalis, E. aerogenes, E. cloacae, C. difficile and K. pneumoniae.

Alternatively, the present methods may utilize an antigenic preparation comprising cellular and/or secreted antigens from each of S. aureus, a Streptococcus, E. coli, P. aeruginosa, A. baumannii, E. faecium, E. faecalis, E. aerogenes, E. cloacae, C. difficile and K. pneumoniae. In some embodiments, the present methods utilize an antigenic preparation comprising cellular and/or secreted antigens from each of S. aureus, S. pneumoniae, E. coli, P. aeruginosa, A. baumannii, E. faecalis and K. pneumoniae. In some embodiments, the present methods utilize an antigenic preparation comprising cellular and/or secreted antigens from each of S. aureus, S. pneumoniae, E. coli, P. aeruginosa, A. baumannii, E. faecium and K. pneumoniae .

The methods of the present invention are useful for the treatment and prophylaxis of human subjects, particularly, infants, children, teenagers, young adults, adults, seniors, nursing mothers, surgical patients, individuals with foreign implanted medical devices or parts (e.g., catheters, prostheses, artificial hips, knees or limbs, dialysis access grafts, pacemakers and implantable defibrillators), patients with fistulas, immunocompromised patients, such as chemotherapy patients or patients taking steroids or immunosuppressive drugs (e.g., transplant patients, cancer patients and HIV positive individuals), patients with chronic illnesses, patients being cared for in health care facilities (e.g., hospitals, nursing homes, or dialysis centers), patients with an indwelling catheter, and patients who previously suffered from an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus infection.

In some embodiments, the human subjects may be healthy individuals. In some embodiments, the human subjects may suffer, be suspected of suffering or be at risk of suffering from an Influenza A viral infection and/or from bacteremia, such as S. aureus bacteremia, a Streptococcus bacteremia, E. coli bacteremia, P. aeruginosa bacteremia, A. baumannii bacteremia, E. faecium bacteremia, E. faecalis bacteremia, E. aerogenes bacteremia, E. cloacae bacteremia, C. difficile bacteremia and/or K. pneumoniae bacteremia.

In addition to bacteremia, S. aureus infection may cause a broad range of illnesses from minor skin infections, such as atopic dermatitis, impetigo, boils, cellulitis, folliculitis, furuncles, carbuncles, scalded skin syndrome and abscesses, to life-threatening diseases such as staphylococcal pneumonia, staphylococcal meningitis, osteomyelitis, endocarditis, staphylococcal toxic shock syndrome (TSS) and septicemia. A Streptococcus infection may cause streptococcal pneumonia, streptococcal meningitis, streptococcal pharyngitis (“strep throat”), otitis media, scarlet fever, acute rheumatic fever, cellulitis, endocarditis, streptococcal TSS and perinatal Group B streptococcal disease. An E. coli infection may cause gastroenteritis, a urinary tract infection, neonatal meningitis, hemolytic-uremic syndrome (HUS), peritonitis, mastitis, septicemia and Gram-negative pneumonia. A P. aeruginosa infection may cause pneumonia, bacteremia, septicemia, a urinary tract infection, a gastrointestinal infection, ear and eye infections, a chronic lung infection, endocarditis, dermatitis and osteochondritis. An A. baumannii infection may cause nosocomial pneumonia and various other infections, such as skin and wound infections, bacteremia and meningitis. An Enterococcus infection (e.g., E. faecium or E. faecalis) may cause urinary tract infections, bacteremia, bacterial endocarditis, diverticulitis, wound infections, intra-abdominal and pelvic infections, hepatobiliary sepsis, neonatal sepsis and meningitis. Enterobacter infections (e.g., E. aerogenes or E. cloacae) are associated with bacteremia, lower respiratory tract infections, skin and soft-tissue infections, gastrointestinal and urinary tract infections, septic arthritis, osteomyelitis, ophthalmic infections, central nervous system infections and meningitis, particularly in immunocompromised patients and those who are on mechanical ventilation. A C. difficile infection is a common cause of colitis and pseudomembranous colitis in patients receiving antibiotic treatments for extended periods of time. In addition to colitis and pseudomembranous colitis, a C. difficile infection may cause severe diarrhea, toxic megacolon, intestinal perforation and even death. A K. pneumoniae infection typically causes pneumonia and urinary tract infections.

Accordingly, the methods of the present invention are useful for treating and preventing any of the above diseases associated with Influenza A virus, S. aureus, Streptococcus, E. coli, P. aeruginosa, A. baumannii, E. faecium, E. faecalis, E. aerogenes, E. cloacae, C. difficile and/or K. pneumoniae infections.

In some embodiments, the Influenza A virus has one of the following subtypes: H1N1, H2N2, H3N2, H5N1, H7N7, H1N2, H9N2, H7N2, H3N2, H7N3, H5N2 or H10N7. In some embodiments, the Influenza A virus is a strain that caused the “Spanish Flu” and the 2009 swine flu outbreak (H1N1), caused the “Asian Flu” in the late 1950s (H2N2), or caused the “Hong Kong Flu” in the late 1960s (H3N2). In some embodiments, the Influenza A virus is resistant to an antiviral drug, such as amantadine, rimantadine, oseltamivir, or zanamivir.

In some embodiments, the S. aureus infection accompanying the primary Influenza A viral infection may be caused by a S. aureus strain that is resistant to an antibiotic, such as a methicillin-resistant strain (MRSA), a vancomycin intermediate strain (VISA) or vancomycin resistant strain (VRSA). In some embodiments, the S. aureus may be selected from methicillin resistant strains USA300 (also known as FPR 3757; ATCC # BAA-1556) and NYBK2464 (ATCC # BAA-51). In some embodiments, the Streptococcus infection accompanying the primary Influenza A viral infection may be caused by a S. pneumoniae strain that is resistant to an antibiotic, such as penicillin, tetracycline, clindamycin, a cephalosporin, a macrolide or a quinolone. In some embodiments, the E. coli infection accompanying the primary Influenza A viral infection may be caused by an E. coli strain that is resistant to an antibiotic, such as penicillin, streptomycin, chloramphenicol, ampicillin, cephalosporin or tetracycline. In some embodiments, the P. aeruginosa infection accompanying the primary Influenza A viral infection may be caused by a P. aeruginosa strain that is resistant to an antibiotic, such as a β-lactam antibiotic (e.g., penicillin), piperacillin, imipenem, tobramycin or ciprofloxacin. In some embodiments, the A. baumannii infection accompanying the primary Influenza A viral infection may be caused by an A. baumannii strain that is resistant to an antibiotic, such as ceftazidime, gentamicin, ticarcillin, piperacillin, aztreonam, cefepime, ciprofloxacin, imipenem or meropenem. In some embodiments, the E. faecium or E. faecalis infection accompanying the primary Influenza A viral infection may be caused by an E. faecium or E. faecalis strain that is resistant to an antibiotic, such as a penicillin, a cephalosporin, an aminoglycoside, aztreonam, clindamycin or vancomycin. In some embodiments, the E. aerogenes or E. cloacae infection accompanying the primary Influenza A viral infection may be caused by an E. aerogenes or E. cloacae strain that is resistant to an antibiotic, such as a penicillin, a cephalosporin, an aminoglycoside, a carbapenem, ciprofloxacin, trimethoprim-sufamethoxazole or a quinolone. In some embodiments, the K. pneumoniae infection accompanying the primary Influenza A viral infection may be caused by a K. pneumoniae strain that is resistant to an antibiotic, such as an aminoglycoside, penicillin or a cephalosporin (e.g., ceftazidime or carbapenem).

It is known in the art that certain bacterial antigens are conserved among different bacterial species and genera. Accordingly, the affinity purified human polyclonal antibodies of the present invention may also be useful for treating those humans who may be suffering, be suspected of suffering or be at risk of suffering from an additional bacterial infection. In some embodiments, the additional bacterial infection may be a Bacillus infection (e.g., B. anthracis), a Campylobacter infection (e.g., C. jejuni), a Clostridium infection (e.g., C. botulinum, C. perfringens or C. tetani), an Enterobacter infection (e.g., E. sakazakii), a Pantoea infection (e.g., P. agglomerans), a Helibacter infection (e.g., H. pylori), a Listeria infection (e.g., L. monocytogenes), a Mycobacterium infection (e.g., M. leprae or M. tuberculosis), a Salmonella infection (e.g., S. enterica) or a Shigella infection (e.g., S. flexneri, S. sonnei or S. dysenteriae).

As noted above, the present invention also provides a method for immunizing or treating a subject, which method comprises administering to a subject, preferably a human, for whom such immunization or treatment is needed or desirable, an effective amount of a vaccine comprising an immunological composition according to the present invention.

In some embodiments, the method is used to immunize or treat the human from a bacterial infection, especially an antibiotic-resistant bacterial infection, a tumor, or a cancer. In some embodiments, the human is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, a patient in an emergency room, a dialysis patient, a surgery patient, e.g., a patient with elective surgery, especially orthopedic surgery patient for hip, knee, shoulder, or other body part replacement, an athlete, a healthcare worker, or a tumor or cancer patient. In some embodiments, the human suffers, is suspected of suffering, or is at risk of suffering from bacteremia.

In some embodiments, the vaccine comprises an antigenic preparation comprising cellular and secreted antigens from S. aureus, and the human suffers, is suspected of suffering, or is at risk of suffering from toxic shock syndrome (TSS), Staphylococcal scalded skin syndrome (SSSS, also known as pemphigus neonatorum or Ritter's disease, or localized bullous impetigo), pyaemia (or pyemia), a boil (or furuncle), a carbuncle, staphylococcal endocarditis, staphylococcal pneumonia or atopic dermatitis.

In some embodiments, the vaccine comprises an antigenic preparation comprising cellular and secreted antigens from a Streptococcus, especially S. pneumoniae, and the human suffers, is suspected of suffering, or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease.

In some embodiments, the vaccine comprises an antigenic preparation comprising cellular and secreted antigens from E. coli, and the human suffers, is suspected of suffering, or is at risk of suffering from gastroenteritis, a urinary tract infection, neonatal meningitis, hemolytic-uremic syndrome (HUS), peritonitis, mastitis, septicemia or Gram-negative pneumonia.

In some embodiments, the vaccine comprises an antigenic preparation comprising cellular and secreted antigens from K. pneumoniae, and the human suffers, is suspected of suffering, or is at risk of suffering from Klebsiella pneumonia, ankylosing spondylitis (AS, previously known as Bekhterev's disease, Bekhterev syndrome, and Marie-Strümpell disease, a form of Spondyloarthritis), a urinary tract infection, a patient with chronic pulmonary disease, enteric pathogenicity, nasal mucosa atrophy, and rhinoscleroma.

In some embodiments, the vaccine comprises an antigenic preparation comprising cellular and secreted antigens from E. faecium, and the human suffers, is suspected of suffering, or is at risk of suffering from neonatal meningitis.

In some embodiments, the vaccine comprises an antigenic preparation comprising cellular and secreted antigens from H. influenzae, and the human suffers, is suspected of suffering, or is at risk of suffering from bacteremia, pneumonia, acute bacterial meningitis, cellulitis, osteomyelitis, epiglottitis, infectious arthritis, ear infection (otitis media), eye infection (conjunctivitis), sinusitis or pneumonia.

In some embodiments, the vaccine comprises an antigenic preparation comprising cellular and secreted antigens from P. aeruginosa, and the human suffers, is suspected of suffering, or is at risk of suffering from pneumonia, septic shock, urinary tract infection, gastrointestinal infection, skin and soft tissue infection, infection of a burn injury, infection of an external ear (otitis externa), hot-tub rash (dermatitis), post-operative infection in a radial keratotomy surgery patient, ecthyma gangrenosum, osteomyelitis involving puncture wound of the foot. In some embodiments, the vaccine comprises an antigenic preparation comprising cellular and secreted antigens from P. aeruginosa, and the human is a cystic fibrosis patient, a neutropenic patient, a premature infant, a neutropaenic cancer patient, a burns victim or a patient with wound infection. In some embodiments, the vaccine comprises an antigenic preparation comprising cellular and secreted antigens from BCC (i.e., a combination of B. cepacia, B. multivorans, B. cenocepacia, B. vietnamiensis, B. stabilis, B. ambifaria, B. dolosa, B. anthina, and B. pyrrocinia), and the human is a cystic fibrosis patient.

In some embodiments, the vaccine comprises an antigenic preparation comprising cellular and secreted antigens from A. baumannii, and the human suffers, is suspected of suffering, or is at risk of suffering from pneumonia, infection of the urinary tract, bloodstream or other part of the body, wound, necrotizing fasciitis, or nosocomial A. baumannii bacteremia.

In some embodiments, the bacterial infection is caused by at least two different bacteria selected from the group consisting of S. aureus, E. coli, a Streptococcus, e.g., S. pneumoniae, K. pneumoniae, an Enterococcus, e.g., E. faecium, H. influenzae, P. aeruginosa, and A. baumannii. In some embodiments, the vaccine comprises an antigenic preparation comprising cellular and secreted antigens from at least two different bacteria selected from the group consisting of S. aureus, E. coli, a Streptococcus, e.g., S. pneumoniae, K. pneumoniae, an Enterococcus, e.g., E. faecium, H. influenzae, P. aeruginosa, and A. baumannii, and the bacterial infection is caused by at least two different bacteria selected from the group consisting of S. aureus, E. coli, a Streptococcus, e.g., S. pneumoniae, K pneumoniae, an Enterococcus, e.g., E. faecium, H. influenzae, P. aeruginosa, and A. baumannii.

The above therapeutic and prophylactic approaches may be combined with any one of a wide variety of therapeutic regimens for the treatment or prevention of viral and bacterial infections. For example, the affinity purified human polyclonal antibodies and/or vaccines of the present invention may be administered in conjunction with an additional therapeutic or preventive agent. The additional therapeutic or preventive agent may be an antiviral, such as amantadine, rimantadine, oseltamivir or zanamivir, an antibiotic, such as penicillin, a penicillinase-resistant penicillin (e.g., methicillin, oxacillin, cloxacillin, dicloxacillin or flucloxacillin), a glycopeptide (e.g., vancomycin) or an aminoglycoside (e.g., kanamycin, gentamicin or streptomycin), an antimicrobial agent, a bactericidal agent (e.g., lysostaphin), a bacteriostatic agent, or an immunostimulatory compound, such as a β-glucan or granulocyte macrophage colony-stimulating factor (GM-CSF).

### VIII. Immunological Testing

Another important aspect of the present invention concerns the use of the affinity purified human polyclonal antibodies for identifying those human subjects who may be suitable for polyclonal antibody therapy or prophylaxis of viral, bacterial, eukaryotic protist and/or fungal infection, for monitoring the progress and/or efficacy of the therapeutic or prophylactic treatment, and for determining an optimal therapeutic or prophylactic dose based on a human subject's initial response to the treatment with affinity purified human polyclonal antibodies. The antibodies that can be used in the immunological testing include human polyclonal antibodies affinity purified from a human blood sample with one of the viral, bacterial, eukaryotic protist and/or fungal antigenic compositions according to the present invention. The viral, bacterial, eukaryotic protist or fungal antigenic compositions include an influenza A virus, a Variola virus, a respiratory syncytial virus (RSV), a Cytomegalovirus (CMV), Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae, Campylobacter jejuni, a Plasmodium, Pneumocystis jirovecii, Histoplasma capsulatum, Blastomyces dermatitidis, a Coccidioides and/or an Aspergillus antigenic composition(s) according to the present invention. The antigenic compositions can further include a TNF-α antigen.

In some embodiments, the therapeutic and preventive methods of the present invention comprise conducting an immunotest prior to administering the affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigen to a human subject, in order to assess the suitability of the human subject for the therapeutic or preventive treatment. The same affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens are used to determine the presence, absence and/or amount of the antigens in a suitable sample, e.g., a blood sample, from a candidate for the polyclonal antibody treatment. A positive immunotest result indicates that the candidate is suitable for therapy or prevention of an Influenza A virus, S. aureus, a Streptococcus, E. coli, P. aeruginosa, A. baumannii, E. faecium, E. faecalis, E. aerogenes, E. cloacae, C. difficile, and/or K. pneumoniae infection using the affinity purified human polyclonal antibodies.

In some embodiments, the immunotest is conducted to determine the presence, absence and/or amount of an Influenza A virus, S. aureus, S. pneumoniae, E. coli, P. aeruginosa, A. baumannii, E. faecalis, K. pneumoniae and TNF-α antigens, and a positive immunotest result indicates that the human subject is suitable for therapy or prevention of Influenza A virus, S. aureus, S. pneumoniae, E. coli, P. aeruginosa, A. baumannii, E. faecalis and K. pneumoniae infections using the affinity purified human polyclonal antibodies.

In some embodiments, the immunotest is conducted to determine the presence, absence and/or amount of an Influenza A virus, S. aureus, S. pneumoniae, E. coli, P. aeruginosa, A. baumannii, E. faecium, K. pneumoniae and TNF-α antigens, and a positive immunotest result indicates that the human subject is suitable for therapy or prevention of Influenza A virus, S. aureus, S. pneumoniae, E. coli, P. aeruginosa, A. baumannii, E. faecium and K. pneumoniae infections using the affinity purified human polyclonal antibodies.

In some embodiments, the therapeutic and preventive methods of the present invention comprise conducting an immunotest before and after administering the affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens to a human subject, in order to monitor the efficacy of the therapeutic or preventive treatment. The same affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens are used to determine the presence, absence and/or amount of the antigens in a suitable sample, e.g., a blood sample, taken from the treated human subject before and after the administration of the antibodies. The absence or reduction in the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens after administering the affinity purified human polyclonal antibodies to the patient relative to the amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens before the administration indicates efficacy of the therapeutic or preventive treatment.

In some embodiments, the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, and the absence or reduction in the Influenza A virus, S. aureus, S. pneumoniae, E. coli, P. aeruginosa, A. baumannii, E. faecalis, E. faecalis, K. pneumoniae and TNF-α antigens after administering the affinity purified human polyclonal antibodies to the human subject relative to the amount of the Influenza A virus, S. aureus, S. pneumoniae, E. coli, P. aeruginosa, A. baumannii, E. faecalis, K. pneumoniae and TNF-α antigens before the administration indicates efficacy of the therapeutic or preventive treatment.

In some embodiments, the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, and the absence or reduction in the Influenza A virus, S. aureus, S. pneumoniae, E. coli, P. aeruginosa, A. baumannii, E. faecium, E. faecalis, K. pneumoniae and TNF-α antigens after administering the affinity purified human polyclonal antibodies to the human subject relative to the amount of the Influenza A virus, S. aureus, S. pneumoniae, E. coli, P. aeruginosa, A. baumannii, E. faecium, K. pneumoniae and TNF-α antigens before the administration indicates efficacy of the therapeutic or preventive treatment.

In some embodiments, the therapeutic or preventive methods of the present invention comprise conducting an immunotest before and after administering the affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens to a human subject, in order to determine an optimal therapeutic or prophylactic dose based on the subject's response to the treatment with affinity purified human polyclonal antibodies. The same affinity purified human polyclonal antibodies against Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens are used to determine the presence, absence and/or amount of the antigens in a suitable sample, e.g., a blood sample, taken from the treated human subject before and after the administration of the antibodies. The optimal therapeutic, removal or prophylactic dose of the affinity purified human polyclonal antibodies is determined based on the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens remaining after administering the affinity purified human polyclonal antibodies to the individual and the extent of reduction in the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens after administering the affinity purified human polyclonal antibodies relative to the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens before the administration.

In some embodiments, the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecalis, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecalis, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecalis, K. pneumoniae and TNF-α antigens before the administration.

In some embodiments, the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecium, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecium, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecium, K. pneumoniae and TNF-α antigens before the administration.

In some embodiments, the method of vaccination also comprises assaying S. aureus infection, a Streptococcus infection, E. coli infection, K. pneumoniae infection, E. faecium infection, H. influenzae infection, P. aeruginosa infection, and/or A. baumannii infection in the human before, during and/or after the vaccination.

A variety of immunotests are contemplated. In some embodiments, the present methods assess the complex formed between bacterial antigens and affinity purified human polyclonal antibodies via a sandwich or competitive assay format. In other embodiments, the complex is assessed in a homogeneous or a heterogeneous assay format. In some embodiments, the complex is assessed by a format selected from an enzyme-linked immunosorbent assay (ELISA), chemiluminescent assay, immunoblotting, immunoprecipitation, radioimmunoassay (RIA), immunostaining, latex agglutination, indirect hemagglutination assay (IHA), complement fixation, indirect immunofluorescence assay (IFA), nephelometry, flow cytometry assay, plasmon resonance assay, chemiluminescence assay, lateral flow immunoassay, μ-capture assay, inhibition assay and avidity assay. In other embodiments, the immunotest is conducted as a precipitation or an agglutination assay.

### IX. Administration and Dosage

Single or multiple doses of the affinity purified human polyclonal antibodies may be delivered to a human subject using any convenient mode of administration, including but not limited to intravenous, intraperitoneal, intracorporeal, intra-articular, intraventricular, intrathecal, intramuscular, subcutaneous, intranasal, intravaginal, topical and oral administration. In one embodiment, single or multiple doses of the affinity purified human polyclonal antibodies may be delivered to a human subject by intravenous administration.

A therapeutically effective amount of the affinity purified human polyclonal antibodies administered to a given individual will, of course, be dependent on a number of factors, including the concentration of the affinity purified human polyclonal antibodies, composition or dosage form, the selected mode of administration, the age and general condition of the individual being treated, the sex of the individual, the severity of the individual's condition, and other factors known to the prescribing physician.

In some embodiments, the affinity purified human polyclonal antibodies of the present invention are administered in a dosage from about 0.1 mg per kg bodyweight to about 10 mg per kg bodyweight, preferably from about 0.3 mg per kg bodyweight to about 3 mg per kg bodyweight, more preferably from about 0.6 mg per kg bodyweight to about 2 mg per kg bodyweight, and most preferably from about 1 mg per kg bodyweight to about 1.5 mg per kg bodyweight. The above mentioned mg per kg dosage refers to the mg of specific antibody against the bacterial antigens, and not necessarily to the total mg of antibody in the preparation which may include antibodies that are not specific to bacterial antigens.

In other embodiments, the affinity purified human polyclonal antibodies are administered with a frequency preferably ranging from approximately once a day to approximately once a month, more preferably from approximately once a week to approximately once every two weeks, most preferably approximately once every two weeks. The dosages for treating chronic infection, e.g., patients with indwelling catheters, post surgical difficult infections and knee replacements, may be different from dosages for treating acute infection, e.g., ICU septic patients. The dosages for prophylactic use may also be different. For prophylactic use, the antibodies may be added to locks on catheters in place of antibiotic locks, or the antibodies may be to peritoneal dialysis solutions, etc. Treating chronic infection, acute infection or prophylactic may use different doses and dosing schedules.

In some embodiments, the vaccine of the present invention is administered to the subject via intravenous, intraperitoneal, intracorporeal, intra-articular, intraventricular, intrathecal, intramuscular, subcutaneous, intranasal, intravaginal, topical or oral route. In some embodiments, the vaccine is administered to a tumor or cancer site (e.g., bladder or colorectal cancer, particularly a superficial form of bladder cancer). In some embodiments, the vaccine is administered to the subject as a solid (e.g., a tablet), a semi-solid, a gel, a liquid, a semi-liquid, a skin patch or an aerosol. In some embodiments, the vaccine is administered to the subject with a liposome, an immune stimulating complex (ISCOM), or a micro-needle. In some embodiments, the vaccine is administered to the subject with a pharmaceutically acceptable carrier or excipient.

A therapeutically effective amount of the vaccine administered to a given individual will also be dependent on a number of factors, including the concentration of the affinity purified human polyclonal antibodies, composition or dosage form, the selected mode of administration, the age and general condition of the individual being treated, the sex of the individual, the severity of the individual's condition, and other factors known to the prescribing physician.

### EXAMPLES

### Example 1

### Synthesis and Immobilization of Influenza A Virus Peptides

Forty nine Influenza A virus (H1N1) peptides having amino acid sequences of SEQ ID NOS: 1-49 were selected from the H1N1 viral components of PB1, PB2, PA, HA, NP, NA, M1, M2, NS1 and NS2. The peptides were synthesized with a cysteine added to the N-terminus as a spacer for immobilization to the solid phase, the CNBr-activated Sepharose 4B (GE Healthcare Bio-Science Corp., Piscataway, N.J.).

The lyophilized peptides were dissolved in deionized water, pH 3, before addition of the immobilization buffer, 0.05 M borate buffer, pH 8.5, containing 0.5M NaCl. The lyophilized CNBr-activated Sepharose 4B was suspended in 70% ethanol, made in deionized water, pH 3. The Sepharose 4B suspended in the 70% ethanol, pH 3, was incubated at room temperature (RT, 20-25° C.) on an orbital shaker at 75 rpm for gel sanitization. The ethanol was removed by washing the gel in a Büchner glass funnel with deionized water, pH 3.0. The washed gel was brought up to pH 8.5 with 0.05M borate buffer, pH 8.5, containing 0.5M NaCl before addition of the reconstituted H1N1 peptides at a ratio of approximately 1.0-1.5 mg peptides/ml gel slurry. The peptide-gel mixture was incubated at RT for 4 hours at 75 rpm, followed by incubation at 2-8° C. overnight on an orbital shaker at 75 rpm. The gel was drained after the overnight incubation and blocked with 1.0 M glycine, made in 0.05M borate, pH 8.5, containing 0.5M NaCl, for 1 hour at RT on an orbital shaker at 75 rpm. The gel was washed alternately with 0.05M acetate, pH 4.0, containing 0.5M NaCl and 0.05M borate buffer, pH 8.5, containing 0.5M NaCl. All the operations were performed in a Biological Safety Cabinet, using freshly made buffers that were filtered through 0.2 μM sterile filter.

### Example 2

### Affinity Purification of Human Polyclonal Antibodies from Human Plasma

Normal human source plasma of injectable grade was obtained from licensed U.S. blood collection centers. The plasma was thawed at room temperature and pooled before fractionation with saturated ammonium sulfate. Briefly, an equal volume of the 100% saturated ammonium sulfate (SAS), made in 0.01M phosphate-buffered saline (PBS), pH 7.4, was added slowly into the pooled plasma at room temperature with stirring. The mixture was stirred and incubated at room temperature for 2 hours before centrifugation at 4,500 rpm in a Beckman centrifuge. The pellets were reconstituted in PBS. Pre-mixed and pre-diluted in PBS solvent/detergent (S/D), Triton X-100 and tri-N-butyl phosphate (TNBP), was added into the reconstituted, fractionated plasma to yield 1% and 0.3% respectively. The plasma was stirred at RT for 4 hours before being buffer exchanged 5 times into PBS.

The SAS-fractionated, S/D treated human plasma was charged over the H1N1 peptide column at a liner flow rate of approximately 0.4 cm/min. The unbound plasma components and reagents were washed off the column with PBS at approximately 0.8 cm/min until the absorption at 280 nm dropped to the baseline. The captured antibodies were eluted off the column by application of the elution buffer, 0.1M glycine, pH 2.5-2.75. The pH of the eluted antibodies was neutralized by simultaneous addition of 2.0 M potassium phosphate, pH 8.5. The resulting antibodies were filtered through a 0.2 μM sterile filter, concentrated to 6-10 mg/ml and stored at 2-8° C. until further processing.

Ceramic hydroxyapatite (CHT), Type I , 40 μM in particle size, was purchased from BioRad (Hercules, Calif.). A column with packed CHT was sanitized with 0.5-1.0 M NaOH for at least 30 minutes followed by equilibration with CHT Wash Buffer, 0.05M glycine, pH 5.5. The human polyclonal antibodies affinity purified with immobilized H1N1 antigens were buffer exchanged into the CTH Wash Buffer and concentrated to yield approximately 8-10 mg/ml. The antibodies were then loaded over the CHT column. The antibody-loaded column was washed with the CHT Wash Buffer to the baseline. The monomeric IgG was eluted with 0.30-0.35 M NaCl, made in the wash buffer. The antibody fractions were analyzed by HPLC gel filtration, and the fractions with monomeric IgG below 95% were rejected.

The antibodies purified over the antigen column and the CHT column (>95% in monomeric IgG) were further buffer exchanged into the final product storage buffer, 10% maltose/0.03% polysorbate 80, pH 5.5±0.5. The purified antibodies were then filtered through a 0.2 μM sterile filter. The amount of antibodies purified from 6 L of human plasma was bottled as a single dose for patient treatment. The product was analyzed by a series of tests designed to assess the safety, efficacy, reproducibility, stability and endotoxin level, as required by the FDA for human plasma derivatives.

### Example 3

### Immune Protection Using Affinity Purified Human Polyclonal Antibodies

Madin-Darby Canine Kidney (MDCK) cells were cultured in Eagles' Minimal Essential Medium, supplemented with 2 mM L-glutamine, 10% fetal bovine serum (non-heat inactivated), 1% non-essential amino acid solution, 0.15% sodium bicarbonate, 1% sodium pyruvate and 1% penicillin/streptomycin. The culture medium was replaced by live H1N1 viral culture 3-5 days after the initial MDCK. culture. The viral culture was continued for 5-7 days until it reached viral confluence by centrifugation at 1,500 rpm for 5 minutes at 2-8° C.

Fifteen BALB/C mice, 18-20 g body weight, were used in this study. The mice were divided into three experimental groups, five animals each. The animals in Group 1 (control) received intranasal inhalation of 50 μl of culture medium, whereas the animals in Groups 2 and 3 received intranasal inhalation of 50 μl of H1N1 virus. The animals were weighed daily. Three days after the viral inhalation, the animals in Groups 1 and 2 received an intraperitoneal injection of 1 ml of buffer, whereas the animals in Group 3 received an intraperitoneal injection of 1 ml of 2 mg/ml human anti-H1N1 antibodies.

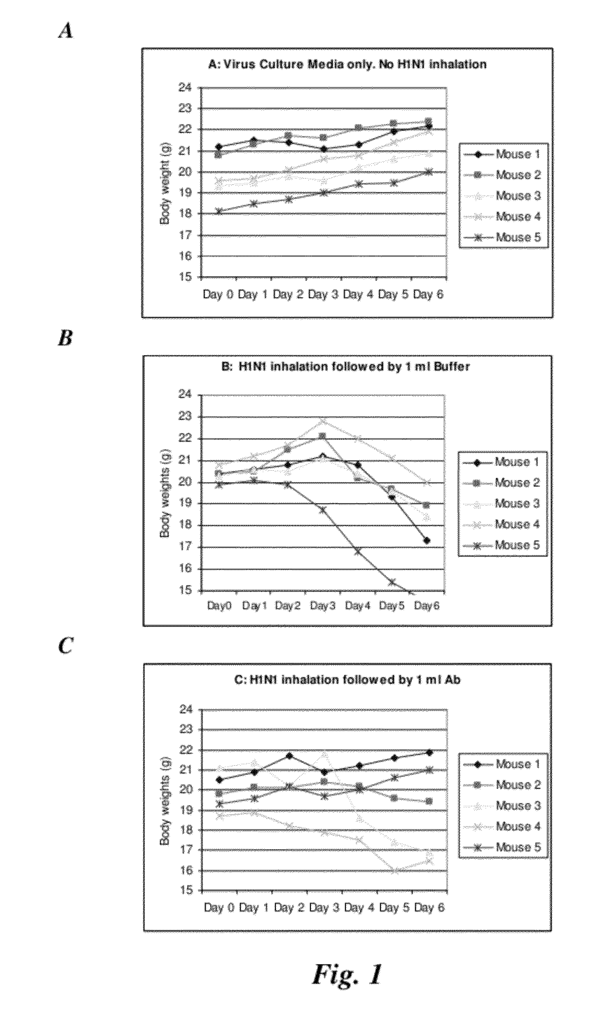

The weights of the animals in Groups 1-3 are shown in FIGS. 1A-C, respectively. As one can see from FIG. 1A, the body weights of the animals in Group 1 increased steadily throughout the duration of the experiment. The body weights of the animals in Group 2 started to decrease three days after the viral inhalation (FIG. 1B). In contrast, three out of five animals in Group 3 did not lose weight following the viral challenge (FIG. 1C), suggesting an immunoprotective mechanism by the human polyclonal anti-H1N1 antibodies.

Unless indicated otherwise, all publications and documents cited herein are incorporated by reference in their entireties. Citation of publications or documents is not intended as an admission that any of such publications or documents are pertinent prior art, nor does it constitute any admission as to the contents or date of these publications or documents.

The present invention is further illustrated by the following exemplary embodiments:

### Exemplary Embodiments Section A

1. A pharmaceutical composition for treating or preventing a Salmonella infection, which composition comprises an effective amount of human polyclonal antibodies affinity purified from a human blood sample with an antigenic preparation comprising a cellular antigen and/or a secreted antigen of Salmonella cells.

2. The pharmaceutical composition of embodiment 1, wherein the affinity purified human polyclonal antibodies are purified relative to the same human polyclonal antibodies in the unpurified or non-affinity-purified human blood sample.

3. The pharmaceutical composition of embodiment 1, wherein the affinity purified human polyclonal antibodies are specific for the Salmonella antigen(s) used in the affinity purification.

4. The pharmaceutical composition of embodiment 1, wherein the affinity purified human polyclonal antibodies are substantially free of human antibodies that specifically bind to non-Salmonella antigens in said human blood sample.

5. The pharmaceutical composition of any of embodiments 1-4, wherein the affinity purified human polyclonal antibodies specific to the Salmonella antigen(s) have a concentration ranging from about 10 μg/ml to about 10 mg/ml.

6. The pharmaceutical composition of any of embodiments 1-5, wherein the affinity purified human polyclonal antibodies are purified from about 2 fold to about 50,000 fold relative to the same human polyclonal antibodies in the unpurified or non-affinity-purified human blood sample.

7. The pharmaceutical composition of any of embodiments 1-6, wherein the human blood sample is from a normal human.

8. The pharmaceutical composition of any of embodiments 1-6, wherein the human blood sample is from a human infected with Salmonella.

9. The pharmaceutical composition of any of embodiments 1-8, wherein the human blood sample is pooled from at least 2 humans.

10. The pharmaceutical composition of any of embodiments 1-9, wherein the antigenic preparation comprises an antigen from Salmonella bongori (S. bongori) and/or Salmonella enterica (S. enterica).

11. The pharmaceutical composition of any of embodiments 1-10, wherein the antigenic preparation comprises an antigen from Salmonella enterica enterica, Salmonella enterica salamae, Salmonella enterica arizonae, Salmonella enterica diarizonae, Salmonella enterica houtenae, and/or Salmonella enterica indica.

12. The pharmaceutical composition of any of embodiment 11, wherein the Salmonella enterica enterica has serovars selected from the group consisting of Salmonella Choleraesuis, Salmonella Dublin, Salmonella Enteritidis, Salmonella Gallinarum, Salmonella Hadar, Salmonella Heidelberg, Salmonella Infantis, Salmonella Paratyphi, Salmonella Typhi and Salmonella Typhimurium.

13. The pharmaceutical composition of any of embodiments 1-12, wherein the antigenic preparation comprises a Salmonella antigen that confers antibiotic resistance.

14. The pharmaceutical composition of any of embodiment 13, wherein the antibiotic resistant strain is Salmonella Typhimurium DT104 (DT104), multidrug-resistant typhoid (MDR typhoid), or MDR-AmpC.

15. The pharmaceutical composition of any of embodiments 1-14, wherein the antigenic preparation comprises a Salmonella toxin, O-somatic antigen and/or H-flagellar antigen.

16. The pharmaceutical composition of any of embodiments 1-15, wherein the antigenic preparation comprises a whole cell extract and a secreted antigen of Salmonella.

17. The pharmaceutical composition of any of embodiments 1-16, wherein the antigenic preparation is prepared by the following steps:

a) growing Salmonella cells in a first protein containing culture medium;

b) collecting and resuspending the Salmonella cells in a second non-protein containing culture medium;

c) growing the Salmonella cells in the second non-protein containing culture medium; and

d) disrupting the bacterial cells and collecting a whole cell extract from the disrupted Salmonella cells.

18. The pharmaceutical composition of embodiment 17, which further comprises a step of removing toxin from the whole cell extract.

19. The pharmaceutical composition of any of embodiments 17-18, which further comprises a step of collecting a secreted antigen from the second non-protein containing culture medium in which the Salmonella cells have grown.

20. A method for treating or preventing a Salmonella infection, which method comprises administering to a human suffering, suspected of suffering or at risk of suffering from a Salmonella infection, an effective amount of the pharmaceutical composition of any of embodiments 1-19.

21. The method of embodiment 20, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Salmonella infection.

22. The method of embodiment 20, wherein the human for treatment has a weakened immune system, typhoid fever (also known as Salmonella typhi or commonly just typhoid), paratyphoid fevers (or enteric fevers), foodborne illness (also foodborne disease and colloquially referred to as food poisoning) or salmonellosis.

23. The method of any of embodiments 20-22, wherein the Salmonella infection is caused by a Salmonella strain that is resistant to an anti-bacterial drug or treatment.

24. The method of any of embodiments 20-23, further comprising, prior to administering the affinity purified human polyclonal antibodies to the human, conducting an immunotest to determine the presence, absence and/or amount of a Salmonella antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Salmonella infection using the affinity purified human polyclonal antibodies.

25. The method of any of embodiments 20-23, further comprising, before and after administering the affinity purified human polyclonal antibodies to the human, conducting an immunotest to determine the presence, absence and/or amount of a Salmonella antigen in a blood sample of the human using the same affinity purified human polyclonal antibodies, to monitor the efficacy of the therapeutic, removal or preventive treatment, wherein the absence or reduction in the Salmonella antigen after administering the affinity purified human polyclonal antibodies to the human relative to the amount of the Salmonella antigen before the administration indicates efficacy of the therapeutic, removal or preventive treatment.

26. The method of any of embodiments 20-23, further comprising, before and after administering the affinity purified human polyclonal antibodies to the human, conducting an immunotest to determine the presence, absence and/or amount of a Salmonella antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Salmonella antigen remaining after administering the affinity purified human polyclonal antibodies to the human and the extent of reduction in the Salmonella antigen after administering the affinity purified human polyclonal antibodies to the human relative to the amount of the Salmonella antigen before the administration.

27. A pharmaceutical composition for treating or preventing tuberculosis (TB), which composition comprises an effective amount of human polyclonal antibodies affinity purified from a human blood sample with an antigenic preparation comprising a cellular antigen and/or a secreted antigen of TB-causing Mycobacterium cells.

28. The pharmaceutical composition of embodiment 27, wherein the affinity purified human polyclonal antibodies are purified relative to the same human polyclonal antibodies in the unpurified or non-affinity-purified human blood sample.

29. The pharmaceutical composition of embodiment 27, wherein the affinity purified human polyclonal antibodies are specific for the Mycobacterium antigen(s) used in the affinity purification.

30. The pharmaceutical composition of embodiment 27, wherein the affinity purified human polyclonal antibodies are substantially free of human antibodies that specifically bind to non-Mycobacterium antigens in said human blood sample.

31. The pharmaceutical composition of any of embodiments 27-30, wherein the affinity purified human polyclonal antibodies specific to the Mycobacterium antigen(s) have a concentration ranging from about 10 μg/ml to about 10 mg/ml.

32. The pharmaceutical composition of any of embodiments 27-31, wherein the affinity purified human polyclonal antibodies are purified from about 2 fold to about 50,000 fold relative to the same human polyclonal antibodies in the unpurified or non-affinity-purified human blood sample.

33. The pharmaceutical composition of any of embodiments 27-32, wherein the human blood sample is from a normal human.

34. The pharmaceutical composition of any of embodiments 27-32, wherein the human blood sample is from a human infected with the Mycobacterium.

35. The pharmaceutical composition of any of embodiments 27-34, wherein the human blood sample is pooled from at least 2 humans.

36. The pharmaceutical composition of any of embodiments 27-35, wherein the antigenic preparation comprises an antigen from Mycobacterium tuberculosis (MTB), M. bovis, M. africanum, M. canetti, and/or M. microti.

37. The pharmaceutical composition of any of embodiments 27-36, wherein the antigenic preparation comprises an antigen from a hypervirulent strain of M. tuberculosis.

38. The pharmaceutical composition of any of embodiments 27-37, wherein the antigenic preparation comprises a Mycobacterium antigen that confers antibiotic resistance.

39. The pharmaceutical composition of any of embodiment 38, wherein the antibiotic resistant strain causes multi-drug-resistant tuberculosis (MDR-TB) or extensively drug-resistant TB (XDR-TB).

40. The pharmaceutical composition of any of embodiments 27-39, wherein the antigenic preparation comprises a Mycobacterium toxin.

41. The pharmaceutical composition of any of embodiments 27-40, wherein the antigenic preparation comprises a whole cell extract and a secreted antigen of Mycobacterium.

42. The pharmaceutical composition of any of embodiments 27-41, wherein the antigenic preparation is prepared by the following steps:

a) growing Mycobacterium cells in a first protein containing culture medium;

b) collecting and resuspending the Mycobacterium cells in a second non-protein containing culture medium;

c) growing the Mycobacterium a cells in the second non-protein containing culture medium; and

d) disrupting the bacterial cells and collecting a whole cell extract from the disrupted Mycobacterium cells.

43. The pharmaceutical composition of embodiment 42, which further comprises a step of removing toxin from the whole cell extract.

44. The pharmaceutical composition of any of embodiments 42-43, which further comprises a step of collecting a secreted antigen from the second non-protein containing culture medium in which the Mycobacterium cells have grown.

45. A method for treating or preventing tuberculosis, which method comprises administering to a human suffering, suspected of suffering or at risk of suffering from a Mycobacterium infection, an effective amount of the pharmaceutical composition of any of embodiments 27-44.

46. The method of embodiment 45, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Mycobacterium infection.

47. The method of embodiment 46, wherein the human for treatment has a weakened immune system, is a woman of reproductive age or a person with HIV/AIDS.

48. The method of any of embodiments 45-47, wherein the Mycobacterium infection is caused by a Mycobacterium strain that is resistant to an anti-bacterial drug or treatment.

49. The method of any of embodiments 45-48, further comprising, prior to administering the affinity purified human polyclonal antibodies to the human, conducting an immunotest to determine the presence, absence and/or amount of a Mycobacterium antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Mycobacterium infection using the affinity purified human polyclonal antibodies.

50. The method of any of embodiments 45-48, further comprising, before and after administering the affinity purified human polyclonal antibodies to the human, conducting an immunotest to determine the presence, absence and/or amount of a Mycobacterium antigen in a blood sample of the human using the same affinity purified human polyclonal antibodies, to monitor the efficacy of the therapeutic, removal or preventive treatment, wherein the absence or reduction in the Mycobacterium antigen after administering the affinity purified human polyclonal antibodies to the human relative to the amount of the Mycobacterium antigen before the administration indicates efficacy of the therapeutic, removal or preventive treatment.

51. The method of any of embodiments 45-48, further comprising, before and after administering the affinity purified human polyclonal antibodies to the human, conducting an immunotest to determine the presence, absence and/or amount of a Mycobacterium antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycobacterium antigen remaining after administering the affinity purified human polyclonal antibodies to the human and the extent of reduction in the Mycobacterium antigen after administering the affinity purified human polyclonal antibodies to the human relative to the amount of the Mycobacterium antigen before the administration.

52. A pharmaceutical composition for treating or preventing anthrax, which composition comprises an effective amount of human polyclonal antibodies affinity purified from a human blood sample with an antigenic preparation comprising a cellular antigen and/or a secreted antigen of Bacillus anthracis cells.

53. The pharmaceutical composition of embodiment 52, wherein the affinity purified human polyclonal antibodies are purified relative to the same human polyclonal antibodies in the unpurified or non-affinity-purified human blood sample.

54. The pharmaceutical composition of embodiment 52, wherein the affinity purified human polyclonal antibodies are specific for the Bacillus anthracis antigen(s) used in the affinity purification.

55. The pharmaceutical composition of embodiment 52, wherein the affinity purified human polyclonal antibodies are substantially free of human antibodies that specifically bind to non-Bacillus anthracis antigens in said human blood sample.

56. The pharmaceutical composition of any of embodiments 52-55, wherein the affinity purified human polyclonal antibodies specific to the Bacillus anthracis antigen(s) have a concentration ranging from about 10 μg/ml to about 10 mg/ml.

57. The pharmaceutical composition of any of embodiments 52-56, wherein the affinity purified human polyclonal antibodies are purified from about 2 fold to about 50,000 fold relative to the same human polyclonal antibodies in the unpurified or non-affinity-purified human blood sample.

58. The pharmaceutical composition of any of embodiments 52-57, wherein the human blood sample is from a normal human.

59. The pharmaceutical composition of any of embodiments 52-57, wherein the human blood sample is from a human infected with Bacillus anthracis.

60. The pharmaceutical composition of any of embodiments 52-59, wherein the human blood sample is pooled from at least 2 humans.

61. The pharmaceutical composition of any of embodiments 52-60, wherein the antigenic preparation comprises an antigen from the Ames strain, the Vollum strain (also incorrectly referred to as Vellum) strain and/or the Vollum 1B strain.

62. The pharmaceutical composition of any of embodiments 52-61, wherein the antigenic preparation comprises an exotoxin.

63. The pharmaceutical composition of any of embodiment 62, wherein the exotoxin is edema toxin or lethal toxin.

64. The pharmaceutical composition of any of embodiments 52-61, wherein the antigenic preparation comprises the poly-D-glutamic acid capsule, the protective antigen (PA), the edema factor (EF), and/or the lethal factor (LF).

65. The pharmaceutical composition of any of embodiments 52-64, wherein the antigenic preparation comprises a Bacillus anthracis antigen that confers antibiotic resistance.

66. The pharmaceutical composition of any of embodiments 52-65, wherein the antigenic preparation comprises a whole cell extract and a secreted antigen of Bacillus anthracis.

67. The pharmaceutical composition of any of embodiments 52-66, wherein the antigenic preparation is prepared by the following steps:

a) growing Bacillus anthracis cells in a first protein containing culture medium;

b) collecting and resuspending the Bacillus anthracis cells in a second non-protein containing culture medium;

c) growing the Bacillus anthracis cells in the second non-protein containing culture medium; and

d) disrupting the bacterial cells and collecting a whole cell extract from the disrupted Bacillus anthracis cells.

68. The pharmaceutical composition of embodiment 67, which further comprises a step of removing toxin from the whole cell extract.

69. The pharmaceutical composition of any of embodiments 67-68, which further comprises a step of collecting a secreted antigen from the second non-protein containing culture medium in which the Bacillus anthracis cells have grown.

70. A method for treating or preventing anthrax, which method comprises administering to a human suffering, suspected of suffering or at risk of suffering from anthrax, an effective amount of the pharmaceutical composition of any of embodiments 52-69.

71. The method of embodiment 70, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Bacillus anthracis infection.

72. The method of embodiment 70, wherein the human for treatment has a weakened immune system, pulmonary infection, gastrointestinal infection, or cutaneous infection.

73. The method of any of embodiments 70-72, wherein the Bacillus anthracis infection is caused by a Bacillus anthracis strain that is resistant to an anti-bacterial drug or treatment.

74. The method of any of embodiments 70-73, further comprising, prior to administering the affinity purified human polyclonal antibodies to the human, conducting an immunotest to determine the presence, absence and/or amount of a Bacillus anthracis antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Bacillus anthracis infection using the affinity purified human polyclonal antibodies.

75. The method of any of embodiments 70-73, further comprising, before and after administering the affinity purified human polyclonal antibodies to the human, conducting an immunotest to determine the presence, absence and/or amount of a Bacillus anthracis antigen in a blood sample of the human using the same affinity purified human polyclonal antibodies, to monitor the efficacy of the therapeutic, removal or preventive treatment, wherein the absence or reduction in the Bacillus anthracis antigen after administering the affinity purified human polyclonal antibodies to the human relative to the amount of the Bacillus anthracis antigen before the administration indicates efficacy of the therapeutic, removal or preventive treatment.

76. The method of any of embodiments 70-73, further comprising, before and after administering the affinity purified human polyclonal antibodies to the human, conducting an immunotest to determine the presence, absence and/or amount of a Bacillus anthracis antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Bacillus anthracis antigen remaining after administering the affinity purified human polyclonal antibodies to the human and the extent of reduction in the Bacillus anthracis antigen after administering the affinity purified human polyclonal antibodies to the human relative to the amount of the Bacillus anthracis antigen before the administration.

77. A pharmaceutical composition for treating or preventing listeriosis, which composition comprises an effective amount of human polyclonal antibodies affinity purified from a human blood sample with an antigenic preparation comprising a cellular antigen and/or a secreted antigen of Listeria monocytogenes cells.

78. The pharmaceutical composition of embodiment 77, wherein the affinity purified human polyclonal antibodies are purified relative to the same human polyclonal antibodies in the unpurified or non-affinity-purified human blood sample.

79. The pharmaceutical composition of embodiment 77, wherein the affinity purified human polyclonal antibodies are specific for the Listeria monocytogenes antigen(s) used in the affinity purification.

80. The pharmaceutical composition of embodiment 77, wherein the affinity purified human polyclonal antibodies are substantially free of human antibodies that specifically bind to non-Listeria monocytogenes antigens in said human blood sample.

81. The pharmaceutical composition of any of embodiments 77-80, wherein the affinity purified human polyclonal antibodies specific to the Listeria monocytogenes antigen(s) have a concentration ranging from about 10 μg/ml to about 10 mg/ml.

82. The pharmaceutical composition of any of embodiments 77-81, wherein the affinity purified human polyclonal antibodies are purified from about 2 fold to about 50,000 fold relative to the same human polyclonal antibodies in the unpurified or non-affinity-purified human blood sample.

83. The pharmaceutical composition of any of embodiments 77-82, wherein the human blood sample is from a normal human.

84. The pharmaceutical composition of any of embodiments 77-82, wherein the human blood sample is from a human infected with Listeria monocytogenes.

85. The pharmaceutical composition of any of embodiments 77-84, wherein the human blood sample is pooled from at least 2 humans.

86. The pharmaceutical composition of any of embodiments 77-85, wherein the Listeria monocytogenes has serotypes: 1/2a, 1/2b, and/or 4b.

87. The pharmaceutical composition of any of embodiments 77-86, wherein the antigenic preparation comprises a Listeria monocytogenes antigen that confers antibiotic resistance.

88. The pharmaceutical composition of any of embodiment 87, wherein the antibiotic resistant strain is Listeria monocytogenes BM4210 or BM4293.

89. The pharmaceutical composition of any of embodiments 77-88, wherein the antigenic preparation comprises a Listeria monocytogenes toxin, listerial internalins (Inl listeriolysin O (LLO—encoded by hly), phospholipase A (encoded by plcA) and/or phospholipase B (plcB).

90. The pharmaceutical composition of any of embodiments 77-89, wherein the antigenic preparation comprises a whole cell extract and a secreted antigen of Listeria monocytogenes.

91. The pharmaceutical composition of any of embodiments 77-90, wherein the antigenic preparation is prepared by the following steps:

a) growing Listeria monocytogenes cells in a first protein containing culture medium;

b) collecting and resuspending the Listeria monocytogenes cells in a second non-protein containing culture medium;

c) growing the Listeria monocytogenes cells in the second non-protein containing culture medium; and

d) disrupting the bacterial cells and collecting a whole cell extract from the disrupted Listeria monocytogenes cells.

92. The pharmaceutical composition of embodiment 91, which further comprises a step of removing toxin from the whole cell extract.

93. The pharmaceutical composition of any of embodiments 91-92, which further comprises a step of collecting a secreted antigen from the second non-protein containing culture medium in which the Listeria monocytogenes cells have grown.

94. A method for treating or preventing listeriosis, which method comprises administering to a human suffering, suspected of suffering or at risk of suffering from listeriosis, an effective amount of the pharmaceutical composition of any of embodiments 77-93.

95. The method of embodiment 94, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Listeria monocytogenes infection.

96. The method of embodiment 94, wherein the human for treatment has a weakened immune system, septicemia, meningitis (or meningoencephalitis), encephalitis, corneal ulcer, pneumonia, or intrauterine or cervical infectious in pregnant women.

97. The method of any of embodiments 94-96, wherein the Listeria monocytogenes infection is caused by a Listeria monocytogenes strain that is resistant to an anti-bacterial drug or treatment.

98. The method of any of embodiments 94-97, further comprising, prior to administering the affinity purified human polyclonal antibodies to the human, conducting an immunotest to determine the presence, absence and/or amount of a Listeria monocytogenes antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Listeria monocytogenes infection using the affinity purified human polyclonal antibodies.

99. The method of any of embodiments 94-97, further comprising, before and after administering the affinity purified human polyclonal antibodies to the human, conducting an immunotest to determine the presence, absence and/or amount of a Listeria monocytogenes antigen in a blood sample of the human using the same affinity purified human polyclonal antibodies, to monitor the efficacy of the therapeutic, removal or preventive treatment, wherein the absence or reduction in the Listeria monocytogenes antigen after administering the affinity purified human polyclonal antibodies to the human relative to the amount of the Listeria monocytogenes antigen before the administration indicates efficacy of the therapeutic, removal or preventive treatment.

100. The method of any of embodiments 94-97, further comprising, before and after administering the affinity purified human polyclonal antibodies to the human, conducting an immunotest to determine the presence, absence and/or amount of a Listeria monocytogenes antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Listeria monocytogenes antigen remaining after administering the affinity purified human polyclonal antibodies to the human and the extent of reduction in the Listeria monocytogenes antigen after administering the affinity purified human polyclonal antibodies to the human relative to the amount of the Listeria monocytogenes antigen before the administration.

101. A pharmaceutical composition for treating or preventing a Chlamydophila pneumoniae infection, which composition comprises an effective amount of human polyclonal antibodies affinity purified from a human blood sample with an antigenic preparation comprising a cellular antigen and/or a secreted antigen of Chlamydophila pneumoniae cells.

102. The pharmaceutical composition of embodiment 101, wherein the affinity purified human polyclonal antibodies are purified relative to the same human polyclonal antibodies in the unpurified or non-affinity-purified human blood sample.

103. The pharmaceutical composition of embodiment 101, wherein the affinity purified human polyclonal antibodies are specific for the Chlamydophila pneumoniae antigen(s) used in the affinity purification.

104. The pharmaceutical composition of embodiment 101, wherein the affinity purified human polyclonal antibodies are substantially free of human antibodies that specifically bind to non-Chlamydophila pneumoniae antigens in said human blood sample.

105. The pharmaceutical composition of any of embodiments 101-104, wherein the affinity purified human polyclonal antibodies specific to the Chlamydophila pneumoniae antigen(s) have a concentration ranging from about 10 μg/ml to about 10 mg/ml.

106. The pharmaceutical composition of any of embodiments 101-105, wherein the affinity purified human polyclonal antibodies are purified from about 2 fold to about 50,000 fold relative to the same human polyclonal antibodies in the unpurified or non-affinity-purified human blood sample.

107. The pharmaceutical composition of any of embodiments 101-106, wherein the human blood sample is from a normal human.

108. The pharmaceutical composition of any of embodiments 101-106, wherein the human blood sample is from a human infected with Chlamydophila pneumoniae.

109. The pharmaceutical composition of any of embodiments 101-108, wherein the human blood sample is pooled from at least 2 humans.

110. The pharmaceutical composition of any of embodiments 101-109, wherein the Chlamydophila pneumoniae is strain TWAR, A-03, BAL-16, TW-183, T-2634, or AR-39.

111. The pharmaceutical composition of any of embodiments 101-110, wherein the antigenic preparation comprises a Chlamydophila pneumoniae antigen that confers antibiotic resistance.

112. The pharmaceutical composition of any of embodiments 101-111, wherein the antigenic preparation comprises a Chlamydophila pneumoniae toxin, Omp11, type III secretion system ATPase, PmpG and/or IncA.

113. The pharmaceutical composition of any of embodiments 101-112, wherein the antigenic preparation comprises a whole cell extract and a secreted antigen of Chlamydophila pneumoniae.

114. The pharmaceutical composition of any of embodiments 101-113, wherein the antigenic preparation is prepared by the following steps:

a) growing Chlamydophila pneumoniae cells in a first protein containing culture medium;

b) collecting and resuspending the Chlamydophila pneumoniae cells in a second non-protein containing culture medium;

c) growing the Chlamydophila pneumoniae cells in the second non-protein containing culture medium; and

d) disrupting the bacterial cells and collecting a whole cell extract from the disrupted Chlamydophila pneumoniae cells.

115. The pharmaceutical composition of embodiment 114, which further comprises a step of removing toxin from the whole cell extract.

116. The pharmaceutical composition of any of embodiments 114-115, which further comprises a step of collecting a secreted antigen from the second non-protein containing culture medium in which the Chlamydophila pneumoniae cells have grown.

117. A method for treating or preventing a Chlamydophila pneumoniae infection, which method comprises administering to a human suffering, suspected of suffering or at risk of suffering from a Chlamydophila pneumoniae infection, an effective amount of the pharmaceutical composition of any of embodiments 101-116.

118. The method of embodiment 117, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Chlamydophila pneumoniae infection.

119. The method of embodiment 117, wherein the human for treatment has a weakened immune system, pneumoniae (also known as Chlamydia pneumonia), atherosclerosis, Alzheimer's disease and/or asthma.

120. The method of any of embodiments 117-119, wherein the Chlamydophila pneumoniae infection is caused by a Chlamydophila pneumoniae strain that is resistant to an anti-bacterial drug or treatment.

121. The method of any of embodiments 117-120, further comprising, prior to administering the affinity purified human polyclonal antibodies to the human, conducting an immunotest to determine the presence, absence and/or amount of a Chlamydophila pneumoniae antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Chlamydophila pneumoniae infection using the affinity purified human polyclonal antibodies.

122. The method of any of embodiments 117-120, further comprising, before and after administering the affinity purified human polyclonal antibodies to the human, conducting an immunotest to determine the presence, absence and/or amount of a Chlamydophila pneumoniae antigen in a blood sample of the human using the same affinity purified human polyclonal antibodies, to monitor the efficacy of the therapeutic, removal or preventive treatment, wherein the absence or reduction in the Chlamydophila pneumoniae antigen after administering the affinity purified human polyclonal antibodies to the human relative to the amount of the Chlamydophila pneumoniae antigen before the administration indicates efficacy of the therapeutic, removal or preventive treatment.

123. The method of any of embodiments 117-120, further comprising, before and after administering the affinity purified human polyclonal antibodies to the human, conducting an immunotest to determine the presence, absence and/or amount of a Chlamydophila pneumoniae antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Chlamydophila pneumoniae antigen remaining after administering the affinity purified human polyclonal antibodies to the human and the extent of reduction in the Chlamydophila pneumoniae antigen after administering the affinity purified human polyclonal antibodies to the human relative to the amount of the Chlamydophila pneumoniae antigen before the administration.

124. A pharmaceutical composition for treating or preventing a Ureaplasma urealyticum infection, which composition comprises an effective amount of human polyclonal antibodies affinity purified from a human blood sample with an antigenic preparation comprising a cellular antigen and/or a secreted antigen of Ureaplasma urealyticum cells.

125. The pharmaceutical composition of embodiment 124, wherein the affinity purified human polyclonal antibodies are purified relative to the same human polyclonal antibodies in the unpurified or non-affinity-purified human blood sample.

126. The pharmaceutical composition of embodiment 124, wherein the affinity purified human polyclonal antibodies are specific for the Ureaplasma urealyticum antigen(s) used in the affinity purification.

127. The pharmaceutical composition of embodiment 124, wherein the affinity purified human polyclonal antibodies are substantially free of human antibodies that specifically bind to non-Ureaplasma urealyticum antigens in said human blood sample.

128. The pharmaceutical composition of any of embodiments 124-127, wherein the affinity purified human polyclonal antibodies specific to the Ureaplasma urealyticum antigen(s) have a concentration ranging from about 10 μg/ml to about 10 mg/ml.

129. The pharmaceutical composition of any of embodiments 124-128, wherein the affinity purified human polyclonal antibodies are purified from about 2 fold to about 50,000 fold relative to the same human polyclonal antibodies in the unpurified or non-affinity-purified human blood sample.

130. The pharmaceutical composition of any of embodiments 124-129, wherein the human blood sample is from a normal human.

131. The pharmaceutical composition of any of embodiments 124-129, wherein the human blood sample is from a human infected with Ureaplasma urealyticum.

132. The pharmaceutical composition of any of embodiments 124-131, wherein the human blood sample is pooled from at least 2 humans.

133. The pharmaceutical composition of any of embodiments 124-132, wherein the Ureaplasma urealyticum has serovars 1-14.

134. The pharmaceutical composition of any of embodiments 124-133, wherein the Ureaplasma urealyticum antigenic preparation comprises multiple banded (MB) antigen.

135. The pharmaceutical composition of any of embodiments 124-134, wherein the antigenic preparation comprises a Ureaplasma urealyticum antigen that confers antibiotic resistance.

136. The pharmaceutical composition of any of embodiments 124-135, wherein the antigenic preparation comprises a whole cell extract and a secreted antigen of Ureaplasma urealyticum.

137. The pharmaceutical composition of any of embodiments 124-136, wherein the antigenic preparation is prepared by the following steps:

a) growing Ureaplasma urealyticum cells in a first protein containing culture medium;

b) collecting and resuspending the Ureaplasma urealyticum cells in a second non-protein containing culture medium;

c) growing the Ureaplasma urealyticum cells in the second non-protein containing culture medium; and

d) disrupting the bacterial cells and collecting a whole cell extract from the disrupted Ureaplasma urealyticum cells.

138. The pharmaceutical composition of embodiment 137, which further comprises a step of removing toxin from the whole cell extract.

139. The pharmaceutical composition of any of embodiments 137-138, which further comprises a step of collecting a secreted antigen from the second non-protein containing culture medium in which the Ureaplasma urealyticum cells have grown.

140. A method for treating or preventing a Ureaplasma urealyticum infection, which method comprises administering to a human suffering, suspected of suffering or at risk of suffering from a Ureaplasma urealyticum infection, an effective amount of the pharmaceutical composition of any of embodiments 124-139.

141. The method of embodiment 140, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Ureaplasma urealyticum infection.

142. The method of embodiment 140, wherein the human for treatment has a weakened immune system, non-specific urethritis (NSU), infertility, chorioamnionitis, stillbirth, premature birth, pneumonia, bronchopulmonary dysplasia and meningitis.

143. The method of any of embodiments 140-142, wherein the Ureaplasma urealyticum infection is caused by a Ureaplasma urealyticum strain that is resistant to an anti-bacterial drug or treatment.

144. The method of any of embodiments 140-143, further comprising, prior to administering the affinity purified human polyclonal antibodies to the human, conducting an immunotest to determine the presence, absence and/or amount of a Ureaplasma urealyticum antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Ureaplasma urealyticum infection using the affinity purified human polyclonal antibodies.

145. The method of any of embodiments 140-143, further comprising, before and after administering the affinity purified human polyclonal antibodies to the human, conducting an immunotest to determine the presence, absence and/or amount of a Ureaplasma urealyticum antigen in a blood sample of the human using the same affinity purified human polyclonal antibodies, to monitor the efficacy of the therapeutic, removal or preventive treatment, wherein the absence or reduction in the Ureaplasma urealyticum antigen after administering the affinity purified human polyclonal antibodies to the human relative to the amount of the Ureaplasma urealyticum antigen before the administration indicates efficacy of the therapeutic, removal or preventive treatment.

146. The method of any of embodiments 140-143, further comprising, before and after administering the affinity purified human polyclonal antibodies to the human, conducting an immunotest to determine the presence, absence and/or amount of a Ureaplasma urealyticum antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Ureaplasma urealyticum antigen remaining after administering the affinity purified human polyclonal antibodies to the human and the extent of reduction in the Ureaplasma urealyticum antigen after administering the affinity purified human polyclonal antibodies to the human relative to the amount of the Ureaplasma urealyticum antigen before the administration.

147. A pharmaceutical composition for treating or preventing a Mycoplasma hominis infection, which composition comprises an effective amount of human polyclonal antibodies affinity purified from a human blood sample with an antigenic preparation comprising a cellular antigen and/or a secreted antigen of Mycoplasma hominis cells.

148. The pharmaceutical composition of embodiment 147, wherein the affinity purified human polyclonal antibodies are purified relative to the same human polyclonal antibodies in the unpurified or non-affinity-purified human blood sample.

149. The pharmaceutical composition of embodiment 147, wherein the affinity purified human polyclonal antibodies are specific for the Mycoplasma hominis antigen(s) used in the affinity purification.

150. The pharmaceutical composition of embodiment 147, wherein the affinity purified human polyclonal antibodies are substantially free of human antibodies that specifically bind to non-Mycoplasma hominis antigens in said human blood sample.

151. The pharmaceutical composition of any of embodiments 147-150, wherein the affinity purified human polyclonal antibodies specific to the Mycoplasma hominis antigen(s) have a concentration ranging from about 10 μg/ml to about 10 mg/ml.

152. The pharmaceutical composition of any of embodiments 147-151, wherein the affinity purified human polyclonal antibodies are purified from about 2 fold to about 50,000 fold relative to the same human polyclonal antibodies in the unpurified or non-affinity-purified human blood sample.

153. The pharmaceutical composition of any of embodiments 147-152, wherein the human blood sample is from a normal human.

154. The pharmaceutical composition of any of embodiments 147-152, wherein the human blood sample is from a human infected with Mycoplasma hominis .

155. The pharmaceutical composition of any of embodiments 147-154, wherein the human blood sample is pooled from at least 2 humans.

156. The pharmaceutical composition of any of embodiments 147-155, wherein the Mycoplasma hominis is a strain selected from the group consisting of 1620, 2101, PG21, LBD4, r. Taub, W1458, 1611, F4238, M5039, H5488, 11085, 13428, 1184, 1888, 11932 and 13408.

157. The pharmaceutical composition of any of embodiments 147-156, wherein the Mycoplasma hominis antigenic preparation comprises a surface antigen.

158. The pharmaceutical composition of any of embodiments 147-157, wherein the antigenic preparation comprises a Mycoplasma hominis antigen that confers antibiotic resistance.

159. The pharmaceutical composition of any of embodiments 147-158, wherein the antigenic preparation comprises a whole cell extract and a secreted antigen of Mycoplasma hominis .

160. The pharmaceutical composition of any of embodiments 147-159, wherein the antigenic preparation is prepared by the following steps:

a) growing Mycoplasma hominis cells in a first protein containing culture medium;

b) collecting and resuspending the Mycoplasma hominis cells in a second non-protein containing culture medium;

c) growing the Mycoplasma hominis cells in the second non-protein containing culture medium; and

d) disrupting the bacterial cells and collecting a whole cell extract from the disrupted Mycoplasma hominis cells.

161. The pharmaceutical composition of embodiment 160, which further comprises a step of removing toxin from the whole cell extract.

162. The pharmaceutical composition of any of embodiments 160-161, which further comprises a step of collecting a secreted antigen from the second non-protein containing culture medium in which the Mycoplasma hominis cells have grown.

163. A method for treating or preventing a Mycoplasma hominis infection, which method comprises administering to a human suffering, suspected of suffering or at risk of suffering from a Mycoplasma hominis infection, an effective amount of the pharmaceutical composition of any of embodiments 147-162.

164. The method of embodiment 163, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Mycoplasma hominis infection.

165. The method of embodiment 163, wherein the human for treatment has a weakened immune system, pelvic inflammatory disease, post-abortal fever, post-partum fever, septic arthritis and nongonococcal urethritis.

166. The method of any of embodiments 163-165, wherein the Mycoplasma hominis infection is caused by a Mycoplasma hominis strain that is resistant to an anti-bacterial drug or treatment.

167. The method of any of embodiments 163-166, further comprising, prior to administering the affinity purified human polyclonal antibodies to the human, conducting an immunotest to determine the presence, absence and/or amount of a Mycoplasma hominis antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Mycoplasma hominis infection using the affinity purified human polyclonal antibodies.

168. The method of any of embodiments 163-166, further comprising, before and after administering the affinity purified human polyclonal antibodies to the human, conducting an immunotest to determine the presence, absence and/or amount of a Mycoplasma hominis antigen in a blood sample of the human using the same affinity purified human polyclonal antibodies, to monitor the efficacy of the therapeutic, removal or preventive treatment, wherein the absence or reduction in the Mycoplasma hominis antigen after administering the affinity purified human polyclonal antibodies to the human relative to the amount of the Mycoplasma hominis antigen before the administration indicates efficacy of the therapeutic, removal or preventive treatment.

169. The method of any of embodiments 163-166, further comprising, before and after administering the affinity purified human polyclonal antibodies to the human, conducting an immunotest to determine the presence, absence and/or amount of a Mycoplasma hominis antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycoplasma hominis antigen remaining after administering the affinity purified human polyclonal antibodies to the human and the extent of reduction in the Mycoplasma hominis antigen after administering the affinity purified human polyclonal antibodies to the human relative to the amount of the Mycoplasma hominis antigen before the administration.

170. A pharmaceutical composition for treating or preventing a Mycoplasma pneumoniae infection, which composition comprises an effective amount of human polyclonal antibodies affinity purified from a human blood sample with an antigenic preparation comprising a cellular antigen and/or a secreted antigen of Mycoplasma pneumoniae cells.

171. The pharmaceutical composition of embodiment 170, wherein the affinity purified human polyclonal antibodies are purified relative to the same human polyclonal antibodies in the unpurified or non-affinity-purified human blood sample.

172. The pharmaceutical composition of embodiment 170, wherein the affinity purified human polyclonal antibodies are specific for the Mycoplasma pneumoniae antigen(s) used in the affinity purification.

173. The pharmaceutical composition of embodiment 170, wherein the affinity purified human polyclonal antibodies are substantially free of human antibodies that specifically bind to non-Mycoplasma pneumoniae antigens in said human blood sample.

174. The pharmaceutical composition of any of embodiments 170-173, wherein the affinity purified human polyclonal antibodies specific to the Mycoplasma pneumoniae antigen(s) have a concentration ranging from about 10 μg/ml to about 10 mg/ml.

175. The pharmaceutical composition of any of embodiments 170-174, wherein the affinity purified human polyclonal antibodies are purified from about 2 fold to about 50,000 fold relative to the same human polyclonal antibodies in the unpurified or non-affinity-purified human blood sample.

176. The pharmaceutical composition of any of embodiments 170-175, wherein the human blood sample is from a normal human.

177. The pharmaceutical composition of any of embodiments 170-175, wherein the human blood sample is from a human infected with Mycoplasma pneumoniae.

178. The pharmaceutical composition of any of embodiments 170-177, wherein the human blood sample is pooled from at least 2 humans.

179. The pharmaceutical composition of any of embodiments 170-178, wherein the Mycoplasma pneumoniae is strain M129 (ATCC 29342), strain FH or strain MPN372.

180. The pharmaceutical composition of any of embodiments 170-179, wherein the Mycoplasma pneumoniae antigenic preparation comprises a surface antigen, adhesin P1, the 30 Kda adhesin-related protein on the tip structure of Mycoplasma pneumoniae cells or CARDS toxin.

181. The pharmaceutical composition of any of embodiments 170-180, wherein the antigenic preparation comprises a Mycoplasma pneumoniae antigen that confers antibiotic resistance.

182. The pharmaceutical composition of any of embodiments 170-181, wherein the antigenic preparation comprises a whole cell extract and a secreted antigen of Mycoplasma pneumoniae.

183. The pharmaceutical composition of any of embodiments 170-182, wherein the antigenic preparation is prepared by the following steps:

a) growing Mycoplasma pneumoniae cells in a first protein containing culture medium;

b) collecting and resuspending the Mycoplasma pneumoniae cells in a second non-protein containing culture medium;

c) growing the Mycoplasma pneumoniae cells in the second non-protein containing culture medium; and

d) disrupting the bacterial cells and collecting a whole cell extract from the disrupted Mycoplasma pneumoniae cells.

184. The pharmaceutical composition of embodiment 183, which further comprises a step of removing toxin from the whole cell extract.

185. The pharmaceutical composition of any of embodiments 183-184, which further comprises a step of collecting a secreted antigen from the second non-protein containing culture medium in which the Mycoplasma pneumoniae cells have grown.

186. A method for treating or preventing a Mycoplasma pneumoniae infection, which method comprises administering to a human suffering, suspected of suffering or at risk of suffering from a Mycoplasma pneumoniae infection, an effective amount of the pharmaceutical composition of any of embodiments 170-185.

187. The method of embodiment 186, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Mycoplasma pneumoniae infection.

188. The method of embodiment 186, wherein the human for treatment has a weakened immune system, acute or chronic respiratory infection, asthma or fulminant disease.

189. The method of embodiment 188, wherein the acute or chronic respiratory infection is human primary atypical pneumonia (PAP) (walking pneumonia), tracheobronchitis, pharyngitis or community acquired pneumonia.

190. The method of any of embodiments 186-189, wherein the Mycoplasma pneumoniae infection is caused by a Mycoplasma pneumoniae strain that is resistant to an anti-bacterial drug or treatment.

191. The method of any of embodiments 186-190, further comprising, prior to administering the affinity purified human polyclonal antibodies to the human, conducting an immunotest to determine the presence, absence and/or amount of a Mycoplasma pneumoniae antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Mycoplasma pneumoniae infection using the affinity purified human polyclonal antibodies.

192. The method of any of embodiments 186-190, further comprising, before and after administering the affinity purified human polyclonal antibodies to the human, conducting an immunotest to determine the presence, absence and/or amount of a Mycoplasma pneumoniae antigen in a blood sample of the human using the same affinity purified human polyclonal antibodies, to monitor the efficacy of the therapeutic, removal or preventive treatment, wherein the absence or reduction in the Mycoplasma pneumoniae antigen after administering the affinity purified human polyclonal antibodies to the human relative to the amount of the Mycoplasma pneumoniae antigen before the administration indicates efficacy of the therapeutic, removal or preventive treatment.

193. The method of any of embodiments 186-190, further comprising, before and after administering the affinity purified human polyclonal antibodies to the human, conducting an immunotest to determine the presence, absence and/or amount of a Mycoplasma pneumoniae antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Mycoplasma pneumoniae antigen remaining after administering the affinity purified human polyclonal antibodies to the human and the extent of reduction in the Mycoplasma pneumoniae antigen after administering the affinity purified human polyclonal antibodies to the human relative to the amount of the Mycoplasma pneumoniae antigen before the administration.

194. A pharmaceutical composition for treating or preventing malaria, which composition comprises an effective amount of human polyclonal antibodies affinity purified from a human blood sample with an antigenic preparation comprising a cellular antigen and/or a secreted antigen of malaria-causing Plasmodium cells.

195. The pharmaceutical composition of embodiment 194, wherein the affinity purified human polyclonal antibodies are purified relative to the same human polyclonal antibodies in the unpurified or non-affinity-purified human blood sample.

196. The pharmaceutical composition of embodiment 194, wherein the affinity purified human polyclonal antibodies are specific for the Plasmodium antigen(s) used in the affinity purification.

197. The pharmaceutical composition of embodiment 194, wherein the affinity purified human polyclonal antibodies are substantially free of human antibodies that specifically bind to non-Plasmodium antigens in said human blood sample.

198. The pharmaceutical composition of any of embodiments 194-197, wherein the affinity purified human polyclonal antibodies specific to the Plasmodium antigen(s) have a concentration ranging from about 10 μg/ml to about 10 mg/ml.

199. The pharmaceutical composition of any of embodiments 194-198, wherein the affinity purified human polyclonal antibodies are purified from about 2 fold to about 50,000 fold relative to the same human polyclonal antibodies in the unpurified or non-affinity-purified human blood sample.

200. The pharmaceutical composition of any of embodiments 194-199, wherein the human blood sample is from a normal human.

201. The pharmaceutical composition of any of embodiments 194-199, wherein the human blood sample is from a human infected with the Plasmodium.

202. The pharmaceutical composition of any of embodiments 194-201, wherein the human blood sample is pooled from at least 2 humans.

203. The pharmaceutical composition of any of embodiments 194-202, wherein the antigenic preparation comprises an antigen from P. falciparum, P. malariae, P. ovale, P. vivax and P. knowlesi, P. inui, P. cynomolgi, P. simiovale, P. brazilianum, P. schwetzi and P. simium.

204. The pharmaceutical composition of any of embodiments 194-203, wherein the antigenic preparation comprises a surface antigen from a Plasmodium cell, Plasmodium glutamate dehydrogenase or Plasmodium lactate dehydrogenase.

205. The pharmaceutical composition of any of embodiments 194-204, wherein the antigenic preparation comprises a Plasmodium strain or antigen that confers resistance to an anti-malaria drug.

206. The pharmaceutical composition of any of embodiments 194-205, wherein the antigenic preparation comprises a Plasmodium toxin.

207. The pharmaceutical composition of any of embodiments 194-206, wherein the antigenic preparation comprises a whole cell extract and a secreted antigen of Plasmodium.

208. The pharmaceutical composition of any of embodiments 194-207, wherein the antigenic preparation is prepared by the following steps:

a) growing Plasmodium cells in a first protein containing culture medium;

b) collecting and resuspending the Plasmodium cells in a second non-protein containing culture medium;

c) growing the Plasmodium cells in the second non-protein containing culture medium; and

d) disrupting the bacterial cells and collecting a whole cell extract from the disrupted Plasmodium cells.

209. The pharmaceutical composition of embodiment 208, which further comprises a step of removing toxin from the whole cell extract.

210. The pharmaceutical composition of any of embodiments 208-209, which further comprises a step of collecting a secreted antigen from the second non-protein containing culture medium in which the Plasmodium cells have grown.

211. A method for treating or preventing malaria, which method comprises administering to a human suffering, suspected of suffering or at risk of suffering from malaria, an effective amount of the pharmaceutical composition of any of embodiments 194-210.

212. The method of embodiment 211, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Plasmodium infection.

213. The method of embodiment 211, wherein the human for treatment has a weakened immune system, or have fever, shivering, arthralgia (joint pain), vomiting, anemia (caused by hemolysis), hemoglobinuria, retinal damage, and/or convulsions.

214. The method of any of embodiments 211-213, wherein the Plasmodium infection is caused by a Plasmodium strain that is resistant to an anti-malaria drug or treatment.

215. The method of any of embodiments 211-214, further comprising, prior to administering the affinity purified human polyclonal antibodies to the human, conducting an immunotest to determine the presence, absence and/or amount of a Plasmodium antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Plasmodium infection using the affinity purified human polyclonal antibodies.

216. The method of any of embodiments 211-214, further comprising, before and after administering the affinity purified human polyclonal antibodies to the human, conducting an immunotest to determine the presence, absence and/or amount of a Plasmodium antigen in a blood sample of the human using the same affinity purified human polyclonal antibodies, to monitor the efficacy of the therapeutic, removal or preventive treatment, wherein the absence or reduction in the Plasmodium antigen after administering the affinity purified human polyclonal antibodies to the human relative to the amount of the Plasmodium antigen before the administration indicates efficacy of the therapeutic, removal or preventive treatment.

217. The method of any of embodiments 211-214, further comprising, before and after administering the affinity purified human polyclonal antibodies to the human, conducting an immunotest to determine the presence, absence and/or amount of a Plasmodium antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Plasmodium antigen remaining after administering the affinity purified human polyclonal antibodies to the human and the extent of reduction in the Plasmodium antigen after administering the affinity purified human polyclonal antibodies to the human relative to the amount of the Plasmodium antigen before the administration.

218. A pharmaceutical composition for treating or preventing a Pneumocystis jirovecii infection, which composition comprises an effective amount of human polyclonal antibodies affinity purified from a human blood sample with an antigenic preparation comprising a cellular antigen and/or a secreted antigen of Pneumocystis jirovecii cells.

219. The pharmaceutical composition of embodiment 218, wherein the affinity purified human polyclonal antibodies are purified relative to the same human polyclonal antibodies in the unpurified or non-affinity-purified human blood sample.

220. The pharmaceutical composition of embodiment 218, wherein the affinity purified human polyclonal antibodies are specific for the Pneumocystis jirovecii antigen(s) used in the affinity purification.

221. The pharmaceutical composition of embodiment 218, wherein the affinity purified human polyclonal antibodies are substantially free of human antibodies that specifically bind to non-Pneumocystis jirovecii antigens in said human blood sample.

222. The pharmaceutical composition of any of embodiments 218-221, wherein the affinity purified human polyclonal antibodies specific to the Pneumocystis jirovecii antigen(s) have a concentration ranging from about 10 μg/ml to about 10 mg/ml.

223. The pharmaceutical composition of any of embodiments 218-222, wherein the affinity purified human polyclonal antibodies are purified from about 2 fold to about 50,000 fold relative to the same human polyclonal antibodies in the unpurified or non-affinity-purified human blood sample.

224. The pharmaceutical composition of any of embodiments 218-223, wherein the human blood sample is from a normal human.

225. The pharmaceutical composition of any of embodiments 218-223, wherein the human blood sample is from a human infected with Pneumocystis jirovecii.

226. The pharmaceutical composition of any of embodiments 218-225, wherein the human blood sample is pooled from at least 2 humans.

227. The pharmaceutical composition of any of embodiments 218-226, wherein the Pneumocystis jirovecii antigenic preparation comprises a major surface glycoprotein (MSG), e.g., MSG-14 of Pneumocystis jirovecii.

228. The pharmaceutical composition of any of embodiments 218-227, wherein the antigenic preparation comprises a Pneumocystis jirovecii antigen that confers resistance to an anti-fungal drug.

229. The pharmaceutical composition of any of embodiment 228, wherein the Pneumocystis jirovecii antigen that confers resistance to an anti-fungal drug is an antigen from dihyropteroate synthase (MPS) and/or dihydrofolate reductase (DHFR).

230. The pharmaceutical composition of any of embodiments 228-229, wherein the anti-fungal drug is a sulfa drug.

231. The pharmaceutical composition of any of embodiments 218-230, wherein the antigenic preparation comprises a whole cell extract and a secreted antigen of Pneumocystis jirovecii.

232. The pharmaceutical composition of any of embodiments 218-231, wherein the antigenic preparation is prepared by the following steps:

a) growing Pneumocystis jirovecii cells in a first protein containing culture medium;

b) collecting and resuspending the Pneumocystis jirovecii cells in a second non-protein containing culture medium;

c) growing the Pneumocystis jirovecii cells in the second non-protein containing culture medium; and

d) disrupting the bacterial cells and collecting a whole cell extract from the disrupted Pneumocystis jirovecii cells.

233. The pharmaceutical composition of embodiment 232, which further comprises a step of removing toxin from the whole cell extract.

234. The pharmaceutical composition of any of embodiments 232-233, which further comprises a step of collecting a secreted antigen from the second non-protein containing culture medium in which the Pneumocystis jirovecii cells have grown.

235. A method for treating or preventing a Pneumocystis jirovecii infection, which method comprises administering to a human suffering, suspected of suffering or at risk of suffering from a Pneumocystis jirovecii infection, an effective amount of the pharmaceutical composition of any of embodiments 218-234.

236. The method of embodiment 235, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Pneumocystis jirovecii infection.

237. The method of embodiment 235, wherein the human for treatment has a weakened immune system, pneumocystis pneumonia (PCP or pneumocystosis), cancer, and/or HIV/AIDS.

238. The method of any of embodiments 235-237, wherein the Pneumocystis jirovecii infection is caused by a Pneumocystis jirovecii strain that is resistant to an anti-fungal drug or treatment.

239. The method of any of embodiment 238, wherein the anti-fungal drug is a sulfa drug.

240. The method of any of embodiments 235-239, further comprising, prior to administering the affinity purified human polyclonal antibodies to the human, conducting an immunotest to determine the presence, absence and/or amount of a Pneumocystis jirovecii antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Pneumocystis jirovecii infection using the affinity purified human polyclonal antibodies.

241. The method of any of embodiments 235-239, further comprising, before and after administering the affinity purified human polyclonal antibodies to the human, conducting an immunotest to determine the presence, absence and/or amount of a Pneumocystis jirovecii antigen in a blood sample of the human using the same affinity purified human polyclonal antibodies, to monitor the efficacy of the therapeutic, removal or preventive treatment, wherein the absence or reduction in the Pneumocystis jirovecii antigen after administering the affinity purified human polyclonal antibodies to the human relative to the amount of the Pneumocystis jirovecii antigen before the administration indicates efficacy of the therapeutic, removal or preventive treatment.

242. The method of any of embodiments 235-239, further comprising, before and after administering the affinity purified human polyclonal antibodies to the human, conducting an immunotest to determine the presence, absence and/or amount of a Pneumocystis jirovecii antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Pneumocystis jirovecii antigen remaining after administering the affinity purified human polyclonal antibodies to the human and the extent of reduction in the Pneumocystis jirovecii antigen after administering the affinity purified human polyclonal antibodies to the human relative to the amount of the Pneumocystis jirovecii antigen before the administration.

243. A pharmaceutical composition for treating or preventing a Histoplasma capsulatum infection, which composition comprises an effective amount of human polyclonal antibodies affinity purified from a human blood sample with an antigenic preparation comprising a cellular antigen and/or a secreted antigen of Histoplasma capsulatum cells.

244. The pharmaceutical composition of embodiment 243, wherein the affinity purified human polyclonal antibodies are purified relative to the same human polyclonal antibodies in the unpurified or non-affinity-purified human blood sample.

245. The pharmaceutical composition of embodiment 243, wherein the affinity purified human polyclonal antibodies are specific for the Histoplasma capsulatum antigen(s) used in the affinity purification.

246. The pharmaceutical composition of embodiment 243, wherein the affinity purified human polyclonal antibodies are substantially free of human antibodies that specifically bind to non-Histoplasma capsulatum antigens in said human blood sample.

247. The pharmaceutical composition of any of embodiments 243-246, wherein the affinity purified human polyclonal antibodies specific to the Histoplasma capsulatum antigen(s) have a concentration ranging from about 10 μg/ml to about 10 mg/ml.

248. The pharmaceutical composition of any of embodiments 243-247, wherein the affinity purified human polyclonal antibodies are purified from about 2 fold to about 50,000 fold relative to the same human polyclonal antibodies in the unpurified or non-affinity-purified human blood sample.

249. The pharmaceutical composition of any of embodiments 243-248, wherein the human blood sample is from a normal human.

250. The pharmaceutical composition of any of embodiments 243-248, wherein the human blood sample is from a human infected with Histoplasma capsulatum.

251. The pharmaceutical composition of any of embodiments 243-250, wherein the human blood sample is pooled from at least 2 humans.

252. The pharmaceutical composition of any of embodiments 243-251, wherein the Histoplasma capsulatum is Histoplasma capsulatum var. capsulatum.

253. The pharmaceutical composition of any of embodiments 243-252, wherein the Histoplasma capsulatum antigenic preparation comprises a 69- to 70-kDa H. capsulatum var. capsulatum-specific antigen.

254. The pharmaceutical composition of any of embodiments 243-253, wherein the antigenic preparation comprises a Histoplasma capsulatum antigen that confers resistance to an anti-fungal drug.

255. The pharmaceutical composition of any of embodiments 253-254, wherein the anti-fungal drug is fluconazole.

256. The pharmaceutical composition of any of embodiments 243-255, wherein the antigenic preparation comprises a whole cell extract and a secreted antigen of Histoplasma capsulatum.

257. The pharmaceutical composition of any of embodiments 243-256, wherein the antigenic preparation is prepared by the following steps:

a) growing Histoplasma capsulatum cells in a first protein containing culture medium;

b) collecting and resuspending the Histoplasma capsulatum cells in a second non-protein containing culture medium;

c) growing the Histoplasma capsulatum cells in the second non-protein containing culture medium; and

d) disrupting the bacterial cells and collecting a whole cell extract from the disrupted Histoplasma capsulatum cells.

258. The pharmaceutical composition of embodiment 257, which further comprises a step of removing toxin from the whole cell extract.

259. The pharmaceutical composition of any of embodiments 257-258, which further comprises a step of collecting a secreted antigen from the second non-protein containing culture medium in which the Histoplasma capsulatum cells have grown.

260. A method for treating or preventing a Histoplasma capsulatum infection, which method comprises administering to a human suffering, suspected of suffering or at risk of suffering from a Histoplasma capsulatum infection, an effective amount of the pharmaceutical composition of any of embodiments 243-259.

261. The method of embodiment 260, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Histoplasma capsulatum infection.

262. The method of embodiment 260, wherein the human for treatment has a weakened immune system, histoplasmosis (also known as Cave disease, Darling's disease, Ohio valley disease, and “Reticuloendotheliosis) and/or HIV/AIDS.

263. The method of any of embodiments 260-262, wherein the Histoplasma capsulatum infection is caused by a Histoplasma capsulatum strain that is resistant to an anti-fungal drug or treatment.

264. The method of any of embodiment 263, wherein the anti-fungal drug is a fluconazole.

265. The method of any of embodiments 260-264, further comprising, prior to administering the affinity purified human polyclonal antibodies to the human, conducting an immunotest to determine the presence, absence and/or amount of a Histoplasma capsulatum antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Histoplasma capsulatum infection using the affinity purified human polyclonal antibodies.

266. The method of any of embodiments 260-264, further comprising, before and after administering the affinity purified human polyclonal antibodies to the human, conducting an immunotest to determine the presence, absence and/or amount of a Histoplasma capsulatum antigen in a blood sample of the human using the same affinity purified human polyclonal antibodies, to monitor the efficacy of the therapeutic, removal or preventive treatment, wherein the absence or reduction in the Histoplasma capsulatum antigen after administering the affinity purified human polyclonal antibodies to the human relative to the amount of the Histoplasma capsulatum antigen before the administration indicates efficacy of the therapeutic, removal or preventive treatment.

267. The method of any of embodiments 260-264, further comprising, before and after administering the affinity purified human polyclonal antibodies to the human, conducting an immunotest to determine the presence, absence and/or amount of a Histoplasma capsulatum antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Histoplasma capsulatum antigen remaining after administering the affinity purified human polyclonal antibodies to the human and the extent of reduction in the Histoplasma capsulatum antigen after administering the affinity purified human polyclonal antibodies to the human relative to the amount of the Histoplasma capsulatum antigen before the administration.

268. A pharmaceutical composition for treating or preventing a Blastomyces dermatitidis infection, which composition comprises an effective amount of human polyclonal antibodies affinity purified from a human blood sample with an antigenic preparation comprising a cellular antigen and/or a secreted antigen of Blastomyces dermatitidis cells.

269. The pharmaceutical composition of embodiment 268, wherein the affinity purified human polyclonal antibodies are purified relative to the same human polyclonal antibodies in the unpurified or non-affinity-purified human blood sample.

270. The pharmaceutical composition of embodiment 268, wherein the affinity purified human polyclonal antibodies are specific for the Blastomyces dermatitidis antigen(s) used in the affinity purification.

271. The pharmaceutical composition of embodiment 268, wherein the affinity purified human polyclonal antibodies are substantially free of human antibodies that specifically bind to non-Blastomyces dermatitidis antigens in said human blood sample.

272. The pharmaceutical composition of any of embodiments 268-271, wherein the affinity purified human polyclonal antibodies specific to the Blastomyces dermatitidis antigen(s) have a concentration ranging from about 10 μg/ml to about 10 mg/ml.

273. The pharmaceutical composition of any of embodiments 268-272, wherein the affinity purified human polyclonal antibodies are purified from about 2 fold to about 50,000 fold relative to the same human polyclonal antibodies in the unpurified or non-affinity-purified human blood sample.

274. The pharmaceutical composition of any of embodiments 268-273, wherein the human blood sample is from a normal human.

275. The pharmaceutical composition of any of embodiments 268-273, wherein the human blood sample is from a human infected with Blastomyces dermatitidis.

276. The pharmaceutical composition of any of embodiments 268-275, wherein the human blood sample is pooled from at least 2 humans.

277. The pharmaceutical composition of any of embodiments 268-276, wherein the Blastomyces dermatitidis is strain SLH-14081, ER-3, ATCC18188 or ATCC26199.

278. The pharmaceutical composition of any of embodiments 268-277, wherein the Histoplasma capsulatum antigenic preparation comprises an immunodominant cell wall antigen,

279. The pharmaceutical composition of any of embodiment 278, wherein the immunodominant cell wall antigen is WI-1 (Newman et al., J. Immunol., 1995, 154(2):753-761).

280. The pharmaceutical composition of any of embodiments 268-279, wherein the antigenic preparation comprises a Blastomyces dermatitidis antigen that confers resistance to an anti-fungal drug.

281. The pharmaceutical composition of any of embodiments 268-280, wherein the antigenic preparation comprises a whole cell extract and a secreted antigen of Blastomyces dermatitidis.

282. The pharmaceutical composition of any of embodiments 268-281, wherein the antigenic preparation is prepared by the following steps:

a) growing Blastomyces dermatitidis cells in a first protein containing culture medium;

b) collecting and resuspending the Blastomyces dermatitidis cells in a second non-protein containing culture medium;

c) growing the Blastomyces dermatitidis cells in the second non-protein containing culture medium; and

d) disrupting the bacterial cells and collecting a whole cell extract from the disrupted Blastomyces dermatitidis cells.

283. The pharmaceutical composition of embodiment 282, which further comprises a step of removing toxin from the whole cell extract.

284. The pharmaceutical composition of any of embodiments 282-283, which further comprises a step of collecting a secreted antigen from the second non-protein containing culture medium in which the Blastomyces dermatitidis cells have grown.

285. The pharmaceutical composition of any of embodiment 284, wherein the secreted antigen WI-1 (Audet et al., Protein Expr. Purif., 1997, 11:219-26),

286. A method for treating or preventing a Blastomyces dermatitidis infection, which method comprises administering to a human suffering, suspected of suffering or at risk of suffering from a Blastomyces dermatitidis infection, an effective amount of the pharmaceutical composition of any of embodiments 268-285.

287. The method of embodiment 286, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Blastomyces dermatitidis infection.

288. The method of embodiment 286, wherein the human for treatment has a weakened immune system, blastomycosis (also known as “North American blastomycosis,” “blastomycetic dermatitis,” and “Gilchrist's disease), and/or HIV/AIDS.

289. The method of any of embodiments 286-288, wherein the Blastomyces dermatitidis infection is caused by a Blastomyces dermatitidis strain that is resistant to an anti-fungal drug or treatment.

290. The method of any of embodiments 286-289, further comprising, prior to administering the affinity purified human polyclonal antibodies to the human, conducting an immunotest to determine the presence, absence and/or amount of a Blastomyces dermatitidis antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Blastomyces dermatitidis infection using the affinity purified human polyclonal antibodies.

291. The method of any of embodiments 286-289, further comprising, before and after administering the affinity purified human polyclonal antibodies to the human, conducting an immunotest to determine the presence, absence and/or amount of a Blastomyces dermatitidis antigen in a blood sample of the human using the same affinity purified human polyclonal antibodies, to monitor the efficacy of the therapeutic, removal or preventive treatment, wherein the absence or reduction in the Blastomyces dermatitidis antigen after administering the affinity purified human polyclonal antibodies to the human relative to the amount of the Blastomyces dermatitidis antigen before the administration indicates efficacy of the therapeutic, removal or preventive treatment.

292. The method of any of embodiments 286-289, further comprising, before and after administering the affinity purified human polyclonal antibodies to the human, conducting an immunotest to determine the presence, absence and/or amount of a Blastomyces dermatitidis antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Blastomyces dermatitidis antigen remaining after administering the affinity purified human polyclonal antibodies to the human and the extent of reduction in the Blastomyces dermatitidis antigen after administering the affinity purified human polyclonal antibodies to the human relative to the amount of the Blastomyces dermatitidis antigen before the administration.

293. A pharmaceutical composition for treating or preventing a Coccidioides infection, which composition comprises an effective amount of human polyclonal antibodies affinity purified from a human blood sample with an antigenic preparation comprising a cellular antigen and/or a secreted antigen of Coccidioides cells.

294. The pharmaceutical composition of embodiment 293, wherein the affinity purified human polyclonal antibodies are purified relative to the same human polyclonal antibodies in the unpurified or non-affinity-purified human blood sample.

295. The pharmaceutical composition of embodiment 293, wherein the affinity purified human polyclonal antibodies are specific for the Coccidioides antigen(s) used in the affinity purification.

296. The pharmaceutical composition of embodiment 293, wherein the affinity purified human polyclonal antibodies are substantially free of human antibodies that specifically bind to non-Coccidioides antigens in said human blood sample.

297. The pharmaceutical composition of any of embodiments 293-296, wherein the affinity purified human polyclonal antibodies specific to the Coccidioides antigen(s) have a concentration ranging from about 10 μg/ml to about 10 mg/ml.

298. The pharmaceutical composition of any of embodiments 293-297, wherein the affinity purified human polyclonal antibodies are purified from about 2 fold to about 50,000 fold relative to the same human polyclonal antibodies in the unpurified or non-affinity-purified human blood sample.

299. The pharmaceutical composition of any of embodiments 293-298, wherein the human blood sample is from a normal human.

300. The pharmaceutical composition of any of embodiments 293-298, wherein the human blood sample is from a human infected with Coccidioides.

301. The pharmaceutical composition of any of embodiments 293-300, wherein the human blood sample is pooled from at least 2 humans.

302. The pharmaceutical composition of any of embodiments 293-301, wherein the Coccidioides is C. immitis or C. posadasii.

303. The pharmaceutical composition of any of embodiments 293-302, wherein the Coccidioides antigenic preparation comprises galactomannan (Durkin et al., Clin. Infect. Dis., 2008, 47(8):e69-73), the tube precipitin (TP) antigen (Gade et al., J. Clin. Microbiol., 1992, 30(8):1907-1912) or a Coccidioides-specific antigen (CS-Ag) (Pan and Cole, Infect. Immun., 1995, 63:3994-4002).

304. The pharmaceutical composition of any of embodiments 293-303, wherein the antigenic preparation comprises a Coccidioides antigen that confers resistance to an anti-fungal drug.

305. The pharmaceutical composition of any of embodiments 293-304, wherein the antigenic preparation comprises a whole cell extract and a secreted antigen of Coccidioides.

306. The pharmaceutical composition of any of embodiments 293-305, wherein the antigenic preparation is prepared by the following steps:

a) growing Coccidioides cells in a first protein containing culture medium;

b) collecting and resuspending the Coccidioides cells in a second non-protein containing culture medium;

c) growing the Coccidioides cells in the second non-protein containing culture medium; and

d) disrupting the bacterial cells and collecting a whole cell extract from the disrupted Coccidioides cells.

307. The pharmaceutical composition of embodiment 306, which further comprises a step of removing toxin from the whole cell extract.

308. The pharmaceutical composition of any of embodiments 306-307, which further comprises a step of collecting a secreted antigen from the second non-protein containing culture medium in which the Coccidioides cells have grown.

309. The pharmaceutical composition of any of embodiment 308, wherein the secreted antigen is a Coccidioides-specific antigen (CS-Ag) (Pan and Cole, Infect. Immun., 1995, 63:3994-4002).

310. A method for treating or preventing a Coccidioides infection, which method comprises administering to a human suffering, suspected of suffering or at risk of suffering from a Coccidioides infection, an effective amount of the pharmaceutical composition of any of embodiments 293-309.

311. The method of embodiment 310, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Coccidioides infection.

312. The method of embodiment 310, wherein the human for treatment has a weakened immune system, coccidioidomycosis (also known as “California disease,” “Desert rheumatism,” “San Joaquin valley fever,” and “Valley fever”), and/or HIV/AIDS.

313. The method of embodiment 312, wherein the coccidioidomycosis is primary pulmonary coccidioidomycosis, disseminated coccidioidomycosis or primary cutaneous coccidioidomycosis.

314. The method of any of embodiments 310-313, wherein the Coccidioides infection is caused by a Coccidioides strain that is resistant to an anti-fungal drug or treatment.

315. The method of any of embodiments 310-314, further comprising, prior to administering the affinity purified human polyclonal antibodies to the human, conducting an immunotest to determine the presence, absence and/or amount of a Coccidioides antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Coccidioides infection using the affinity purified human polyclonal antibodies.

316. The method of any of embodiments 310-314, further comprising, before and after administering the affinity purified human polyclonal antibodies to the human, conducting an immunotest to determine the presence, absence and/or amount of a Coccidioides antigen in a blood sample of the human using the same affinity purified human polyclonal antibodies, to monitor the efficacy of the therapeutic, removal or preventive treatment, wherein the absence or reduction in the Coccidioides antigen after administering the affinity purified human polyclonal antibodies to the human relative to the amount of the Coccidioides antigen before the administration indicates efficacy of the therapeutic, removal or preventive treatment.

317. The method of any of embodiments 310-314, further comprising, before and after administering the affinity purified human polyclonal antibodies to the human, conducting an immunotest to determine the presence, absence and/or amount of a Coccidioides antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Coccidioides antigen remaining after administering the affinity purified human polyclonal antibodies to the human and the extent of reduction in the Coccidioides antigen after administering the affinity purified human polyclonal antibodies to the human relative to the amount of the Coccidioides antigen before the administration.

318. A pharmaceutical composition for treating or preventing an Aspergillus infection, which composition comprises an effective amount of human polyclonal antibodies affinity purified from a human blood sample with an antigenic preparation comprising a cellular antigen and/or a secreted antigen of Aspergillus cells.

319. The pharmaceutical composition of embodiment 318, wherein the affinity purified human polyclonal antibodies are purified relative to the same human polyclonal antibodies in the unpurified or non-affinity-purified human blood sample.

320. The pharmaceutical composition of embodiment 318, wherein the affinity purified human polyclonal antibodies are specific for the Aspergillus antigen(s) used in the affinity purification.

321. The pharmaceutical composition of embodiment 318, wherein the affinity purified human polyclonal antibodies are substantially free of human antibodies that specifically bind to non-Aspergillus antigens in said human blood sample.

322. The pharmaceutical composition of any of embodiments 318-321, wherein the affinity purified human polyclonal antibodies specific to the Aspergillus antigen(s) have a concentration ranging from about 10 μg/ml to about 10 mg/ml.

323. The pharmaceutical composition of any of embodiments 318-322, wherein the affinity purified human polyclonal antibodies are purified from about 2 fold to about 50,000 fold relative to the same human polyclonal antibodies in the unpurified or non-affinity-purified human blood sample.

324. The pharmaceutical composition of any of embodiments 318-323, wherein the human blood sample is from a normal human.

325. The pharmaceutical composition of any of embodiments 318-323, wherein the human blood sample is from a human infected with an Aspergillus.

326. The pharmaceutical composition of any of embodiments 318-325, wherein the human blood sample is pooled from at least 2 humans.

327. The pharmaceutical composition of any of embodiments 318-326, wherein the Aspergillus is Aspergillus fumigatus, Aspergillus flavus or Aspergillus clavatus.

328. The pharmaceutical composition of any of embodiments 318-327, wherein the Aspergillus antigenic preparation comprises an Aspergillus toxin or galactomannan (Klont et al., Clin. Infect. Dis., 2004, 39:1467-74).

329. The pharmaceutical composition of any of embodiment 328, wherein the Aspergillus toxin is aflatoxin.

330. The pharmaceutical composition of any of embodiments 318-329, wherein the antigenic preparation comprises an Aspergillus antigen that confers resistance to an anti-fungal drug.

331. The pharmaceutical composition of any of embodiments 318-330, wherein the antigenic preparation comprises a whole cell extract and a secreted antigen of an Aspergillus. (Medina et al., Proteomics, 2005, 5(12):3153-61).

332. The pharmaceutical composition of any of embodiments 318-331, wherein the antigenic preparation is prepared by the following steps:

a) growing Aspergillus cells in a first protein containing culture medium;

b) collecting and resuspending the Aspergillus cells in a second non-protein containing culture medium;

c) growing the Aspergillus cells in the second non-protein containing culture medium; and

d) disrupting the bacterial cells and collecting a whole cell extract from the disrupted Aspergillus cells.

333. The pharmaceutical composition of embodiment 332, which further comprises a step of removing toxin from the whole cell extract.

334. The pharmaceutical composition of any of embodiments 332-333, which further comprises a step of collecting a secreted antigen from the second non-protein containing culture medium in which the Aspergillus cells have grown.

335. A method for treating or preventing an Aspergillus infection, which method comprises administering to a human suffering, suspected of suffering or at risk of suffering from an Aspergillus infection, an effective amount of the pharmaceutical composition of any of embodiments 318-334.

336. The method of embodiment 335, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Aspergillus infection.

337. The method of embodiment 335, wherein the human for treatment has a weakened immune system, aspergillosis, and/or HIV/AIDS.

338. The method of embodiment 337, wherein the aspergillosis is allergic bronchopulmonary aspergillosis (or ABPA), acute invasive aspergillosis, disseminated invasive aspergillosis, or aspergilloma.

339. The method of any of embodiments 335-338, wherein the Aspergillus infection is caused by an Aspergillus strain that is resistant to an anti-fungal drug or treatment.

340. The method of any of embodiments 335-339, further comprising, prior to administering the affinity purified human polyclonal antibodies to the human, conducting an immunotest to determine the presence, absence and/or amount of an Aspergillus antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Aspergillus infection using the affinity purified human polyclonal antibodies.

341. The method of any of embodiments 335-339, further comprising, before and after administering the affinity purified human polyclonal antibodies to the human, conducting an immunotest to determine the presence, absence and/or amount of an Aspergillus antigen in a blood sample of the human using the same affinity purified human polyclonal antibodies, to monitor the efficacy of the therapeutic, removal or preventive treatment, wherein the absence or reduction in the Aspergillus antigen after administering the affinity purified human polyclonal antibodies to the human relative to the amount of the Aspergillus antigen before the administration indicates efficacy of the therapeutic, removal or preventive treatment.

342. The method of any of embodiments 335-339, further comprising, before and after administering the affinity purified human polyclonal antibodies to the human, conducting an immunotest to determine the presence, absence and/or amount of an Aspergillus antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Aspergillus antigen remaining after administering the affinity purified human polyclonal antibodies to the human and the extent of reduction in the Aspergillus antigen after administering the affinity purified human polyclonal antibodies to the human relative to the amount of the Aspergillus antigen before the administration.

343. A pharmaceutical composition for treating or preventing a Variola virus infection, which composition comprises an effective amount of human polyclonal antibodies affinity purified from a human blood sample with an antigenic preparation comprising an antigen of Variola virus.

344. The pharmaceutical composition of embodiment 343, wherein the affinity purified human polyclonal antibodies are purified from a human blood sample with an antigenic preparation comprising at least two antigens of Variola virus.

345. The pharmaceutical composition of any of embodiments 343-344, wherein the affinity purified human polyclonal antibodies are purified relative to the same human polyclonal antibodies in the unpurified or non-affinity-purified human blood sample.

346. The pharmaceutical composition of any of embodiments 343-344, wherein the affinity purified human polyclonal antibodies are specific for the Variola antigen(s) used in the affinity purification.

347. The pharmaceutical composition of any of embodiments 343-344, wherein the affinity purified human polyclonal antibodies are substantially free of human antibodies that specifically bind to non-Variola antigen in said human blood sample.

348. The pharmaceutical composition of any of embodiments 343-347, wherein the affinity purified human polyclonal antibodies specific to the Variola antigen(s) have a concentration ranging from about 10 μg/ml to about 10 mg/ml.

349. The pharmaceutical composition of any of embodiments 343-348, wherein the affinity purified human polyclonal antibodies are purified from about 2 fold to about 50,000 fold relative to the same human polyclonal antibodies in the unpurified or non-affinity-purified human blood sample.

350. The pharmaceutical composition of any of embodiments 343-349, wherein the human blood sample is from a normal human.

351. The pharmaceutical composition of any of embodiments 343-349, wherein the human blood sample is from a human infected with Variola virus.

352. The pharmaceutical composition of any of embodiments 343-351, wherein the human blood sample is pooled from at least 2 humans.

353. The pharmaceutical composition of any of embodiments 343-352, wherein the antigenic preparation comprises antigen(s) from Variola major and/or Variola minor.

354. The pharmaceutical composition of any of embodiments 343-353, wherein the Variola antigen(s) comprise A30, B7, F8 (Sakhatskyy et al., Virology, 2008, 371(1):98-107), an antigen from Variola-associated DNA-dependent RNA polymerase and/or a viral surface protein.

355. The pharmaceutical composition of any of embodiments 343-354, wherein the Variola viral surface protein is hemagglutinin.

356. The pharmaceutical composition of any of embodiments 343-355, wherein the antigenic preparation comprises a Variola antigen that confers resistance to anti-viral drug or treatment.

357. A method for treating or preventing a Variola virus infection, which method comprises administering to a human suffering, suspected of suffering or at risk of suffering from a Variola virus infection, an effective amount of the pharmaceutical composition of any of embodiments 343-356.

358. The method of embodiment 357, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Variola virus infection.

359. The method of embodiment 357, wherein the human for treatment has a weakened immune system or smallpox.

360. The method of embodiment 359, wherein the smallpox is caused by Variola major infection.

361. The method of embodiment 360, wherein the smallpox is selected from the group consisting of ordinary, modified, flat, and hemorrhagic smallpox.

362. The method of any of embodiments 357-361, wherein the Variola virus infection is caused by a Variola virus strain that is resistant to anti-viral drug or treatment.

363. The method of any of embodiments 357-362, further comprising, prior to administering the affinity purified human polyclonal antibodies to the human, conducting an immunotest to determine the presence, absence and/or amount of a Variola antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Variola virus infection using the affinity purified human polyclonal antibodies.

364. The method of any of embodiments 357-362, further comprising, before and after administering the affinity purified human polyclonal antibodies to the human, conducting an immunotest to determine the presence, absence and/or amount of a Variola antigen(s) in a blood sample of the human using the same affinity purified human polyclonal antibodies, to monitor the efficacy of the therapeutic, removal or preventive treatment, wherein the absence or reduction in the Variola antigen(s) after administering the affinity purified human polyclonal antibodies to the human relative to the amount of the Variola antigen(s) before the administration indicates efficacy of the therapeutic, removal or preventive treatment.

365. The method of any of embodiments 357-362, further comprising, before and after administering the affinity purified human polyclonal antibodies to the human, conducting an immunotest to determine the presence, absence and/or amount of a Variola antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Variola antigen(s) remaining after administering the affinity purified human polyclonal antibodies to the human and the extent of reduction in the Variola antigen(s) after administering the affinity purified human polyclonal antibodies to the human relative to the amount of the Variola antigen(s) before the administration.

366. A pharmaceutical composition for treating or preventing a respiratory syncytial virus (RSV) infection, which composition comprises an effective amount of human polyclonal antibodies affinity purified from a human blood sample with an antigenic preparation comprising an antigen of RSV.

367. The pharmaceutical composition of embodiment 366, wherein the affinity purified human polyclonal antibodies are purified from a human blood sample with an antigenic preparation comprising at least two antigens of RSV.

368. The pharmaceutical composition of any of embodiments 366-367, wherein the affinity purified human polyclonal antibodies are purified relative to the same human polyclonal antibodies in the unpurified or non-affinity-purified human blood sample.

369. The pharmaceutical composition of any of embodiments 366-367, wherein the affinity purified human polyclonal antibodies are specific for the RSV antigen(s) used in the affinity purification.

370. The pharmaceutical composition of any of embodiments 366-377, wherein the affinity purified human polyclonal antibodies are substantially free of human antibodies that specifically bind to non-RSV antigen in said human blood sample.

371. The pharmaceutical composition of any of embodiments 366-370, wherein the affinity purified human polyclonal antibodies specific to the RSV antigen(s) have a concentration ranging from about 10 μg/ml to about 10 mg/ml.

372. The pharmaceutical composition of any of embodiments 366-371, wherein the affinity purified human polyclonal antibodies are purified from about 2 fold to about 50,000 fold relative to the same human polyclonal antibodies in the unpurified or non-affinity-purified human blood sample.

373. The pharmaceutical composition of any of embodiments 366-372, wherein the human blood sample is from a normal human.

374. The pharmaceutical composition of any of embodiments 366-372, wherein the human blood sample is from a human infected with RSV.

375. The pharmaceutical composition of any of embodiments 366-374, wherein the human blood sample is pooled from at least 2 humans.

376. The pharmaceutical composition of any of embodiments 366-375, wherein the RSV is subgroup A and B virus.

377. The pharmaceutical composition of any of embodiments 1-11, wherein the RSV antigen(s) comprise an antigen from a RSV protein selected from the group consisting of NS1, NS2, N (nucleocapsid protein), M (matrix protein), SH (viral coat), G (viral coat), F (viral coat), M2 (the second matrix protein), L (the RNA polymerase) and the P (phosphoprotein).

378. The pharmaceutical composition of any of embodiments 366-377, wherein the antigenic preparation comprises a RSV antigen that confers resistance to anti-viral drug or treatment.

379. A method for treating or preventing an RSV infection, which method comprises administering to a human suffering, suspected of suffering or at risk of suffering from an RSV infection, an effective amount of the pharmaceutical composition of any of embodiments 366-378.

380. The method of embodiment 379, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the RSV infection.

381. The method of embodiment 380, wherein the human for treatment has a weakened immune system or bronchiolitis.

382. The method of any of embodiments 379-381, wherein the RSV infection is caused by an RSV strain that is resistant to anti-viral drug or treatment.

383. The method of any of embodiments 379-382, further comprising, prior to administering the affinity purified human polyclonal antibodies to the human, conducting an immunotest to determine the presence, absence and/or amount of an RSV antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the RSV infection using the affinity purified human polyclonal antibodies.

384. The method of any of embodiments 379-382, further comprising, before and after administering the affinity purified human polyclonal antibodies to the human, conducting an immunotest to determine the presence, absence and/or amount of an RSV antigen(s) in a blood sample of the human using the same affinity purified human polyclonal antibodies, to monitor the efficacy of the therapeutic, removal or preventive treatment, wherein the absence or reduction in the RSV antigen(s) after administering the affinity purified human polyclonal antibodies to the human relative to the amount of the RSV antigen(s) before the administration indicates efficacy of the therapeutic, removal or preventive treatment.

385. The method of any of embodiments 379-382, further comprising, before and after administering the affinity purified human polyclonal antibodies to the human, conducting an immunotest to determine the presence, absence and/or amount of an RSV antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the RSV antigen(s) remaining after administering the affinity purified human polyclonal antibodies to the human and the extent of reduction in the RSV antigen(s) after administering the affinity purified human polyclonal antibodies to the human relative to the amount of the RSV antigen(s) before the administration.

386. A pharmaceutical composition for treating or preventing a Cytomegalovirus (CMV) infection, which composition comprises an effective amount of human polyclonal antibodies affinity purified from a human blood sample with an antigenic preparation comprising an antigen of CMV.

387. The pharmaceutical composition of embodiment 386, wherein the affinity purified human polyclonal antibodies are purified from a human blood sample with an antigenic preparation comprising at least two antigens of CMV.

388. The pharmaceutical composition of any of embodiments 386-387, wherein the affinity purified human polyclonal antibodies are purified relative to the same human polyclonal antibodies in the unpurified or non-affinity-purified human blood sample.

389. The pharmaceutical composition of any of embodiments 386-387, wherein the affinity purified human polyclonal antibodies are specific for the CMV antigen(s) used in the affinity purification.

390. The pharmaceutical composition of any of embodiments 386-387, wherein the affinity purified human polyclonal antibodies are substantially free of human antibodies that specifically bind to non-CMV antigen in said human blood sample.

391. The pharmaceutical composition of any of embodiments 386-390, wherein the affinity purified human polyclonal antibodies specific to the CMV antigen(s) have a concentration ranging from about 10 μg/ml to about 10 mg/ml.

392. The pharmaceutical composition of any of embodiments 386-391, wherein the affinity purified human polyclonal antibodies are purified from about 2 fold to about 50,000 fold relative to the same human polyclonal antibodies in the unpurified or non-affinity-purified human blood sample.

393. The pharmaceutical composition of any of embodiments 386-392, wherein the human blood sample is from a normal human.

394. The pharmaceutical composition of any of embodiments 386-392, wherein the human blood sample is from a human infected with CMV.

395. The pharmaceutical composition of any of embodiments 386-394, wherein the human blood sample is pooled from at least 2 humans.

396. The pharmaceutical composition of any of embodiments 386-395, wherein the antigenic preparation comprises antigen(s) from HCMV (or Human Herpesvirus 5 or HHV-5).

397. The pharmaceutical composition of any of embodiments 386-396, wherein the Variola antigen(s) comprise an antigen from CMV glycoprotein I, glycoprotein III, a capsid protein, a coat protein or pp 65 protein.

398. The pharmaceutical composition of any of embodiments 386-397, wherein the antigenic preparation comprises a CMV antigen that confers resistance to anti-viral drug or treatment.

399. A method for treating or preventing a CMV infection, which method comprises administering to a human suffering, suspected of suffering or at risk of suffering from a CMV infection, an effective amount of the pharmaceutical composition of any of embodiments 386-398.

400. The method of embodiment 399, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the CMV infection.

401. The method of embodiment 399, wherein the human for treatment has a weakened immune system, leukemia, HIV and/or is an organ transplant recipient.

402. The method of embodiment 399, wherein the CMV infection is caused by HCMV.

403. The method of embodiment 402, wherein the HCMV is strain AD169.

404. The method of embodiment 399, wherein the human for treatment is a fetus or infant having congenital CMV infection or perinatal CMV infection.

405. The method of embodiment 399, wherein the human for treatment is an immunocompetent patient having CMV mononucleosis or post-transfusion CMV infection.

406. The method of embodiment 399, wherein the human for treatment is an immunocompromised patient having CMV pneumonitis, CMV GI disease, CMV retinitis, polyradiculopathy, transverse myelitis, and/or subacute encephalitis.

407. The method of embodiment 399, wherein the human for treatment has atherosclerosis.

408. The method of any of embodiments 399-407, wherein the CMV infection is caused by a CMV strain that is resistant to anti-viral drug or treatment.

409. The method of any of embodiments 399-408, further comprising, prior to administering the affinity purified human polyclonal antibodies to the human, conducting an immunotest to determine the presence, absence and/or amount of a CMV antigen(s) in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the CMV infection using the affinity purified human polyclonal antibodies.

410. The method of any of embodiments 399-408, further comprising, before and after administering the affinity purified human polyclonal antibodies to the human, conducting an immunotest to determine the presence, absence and/or amount of a CMV antigen(s) in a blood sample of the human using the same affinity purified human polyclonal antibodies, to monitor the efficacy of the therapeutic, removal or preventive treatment, wherein the absence or reduction in the CMV antigen(s) after administering the affinity purified human polyclonal antibodies to the human relative to the amount of the CMV antigen(s) before the administration indicates efficacy of the therapeutic, removal or preventive treatment.

411. The method of any of embodiments 399-408, further comprising, before and after administering the affinity purified human polyclonal antibodies to the human, conducting an immunotest to determine the presence, absence and/or amount of a CMV antigen(s) in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the CMV antigen(s) remaining after administering the affinity purified human polyclonal antibodies to the human and the extent of reduction in the CMV antigen(s) after administering the affinity purified human polyclonal antibodies to the human relative to the amount of the CMV antigen(s) before the administration.

412. A pharmaceutical composition for treating or preventing a viral, bacterial, eukaryotic protist and/or fungal infection, which composition comprises at least two of the following:

a) an effective amount of human polyclonal antibodies affinity purified from a human blood sample with an antigenic preparation comprising an antigen of an Influenza A virus;

b) an effective amount of human polyclonal antibodies affinity purified from a human blood sample with an antigenic preparation comprising an antigen of a Variola virus;

c) an effective amount of human polyclonal antibodies affinity purified from a human blood sample with an antigenic preparation comprising an antigen of a respiratory syncytial virus (RSV);

d) an effective amount of human polyclonal antibodies affinity purified from a human blood sample with an antigenic preparation comprising an antigen of a Cytomegalovirus (CMV);

e) an effective amount of human polyclonal antibodies affinity purified from a human blood sample with an antigenic preparation comprising an antigen of Staphylococcus aureus (S. aureus);

f) an effective amount of human polyclonal antibodies affinity purified from a human blood sample with an antigenic preparation comprising an antigen of a Streptococcus;

g) an effective amount of human polyclonal antibodies affinity purified from a human blood sample with an antigenic preparation comprising an antigen of Escherichia coli (E. coli);

h) an effective amount of human polyclonal antibodies affinity purified from a human blood sample with an antigenic preparation comprising an antigen of Pseudomonas aeruginosa (P. aeruginosa);

i) an effective amount of human polyclonal antibodies affinity purified from a human blood sample with an antigenic preparation comprising an antigen of Acinetobacter baumannii (A. baumannii);

j) an effective amount of human polyclonal antibodies affinity purified from a human blood sample with an antigenic preparation comprising an antigen of Enterococcus faecium (E. faecium);

k) an effective amount of human polyclonal antibodies affinity purified from a human blood sample with an antigenic preparation comprising an antigen of Enterococcus faecalis (E. faecalis);

l) an effective amount of human polyclonal antibodies affinity purified from a human blood sample with an antigenic preparation comprising an antigen of Enterobacter aerogenes (E. aerogenes);

m) an effective amount of human polyclonal antibodies affinity purified from a human blood sample with an antigenic preparation comprising an antigen of Enterobacter cloacae (E. cloacae);

n) an effective amount of human polyclonal antibodies affinity purified from a human blood sample with an antigenic preparation comprising an antigen of Clostridium difficile (C. difficile);

o) an effective amount of human polyclonal antibodies affinity purified from a human blood sample with an antigenic preparation comprising an antigen of Klebsiella pneumoniae (K. pneumoniae);

p) an effective amount of human polyclonal antibodies affinity purified from a human blood sample with an antigenic preparation comprising an antigen of a Salmonella;

q) an effective amount of human polyclonal antibodies affinity purified from a human blood sample with an antigenic preparation comprising an antigen of a TB-causing Mycobacterium;

r) an effective amount of human polyclonal antibodies affinity purified from a human blood sample with an antigenic preparation comprising an antigen of Bacillus anthracis;

s) an effective amount of human polyclonal antibodies affinity purified from a human blood sample with an antigenic preparation comprising an antigen of Listeria monocytogenes;

t) an effective amount of human polyclonal antibodies affinity purified from a human blood sample with an antigenic preparation comprising an antigen of Chlamydophila pneumoniae;

u) an effective amount of human polyclonal antibodies affinity purified from a human blood sample with an antigenic preparation comprising an antigen of Ureaplasma urealyticum;

v) an effective amount of human polyclonal antibodies affinity purified from a human blood sample with an antigenic preparation comprising an antigen of Mycoplasma hominis;

w) an effective amount of human polyclonal antibodies affinity purified from a human blood sample with an antigenic preparation comprising an antigen of Mycoplasma pneumoniae;

x) an effective amount of human polyclonal antibodies affinity purified from a human blood sample with an antigenic preparation comprising an antigen of a Plasmodium;

y) an effective amount of human polyclonal antibodies affinity purified from a human blood sample with an antigenic preparation comprising an antigen of Pneumocystis jirovecii;

z) an effective amount of human polyclonal antibodies affinity purified from a human blood sample with an antigenic preparation comprising an antigen of Histoplasma capsulatum;

aa) an effective amount of human polyclonal antibodies affinity purified from a human blood sample with an antigenic preparation comprising an antigen of Blastomyces dermatitidis;

bb) an effective amount of human polyclonal antibodies affinity purified from a human blood sample with an antigenic preparation comprising an antigen of a Coccidioides;

cc) an effective amount of human polyclonal antibodies affinity purified from a human blood sample with an antigenic preparation comprising an antigen of an Aspergillus;

dd) an effective amount of human polyclonal antibodies affinity purified from a human blood sample with an antigenic preparation comprising an antigen of Haemophilus influenzae; and

ee) an effective amount of human polyclonal antibodies affinity purified from a human blood sample with an antigenic preparation comprising an antigen of Campylobacter jejuni.

413. The pharmaceutical composition of embodiment 412, wherein the affinity purified human polyclonal antibodies are purified relative to the same human polyclonal antibodies in the unpurified or non-affinity-purified human blood sample.

414. The pharmaceutical composition of embodiment 412, wherein the affinity purified human polyclonal antibodies are specific for the viral, bacterial, eukaryotic protist and/or fungal antigens used in the affinity purification.

415. The pharmaceutical composition of embodiment 412, wherein the affinity purified human polyclonal antibodies are substantially free of human antibodies that specifically bind to non-viral, non-bacterial, non-eukaryotic protist and/or non-fungal antigens in said human blood sample.

416. The pharmaceutical composition of any of embodiments 412-415, wherein the affinity purified human polyclonal antibodies specific to the viral, bacterial, eukaryotic protist and/or fungal antigens have a concentration ranging from about 10 μg/ml to about 10 mg/ml.

417. The pharmaceutical composition of any of embodiments 412-416, wherein the affinity purified human polyclonal antibodies are purified from about 2 fold to about 50,000 fold relative to the same human polyclonal antibodies in the unpurified or non-affinity-purified human blood sample.

418. The pharmaceutical composition of any of embodiments 412-417, wherein the human blood sample is from a normal human.

419. The pharmaceutical composition of any of embodiments 412-417, wherein the human blood sample is from a human having the viral, bacterial, eukaryotic protist and/or fungal infection.

420. The pharmaceutical composition of any of embodiments 412-419, wherein the human blood sample is pooled from at least 2 humans.

421. The pharmaceutical composition of any of embodiments 412-420, wherein the antigenic preparation comprises an antigen from a virus selected from the group consisting of an influenza A virus, a Variola virus, a respiratory syncytial virus (RSV) and a Cytomegalovirus (CMV).

422. The pharmaceutical composition of any of embodiment 421, wherein the virus is an Influenza A virus.

423. The pharmaceutical composition of any of embodiments 412-422, wherein the antigenic preparation comprises a viral antigen that confers resistance to an anti-viral drug or treatment.

424. The pharmaceutical composition of any of embodiments 412-420, wherein the antigenic preparation comprises an antigen from a bacterium selected from the group consisting of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, a TB-causing Mycobacterium, Bacillus anthracis, Listeria monocytogenes, Chlamydophila pneumoniae, Ureaplasma urealyticum, Mycoplasma hominis, Mycoplasma pneumoniae, Haemophilus influenzae and Campylobacter jejuni.

425. The pharmaceutical composition of any of embodiments 412-424, wherein the antigenic preparation comprises a bacterial antigen that confers resistance to an antibiotic or anti-bacterial drug or treatment.

426. The pharmaceutical composition of any of embodiments 424-425, wherein the antigenic preparation comprises a whole cell extract and a secreted antigen of a bacterium.

427. The pharmaceutical composition of any of embodiments 424-426, wherein the antigenic preparation is prepared by the following steps:

a) growing bacterial cells in a first protein containing culture medium;

b) collecting and resuspending the bacterial cells in a second non-protein containing culture medium;

c) growing the bacterial cells in the second non-protein containing culture medium; and

d) disrupting the bacterial cells and collecting a whole cell extract from the disrupted bacterial cells.

428. The pharmaceutical composition of embodiment 427, which further comprises a step of removing toxin from the whole cell extract.

429. The pharmaceutical composition of any of embodiments 427-428, which further comprises a step of collecting a secreted antigen from the second non-protein containing culture medium in which the bacterial cells have grown.

430. The pharmaceutical composition of any of embodiments 412-420, wherein the antigenic preparation comprises an antigen from a eukaryotic protist or a fungus selected from the group consisting of a Plasmodium, Pneumocystis jirovecii, Histoplasma capsulatum, Blastomyces dermatitidis, a Coccidioides and an Aspergillus.

431. The pharmaceutical composition of any of embodiments 412-420 and 430, wherein the antigenic preparation comprises a eukaryotic protist or a fungal antigen that confers resistance to an anti-protist or anti-fungal drug or treatment.

432. The pharmaceutical composition of any of embodiments 430-431, wherein the antigenic preparation comprises a whole cell extract and a secreted antigen of a eukaryotic protist or a fungus.

433. The pharmaceutical composition of any of embodiments 430-432, wherein the antigenic preparation is prepared by the following steps:

a) growing eukaryotic protist or fungal cells in a first protein containing culture medium;

b) collecting and resuspending the eukaryotic protist or fungal cells in a second non-protein containing culture medium;

c) growing the eukaryotic protist or fungal cells in the second non-protein containing culture medium; and

d) disrupting the eukaryotic protist or fungal cells and collecting a whole cell extract from the disrupted eukaryotic protist or fungal cells.

434. The pharmaceutical composition of embodiment 433, which further comprises a step of removing toxin from the whole cell extract.

435. The pharmaceutical composition of any of embodiments 432-434, which further comprises a step of collecting a secreted antigen from the second non-protein containing culture medium in which the eukaryotic protist or fungal cells have grown.

436. The pharmaceutical composition of any of embodiments 412-435, wherein the antigenic preparation comprises at least two antigens from a virus, a bacterium and/or a eukaryotic protest or a fungus.

437. The pharmaceutical composition of any of embodiments 1-25, which composition comprises at least 3, 4, 5, 6, 7, 8, 9, 10, 11, 12, 13, 14, 15, 16, 17, 18, 19, 20, 21, 22, 23, 24, 25, 26, 27, 28, 29, 30 or all 31 of the following:

a) an effective amount of human polyclonal antibodies affinity purified from a human blood sample with an antigenic preparation comprising an antigen of an Influenza A virus;

b) an effective amount of human polyclonal antibodies affinity purified from a human blood sample with an antigenic preparation comprising an antigen of a Variola virus;

c) an effective amount of human polyclonal antibodies affinity purified from a human blood sample with an antigenic preparation comprising an antigen of a respiratory syncytial virus (RSV);

d) an effective amount of human polyclonal antibodies affinity purified from a human blood sample with an antigenic preparation comprising an antigen of a Cytomegalovirus (CMV);

e) an effective amount of human polyclonal antibodies affinity purified from a human blood sample with an antigenic preparation comprising an antigen of Staphylococcus aureus (S. aureus);

f) an effective amount of human polyclonal antibodies affinity purified from a human blood sample with an antigenic preparation comprising an antigen of a Streptococcus;

g) an effective amount of human polyclonal antibodies affinity purified from a human blood sample with an antigenic preparation comprising an antigen of Escherichia coli (E. coli);

h) an effective amount of human polyclonal antibodies affinity purified from a human blood sample with an antigenic preparation comprising an antigen of Pseudomonas aeruginosa (P. aeruginosa);

i) an effective amount of human polyclonal antibodies affinity purified from a human blood sample with an antigenic preparation comprising an antigen of Acinetobacter baumannii (A. baumannii);

j) an effective amount of human polyclonal antibodies affinity purified from a human blood sample with an antigenic preparation comprising an antigen of Enterococcus faecium (E. faecium);

k) an effective amount of human polyclonal antibodies affinity purified from a human blood sample with an antigenic preparation comprising an antigen of Enterococcus faecalis (E. faecalis);

l) an effective amount of human polyclonal antibodies affinity purified from a human blood sample with an antigenic preparation comprising an antigen of Enterobacter aerogenes (E. aerogenes);

m) an effective amount of human polyclonal antibodies affinity purified from a human blood sample with an antigenic preparation comprising an antigen of Enterobacter cloacae (E. cloacae);

n) an effective amount of human polyclonal antibodies affinity purified from a human blood sample with an antigenic preparation comprising an antigen of Clostridium difficile (C. difficile);

o) an effective amount of human polyclonal antibodies affinity purified from a human blood sample with an antigenic preparation comprising an antigen of Klebsiella pneumoniae (K. pneumoniae);

p) an effective amount of human polyclonal antibodies affinity purified from a human blood sample with an antigenic preparation comprising an antigen of a Salmonella;

q) an effective amount of human polyclonal antibodies affinity purified from a human blood sample with an antigenic preparation comprising an antigen of a TB-causing Mycobacterium;

r) an effective amount of human polyclonal antibodies affinity purified from a human blood sample with an antigenic preparation comprising an antigen of Bacillus anthracis;

s) an effective amount of human polyclonal antibodies affinity purified from a human blood sample with an antigenic preparation comprising an antigen of Listeria monocytogenes;

t) an effective amount of human polyclonal antibodies affinity purified from a human blood sample with an antigenic preparation comprising an antigen of Chlamydophila pneumoniae;

u) an effective amount of human polyclonal antibodies affinity purified from a human blood sample with an antigenic preparation comprising an antigen of Ureaplasma urealyticum;

v) an effective amount of human polyclonal antibodies affinity purified from a human blood sample with an antigenic preparation comprising an antigen of Mycoplasma hominis;

w) an effective amount of human polyclonal antibodies affinity purified from a human blood sample with an antigenic preparation comprising an antigen of Mycoplasma pneumoniae;

x) an effective amount of human polyclonal antibodies affinity purified from a human blood sample with an antigenic preparation comprising an antigen of a Plasmodium;

y) an effective amount of human polyclonal antibodies affinity purified from a human blood sample with an antigenic preparation comprising an antigen of Pneumocystis jirovecii;

z) an effective amount of human polyclonal antibodies affinity purified from a human blood sample with an antigenic preparation comprising an antigen of Histoplasma capsulatum;

aa) an effective amount of human polyclonal antibodies affinity purified from a human blood sample with an antigenic preparation comprising an antigen of Blastomyces dermatitidis;

bb) an effective amount of human polyclonal antibodies affinity purified from a human blood sample with an antigenic preparation comprising an antigen of a Coccidioides; and

cc) an effective amount of human polyclonal antibodies affinity purified from a human blood sample with an antigenic preparation comprising an antigen of an Aspergillus;

dd) an effective amount of human polyclonal antibodies affinity purified from a human blood sample with an antigenic preparation comprising an antigen of Haemophilus influenzae; and

ee) an effective amount of human polyclonal antibodies affinity purified from a human blood sample with an antigenic preparation comprising an antigen of Campylobacter jejuni.

438. The pharmaceutical composition of any of embodiments 412-437, which composition further comprises an effective amount of affinity purified human polyclonal antibodies specific to an TNF-α antigen.

439. The pharmaceutical composition of any of embodiments 412-438, which composition further comprises a pharmaceutically acceptable carrier or excipient.

440. A method for treating or preventing a viral, bacterial, eukaryotic protist and/or fungal infection, which method comprises administering to a human suffering, suspected of suffering or at risk of suffering from a viral, bacterial, eukaryotic protist and/or fungal infection, an effective amount of the pharmaceutical composition of any of embodiments 412-439.

441. The method of embodiment 440, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the viral, bacterial, eukaryotic protist and/or fungal infection.

442. The method of any of embodiments 440-441, wherein the viral infection is caused by a viral strain that is resistant to an anti-viral drug or treatment.

443. The method of any of embodiments 440-441, wherein the bacterial infection is caused by a bacterial strain that is resistant to an anti-bacterial drug or treatment.

444. The method of any of embodiments 440-441, wherein the eukaryotic protist infection is caused by a eukaryotic protist strain that is resistant to an anti-eukaryotic protist drug or treatment.

445. The method of any of embodiments 440-441, wherein the fungal infection is caused by a bacterial strain that is resistant to an anti-fungal drug or treatment.

446. The method of any of embodiments 440-445, further comprising, prior to administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the viral, bacterial, eukaryotic protist and/or fungal infection using the affinity purified human polyclonal antibodies.

447. The method of any of embodiments 440-445, further comprising, before and after administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to monitor the efficacy of the therapeutic, removal or preventive treatment, wherein the absence or reduction in the viral, bacterial, eukaryotic protist and/or fungal antigens after administering the affinity purified human polyclonal antibodies to the human relative to the amount of the viral, bacterial, eukaryotic protist and/or fungal antigens before the administration indicates efficacy of the therapeutic, removal or preventive treatment.

448. The method of any of embodiments 440-445, further comprising, before and after administering the affinity purified human polyclonal antibodies to the human, conducting an immunotest to determine the presence, absence and/or amount of the viral, bacterial, eukaryotic protist and/or fungal antigens in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the viral, bacterial, eukaryotic protist and/or fungal antigens remaining after administering the affinity purified human polyclonal antibodies to the human and the extent of reduction in the viral, bacterial, eukaryotic protist and/or fungal antigens after administering the affinity purified human polyclonal antibodies to the human relative to the amount of the viral, bacterial, eukaryotic protist and/or fungal antigens before the administration.

449. The method of any of embodiments 440-448, further comprising administering another anti-viral, anti-bacterial, anti-eukaryotic protist and/or anti-fungal drug and/or treatment.

450. A pharmaceutical composition for treating or preventing a Haemophilus influenzae (formerly called Pfeiffer's bacillus or Bacillus influenzae) infection, which composition comprises an effective amount of human polyclonal antibodies affinity purified from a human blood sample with an antigenic preparation comprising a cellular antigen and/or a secreted antigen of Haemophilus influenzae cells.

451. The pharmaceutical composition of embodiment 450, wherein the affinity purified human polyclonal antibodies are purified relative to the same human polyclonal antibodies in the unpurified or non-affinity-purified human blood sample.

452. The pharmaceutical composition of embodiment 450, wherein the affinity purified human polyclonal antibodies are specific for the Haemophilus influenzae antigen(s) used in the affinity purification.

453. The pharmaceutical composition of embodiment 450, wherein the affinity purified human polyclonal antibodies are substantially free of human antibodies that specifically bind to non-Haemophilus influenzae antigens in the human blood sample.

454. The pharmaceutical composition of any of embodiments 450-453, wherein the affinity purified human polyclonal antibodies specific to the Haemophilus influenzae antigen(s) have a concentration ranging from about 10 μg/ml to about 10 mg/ml.

455. The pharmaceutical composition of any of embodiments 450-454, wherein the affinity purified human polyclonal antibodies are purified from about 2 fold to about 50,000 fold relative to the same human polyclonal antibodies in the unpurified or non-affinity-purified human blood sample.

456. The pharmaceutical composition of any of embodiments 450-455, wherein the human blood sample is from a normal human.

457. The pharmaceutical composition of any of embodiments 450-455, wherein the human blood sample is from a human infected with Haemophilus influenzae.

458. The pharmaceutical composition of any of embodiments 450-457, wherein the human blood sample is pooled from at least 2 humans.

459. The pharmaceutical composition of any of embodiments 450-458, wherein the Haemophilus influenzae is an unencapsulated strain or an encapsulated strain.

460. The pharmaceutical composition of any of embodiment 459, wherein the encapsulated strain has the serotype a, b, c, d, e, or f.

461. The pharmaceutical composition of any of embodiments 450-460, wherein the antigenic preparation comprises a Haemophilus influenzae antigen that confers antibiotic resistance.

462. The pharmaceutical composition of any of embodiments 450-461, wherein the antigenic preparation comprises a whole cell extract and a secreted antigen of Haemophilus influenzae.

463. The pharmaceutical composition of any of embodiments 450-462, wherein the antigenic preparation is prepared by the following steps:

a) growing Haemophilus influenzae cells in a first protein containing culture medium;

b) collecting and resuspending the Haemophilus influenzae cells in a second non-protein containing culture medium;

c) growing the Haemophilus influenzae cells in the second non-protein containing culture medium; and

d) disrupting the bacterial cells and collecting a whole cell extract from the disrupted Haemophilus influenzae cells.

464. The pharmaceutical composition of embodiment 463, which further comprises a step of removing toxin from the whole cell extract.

465. The pharmaceutical composition of any of embodiments 463-464, which further comprises a step of collecting a secreted antigen from the second non-protein containing culture medium in which the Haemophilus influenzae cells have grown.

466. A method for treating or preventing a Haemophilus influenzae infection, which method comprises administering to a human suffering, suspected of suffering or at risk of suffering from a Haemophilus influenzae infection, an effective amount of the pharmaceutical composition of any of embodiments 450-465.

467. The method of embodiment 466, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Haemophilus influenzae infection.

468. The method of embodiment 466, wherein the human for treatment has a weakened immune system, bacteremia, pneumonia, acute bacterial meningitis, cellulitis, osteomyelitis, epiglottitis, infectious arthritis, ear infections (otitis media), eye infections (conjunctivitis), and/or sinusitis.

469. The method of any of embodiments 466-468, wherein the Haemophilus influenzae infection is caused by a Haemophilus influenzae strain that is resistant to an anti-bacterial drug or treatment.

470. The method of any of embodiments 466-469, further comprising, prior to administering the affinity purified human polyclonal antibodies to the human, conducting an immunotest to determine the presence, absence and/or amount of a Haemophilus influenzae antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Haemophilus influenzae infection using the affinity purified human polyclonal antibodies.

471. The method of any of embodiments 466-469, further comprising, before and after administering the affinity purified human polyclonal antibodies to the human, conducting an immunotest to determine the presence, absence and/or amount of a Haemophilus influenzae antigen in a blood sample of the human using the same affinity purified human polyclonal antibodies, to monitor the efficacy of the therapeutic, removal or preventive treatment, wherein the absence or reduction in the Haemophilus influenzae antigen after administering the affinity purified human polyclonal antibodies to the human relative to the amount of Haemophilus influenzae antigen before the administration indicates efficacy of the therapeutic, removal or preventive treatment.

472. The method of any of embodiments 466-469, further comprising, before and after administering the affinity purified human polyclonal antibodies to the human, conducting an immunotest to determine the presence, absence and/or amount of a Haemophilus influenzae antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Haemophilus influenzae antigen remaining after administering the affinity purified human polyclonal antibodies to the human and the extent of reduction in the Haemophilus influenzae antigen after administering the affinity purified human polyclonal antibodies to the human relative to the amount of Haemophilus influenzae antigen before the administration.

473. A pharmaceutical composition for treating or preventing a Campylobacter jejuni infection, which composition comprises an effective amount of human polyclonal antibodies affinity purified from a human blood sample with an antigenic preparation comprising a cellular antigen and/or a secreted antigen of Campylobacter jejuni cells.

474. The pharmaceutical composition of embodiment 473, wherein the affinity purified human polyclonal antibodies are purified relative to the same human polyclonal antibodies in the unpurified or non-affinity-purified human blood sample.

475. The pharmaceutical composition of embodiment 473, wherein the affinity purified human polyclonal antibodies are specific for the Campylobacter jejuni antigen(s) used in the affinity purification.

476. The pharmaceutical composition of embodiment 473, wherein the affinity purified human polyclonal antibodies are substantially free of human antibodies that specifically bind to non-Campylobacter jejuni antigens in said human blood sample.

477. The pharmaceutical composition of any of embodiments 473-476, wherein the affinity purified human polyclonal antibodies specific to the Campylobacter jejuni antigen(s) have a concentration ranging from about 10 μg/ml to about 10 mg/ml.

478. The pharmaceutical composition of any of embodiments 473-477, wherein the affinity purified human polyclonal antibodies are purified from about 2 fold to about 50,000 fold relative to the same human polyclonal antibodies in the unpurified or non-affinity-purified human blood sample.

479. The pharmaceutical composition of any of embodiments 473-478, wherein the human blood sample is from a normal human.

480. The pharmaceutical composition of any of embodiments 473-478, wherein the human blood sample is from a human infected with Campylobacter jejuni.

481. The pharmaceutical composition of any of embodiments 473-480, wherein the human blood sample is pooled from at least 2 humans.

482. The pharmaceutical composition of any of embodiments 473-481, wherein the Campylobacter jejuni is strain NCTC11168.

483. The pharmaceutical composition of any of embodiments 473-482, wherein the antigenic preparation comprises a Campylobacter jejuni antigen that confers antibiotic resistance.

484. The pharmaceutical composition of any of embodiments 473-483, wherein the antigenic preparation comprises a whole cell extract and a secreted antigen of Campylobacter jejuni.

485. The pharmaceutical composition of any of embodiments 473-484, wherein the antigenic preparation is prepared by the following steps:

a) growing Campylobacter jejuni cells in a first protein containing culture medium;

b) collecting and resuspending the Campylobacter jejuni cells in a second non-protein containing culture medium;

c) growing the Campylobacter jejuni cells in the second non-protein containing culture medium; and

d) disrupting the bacterial cells and collecting a whole cell extract from the disrupted Campylobacter jejuni cells.

486. The pharmaceutical composition of embodiment 485, which further comprises a step of removing toxin from the whole cell extract.

487. The pharmaceutical composition of any of embodiments 485-486, which further comprises a step of collecting a secreted antigen from the second non-protein containing culture medium in which the Campylobacter jejuni cells have grown.

488. A method for treating or preventing a Campylobacter jejuni infection, which method comprises administering to a human suffering, suspected of suffering or at risk of suffering from a Campylobacter jejuni infection, an effective amount of the pharmaceutical composition of any of embodiments 473-487.

489. The method of embodiment 488, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Campylobacter jejuni infection.

490. The method of embodiment 488, wherein the human for treatment has a weakened immune system, enteritis, food poisoning and/or Guillain-Barré syndrome (GBS).

491. The method of embodiment 490, wherein the enteritis is human gastroenteritis.

492. The method of any of embodiments 488-491, wherein the Campylobacter jejuni infection is caused by a Campylobacter jejuni strain that is resistant to an anti-bacterial drug or treatment.

493. The method of any of embodiments 488-492, further comprising, prior to administering the affinity purified human polyclonal antibodies to the human, conducting an immunotest to determine the presence, absence and/or amount of a Campylobacter jejuni antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Campylobacter jejuni infection using the affinity purified human polyclonal antibodies.

494. The method of any of embodiments 488-492, further comprising, before and after administering the affinity purified human polyclonal antibodies to the human, conducting an immunotest to determine the presence, absence and/or amount of a Campylobacter jejuni antigen in a blood sample of the human using the same affinity purified human polyclonal antibodies, to monitor the efficacy of the therapeutic, removal or preventive treatment, wherein the absence or reduction in the Campylobacter jejuni antigen after administering the affinity purified human polyclonal antibodies to the human relative to the amount of Campylobacter jejuni antigen before the administration indicates efficacy of the therapeutic, removal or preventive treatment.

495. The method of any of embodiments 488-492, further comprising, before and after administering the affinity purified human polyclonal antibodies to the human, conducting an immunotest to determine the presence, absence and/or amount of a Campylobacter jejuni antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Campylobacter jejuni antigen remaining after administering the affinity purified human polyclonal antibodies to the human and the extent of reduction in the Campylobacter jejuni antigen after administering the affinity purified human polyclonal antibodies to the human relative to the amount of Campylobacter jejuni antigen before the administration.

The numbering of the above Exemplary Embodiments 1-495 applies to the Exemplary Embodiments in the Exemplary Embodiments Section A.

### Exemplary Embodiments Section B

1. An antigenic composition comprising at least one, preferably two, Influenza A virus polypeptides, wherein each of said polypeptides comprises an amino acid sequence selected from the group consisting of:

a) polymerase B1 (PB1) sequence, from N-terminus to C-terminus, DAVATTHSWIPKRNRSIL (SEQ ID NO:1),

b) PB1 sequence, from N-terminus to C-terminus, FLKDVMESM (SEQ ID NO:2),

c) PB1 sequence, from N-terminus to C-terminus, FNMLSTVLGV (SEQ ID NO:3),

d) PB1 sequence, from N-terminus to C-terminus, FSMELPSFGV (SEQ ID NO:4),

e) PB1 sequence, from N-terminus to C-terminus, GPATAQMAL (SEQ ID NO:5),

f) PB1 sequence, from N-terminus to C-terminus, DTVNRTHQY (SEQ ID NO:6),

g) polymerase B2 (PB2) sequence, from N-terminus to C-terminus, YMLERELVRKTRFLPVA (SEQ ID NO:7),

h) PB2 sequence, from N-terminus to C-terminus, NFVNRANQRLNPMHQLLR (SEQ ID NO:8),

i) polymerase A (PA) sequence, from N-terminus to C-terminus, FMYSDFHFI (SEQ ID NO:9),

j) PA sequence, from N-terminus to C-terminus, RSKFLLMDALKLSIE (SEQ ID NO:10),

k) PA sequence, from N-terminus to C-terminus, SVKEKDMTK (SEQ ID NO:11),

l) PA sequence, from N-terminus to C-terminus, MRRNYFTAEVSHCRATEY (SEQ ID NO:12),

m) PA sequence, from N-terminus to C-terminus, AESRKLLLI (SEQ ID NO:13),

n) hemagglutinin (HA) sequence, from N-terminus to C-terminus, GLFGAIAGFC (SEQ ID NO:14),

o) HA sequence, from N-terminus to C-terminus, GLFGAIAGFI (SEQ ID NO:15),

p) HA sequence, from N-terminus to C-terminus, TGMVDGWYGYHHQNEQGS (SEQ ID NO:16),

q) HA sequence, from N-terminus to C-terminus, WTYNAELLVLLENERTLD (SEQ ID NO:17),

r) HA sequence, from N-terminus to C-terminus, NKVNSVIEKMNTQFTAVG (SEQ ID NO:18),

s) HA sequence, from N-terminus to C-terminus, GLFGAIAGFIE (SEQ ID NO:19),

t) HA sequence, from N-terminus to C-terminus, YPYDVPDYA (SEQ ID NO:20),

u) HA sequence, from N-terminus to C-terminus, VTGLRNIPSIQCR (SEQ ID NO:21),

v) HA sequence, from N-terminus to C-terminus, SVSSFERFEIFPK (SEQ ID NO:22),

w) nucleoprotein (NP) sequence, from N-terminus to C-terminus, RRSGAAGAAVK (SEQ ID NO:23),

x) NP sequence, from N-terminus to C-terminus, QLVWMACHSAA (SEQ ID NO:24),

y) NP sequence, from N-terminus to C-terminus, YERMCNILKG (SEQ ID NO:25),

z) NP sequence, from N-terminus to C-terminus, TYQRTRALV (SEQ ID NO:26),

aa) NP sequence, from N-terminus to C-terminus, RMVLSAFDER (SEQ ID NO:27),

bb) NP sequence, from N-terminus to C-terminus, LELRSRYWAI (SEQ ID NO:28),

cc) NP sequence, from N-terminus to C-terminus, KLSTRGVQIASNEN (SEQ ID NO:29),

dd) neuraminidase (NA) sequence, from N-terminus to C-terminus, SWPDGAELPF (SEQ ID NO:30),

ee) NA sequence, from N-terminus to C-terminus, PIRGWAI (SEQ ID NO: 31),

ff) NA sequence, from N-terminus to C-terminus, SGSFVQHPELTGL (SEQ ID NO:32),

gg) NA sequence, from N-terminus to C-terminus, VGLISLILQI (SEQ ID NO:33),

hh) matrix protein 1 (M1) sequence, from N-terminus to C-terminus, KTRPILSPLTK (SEQ ID NO:34),

ii) M1 sequence, from N-terminus to C-terminus, QKRMGVQMQRFK (SEQ ID NO:35),

jj) M1 sequence, from N-terminus to C-terminus, AGKNTDLEALMEWLKTR (SEQ ID NO:36),

kk) M1 sequence, from N-terminus to C-terminus, IRHENRMVL (SEQ ID NO:37),

ll) M1 sequence, from N-terminus to C-terminus, GILGFVFTL (SEQ ID NO:38),

mm) M1 sequence, from N-terminus to C-terminus, SLLTEVETYVL (SEQ ID NO:39),

nn) M1 sequence, from N-terminus to C-terminus, KGILGFVFTLTVPSE (SEQ ID NO:40),

oo) M1 sequence, from N-terminus to C-terminus, ILSPLTKGIL (SEQ ID NO:41),

pp) M1 sequence, from N-terminus to C-terminus, RMVLASTTAKAMEQM (SEQ ID NO:42),

qq) matrix protein 2 (M2) sequence, from N-terminus to C-terminus, SLLTEVET (SEQ ID NO:43),

rr) M2 sequence, from N-terminus to C-terminus, EVETPIRN (SEQ ID NO:44),

ss) non-structural protein 1 (NS1) sequence, from N-terminus to C-terminus, GEISPLPSL (SEQ ID NO:45),

tt) NS1 sequence, from N-terminus to C-terminus, DRLRRDQKS (SEQ ID NO:46),

uu) NS1 sequence, from N-terminus to C-terminus, AIMDKNIIL (SEQ ID NO:47),

vv) non-structural protein 2 (NS2) sequence, from N-terminus to C-terminus, ITFMQALQLL (SEQ ID NO:48), and

ww) NS2 sequence, from N-terminus to C-terminus, RTFSFQLI (SEQ ID NO:49),

wherein each of said polypeptides does not comprise any additional amino acid sequence of a naturally occurring Influenza A virus protein besides the amino acid sequences recited in SEQ ID NO:1 to SEQ ID NO:49.

2. The composition of embodiment 1, which comprises at least 5, 10, 15, 20, 25, 30, 35, 40, 45, or all 49 of said Influenza A virus polypeptides.

3. The composition of embodiment 1, wherein at least one of the Influenza A virus polypeptides consists essentially of an amino acid sequence selected from the group consisting of SEQ ID NO:1 to SEQ ID NO:49.

4. The composition of embodiment 3, wherein at least 5, 10, 15, 20, 25, 30, 35, 40, 45, or all 49 of the Influenza A virus polypeptides consist essentially of amino acid sequences selected from the group consisting of SEQ ID NO:1 to SEQ ID NO:49.

5. The composition of embodiment 1, wherein at least one of the Influenza A virus polypeptides consists of an amino acid sequence selected from the group consisting of SEQ ID NO:1 to SEQ ID NO:49.

6. The composition of embodiment 5, wherein at least 5, 10, 15, 20, 25, 30, 35, 40, 45, or all 49 of the Influenza A virus polypeptides consist of amino acid sequences selected from the group consisting of SEQ ID NO:1 to SEQ ID NO:49.

7. An antigenic composition comprising at least one, preferably two, Influenza A virus polypeptides, wherein each of said polypeptides comprises an amino acid sequence selected from the group consisting of:

a) polymerase B1 (PB1) sequence, from N-terminus to C-terminus, FLKDVMESM (SEQ ID NO:2),

b) PB1 sequence, from N-terminus to C-terminus, FNMLSTVLGV (SEQ ID NO:3),

c) PB1 sequence, from N-terminus to C-terminus, FSMELPSFGV (SEQ ID NO:4),

d) polymerase B2 (PB2) sequence, from N-terminus to C-terminus, YMLERELVRKTRFLPVA (SEQ ID NO:7),

e) PB2 sequence, from N-terminus to C-terminus, NFVNRANQRLNPMHQLLR (SEQ ID NO:8),

f) polymerase A (PA) sequence, from N-terminus to C-terminus, MRRNYFTAEVSHCRATEY (SEQ ID NO:12),

g) PA sequence, from N-terminus to C-terminus, AESRKLLLI (SEQ ID NO:13),

h) hemagglutinin (HA) sequence, from N-terminus to C-terminus, GLFGAIAGFC (SEQ ID NO:14),

i) HA sequence, from N-terminus to C-terminus, TGMVDGWYGYHHQNEQGS (SEQ ID NO:16),

j) HA sequence, from N-terminus to C-terminus, WTYNAELLVLLENERTLD (SEQ ID NO:17),

k) HA sequence, from N-terminus to C-terminus, NKVNSVIEKMNTQFTAVG (SEQ ID NO:18),

l) HA sequence, from N-terminus to C-terminus, VTGLRNIPSIQCR (SEQ ID NO:21),

m) nucleoprotein (NP) sequence, from N-terminus to C-terminus, RRSGAAGAAVK (SEQ ID NO:23),

n) NP sequence, from N-terminus to C-terminus, QLVWMACHSAA (SEQ ID NO:24),

o) NP sequence, from N-terminus to C-terminus, YERMCNILKG (SEQ ID NO:25),

p) NP sequence, from N-terminus to C-terminus, KLSTRGVQIASNEN (SEQ ID NO:29),

q) neuraminidase (NA) sequence, from N-terminus to C-terminus, SWPDGAELPF (SEQ ID NO:30),

r) NA sequence, from N-terminus to C-terminus, PIRGWAI (SEQ ID NO: 31),

s) NA sequence, from N-terminus to C-terminus, SGSFVQHPELTGL (SEQ ID NO:32),

t) NA sequence, from N-terminus to C-terminus, VGLISLILQI (SEQ ID NO:33),

u) matrix protein 1 (M1) sequence, from N-terminus to C-terminus, KTRPILSPLTK (SEQ ID NO:34),

v) M1 sequence, from N-terminus to C-terminus, QKRMGVQMQRFK (SEQ ID NO:35),

w) M1 sequence, from N-terminus to C-terminus, AGKNTDLEALMEWLKTR (SEQ ID NO:36), and

x) non-structural protein 2 (NS2) sequence, from N-terminus to C-terminus, ITFMQALQLL (SEQ ID NO:48),

wherein each of said polypeptides does not comprise any additional amino acid sequence of a naturally occurring Influenza A virus protein besides the amino acid sequences recited in parts a) to x).

8. The composition of embodiment 7, which comprises at least 5, 10, 15, 20, or all 24 of said Influenza A virus polypeptides.

9. The composition of embodiment 7, wherein at least one of the Influenza A virus polypeptides consists essentially of an amino acid sequence selected from the group consisting of the amino acid sequences recited in parts a) to x).

10. The composition of embodiment 9, wherein at least 5, 10, 15, 20, or all 24 of the Influenza A virus polypeptides consist essentially of amino acid sequences selected from the group consisting of the amino acid sequences recited in parts a) to x) of embodiment 7.

11. The composition of embodiment 7, wherein at least one of the Influenza A virus polypeptides consists of an amino acid sequence selected from the group consisting of the amino acid sequences recited in parts a) to x).

12. The composition of embodiment 11, wherein at least 5, 10, 15, 20, or all 24 of the Influenza A virus polypeptides consist of amino acid sequences selected from the group consisting of the amino acid sequences recited in parts a) to x) of embodiment 7.

13. The composition of any of embodiments 1-12, wherein at least one of the Influenza A virus polypeptides is conjugated to a solid surface.

14. The composition of any of embodiments 1-12, wherein all of the Influenza A virus polypeptides are conjugated to a solid surface.

15. The composition of embodiment 13, wherein the at least one Influenza A virus polypeptide is conjugated to a solid surface via a linker.

16. The composition of embodiment 15, wherein the linker is an amino acid residue.

17. The composition of embodiment 16, wherein the amino acid residue is Cys.

18. The composition of any of embodiments 15-17, wherein the linker is attached to the N-terminus of the at least one Influenza A virus polypeptide.

19. The composition of any of embodiments 15-17, wherein the linker is attached to the C-terminus of the at least one Influenza A virus polypeptide.

20. The composition of any of embodiments 13-19, wherein the solid surface is suitable to be used in chromatography.

21. The composition of embodiment 20, wherein the chromatography is column or batch chromatography.

22. The composition of any of embodiments 13-19, wherein the solid surface is suitable to be used in polypeptide analysis.

23. The composition of embodiment 13 or 14, wherein the solid surface is a part of a tube or microtiter plate.

24. A pharmaceutical composition for treating or preventing an Influenza A virus infection, which composition comprises an effective amount of human polyclonal antibodies affinity purified from a human blood sample with the composition of any of embodiments 1-23.

25. The pharmaceutical composition of embodiment 24, wherein the affinity purified human polyclonal antibodies are purified relative to the same human polyclonal antibodies in the unpurified or non-affinity-purified human blood sample.

26. The pharmaceutical composition of embodiment 24, wherein the affinity purified human polyclonal antibodies are specific for the Influenza A virus polypeptides used in the affinity purification.

27. The pharmaceutical composition of embodiment 24, wherein the affinity purified human polyclonal antibodies are substantially free of human antibodies that specifically bind to non-Influenza A virus antigens in the human blood sample.

28. The pharmaceutical composition of embodiment 26, wherein the affinity purified human polyclonal antibodies specific to the Influenza A virus polypeptides have a concentration ranging from about 10 μg/ml to about 10 mg/ml.

29. The pharmaceutical composition of embodiment 25, wherein the affinity purified human polyclonal antibodies are purified from about 2 fold to about 50,000 fold relative to the same human polyclonal antibodies in the unpurified or non-affinity-purified human blood sample.

30. The pharmaceutical composition of any of the embodiments 24-29, wherein the human blood sample is from a normal human.

31. The pharmaceutical composition of any of the embodiments 24-29, wherein the human blood sample is from a human infected with an Influenza A virus.

32. The pharmaceutical composition of embodiment 31, wherein the amino acid residue at position 627 of the PB2 protein of the Influenza A virus is lysine.

33. The pharmaceutical composition of embodiment 31, wherein the Influenza A virus hemagglutinin (HA) binds to alpha 2-6 sialic acid receptors.

34. The pharmaceutical composition of embodiment 31, wherein the Influenza A virus has a subtype selected from the group consisting of H1N1, H2N2, H3N2, H5N1, H7N7, H1N2, H9N2, H7N2, H3N2, H7N3, H5N2, and H10N7.

35. The pharmaceutical composition of any of embodiments 24-34, wherein the human blood sample is pooled from at least 2 humans.

36. The pharmaceutical composition of any of embodiments 24-35, wherein the human polyclonal antibodies are affinity purified with the composition of any of the embodiments 2-23.

37. A method for treating or preventing an Influenza A virus infection, which method comprises administering to a human suffering, suspected of suffering or at risk of suffering from an Influenza A virus infection, an effective amount of the pharmaceutical composition of any of embodiments 24-36.

38. The method of embodiment 37, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an Influenza A virus infection.

39. The method of embodiment 37 or 38, wherein the amino acid residue at position 627 of the PB2 protein of the Influenza A virus is lysine.

40. The method of embodiment 37 or 38, wherein the Influenza A virus hemagglutinin (HA) binds to alpha 2-6 sialic acid receptors.

41. The method of embodiment 37 or 38, wherein the Influenza A virus has a subtype selected from the group consisting of H1N1, H2N2, H3N2, H5N1, H7N7, H1N2, H9N2, H7N2, H3N2, H7N3, H5N2, and H10N7.

42. The method of embodiment 37 or 38, wherein the Influenza A virus is a strain that caused the “Spanish Flu” and the 2009 swine flu outbreak (H1N1), caused the “Asian Flu” in the late 1950s (H2N2), or caused the “Hong Kong Flu” in the late 1960s (H3N2).

43. The method of any of embodiments 37-42, wherein the Influenza A virus infection is caused by an Influenza A virus strain that is resistant to an anti-viral drug or treatment.

44. The method of embodiment 43, wherein the antiviral drug is selected from the group consisting of amantadine, rimantadine, oseltamivir and zanamivir.

45. The method of any of embodiments 37-44, further comprising, prior to administering the affinity purified human polyclonal antibodies to the human, conducting an immunotest to determine the presence, absence and/or amount of Influenza A viral antigens in a blood sample of the human using the same affinity purified human polyclonal antibodies, to assess the suitability of the human for the therapeutic or preventive treatment, wherein a positive immunotest result indicates that the human is suitable for therapy or prevention of Influenza A viral infection using the affinity purified human polyclonal antibodies.

46. The method of any of embodiments 37-44, further comprising, before and after administering the affinity purified human polyclonal antibodies to the human, conducting an immunotest to determine the presence, absence and/or amount of Influenza A viral antigens in a blood sample of the human using the same affinity purified human polyclonal antibodies, to monitor the efficacy of the therapeutic or preventive treatment, wherein the absence or reduction in the Influenza A viral antigens after administering the affinity purified human polyclonal antibodies to the human relative to the amount of Influenza A viral antigens before the administration indicates efficacy of the therapeutic or preventive treatment.

47. The method of any of embodiments 37-44, further comprising, before and after administering the affinity purified human polyclonal antibodies to the human, conducting an immunotest to determine the presence, absence and/or amount of Influenza A viral antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the Influenza A viral antigens remaining after administering the affinity purified human polyclonal antibodies to the human and the extent of reduction in the Influenza A viral antigens after administering the affinity purified human polyclonal antibodies to the human relative to the amount of Influenza A viral antigens before the administration.

48. A method for purifying human polyclonal antibodies to an Influenza A virus, which method comprises:

a) binding human polyclonal antibodies to an Influenza A virus in a human blood sample to the composition of any of embodiments 1-23; and

b) recovering the human polyclonal antibodies to an Influenza A virus bound to the composition of any of embodiments 1-23 to produce affinity purified human polyclonal antibodies to an Influenza A virus.

49. The method of embodiment 48, further comprises a step of concentrating or isolating IgG from the human blood sample.

50. The method of embodiment 49, wherein the IgG is concentrated or isolated from the human blood sample by contacting the human blood sample with ammonium or sodium sulfate.

51. The method of any of embodiments 48-50, wherein the IgG is concentrated or isolated from the human blood sample before the human polyclonal antibodies to an Influenza A virus in the human blood sample are bound to the composition of any of embodiments 1-23.

52. The method of any of embodiments 48-51, which further comprises a step of purifying IgG from the affinity purified human polyclonal antibodies to an Influenza A virus.

53. The method of embodiment 52, wherein the IgG is purified from the affinity purified human polyclonal antibodies to an Influenza A virus by chromatography, e.g., ion exchange column chromatography.

54. Affinity purified human polyclonal antibodies to an Influenza A virus produced by any of embodiments 48-53.

55. A pharmaceutical composition for treating or preventing an Influenza A virus infection, which composition comprises an effective amount of the affinity human polyclonal antibodies of embodiment 54.

56. The pharmaceutical composition of any of embodiments 24-36 and 55, which further comprises a pharmaceutically acceptable carrier or excipient.

57. A method for treating or preventing an Influenza A virus infection, which method comprises administering to a human suffering, suspected of suffering or at risk of suffering from an Influenza A virus infection, an effective amount of the pharmaceutical composition of embodiment 55 or 56.

58. A pharmaceutical composition for treating or preventing a Klebsiella pneumoniae (K. pneumoniae) infection, which composition comprises an effective amount of human polyclonal antibodies affinity purified from a human blood sample with an antigenic preparation comprising cellular and/or secreted antigens of K. pneumoniae cells.

59. The pharmaceutical composition of embodiment 58, wherein the affinity purified human polyclonal antibodies are purified relative to the same human polyclonal antibodies in the unpurified or non-affinity-purified human blood sample.

60. The pharmaceutical composition of embodiment 58, wherein the affinity purified human polyclonal antibodies are specific for the K. pneumoniae antigens used in the affinity purification.

61. The pharmaceutical composition of embodiment 58, wherein the affinity purified human polyclonal antibodies are substantially free of human antibodies that specifically bind to non-K. pneumoniae antigens in said human blood sample.

62. The pharmaceutical composition of any of embodiments 58-61, wherein the affinity purified human polyclonal antibodies specific to the K. pneumoniae antigens have a concentration ranging from about 10 μg/ml to about 10 mg/ml.

63. The pharmaceutical composition of any of embodiments 58-62, wherein the affinity purified human polyclonal antibodies are purified from about 2 fold to about 50,000 fold relative to the same human polyclonal antibodies in the unpurified or non-affinity-purified human blood sample.

64. The pharmaceutical composition of any of embodiments 58-63, wherein the human blood sample is from a normal human.

65. The pharmaceutical composition of any of embodiments 58-63, wherein the human blood sample is from a human infected with K. pneumoniae.

66. The pharmaceutical composition of any of embodiments 58-65, wherein the human blood sample is pooled from at least 2 humans.

67. The pharmaceutical composition of any of embodiments 58-66, wherein the antigenic preparation comprises K. pneumoniae O antigen and/or K antigen.

68. The pharmaceutical composition of any of embodiments 58-66, wherein the antigenic preparation comprises a K. pneumoniae antigen that confers antibiotic resistance.

69. The pharmaceutical composition of any of embodiments 58-66, wherein the antigenic preparation comprises a K. pneumoniae toxin.

70. The pharmaceutical composition of any of embodiments 58-66, wherein the antigenic preparation comprises a whole cell extract and a secreted antigen of K. pneumoniae.

71. The pharmaceutical composition of any of embodiments 58-70, wherein the antigenic preparation is prepared by the following steps:

a) growing K. pneumoniae cells in a first protein containing culture medium;

b) collecting and resuspending the K. pneumoniae cells in a second non-protein containing culture medium;

c) growing the K. pneumoniae cells in the second non-protein containing culture medium; and

d) disrupting the K. pneumoniae cells and collecting a whole cell extract from the disrupted K. pneumoniae cells.

72. The pharmaceutical composition of embodiment 71, wherein the process of making said antigenic preparation further comprises a step of removing an exotoxin from the whole cell extract.

73. The pharmaceutical composition of embodiment 71 or 72, wherein the process of making said antigenic preparation further comprises a step of collecting a secreted antigen from the second non-protein containing culture medium in which the K. pneumoniae cells were grown.

74. A method for treating or preventing a K. pneumoniae infection, which method comprises administering to a human suffering, suspected of suffering or at risk of suffering from a K. pneumoniae infection, an effective amount of the pharmaceutical composition of any of embodiments 58-73.

75. The method of embodiment 74, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from a K. pneumoniae infection.

76. The method of embodiment 74, wherein the human for treatment has a weakened immune system, pneumonia or urinary tract infection.

77. The method of any of embodiments 74-76, wherein the K. pneumoniae infection is caused by a K. pneumoniae strain that is resistant to an anti-bacterial drug or treatment.

78. The method of any of embodiments 74-77, further comprising, prior to administering the affinity purified human polyclonal antibodies to the human, conducting an immunotest to determine the presence, absence and/or amount of K. pneumoniae antigens in a blood sample of the human using the same affinity purified human polyclonal antibodies, to assess the suitability of the human for the therapeutic or preventive treatment, wherein a positive immunotest result indicates that the human is suitable for therapy or prevention of a K. pneumoniae infection using the affinity purified human polyclonal antibodies.

79. The method of any of embodiments 74-77, further comprising, before and after administering the affinity purified human polyclonal antibodies to the human, conducting an immunotest to determine the presence, absence and/or amount of K. pneumoniae antigens in a blood sample of the human using the same affinity purified human polyclonal antibodies, to monitor the efficacy of the therapeutic or preventive treatment, wherein the absence or reduction in the K. pneumoniae antigens after administering the affinity purified human polyclonal antibodies to the human relative to the amount of K. pneumoniae antigens before the administration indicates efficacy of the therapeutic or preventive treatment.

80. The method of any of embodiments 74-77, further comprising, before and after administering the affinity purified human polyclonal antibodies to the human, conducting an immunotest to determine the presence, absence and/or amount of K. pneumoniae antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the K. pneumoniae antigens remaining after administering the affinity purified human polyclonal antibodies to the human and the extent of reduction in the K. pneumoniae antigens after administering the affinity purified human polyclonal antibodies to the human relative to the amount of K. pneumoniae antigens before the administration.

81. A pharmaceutical composition for treating or preventing an Enterococcus faecalis (E. faecalis) infection, which composition comprises an effective amount of human polyclonal antibodies affinity purified from a human blood sample with an antigenic preparation comprising cellular and/or secreted antigens of E. faecalis cells.

82. The pharmaceutical composition of embodiment 81, wherein the affinity purified human polyclonal antibodies are purified relative to the same human polyclonal antibodies in the unpurified or non-affinity-purified human blood sample.

83. The pharmaceutical composition of embodiment 81, wherein the affinity purified human polyclonal antibodies are specific for the E. faecalis antigens used in the affinity purification.

84. The pharmaceutical composition of embodiment 81, wherein the affinity purified human polyclonal antibodies are substantially free of human antibodies that specifically bind to non-E. faecalis antigens in said human blood sample.

85. The pharmaceutical composition of any of embodiments 81-84, wherein the affinity purified human polyclonal antibodies specific to the E. faecalis antigens have a concentration ranging from about 10 μg/ml to about 10 mg/ml.

86. The pharmaceutical composition of any of embodiments 81-85, wherein the affinity purified human polyclonal antibodies are purified from about 2 fold to about 50,000 fold relative to the same human polyclonal antibodies in the unpurified or non-affinity-purified human blood sample.

87. The pharmaceutical composition of any of embodiments 81-86, wherein the human blood sample is from a normal human.

88. The pharmaceutical composition of any of embodiments 81-86, wherein the human blood sample is from a human infected with E. faecalis.

89. The pharmaceutical composition of any of embodiments 81-88, wherein the human blood sample is pooled from at least 2 humans.

90. The pharmaceutical composition of any of embodiments 81-89, wherein the antigenic preparation comprises E. faecalis gelatinase, enterococcal surface protein, aggregation substance, serine protease, capsular polysaccharide, cell wall polysaccharide, hemagglutinin and/or hemolysin/cytolysin.

91. The pharmaceutical composition of any of embodiments 81-89, wherein the antigenic preparation comprises an E. faecalis antigen that confers antibiotic resistance.

92. The pharmaceutical composition of any of embodiments 81-89, wherein the antigenic preparation comprises an E. faecalis toxin.

93. The pharmaceutical composition of any of embodiments 81-89, wherein the antigenic preparation comprises a whole cell extract and a secreted antigen of E. faecalis.

94. The pharmaceutical composition of any of embodiments 81-93, wherein the antigenic preparation is prepared by the following steps:

a) growing E. faecalis cells in a first protein containing culture medium;

b) collecting and resuspending the E. faecalis cells in a second non-protein containing culture medium;

c) growing the E. faecalis cells in the second non-protein containing culture medium; and

d) disrupting the E. faecalis cells and collecting a whole cell extract from the disrupted E. faecalis cells.

95. The pharmaceutical composition of embodiment 94, wherein the process of making said antigenic preparation further comprises a step of removing an exotoxin from the whole cell extract.

96. The pharmaceutical composition of embodiment 94 or 95, wherein the process of making said antigenic preparation further comprises a step of collecting a secreted antigen from the second non-protein containing culture medium in which the E. faecalis cells were grown.

97. A method for treating or preventing an E. faecalis infection, which method comprises administering to a human suffering, suspected of suffering or at risk of suffering from an E. faecalis infection, an effective amount of the pharmaceutical composition of any of embodiments 81-96.

98. The method of embodiment 97, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. faecalis infection.

99. The method of embodiment 97, wherein the human for treatment has a weakened immune system, pneumonia or urinary tract infection.

100. The method of any of embodiments 97-99, wherein the E. faecalis infection is caused by an E. faecalis strain that is resistant to an anti-bacterial drug or treatment.

101. The method of any of embodiments 97-100, further comprising, prior to administering the affinity purified human polyclonal antibodies to the human, conducting an immunotest to determine the presence, absence and/or amount of E. faecalis antigens in a blood sample of the human using the same affinity purified human polyclonal antibodies, to assess the suitability of the human for the therapeutic or preventive treatment, wherein a positive immunotest result indicates that the human is suitable for therapy or prevention of an E. faecalis infection using the affinity purified human polyclonal antibodies.

102. The method of any of embodiments 97-100, further comprising, before and after administering the affinity purified human polyclonal antibodies to the human, conducting an immunotest to determine the presence, absence and/or amount of E. faecalis antigens in a blood sample of the human using the same affinity purified human polyclonal antibodies, to monitor the efficacy of the therapeutic or preventive treatment, wherein the absence or reduction in the E. faecalis antigens after administering the affinity purified human polyclonal antibodies to the human relative to the amount of E. faecalis antigens before the administration indicates efficacy of the therapeutic or preventive treatment.

103. The method of any of embodiments 97-100, further comprising, before and after administering the affinity purified human polyclonal antibodies to the human, conducting an immunotest to determine the presence, absence and/or amount of E. faecalis antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the E. faecalis antigens remaining after administering the affinity purified human polyclonal antibodies to the human and the extent of reduction in the E. faecalis antigens after administering the affinity purified human polyclonal antibodies to the human relative to the amount of E. faecalis antigens before the administration.

104. A pharmaceutical composition for treating or preventing an Enterobacter aerogenes (E. aerogenes) infection, which composition comprises an effective amount of human polyclonal antibodies affinity purified from a human blood sample with an antigenic preparation comprising cellular and/or secreted antigens of E. aerogenes cells.

105. The pharmaceutical composition of embodiment 104, wherein the affinity purified human polyclonal antibodies are purified relative to the same human polyclonal antibodies in the unpurified or non-affinity-purified human blood sample.

106. The pharmaceutical composition of embodiment 104, wherein the affinity purified human polyclonal antibodies are specific for the E. aerogenes antigens used in the affinity purification.

107. The pharmaceutical composition of embodiment 104, wherein the affinity purified human polyclonal antibodies are substantially free of human antibodies that specifically bind to non-E. aerogenes antigens in said human blood sample.

108. The pharmaceutical composition of any of embodiments 104-107, wherein the affinity purified human polyclonal antibodies specific to the E. aerogenes antigens have a concentration ranging from about 10 μg/ml to about 10 mg/ml.

109. The pharmaceutical composition of any of embodiments 104-108, wherein the affinity purified human polyclonal antibodies are purified from about 2 fold to about 50,000 fold relative to the same human polyclonal antibodies in the unpurified or non-affinity-purified human blood sample.

110. The pharmaceutical composition of any of embodiments 104-109, wherein the human blood sample is from a normal human.

111. The pharmaceutical composition of any of embodiments 104-109, wherein the human blood sample is from a human infected with E. aerogenes.

112. The pharmaceutical composition of any of embodiments 104-111, wherein the human blood sample is pooled from at least 2 humans.

113. The pharmaceutical composition of any of embodiments 104-112, wherein the antigenic preparation comprises E. aerogenes 0 antigen and/or K antigen.

114. The pharmaceutical composition of any of embodiments 104-112, wherein the antigenic preparation comprises an E. aerogenes antigen that confers antibiotic resistance.

115. The pharmaceutical composition of any of embodiments 104-112, wherein the antigenic preparation comprises an E. aerogenes toxin.

116. The pharmaceutical composition of any of embodiments 104-112, wherein the antigenic preparation comprises a whole cell extract and a secreted antigen of E. aerogenes.

117. The pharmaceutical composition of any of embodiments 104-116, wherein the antigenic preparation is prepared by the following steps:

a) growing E. aerogenes cells in a first protein containing culture medium;

b) collecting and resuspending the E. aerogenes cells in a second non-protein containing culture medium;

c) growing the E. aerogenes cells in the second non-protein containing culture medium; and

d) disrupting the E. aerogenes cells and collecting a whole cell extract from the disrupted E. aerogenes cells.

118. The pharmaceutical composition of embodiment 117, wherein the process of making said antigenic preparation further comprises a step of removing an exotoxin from the whole cell extract.

119. The pharmaceutical composition of embodiment 117 or 118, wherein the process of making said antigenic preparation further comprises a step of collecting a secreted antigen from the second non-protein containing culture medium in which the E. aerogenes cells were grown.

120. A method for treating or preventing an E. aerogenes infection, which method comprises administering to a human suffering, suspected of suffering or at risk of suffering from an E. aerogenes infection, an effective amount of the pharmaceutical composition of any of embodiments 104-119.

121. The method of embodiment 120, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. aerogenes infection.

122. The method of embodiment 120, wherein the human for treatment has a weakened immune system, pneumonia or urinary tract infection.

123. The method of any of embodiments 120-122, wherein the E. aerogenes infection is caused by an E. aerogenes strain that is resistant to an anti-bacterial drug or treatment.

124. The method of any of embodiments 120-123, further comprising, prior to administering the affinity purified human polyclonal antibodies to the human, conducting an immunotest to determine the presence, absence and/or amount of E. aerogenes antigens in a blood sample of the human using the same affinity purified human polyclonal antibodies, to assess the suitability of the human for the therapeutic or preventive treatment, wherein a positive immunotest result indicates that the human is suitable for therapy or prevention of an E. aerogenes infection using the affinity purified human polyclonal antibodies.

125. The method of any of embodiments 120-123, further comprising, before and after administering the affinity purified human polyclonal antibodies to the human, conducting an immunotest to determine the presence, absence and/or amount of E. aerogenes antigens in a blood sample of the human using the same affinity purified human polyclonal antibodies, to monitor the efficacy of the therapeutic or preventive treatment, wherein the absence or reduction in the E. aerogenes antigens after administering the affinity purified human polyclonal antibodies to the human relative to the amount of E. aerogenes antigens before the administration indicates efficacy of the therapeutic or preventive treatment.

126. The method of any of embodiments 120-123, further comprising, before and after administering the affinity purified human polyclonal antibodies to the human, conducting an immunotest to determine the presence, absence and/or amount of E. aerogenes antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the E. aerogenes antigens remaining after administering the affinity purified human polyclonal antibodies to the human and the extent of reduction in the E. aerogenes antigens after administering the affinity purified human polyclonal antibodies to the human relative to the amount of E. aerogenes antigens before the administration.

127. A pharmaceutical composition for treating or preventing an Enterobacter cloacae (E. cloacae) infection, which composition comprises an effective amount of human polyclonal antibodies affinity purified from a human blood sample with an antigenic preparation comprising cellular and/or secreted antigens of E. cloacae cells.

128. The pharmaceutical composition of embodiment 127, wherein the affinity purified human polyclonal antibodies are purified relative to the same human polyclonal antibodies in the unpurified or non-affinity-purified human blood sample.

129. The pharmaceutical composition of embodiment 127, wherein the affinity purified human polyclonal antibodies are specific for the E. cloacae antigens used in the affinity purification.

130. The pharmaceutical composition of embodiment 127, wherein the affinity purified human polyclonal antibodies are substantially free of human antibodies that specifically bind to non-E. cloacae antigens in said human blood sample.

131. The pharmaceutical composition of any of embodiments 127-130, wherein the affinity purified human polyclonal antibodies specific to the E. cloacae antigens have a concentration ranging from about 10 μg/ml to about 10 mg/ml.

132. The pharmaceutical composition of any of embodiments 127-131, wherein the affinity purified human polyclonal antibodies are purified from about 2 fold to about 50,000 fold relative to the same human polyclonal antibodies in the unpurified or non-affinity-purified human blood sample.

133. The pharmaceutical composition of any of embodiments 127-132, wherein the human blood sample is from a normal human.

134. The pharmaceutical composition of any of embodiments 127-132, wherein the human blood sample is from a human infected with E. cloacae.

135. The pharmaceutical composition of any of embodiments 127-132, wherein the human blood sample is pooled from at least 2 humans.

136. The pharmaceutical composition of any of embodiments 127-135, wherein the antigenic preparation comprises E. cloacae O antigen and/or K antigen.

137. The pharmaceutical composition of any of embodiments 127-135, wherein the antigenic preparation comprises an E. cloacae antigen that confers antibiotic resistance.

138. The pharmaceutical composition of any of embodiments 127-135, wherein the antigenic preparation comprises an E. cloacae toxin.

139. The pharmaceutical composition of any of embodiments 127-135, wherein the antigenic preparation comprises a whole cell extract and a secreted antigen of E. cloacae.

140. The pharmaceutical composition of any of embodiments 127-139, wherein the antigenic preparation is prepared by the following steps:

a) growing E. cloacae cells in a first protein containing culture medium;

b) collecting and resuspending the E. cloacae cells in a second non-protein containing culture medium;

c) growing the E. cloacae cells in the second non-protein containing culture medium; and

d) disrupting the E. cloacae cells and collecting a whole cell extract from the disrupted E. cloacae cells.

141. The pharmaceutical composition of embodiment 140, wherein the process of making said antigenic preparation further comprises a step of removing an exotoxin from the whole cell extract.

142. The pharmaceutical composition of embodiment 140 or 141, wherein the process of making said antigenic preparation further comprises a step of collecting a secreted antigen from the second non-protein containing culture medium in which the E. cloacae cells were grown.

143. A method for treating or preventing an E. cloacae infection, which method comprises administering to a human suffering, suspected of suffering or at risk of suffering from an E. cloacae infection, an effective amount of the pharmaceutical composition of any of embodiments 127-132.

144. The method of embodiment 143, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an E. cloacae infection.

145. The method of embodiment 143, wherein the human for treatment has a weakened immune system, pneumonia or urinary tract infection.

146. The method of any of embodiments 143-145, wherein the E. cloacae infection is caused by an E. cloacae strain that is resistant to an anti-bacterial drug or treatment.

147. The method of any of embodiments 143-146, further comprising, prior to administering the affinity purified human polyclonal antibodies to the human, conducting an immunotest to determine the presence, absence and/or amount of E. cloacae antigens in a blood sample of the human using the same affinity purified human polyclonal antibodies, to assess the suitability of the human for the therapeutic or preventive treatment, wherein a positive immunotest result indicates that the human is suitable for therapy or prevention of an E. cloacae infection using the affinity purified human polyclonal antibodies.

148. The method of any of embodiments 143-146, further comprising, before and after administering the affinity purified human polyclonal antibodies to the human, conducting an immunotest to determine the presence, absence and/or amount of E. cloacae antigens in a blood sample of the human using the same affinity purified human polyclonal antibodies, to monitor the efficacy of the therapeutic or preventive treatment, wherein the absence or reduction in the E. cloacae antigens after administering the affinity purified human polyclonal antibodies to the human relative to the amount of E. cloacae antigens before the administration indicates efficacy of the therapeutic or preventive treatment.

149. The method of any of embodiments 143-146, further comprising, before and after administering the affinity purified human polyclonal antibodies to the human, conducting an immunotest to determine the presence, absence and/or amount of E. cloacae antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the E. cloacae antigens remaining after administering the affinity purified human polyclonal antibodies to the human and the extent of reduction in the E. cloacae antigens after administering the affinity purified human polyclonal antibodies to the human relative to the amount of E. cloacae antigens before the administration.

150. A pharmaceutical composition for treating or preventing an Influenza A virus infection and a bacterial infection, which composition comprises:

a) an effective amount of human polyclonal antibodies affinity purified from a human blood sample with the composition of any of embodiments 1-23; and

b) an effective amount of human polyclonal antibodies affinity purified from a human blood sample with an antigenic preparation comprising cellular and/or secreted antigens from bacterial cells selected from the group consisting of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae) and a combination thereof.

151. The pharmaceutical composition of embodiment 150, wherein the affinity purified human polyclonal antibodies for the Influenza A virus and/or the bacterial cells are purified relative to the same human polyclonal antibodies in the unpurified or non-affinity-purified human blood sample.

152. The pharmaceutical composition of embodiment 150, wherein the affinity purified human polyclonal antibodies are specific for the Influenza A virus and/or the bacterial antigens used in the affinity purification.

153. The pharmaceutical composition of embodiment 150, wherein the affinity purified human polyclonal antibodies are substantially free of human antibodies that specifically bind to non-Influenza A virus and/or non-bacterial antigens in said human blood sample.

154. The pharmaceutical composition of any of embodiments 150-153, wherein the affinity purified human polyclonal antibodies specific to the Influenza A virus and/or the bacterial antigens have a concentration ranging from about 10 μg/ml to about 10 mg/ml.

155. The pharmaceutical composition of any of embodiments 150-154, wherein the affinity purified human polyclonal antibodies are purified from about 2 fold to about 50,000 fold relative to the same human polyclonal antibodies in the unpurified or non-affinity-purified human blood sample.

156. The pharmaceutical composition of any of embodiments 150-155, wherein the human blood sample is from a normal human.

157. The pharmaceutical composition of any of embodiments 150-155, wherein the human blood sample is from a human infected with the Influenza A virus and/or a bacterium selected from the group consisting of S. aureus, a Streptococcus, E. coli, P. aeruginosa, A. baumannii, E. faecium, E. faecalis, E. aerogenes, E. cloacae, C. difficile, K. pneumoniae and a combination thereof.

158. The pharmaceutical composition of any of embodiments 150-157, wherein the human blood sample is pooled from at least 2 humans.

159. The pharmaceutical composition of any of embodiments 150-158, wherein the bacterial antigenic preparation comprises cellular or secreted antigens from the bacterial cells.

160. The pharmaceutical composition of any of embodiments 150-158, wherein the bacterial antigenic preparation comprises cellular and secreted antigens from the bacterial cells.

161. The pharmaceutical composition of any of embodiments 150-160, wherein in a) the human polyclonal antibodies are affinity purified from a human blood sample with the composition of embodiment 2 or 8.

162. The pharmaceutical composition of any of embodiments 150-161, wherein the bacterial antigenic preparation comprises cellular and/or secreted antigens from:

a) any two different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

b) any three different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

c) any four different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

d) any five different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

e) any six different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

f) any seven different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

g) any eight different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

h) any nine different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

i) any ten different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

j) each of S. aureus, a Streptococcus, E. coli, P. aeruginosa, A. baumannii, E. faecium, E. faecalis, E. aerogenes, E. cloacae, C. difficile and K. pneumoniae; or

k) each of S. aureus, S. pneumoniae, E. coli, P. aeruginosa, A. baumannii, E. faecalis and K. pneumoniae; or

l) each of S. aureus, S. pneumoniae, E. coli, P. aeruginosa, A. baumannii, E. faecium and K. pneumoniae.

163. The pharmaceutical composition of embodiment 161 or 162, wherein the bacterial antigenic preparation comprises cellular and/or secreted antigens from:

a) each of S. aureus, S. pneumoniae, E. coli, P. aeruginosa, A. baumannii, E. faecalis and K. pneumoniae; or

b) each of S. aureus, S. pneumoniae, E. coli, P. aeruginosa, A. baumannii, E. faecium and K. pneumoniae .

164. The pharmaceutical composition of any of embodiments 150-163, wherein the bacterial antigenic preparation is prepared by the following steps:

a) growing bacterial cells in a first protein containing culture medium;

b) collecting and resuspending the bacterial cells in a second non-protein containing culture medium;

c) growing the bacterial cells in the second non-protein containing culture medium; and

d) disrupting the bacterial cells and collecting a whole cell extract from the disrupted bacterial cells.

165. The pharmaceutical composition of embodiment 164, wherein the process of making said antigenic preparation further comprises a step of removing an exotoxin from the whole cell extract.

166. The pharmaceutical composition of embodiment 164 or 165, wherein the process of making said antigenic preparation further comprises a step of collecting a secreted antigen from the second non-protein containing culture medium in which the bacterial cells were grown.

167. The pharmaceutical composition of any of embodiments 150-166, wherein the bacterial antigenic preparation comprises an antigen selected from the group consisting of a S. aureus capsular polysaccharide antigen, a S. aureus toxin, staphyloxanthin, and a S. aureus antigen that confers antibiotic resistance.

168. The pharmaceutical composition of any of embodiments 150-166, wherein the bacterial antigenic preparation comprises a S. pneumoniae virulence factor selected from the group consisting of a S. pneumoniae capsular polysaccharide antigen, a S. pneumoniae toxin, autolysin (LytA) and choline binding protein A/pneumococcal surface protein A (CbpA/PspA).

169. The pharmaceutical composition of any of embodiments 150-166, wherein the bacterial antigenic preparation comprises an antigen selected from the group consisting of a P. aeruginosa adhesin, a P. aeruginosa invasin and a P. aeruginosa toxin.

170. The pharmaceutical composition of any of embodiments 150-166, wherein the bacterial antigenic preparation comprises an antigen selected from the group consisting of K. pneumoniae O antigen and K antigen.

171. The pharmaceutical composition of any of embodiments 150-170, wherein the bacterial antigenic preparation comprises a whole cell extract and/or a secreted antigen of bacterial cells from:

a) each of S. aureus, S. pneumoniae, E. coli, P. aeruginosa, A. baumannii, E. faecalis and K. pneumoniae; or

b) each of S. aureus, S. pneumoniae, E. coli, P. aeruginosa, A. baumannii, E. faecium and K. pneumoniae .

172. The pharmaceutical composition of embodiment 171, wherein the antigenic preparation comprises a S. aureus whole cell extract and S. aureus enterotoxin A (SEA) and/or S. aureus enterotoxin B (SEB).

173. The pharmaceutical composition of embodiment 171, wherein the antigenic preparation comprises a Streptococcus whole cell extract and Streptococcal pyrogenic exotoxin A (SpeA) and/or Streptococcal pyrogenic exotoxin C (SpeC).

174. A pharmaceutical composition for treating or preventing an Influenza A virus infection and a bacterial infection, which composition comprises an effective amount of human polyclonal antibodies affinity purified from a human blood sample, said antibodies having specificity for an Influenza A viral antigen, a bacterial antigen and an antigen from human tumor necrosis factor alpha (TNF-α).

175. The pharmaceutical composition of embodiment 174, wherein the human polyclonal antibodies having specificity for the Influenza A viral antigen are affinity purified from said human blood sample with the composition of any of embodiments 1-23.

176. The pharmaceutical composition of embodiment 174 or 175, wherein the human polyclonal antibodies having specificity for the bacterial antigen are affinity purified from said human blood sample with an antigenic preparation comprising cellular and/or secreted antigens from bacterial cells selected from the group consisting of Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae) and a combination thereof.

177. The pharmaceutical composition of any of embodiments 174-176, wherein the affinity purified human polyclonal antibodies to the Influenza A viral antigen, the bacterial antigen and the TNF-α antigen are purified relative to the same human polyclonal antibodies in the unpurified or non-affinity-purified human blood sample.

178. The pharmaceutical composition of any of embodiments 174-177, wherein the affinity purified human polyclonal antibodies are substantially free of human antibodies that specifically bind to non-Influenza A virus, non-bacterial and non-TNF-α antigens in said human blood sample.

179. The pharmaceutical composition of any of embodiments 174-178, wherein the affinity purified human polyclonal antibodies specific to the Influenza A viral antigen, the bacterial antigen and the TNF-α antigen have a concentration ranging from about 10 μg/ml to about 10 mg/ml.

180. The pharmaceutical composition of any of embodiments 174-179, wherein the affinity purified human polyclonal antibodies are purified from about 2 fold to about 50,000 fold relative to the same human polyclonal antibodies in the unpurified or non-affinity-purified human blood sample.

181. The pharmaceutical composition of any of embodiments 174-180, wherein the human blood sample is from a normal human.

182. The pharmaceutical composition of any of embodiments 174-180, wherein the human blood sample is from a human infected with the Influenza A virus and/or a bacterium selected from the group consisting of S. aureus, a Streptococcus, E. coli, P. aeruginosa, A. baumannii, E. faecium, E. faecalis, E. aerogenes, E. cloacae, C. difficile, K. pneumoniae and a combination thereof.

83. The pharmaceutical composition of any of embodiments 174-182, wherein the human blood sample is pooled from at least 2 humans.

184. The pharmaceutical composition of any of embodiments 174-183, wherein the human polyclonal antibodies having specificity for the Influenza A viral antigen are affinity purified from said human blood sample with the composition of embodiment 2 or 8.

185. The pharmaceutical composition of any of embodiments 174-184, wherein the human polyclonal antibodies having specificity for the bacterial antigen are affinity purified from said human blood sample with an antigenic preparation comprising cellular or secreted bacterial antigens.

186. The pharmaceutical composition of any of embodiments 174-184, wherein the human polyclonal antibodies having specificity for the bacterial antigen are affinity purified from said human blood sample with an antigenic preparation comprising cellular and secreted bacterial antigens.

187. The pharmaceutical composition of any of embodiments 174-184, wherein the human polyclonal antibodies having specificity for the bacterial antigen are affinity purified from said human blood sample with an antigenic preparation comprising cellular and/or secreted antigens from:

a) any two different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

b) any three different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

c) any four different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

d) any five different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

e) any six different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

f) any seven different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

g) any eight different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or P h) any nine different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

i) any ten different bacterial species selected from the group consisting of S. aureus, a Streptococcus, E. coli, P. aeruginosa, A. baumannii, E. faecium, E. faecalis, E. aerogenes, E. cloacae, C. difficile and K. pneumoniae; or

j) each of S. aureus, a Streptococcus, E. coli, P. aeruginosa, A. baumannii, E. faecium, E. faecalis, E. aerogenes, E. cloacae, C. difficile and K. pneumoniae; or

k) each of S. aureus, S. pneumoniae, E. coli, P. aeruginosa, A. baumannii, E. faecalis and K. pneumoniae; or

l) each of S. aureus, S. pneumoniae, E. coli, P. aeruginosa, A. baumannii, E. faecium and K. pneumoniae.

188. The pharmaceutical composition of embodiment 187, wherein said antigenic preparation comprises cellular and/or secreted antigens from:

a) each of S. aureus, S. pneumoniae, E. coli, P. aeruginosa, A. baumannii, E. faecalis and K. pneumoniae; or

b) each of S. aureus, S. pneumoniae, E. coli, P. aeruginosa, A. baumannii, E. faecium and K. pneumoniae .

189. The pharmaceutical composition of any of embodiments 174-188, wherein the bacterial antigen is prepared by the following steps:

a) growing bacterial cells in a first protein containing culture medium;

b) collecting and resuspending the bacterial cells in a second non-protein containing culture medium;

c) growing the bacterial cells in the second non-protein containing culture medium; and

d) disrupting the bacterial cells and collecting a whole cell extract from the disrupted bacterial cells.

190. The pharmaceutical composition of embodiment 189, wherein the process of making said bacterial antigen further comprises a step of removing an exotoxin from the whole cell extract.

191. The pharmaceutical composition of embodiment 189 or 190, wherein the process of making said bacterial antigen further comprises a step of collecting a secreted antigen from the second non-protein containing culture medium in which the bacterial cells were grown.

192. The pharmaceutical composition of any of embodiments 174-191, wherein the bacterial antigen comprises an antigen selected from the group consisting of a S. aureus capsular polysaccharide antigen, a S. aureus toxin, staphyloxanthin, and a S. aureus antigen that confers antibiotic resistance.

193. The pharmaceutical composition of any of embodiments 174-191, wherein the bacterial antigen comprises a S. pneumoniae virulence factor selected from the group consisting of a S. pneumoniae capsular polysaccharide antigen, a S. pneumoniae toxin, autolysin (LytA) and choline binding protein A/pneumococcal surface protein A (CbpA/PspA).

194. The pharmaceutical composition of any of embodiments 174-191, wherein the bacterial antigen comprises an antigen selected from the group consisting of a P. aeruginosa adhesin, a P. aeruginosa invasin and a P. aeruginosa toxin.

195. The pharmaceutical composition of any of embodiments 174-191, wherein the bacterial antigen comprises an antigen selected from the group consisting of K. pneumoniae O antigen and K antigen.

196. The pharmaceutical composition of any of embodiments 174-195, wherein the bacterial antigen comprises a whole cell extract and/or a secreted antigen of bacterial cells from:

a) each of S. aureus, S. pneumoniae, E. coli, P. aeruginosa, A. baumannii, E. faecalis and K. pneumoniae; or

b) each of S. aureus, S. pneumoniae, E. coli, P. aeruginosa, A. baumannii, E. faecium and K. pneumoniae .

197. The pharmaceutical composition of embodiment 196, wherein the bacterial antigen comprises a S. aureus whole cell extract and S. aureus enterotoxin A (SEA) and/or S. aureus enterotoxin B (SEB).

198. The pharmaceutical composition of embodiment 196, wherein the bacterial antigen comprises a Streptococcus whole cell extract and Streptococcal pyrogenic exotoxin A (SpeA) and/or Streptococcal pyrogenic exotoxin C (SpeC).

199. A method for treating or preventing an Influenza A virus infection and a bacterial infection, which method comprises administering to a human suffering, suspected of suffering, or at risk of suffering from an Influenza A virus infection, a S. aureus infection, a Streptococcus infection, an E. coli infection, a P. aeruginosa infection, an A. baumannii infection, an E. faecium infection, an E. faecalis infection, an E. aerogenes infection, an E. cloacae infection, a C. difficile infection, and/or a K. pneumoniae infection, an effective amount of the pharmaceutical composition of any of embodiments 150-198.

200. The method of embodiment 199, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from an Influenza A virus infection, S. aureus infection, a Streptococcus infection, E. coli infection, P. aeruginosa infection, A. baumannii infection, E. faecium infection, an E. faecalis infection, an E. aerogenes infection, an E. cloacae infection, C. difficile infection, and/or K. pneumoniae infection.

201. The method of embodiment 199 or 200, wherein the amino acid residue at position 627 of the PB2 protein of the Influenza A virus is lysine.

202. The method of embodiment 199 or 200, wherein the Influenza A virus hemagglutinin (HA) binds to alpha 2-6 sialic acid receptors.

203. The method of embodiment 199 or 200, wherein the Influenza A virus has a subtype selected from the group consisting of H1N1, H2N2, H3N2, H5N1, H7N7, H1N2, H9N2, H7N2, H3N2, H7N3, H5N2, and H10N7.

204. The method of embodiment 199 or 200, wherein the Influenza A virus is a strain that caused the “Spanish Flu” and the 2009 swine flu outbreak (H1N1), caused the “Asian Flu” in the late 1950s (H2N2), or caused the “Hong Kong Flu” in the late 1960s (H3N2).

205. The method of any of embodiments 199-204, wherein the Influenza A virus infection is caused by an Influenza A virus strain that is resistant to an anti-viral drug or treatment.

206. The method of embodiment 205, wherein the antiviral drug is selected from the group consisting of amantadine, rimantadine, oseltamivir and zanamivir.

207. The method of any of embodiments 199-206, wherein the human suffers, is suspected of suffering, or is at risk of suffering from bacteremia.

208. The method of any of embodiments 199-207, wherein the S. aureus infection is caused by a S. aureus strain that is resistant to an antibiotic.

209. The method of embodiment 208, wherein the S. aureus infection is caused by a methicillin-resistant strain (MRSA), a vancomycin intermediate strain (VISA) or vancomycin resistant strain (VRSA).

210. The method of any of embodiments 199-209, wherein the human suffers, is suspected of suffering, or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease.

211. The method of any of embodiments 199-210, wherein the Streptococcus infection is caused by Streptococcus pneumoniae (S. pneumoniae), a Group A Streptococcus (GAS) or a Group B Streptococcus (GBS).

212. The method of embodiment 211, wherein the Streptococcus is selected from the group consisting of Streptococcus pneumoniae (S. pneumoniae), Streptococcus pyogenes (S. pyogenes), Streptococcus agalactiae (S. agalactiae) and a combination thereof.

213. The method of any of embodiments 199-212, wherein the human suffers, is suspected of suffering, or is at risk of suffering from gastroenteritis, a urinary tract infection, neonatal meningitis, hemolytic-uremic syndrome (HUS), peritonitis, mastitis, septicemia or Gram-negative pneumonia.

214. The method of any of embodiments 199-213, wherein the human has a weakened immune system, pneumonia or urinary tract infection.

215. The method of any of embodiments 199-214, wherein the K. pneumoniae infection is caused by a K. pneumoniae strain that is resistant to an anti-bacterial drug or treatment.

216. The method of any of embodiments 199-215, further comprising, prior to administering the affinity purified human polyclonal antibodies to the human, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the human using the same affinity purified human polyclonal antibodies, to assess the suitability of the human for the therapeutic or preventive treatment, wherein a positive immunotest result indicates that the human is suitable for therapy or prevention of an Influenza A virus, S. aureus, a Streptococcus, E. coli, P. aeruginosa, A. baumannii, E. faecium, E. faecalis, E. aerogenes, E. cloacae, C. difficile, and/or K. pneumoniae infection using the affinity purified human polyclonal antibodies.

217. The method of embodiment 216, wherein the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, and a positive immunotest result indicates that the human is suitable for therapy or prevention of Influenza A virus, S. aureus, S. pneumoniae, E. coli, P. aeruginosa, A. baumannii, E. faecalis and K. pneumoniae infections using the affinity purified human polyclonal antibodies.

218. The method of embodiment 216, wherein the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, and a positive immunotest result indicates that the human is suitable for therapy or prevention of Influenza A virus, S. aureus, S. pneumoniae, E. coli, P. aeruginosa, A. baumannii, E. faecium and K. pneumoniae infections using the affinity purified human polyclonal antibodies.

219. The method of any of embodiments 199-215, further comprising, before and after administering the affinity purified human polyclonal antibodies to the human, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the human using the same affinity purified human polyclonal antibodies, to monitor the efficacy of the therapeutic or preventive treatment, wherein the absence or reduction in the Influenza A virus, S. aureus, a Streptococcus, E. coli, P. aeruginosa, A. baumannii, E. faecium, E. faecalis, E. aerogenes, E. cloacae, C. difficile, K pneumoniae and/or TNF-α antigens after administering the affinity purified human polyclonal antibodies to the human relative to the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens before the administration indicates efficacy of the therapeutic or preventive treatment.

220. The method of embodiment 219, wherein the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, and the absence or reduction in the Influenza A virus, S. aureus, S. pneumoniae, E. coli, P. aeruginosa, A. baumannii, E. faecalis, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecalis, K. pneumoniae and TNF-α antigens before the administration indicates efficacy of the therapeutic or preventive treatment.

221. The method of embodiment 219, wherein the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, and the absence or reduction in the Influenza A virus, S. aureus, S. pneumoniae, E. coli, P. aeruginosa, A. baumannii, E. faecium, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecium, K. pneumoniae and TNF-α antigens before the administration indicates efficacy of the therapeutic or preventive treatment.

222. The method of any of embodiments 199-215, further comprising, before and after administering the affinity purified human polyclonal antibodies to the human, conducting an immunotest to determine the presence, absence and/or amount of Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens in a blood sample of the human using the same affinity purified human polyclonal antibodies, to determine an optimal therapeutic or preventive dose of the affinity purified human polyclonal antibodies, wherein the optimal therapeutic or preventive dose is determined based on the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens remaining after administering the affinity purified human polyclonal antibodies to the human and the extent of reduction in the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens after administering the affinity purified human polyclonal antibodies to the human relative to the amount of the Influenza A virus, S. aureus, a Streptococcus, E. coli, P. aeruginosa, A. baumannii, E. faecium, E. faecalis, E. aerogenes, E. cloacae, C. difficile, K. pneumoniae and/or TNF-α antigens before the administration.

223. The method of embodiment 222, wherein the immunotest is conducted to determine the presence, absence and/or amount of Influenza A virus, S. aureus, S. pneumoniae, E. coli, P. aeruginosa, A. baumannii, E. faecalis, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecalis, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecalis, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecalis, K. pneumoniae and TNF-α antigens before the administration.

224. The method of embodiment 222, wherein the immunotest is conducted to determine the presence, absence and/or amount of Influenza A virus, S. aureus, S. pneumoniae, E. coli, P. aeruginosa, A. baumannii, E. faecium, K. pneumoniae and TNF-α antigens, to determine an optimal therapeutic or preventive dose of the affinity purified human polyclonal antibodies, wherein the optimal therapeutic or preventive dose is determined based on the amount of the Influenza A virus, S. aureus, S. pneumoniae, E. coli, P. aeruginosa, A. baumannii, E. faecium, K. pneumoniae and TNF-α antigens remaining after administering the affinity purified human polyclonal antibodies to the human and the extent of reduction in the Influenza A virus, S. aureus, S. pneumoniae, E. coli, P. aeruginosa, A. baumannii, E. faecium, K. pneumoniae and TNF-α antigens after administering the affinity purified human polyclonal antibodies to the human relative to the amount of the Influenza A virus, S. aureus, S. pneumoniae, E. coli, P. aeruginosa, A. baumannii, E. faecium, K. pneumoniae and TNF-α antigens before the administration.

225. The method of any of embodiments 45-47, 78-80, 101-103, 124-126, 147-149 and 216-224, wherein the immunotest is conducted in a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescence assay (IFA), nephelometry, flow cytometry assay, plasmon resonance assay, chemiluminescence assay, lateral flow immunoassay, μ-capture assay, inhibition assay and avidity assay.

226. A method for treating or preventing an Influenza A virus infection and a bacterial infection, which method comprises administering to a human suffering, suspected of suffering, or at risk of suffering from an Influenza A virus infection, a S. aureus infection, a S. pneumoniae infection, an E. coli infection, a P. aeruginosa infection, an A. baumannii infection, an E. faecalis infection and/or a K. pneumoniae infection, an effective amount of the pharmaceutical composition of embodiment 188.

227. A pharmaceutical composition for treating or preventing an Influenza A virus infection and a bacterial infection, which composition comprises an effective amount of antibodies that specifically bind to an Influenza A viral antigen, a bacterial antigen and an antigen from human tumor necrosis factor alpha (TNF-α).

228. The pharmaceutical composition of embodiment 227, wherein the Influenza A viral antigen comprises an antigen from hemagglutinin (HA), neuraminidase (NA), nucleoprotein (NP), matrix protein 1 (M1), matrix protein 2 (M2), non-structural protein 1 (NS1), non-structural protein 2 (NS2 or NEP), polymerase A (PA), polymerase B1 (PB1 and PB1-F2) and/or polymerase B2 (PB2).

229. The pharmaceutical composition of embodiment 227, wherein the bacterial antigen comprises an antigen from S. aureus, a Streptococcus, E. coli, P. aeruginosa, A. baumannii, E. faecium, E. faecalis, E. aerogenes, E. cloacae, C. difficile and/or K. pneumoniae.

230. The pharmaceutical composition of embodiment 227, wherein the Influenza A viral antigen comprises an antigen from each of hemagglutinin (HA), neuraminidase (NA), nucleoprotein (NP), matrix protein 1 (M1), matrix protein 2 (M2), non-structural protein 1 (NS1), non-structural protein 2 (NS2 or NEP), polymerase A (PA), polymerase B1 (PB1 and PB1-F2) and/or polymerase B2 (PB2), and the bacterial antigen comprises an antigen from each of S. aureus, S. pneumoniae, E. coli, P. aeruginosa, A. baumannii, E. faecalis and K. pneumoniae.

231. The pharmaceutical composition of embodiment 227, wherein the Influenza A viral antigen comprises an antigen from each of hemagglutinin (HA), neuraminidase (NA), nucleoprotein (NP), matrix protein 1 (M1), matrix protein 2 (M2), non-structural protein 1 (NS1), non-structural protein 2 (NS2 or NEP), polymerase A (PA), polymerase B1 (PB1 and PB1-F2) and/or polymerase B2 (PB2), and the bacterial antigen comprises an antigen from each of S. aureus, S. pneumoniae, E. coli, P. aeruginosa, A. baumannii, E. faecium and K. pneumoniae .

232. A method for treating or preventing an Influenza A virus infection and a bacterial infection, which method comprises administering to a human suffering, suspected of suffering, or at risk of suffering from an Influenza A virus infection, a S. aureus infection, a S. pneumoniae infection, an E. coli infection, a P. aeruginosa infection, an A. baumannii infection, an E. faecalis infection and/or a K. pneumoniae infection, an effective amount of the pharmaceutical composition of any of the embodiments 224-231.

The numbering of the above Exemplary Embodiments 1-232 applies to the Exemplary Embodiments in the Exemplary Embodiments Section B.

### Exemplary Embodiments Section C

1. An immunological composition, which composition comprises an effective amount of an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus, e.g., Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

2. The immunological composition of embodiment 1, wherein the antigenic preparation comprises an endotoxin and/or an exotoxin.

3. The immunological composition of embodiment 1, wherein the antigenic preparation comprises a whole cell extract and a secreted antigen.

4. The immunological composition of embodiment 3, wherein the antigenic preparation comprises an endotoxin and/or an exotoxin.

5. The immunological composition of any of embodiments 1-4, wherein the antigenic preparation comprises cellular and secreted antigens from at least 3, 4, 5, 6, 7, 8, 9, 10, 11, 12, 13, 14, 15, 16, 17, 18, 19, 20, or all 21 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

6. The immunological composition of any of embodiments 1-4, wherein the antigenic preparation comprises cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

7. The immunological composition of embodiment 6, wherein the antigenic preparation comprises cellular and secreted antigens from at least 3, 4, 5, 6, 7, or all 8 different bacteria selected from the group consisting of Staphylococcus aureus (S. aureus), Escherichia coli (E. coli), a Streptococcus, Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

8. The immunological composition of any of embodiments 6 and 7, wherein the Streptococcus is Streptococcus pneumoniae (S. pneumoniae).

9. The immunological composition of embodiment 8, wherein the antigenic preparation comprises cellular and secreted antigens from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae (H. influenzae), Pseudomonas aeruginosa (P. aeruginosa), and Acinetobacter baumannii (A. baumannii).

10. The immunological composition of embodiment 9, wherein the antigenic preparation comprises an endotoxin and/or an exotoxin.

11. The immunological composition of embodiment 9, wherein the antigenic preparation comprises a whole cell extract and a secreted antigen.

12. The immunological composition of embodiment 11, wherein the antigenic preparation comprises an endotoxin and/or an exotoxin.

13. The immunological composition of any of embodiments 1-12, wherein the antigenic preparation comprises cellular and secreted antigens from S. aureus.

14. The immunological composition of embodiment 13, wherein the S. aureus is resistant to an antibiotic.

15. The immunological composition of embodiment 13, wherein the S. aureus is a methicillin-resistant strain (MRSA), a vancomycin intermediate strain (VISA) or vancomycin resistant strain (VRSA).

16. The immunological composition of any of embodiments 13-15, wherein the antigenic preparation comprises a S. aureus capsular polysaccharide antigen.

17. The immunological composition of embodiment 16, wherein the S. aureus capsular polysaccharide antigen is selected from the group consisting of the Type 5 antigen, the Type 8 antigen, and the 336 antigen.

18. The immunological composition of any of embodiments 13-17, wherein the antigenic preparation comprises a S. aureus toxin.

19. The immunological composition of embodiment 18, wherein the S. aureus toxin is selected from the group consisting of a pyrogenic toxin superantigen (PTSAg), an exfoliative toxin and a Staphylococcal toxin.

20. The immunological composition of embodiment 19, wherein the pyrogenic toxin superantigen (PTSAg) is the toxic shock syndrome toxin 1 (TSST-1) and/or a S. aureus enterotoxin.

21. The immunological composition of embodiment 20, wherein the S. aureus enterotoxin is S. aureus enterotoxin A (SEA) and/or S. aureus enterotoxin B (SEB).

22. The immunological composition of embodiment 19, wherein the Staphylococcal toxin is selected from the group consisting of alpha-toxin, beta-toxin, delta-toxin and a bicomponent toxin.

23. The immunological composition of embodiment 22, wherein the bicomponent toxin is Panton-Valentine leukocidin (PVL).

24. The immunological composition of any of embodiments 13-23, wherein the antigenic preparation comprises staphyloxanthin.

25. The immunological composition of any of embodiments 13-24, wherein the antigenic preparation comprises an antigen selected from the group consisting of penicillinase, an altered penicillin-binding protein (PBP2a or PBP2′) encoded by the mecA gene, an aminoglycoside modifying enzyme and an enzyme encoded by the vanA gene.

26. The immunological composition of any of embodiments 13-25, wherein the antigenic preparation comprises two or more antigens selected from the group consisting of a S. aureus capsular polysaccharide antigen, a S. aureus toxin, staphyloxanthin, and a S. aureus antigen that confers antibiotic resistance.

27. The immunological composition of any of embodiments 13-25, wherein the antigenic preparation comprises two or more antigens selected from the group consisting of a S. aureus toxin, staphyloxanthin, and a S. aureus antigen that confers antibiotic resistance.

28. The immunological composition of any of embodiments 13-27, wherein the S. aureus antigenic preparation is prepared by the following steps:

a) growing S. aureus cells in a first protein containing S. aureus culture medium for a first period of time;

b) collecting and resuspending the S. aureus cells in a second non-protein containing S. aureus culture medium;

c) growing the S. aureus cells in the second non-protein containing S. aureus culture medium for a second period of time; and

d) disrupting the S. aureus cells and collecting a whole cell extract from disrupted S. aureus cells.

29. The immunological composition of embodiment 28, which further comprises collecting a secreted antigen from the second non-protein containing S. aureus culture medium in which the S. aureus cells have grown for the second period of time.

30. The immunological composition of any of embodiments 28-29, wherein the first protein containing S. aureus culture medium comprises a pancreatic digest of casein, an enzymatic digest of soybean meal, NaCl, K₂HPO₄ and dextrose.

31. The immunological composition of any of embodiments 28-30, wherein the first period of time is from about 10 hours to about 72 hours.

32. The immunological composition of any of embodiments 28-31, wherein the second non-protein containing S. aureus culture medium comprises an aqueous solution comprising sodium chloride, sodium phosphate, and optionally a source of carbon.

33. The immunological composition of any of embodiments 28-32, wherein the second period of time is from about 10 hours to about 48 hours.

34. The immunological composition of any of embodiments 28-33, wherein the S. aureus cells are disrupted by homogenization, freeze thaw and/or sonication.

35. The immunological composition of any of embodiments 28-34, wherein disrupting the S. aureus cells and collecting a whole cell extract from disrupted S. aureus cells, and collecting a secreted antigen from the second non-protein containing S. aureus culture medium in which the S. aureus cells have grown for the second period of time, are performed in one step, the S. aureus cells are disrupted in the second non-protein containing S. aureus culture medium, and insoluble cellular debris are removed to collect a whole cell extract and secreted antigens of S. aureus.

36. The immunological composition of embodiment 35, wherein the S. aureus cells are disrupted by homogenization, freeze thaw and/or sonication.

37. The immunological composition of any of embodiments 35-36, wherein the insoluble S. aureus cellular debris are removed by centrifugation or filtration.

38. The immunological composition of any of embodiments 1-12, wherein the antigenic preparation comprises cellular and secreted antigens from a Streptococcus.

39. The immunological composition of embodiment 38, wherein the Streptococcus is resistant to an antibiotic.

40. The immunological composition of embodiment 38, wherein the Streptococcus is selected from the group consisting of Streptococcus pneumoniae (S. pneumoniae), Streptococcus pyogenes (S. pyogenes), Streptococcus agalactiae (S. agalactiae) and a combination thereof.

41. The immunological composition of embodiment 40, wherein the Streptococcus is a S. pneumoniae strain that is resistant to an antibiotic.

42. The immunological composition of embodiment 41, wherein the antibiotic is selected from the group consisting of penicillin, tetracycline, clindamycin, a cephalosporin, a macrolide and a quinolone.

43. The immunological composition of embodiment 38, wherein the antigenic preparation comprises two or more S. pneumoniae virulence factors selected from the group consisting of a S. pneumoniae capsular polysaccharide antigen, a S. pneumoniae toxin, autolysin (LytA) and choline binding protein A/pneumococcal surface protein A (CbpA/PspA).

44. The immunological composition of embodiment 43, wherein the S. pneumoniae toxin is pneumolysin (Ply).

45. The immunological composition of any of embodiments 38-44, wherein the Streptococcus antigenic preparation is prepared by the following steps:

a) growing Streptococcus cells in a first protein containing Streptococcus culture medium for a first period of time;

b) collecting and resuspending the Streptococcus cells in a second non-protein containing Streptococcus culture medium;

c) growing the Streptococcus cells in the second non-protein containing Streptococcus culture medium for a second period of time; and

d) disrupting the Streptococcus cells and collecting a whole cell extract from disrupted Streptococcus cells.

46. The immunological composition of embodiment 45, which further comprises collecting a secreted antigen from the second non-protein containing Streptococcus culture medium in which the Streptococcus cells have grown for the second period of time.

47. The immunological composition of any of embodiments 45-46, wherein the first protein containing Streptococcus culture medium comprises a pancreatic digest of casein, an enzymatic digest of soybean meal, NaCl, K₂HPO₄ and dextrose.

48. The immunological composition of any of embodiments 45-47, wherein the first period of time is from about 10 hours to about 72 hours.

49. The immunological composition of any of embodiments 45-48, wherein the second non-protein containing Streptococcus culture medium comprises an aqueous solution comprising sodium chloride, sodium phosphate, and optionally source of sugar or carbon.

50. The immunological composition of any of embodiments 45-49, wherein the second period of time is from about 10 hours to about 48 hours.

51. The immunological composition of any of embodiments 45-50, wherein the Streptococcus cells are disrupted by homogenization, freeze thaw and/or sonication.

52. The immunological composition of any of embodiments 45-51, wherein disrupting the Streptococcus cells and collecting a whole cell extract from disrupted Streptococcus cells and collecting a secreted antigen from the second non-protein containing Streptococcus culture medium in which the Streptococcus cells have grown for the second period of time are performed in one step, the Streptococcus cells are disrupted in the second non-protein containing Streptococcus culture medium, and insoluble cellular debris are removed to collect a whole cell extract and secreted antigens of Streptococcus.

53. The immunological composition of embodiment 52, wherein the Streptococcus cells are disrupted by homogenization, freeze thaw and/or sonication.

54. The immunological composition of embodiment 53, wherein the insoluble Streptococcus cellular debris are removed by centrifugation or filtration.

55. The immunological composition of any of embodiments 1-12, wherein the antigenic preparation comprises cellular and secreted antigens from Escherichia coli (E. coli).

56. The immunological composition of embodiment 55, wherein the E. coli is selected from the group consisting of enterotoxigenic E. coli (ETEC), enteropathogenic E. coli (EPEC), enteroinvasive E. coli (EIEC), enterohemorrhagic E. coli (EHEC), enteroaggregative E. coli (EAggEC) and uropathogenic E. coli (UPEC).

57. The immunological composition of embodiment 56, wherein the EHEC is a Shiga toxin-producing E. coli (STEC).

58. The immunological composition of embodiment 57, wherein the STEC is strain O157:H7.

59. The immunological composition of embodiment 55, wherein the antigenic composition comprises two or more E. coli virulence factors selected from the group consisting of an E. coli capsular polysaccharide antigen, K antigen, an enterotoxin, an adhesin, a hemolysin and a Shiga toxin.

60. The immunological composition of embodiment 59, wherein the enterotoxin is heat-labile LT enterotoxin and/or heat-stable ST enterotoxin.

61. The immunological composition of embodiment 59, wherein the adhesin is a fimbrial adhesin and/or intimin.

62. The immunological composition of embodiment 59, wherein the hemolysin is alpha-hemolysin and/or beta-hemolysin.

63. The immunological composition of embodiment 55, wherein the E. coli is resistant to an antibiotic.

64. The immunological composition of embodiment 63, wherein the antibiotic is selected from the group consisting of penicillin, streptomycin, chloramphenicol, ampicillin, cephalosporin and tetracycline.

65. The immunological composition of any of embodiments 55-64, wherein the E. coli antigenic preparation is prepared by the following steps:

a) growing E. coli cells in a first protein containing E. coli culture medium for a first period of time;

b) collecting and resuspending the E. coli cells in a second non-protein containing E. coli culture medium;

c) growing the E. coli cells in the second non-protein containing E. coli culture medium for a second period of time; and

d) disrupting the E. coli cells and collecting a whole cell extract from disrupted E. coli cells.

66. The immunological composition of embodiment 65, which further comprises collecting a secreted antigen from the second non-protein containing E. coli culture medium in which the E. coli cells have grown for the second period of time.

67. The immunological composition of any of embodiments 65-66, wherein the first protein containing E. coli culture medium comprises a pancreatic digest of casein, an enzymatic digest of soybean meal, NaCl, K₂HPO₄ and dextrose.

68. The immunological composition of any of embodiments 65-67, wherein the first period of time is from about 10 hours to about 72 hours.

69. The immunological composition of any of embodiments 65-68, wherein the second non-protein containing E. coli culture medium comprises an aqueous solution comprising sodium chloride, sodium phosphate, and optionally a source of carbon.

70. The immunological composition of any of embodiments 65-69, wherein the second period of time is from about 10 hours to about 48 hours.

71. The immunological composition of any of embodiments 65-70, wherein the E. coli cells are disrupted by homogenization, freeze thaw and/or sonication.

72. The immunological composition of any of embodiments 65-71, wherein disrupting the E. coli cells and collecting a whole cell extract from disrupted E. coli cells and collecting a secreted antigen from the second non-protein containing E. coli culture medium in which the E. coli cells have grown for the second period of time are performed in one step, the E. coli cells are disrupted in the second non-protein containing E. coli culture medium, and insoluble cellular debris are removed to collect a whole cell extract and secreted antigens of E. coli.

73. The immunological composition of embodiment 72, wherein the E. coli cells are disrupted by homogenization, freeze thaw and/or sonication.

74. The immunological composition of embodiment 73, wherein the insoluble E. coli cellular debris are removed by centrifugation or filtration.

75. The immunological composition of any of embodiments 1-12, wherein the antigenic preparation comprises cellular and secreted antigens from Klebsiella pneumoniae (K. pneumoniae).

76. The immunological composition of embodiment 75, wherein the antigenic preparation comprises K. pneumoniae O antigen and/or K antigen.

77. The immunological composition of embodiment 75, wherein the K. pneumoniae is resistant to an antibiotic.

78. The immunological composition of embodiment 75, wherein the antigenic preparation comprises a K. pneumoniae toxin.

79. The immunological composition of embodiment 75, wherein the antigenic preparation comprises a whole cell extract and a secreted antigen of K. pneumoniae .

80. The immunological composition of any of embodiments 75-79, wherein the K. pneumoniae antigenic preparation is prepared by the following steps:

a) growing K. pneumoniae cells in a first protein containing culture medium for a first period of time;

b) collecting and resuspending the K. pneumoniae cells in a second non-protein containing culture medium;

c) growing the K. pneumoniae cells in the second non-protein containing culture medium for a second period of time; and

d) disrupting the K. pneumoniae cells and collecting a whole cell extract from the disrupted K. pneumoniae cells.

81. The immunological composition of embodiment 80, which further comprises collecting a secreted antigen from the second non-protein containing K. pneumoniae culture medium in which the K. pneumoniae cells have grown for the second period of time.

82. The immunological composition of any of embodiments 80-81, wherein the first protein containing K. pneumoniae culture medium comprises a pancreatic digest of casein, an enzymatic digest of soybean meal, NaCl, K₂HPO₄ and dextrose.

83. The immunological composition of any of embodiments 80-82, wherein the first period of time is from about 10 hours to about 72 hours.

84. The immunological composition of any of embodiments 80-83, wherein the second non-protein containing K. pneumoniae culture medium comprises an aqueous solution comprising sodium chloride, sodium phosphate, and optionally a source of carbon.

85. The immunological composition of any of embodiments 80-84, wherein the second period of time is from about 10 hours to about 48 hours.

86. The immunological composition of any of embodiments 80-84, wherein the K. pneumoniae cells are disrupted by homogenization, freeze thaw and/or sonication.

87. The immunological composition of any of embodiments 80-86, wherein disrupting the K. pneumoniae cells and collecting a whole cell extract from disrupted K. pneumoniae cells and collecting a secreted antigen from the second non-protein containing K. pneumoniae culture medium in which the K. pneumoniae cells have grown for the second period of time are performed in one step, the K. pneumoniae cells are disrupted in the second non-protein containing K. pneumoniae culture medium, and insoluble cellular debris are removed to collect a whole cell extract and secreted antigens of K. pneumoniae.

88. The immunological composition of embodiment 87, wherein the K. pneumoniae cells are disrupted by homogenization, freeze thaw and/or sonication.

89. The immunological composition of embodiment 88, wherein the insoluble K. pneumoniae cellular debris are removed by centrifugation or filtration.

90. The immunological composition of any of embodiments 1-12, wherein the antigenic preparation comprises cellular and secreted antigens from an Enterococcus.

91. The immunological composition of embodiment 90, wherein the Enterococcus is resistant to an antibiotic.

92. The immunological composition of embodiment 90, wherein the antigenic preparation comprises an Enterococcus toxin.

93. The immunological composition of embodiment 90, wherein the antigenic preparation comprises a whole cell extract and a secreted antigen of the Enterococcus.

94. The immunological composition of embodiment 90, wherein the Enterococcus is Enterococcus faecium (E. faecium) or Enterococcus faecalis (E. faecalis).

95. The immunological composition of embodiment 94, wherein the E. faecalis is resistant to an antibiotic.

96. The immunological composition of embodiment 95, wherein the E. faecalis is VRE (vancomycin-resistant Enterococcus).

97. The immunological composition of any of embodiments 90-96, wherein the Enterococcus antigenic preparation is prepared by the following steps:

a) growing Enterococcus cells in a first protein containing culture medium for a first period of time;

b) collecting and resuspending the Enterococcus cells in a second non-protein containing culture medium;

c) growing the Enterococcus cells in the second non-protein containing culture medium for a second period of time; and

d) disrupting the Enterococcus cells and collecting a whole cell extract from the disrupted Enterococcus cells.

98. The immunological composition of embodiment 97, which further comprises collecting a secreted antigen from the second non-protein containing Enterococcus culture medium in which the Enterococcus cells have grown for the second period of time.

99. The immunological composition of any of embodiments 90-98, wherein the first protein containing Enterococcus culture medium comprises a pancreatic digest of casein, an enzymatic digest of soybean meal, NaCl, K₂HPO₄ and dextrose.

100. The immunological composition of any of embodiments 90-99, wherein the first period of time is from about 10 hours to about 72 hours.

101. The immunological composition of any of embodiments 90-100, wherein the second non-protein containing Enterococcus culture medium comprises an aqueous solution comprising sodium chloride, sodium phosphate, and optionally a source of carbon.

102. The immunological composition of any of embodiments 90-101, wherein the second period of time is from about 10 hours to about 48 hours.

103. The immunological composition of any of embodiments 90-102, wherein the Enterococcus cells are disrupted by homogenization, freeze thaw and/or sonication.

104. The immunological composition of any of embodiments 90-103, wherein disrupting the Enterococcus cells and collecting a whole cell extract from disrupted Enterococcus cells and collecting a secreted antigen from the second non-protein containing Enterococcus culture medium in which the Enterococcus cells have grown for the second period of time are performed in one step, the Enterococcus cells are disrupted in the second non-protein containing Enterococcus culture medium, and insoluble cellular debris are removed to collect a whole cell extract and secreted antigens of Enterococcus.

105. The immunological composition of embodiment 104, wherein the Enterococcus cells are disrupted by homogenization, freeze thaw and/or sonication.

106. The immunological composition of embodiment 105, wherein the insoluble Enterococcus cellular debris are removed by centrifugation or filtration.

107. The immunological composition of any of embodiments 1-12, wherein the antigenic preparation comprises cellular and secreted antigens from Haemophilus influenzae (H. influenzae).

108. The immunological composition of embodiment 107, wherein the H. influenzae is resistant to an antibiotic.

109. The immunological composition of embodiment 107, wherein the antigenic preparation comprises a H. influenzae toxin.

110. The immunological composition of embodiment 107, wherein the antigenic preparation comprises a whole cell extract and a secreted antigen of H. influenzae.

111. The immunological composition of embodiment 107, wherein the H. influenzae is an unencapsulated strain or an encapsulated strain.

112. The immunological composition of embodiment 111, wherein the encapsulated strain has the serotype a, b, c, d, e, or f.

113. The immunological composition of any of embodiments 107-112, wherein the H. influenzae antigenic preparation is prepared by the following steps:

a) growing H. influenzae cells in a first protein containing culture medium for a first period of time;

b) collecting and resuspending the H. influenzae cells in a second non-protein containing culture medium;

c) growing the H. influenzae cells in the second non-protein containing culture medium for a second period of time; and

d) disrupting the H. influenzae cells and collecting a whole cell extract from the disrupted H. influenzae cells.

114. The immunological composition of embodiment 113, which further comprises collecting a secreted antigen from the second non-protein containing H. influenzae culture medium in which the H. influenzae cells have grown for the second period of time.

115. The immunological composition of any of embodiments 107-114, wherein the first protein containing H. influenzae culture medium comprises a pancreatic digest of casein, an enzymatic digest of soybean meal, NaCl, K₂HPO₄ and dextrose.

116. The immunological composition of any of embodiments 107-115, wherein the first period of time is from about 10 hours to about 72 hours.

117. The immunological composition of any of embodiments 107-116, wherein the second non-protein containing H. influenzae culture medium comprises an aqueous solution comprising sodium chloride, sodium phosphate, and optionally a source of carbon.

118. The immunological composition of any of embodiments 107-117, wherein the second period of time is from about 10 hours to about 48 hours.

119. The immunological composition of any of embodiments 107-118, wherein the H. influenzae cells are disrupted by homogenization, freeze thaw and/or sonication.

120. The immunological composition of any of embodiments 107-119, wherein disrupting the H. influenzae cells and collecting a whole cell extract from disrupted H. influenzae cells and collecting a secreted antigen from the second non-protein containing H. influenzae culture medium in which the H. influenzae cells have grown for the second period of time are performed in one step, the H. influenzae cells are disrupted in the second non-protein containing H. influenzae culture medium, and insoluble cellular debris are removed to collect a whole cell extract and secreted antigens of H. influenzae.

121. The immunological composition of embodiment 120, wherein the H. influenzae cells are disrupted by homogenization, freeze thaw and/or sonication.

122. The immunological composition of embodiment 121, wherein the insoluble H. influenzae cellular debris are removed by centrifugation or filtration.

123. The immunological composition of any of embodiments 1-12, wherein the antigenic preparation comprises cellular and secreted antigens from Pseudomonas aeruginosa (P. aeruginosa).

124. The immunological composition of embodiment 123, wherein the P. aeruginosa is resistant to an antibiotic.

125. The immunological composition of embodiment 123, wherein the antigenic preparation comprises two or more antigens selected from the group consisting of a P. aeruginosa adhesin, a P. aeruginosa invasin and a P. aeruginosa toxin.

126. The immunological composition of embodiment 125, wherein the P. aeruginosa adhesin is a fimbrial adhesin, a capsular polysaccharide antigen or a mucoid exopolysaccharide antigen.

127. The immunological composition of embodiment 126, wherein the fimbrial adhesin comprises N-methyl-phenylalanine.

128. The immunological composition of embodiment 126, wherein the capsular polysaccharide antigen is glycocalyx.

129. The immunological composition of embodiment 126, wherein the mucoid exopolysaccharide antigen is alginate.

130. The immunological composition of embodiment 125, wherein the P. aeruginosa invasin is a protease, a cytotoxin, a hemolysin, or a diffusible pigment.

131. The immunological composition of embodiment 130, wherein the protease is an elastase or an alkaline protease.

132. The immunological composition of embodiment 130, wherein the cytotoxin is leukocidin.

133. The immunological composition of embodiment 130, wherein the hemolysin is a phospholipase or a lecithinase.

134. The immunological composition of embodiment 130, wherein the diffusible pigment is pyocyanin or pyochelin.

135. The immunological composition of embodiment 125, wherein the P. aeruginosa toxin is lipopolysaccharide (LPS) endotoxin or an extracellular toxin.

136. The immunological composition of embodiment 135, wherein the extracellular toxin is P. aeruginosa exoenzyme S (PES) or P. aeruginosa exotoxin A (PEA).

137. The immunological composition of embodiment 123, wherein the antigenic preparation comprises a whole cell extract and a secreted antigen of P. aeruginosa.

138. The immunological composition of any of embodiments 123-137, wherein the P. aeruginosa antigenic preparation is prepared by the following steps:

a) growing P. aeruginosa cells in a first protein containing culture medium for a first period of time;

b) collecting and resuspending the P. aeruginosa cells in a second non-protein containing culture medium;

c) growing the P. aeruginosa cells in the second non-protein containing culture medium for a second period of time; and

d) disrupting the P. aeruginosa cells and collecting a whole cell extract from the disrupted P. aeruginosa cells.

139. The immunological composition of embodiment 138, which further comprises collecting a secreted antigen from the second non-protein containing P. aeruginosa culture medium in which the P. aeruginosa cells have grown for the second period of time.

140. The immunological composition of any of embodiments 138-139, wherein the first protein containing P. aeruginosa culture medium comprises a pancreatic digest of casein, an enzymatic digest of soybean meal, NaCl, K₂HPO₄ and dextrose.

141. The immunological composition of any of embodiments 138-140, wherein the first period of time is from about 10 hours to about 72 hours.

142. The immunological composition of any of embodiments 138-141, wherein the second non-protein containing P. aeruginosa culture medium comprises an aqueous solution comprising sodium chloride, sodium phosphate, and optionally a source of carbon.

143. The immunological composition of any of embodiments 138-142, wherein the second period of time is from about 10 hours to about 48 hours.

144. The immunological composition of any of embodiments 138-143, wherein the P. aeruginosa cells are disrupted by homogenization, freeze thaw and/or sonication.

145. The immunological composition of any of embodiments 138-144, wherein disrupting the P. aeruginosa cells and collecting a whole cell extract from disrupted P. aeruginosa cells and collecting a secreted antigen from the second non-protein containing P. aeruginosa culture medium in which the P. aeruginosa cells have grown for the second period of time are performed in one step, the P. aeruginosa cells are disrupted in the second non-protein containing P. aeruginosa culture medium, and insoluble cellular debris are removed to collect a whole cell extract and secreted antigens of P. aeruginosa.

146. The immunological composition of embodiment 146, wherein the P. aeruginosa cells are disrupted by homogenization, freeze thaw and/or sonication.

147. The immunological composition of embodiment 146, wherein the insoluble P. aeruginosa cellular debris are removed by centrifugation or filtration.

148. The immunological composition of any of embodiments 1-12, wherein the antigenic preparation comprises cellular and secreted antigens from Acinetobacter baumannii (A. baumannii).

149. The immunological composition of embodiment 148, wherein the A. baumannii is resistant to an antibiotic.

150. The immunological composition of embodiment 148, wherein the antigenic preparation comprises a whole cell extract and a secreted antigen of A. baumannii.

151. The immunological composition of any of embodiments 148-150, wherein the A. baumannii antigenic preparation is prepared by the following steps:

a) growing A. baumannii cells in a first protein containing culture medium for a first period of time;

b) collecting and resuspending the A. baumannii cells in a second non-protein containing culture medium for a second period of time;

c) growing the A. baumannii cells in the second non-protein containing culture medium; and

d) disrupting the A. baumannii cells and collecting a whole cell extract from the disrupted A. baumannii cells.

152. The immunological composition of embodiment 151, which further comprises collecting a secreted antigen from the second non-protein containing A. baumannii culture medium in which the A. baumannii cells have grown for the second period of time.

153. The immunological composition of any of embodiments 151-152, wherein the first protein containing A. baumannii culture medium comprises a pancreatic digest of casein, an enzymatic digest of soybean meal, NaCl, K₂HPO₄ and dextrose.

154. The immunological composition of any of embodiments 151-153, wherein the first period of time is from about 10 hours to about 72 hours.

155. The immunological composition of any of embodiments 151-155, wherein the second non-protein containing A. baumannii culture medium comprises an aqueous solution comprising sodium chloride, sodium phosphate, and optionally a source of carbon.

156. The immunological composition of any of embodiments 151-155, wherein the second period of time is from about 10 hours to about 48 hours.

157. The immunological composition of any of embodiments 151-156, wherein the A. baumannii cells are disrupted by homogenization, freeze thaw and/or sonication.

158. The immunological composition of any of embodiments 151-157, wherein disrupting the A. baumannii cells and collecting a whole cell extract from disrupted A. baumannii cells and collecting a secreted antigen from the second non-protein containing A. baumannii culture medium in which the A. baumannii cells have grown for the second period of time are performed in one step, the A. baumannii cells are disrupted in the second non-protein containing A. baumannii culture medium, and insoluble cellular debris are removed to collect a whole cell extract and secreted antigens of A. baumannii.

159. The immunological composition of embodiment 158, wherein the A. baumannii cells are disrupted by homogenization, freeze thaw and/or sonication.

160. The immunological composition of embodiment 159, wherein the insoluble A. baumannii cellular debris are removed by centrifugation or filtration.

161. The immunological composition of embodiment 9, wherein the antigenic preparation comprises a whole cell extract and a secreted antigen from 8 different bacteria selected from the group consisting of Staphylococcus aureus (S. aureus), Escherichia coli (E. coli), Streptococcus pneumoniae (S. pneumoniae), Klebsiella pneumoniae (K. pneumoniae), Enterococcus faecium (E. faecium), Haemophilus influenzae, Pseudomonas aeruginosa (P. aeruginosa), and Acinetobacter baumannii (A. baumannii).

162. The immunological composition of embodiment 161, wherein the bacterial antigenic preparation is prepared by the following steps:

a) growing the bacterial cells in a first protein containing culture medium for a first period of time;

b) collecting and resuspending the bacterial cells in a second non-protein containing culture medium;

c) growing the bacterial cells in the second non-protein containing culture medium for a second period of time; and

d) disrupting the bacterial cells and collecting a whole cell extract from the disrupted bacterial cells.

163. The immunological composition of embodiment 162, which further comprises collecting a secreted antigen from the second non-protein containing bacterial culture medium in which the bacterial cells have grown for the second period of time.

164. The immunological composition of any of embodiments 161-163, wherein the first protein containing bacterial culture medium comprises a pancreatic digest of casein, an enzymatic digest of soybean meal, NaCl, K₂HPO₄ and dextrose.

165. The immunological composition of any of embodiments 161-164, wherein the first period of time is from about 10 hours to about 72 hours.

166. The immunological composition of any of embodiments 161-165, wherein the second non-protein containing bacterial culture medium comprises an aqueous solution comprising sodium chloride, sodium phosphate, and optionally a source of carbon.

167. The immunological composition of any of embodiments 161-166, wherein the second period of time is from about 10 hours to about 48 hours.

168. The immunological composition of any of embodiments 161-167, wherein the bacterial cells are disrupted by homogenization, freeze thaw and/or sonication.

169. The immunological composition of any of embodiments 161-168, wherein disrupting the bacterial cells and collecting a whole cell extract from disrupted bacterial cells and collecting a secreted antigen from the second non-protein containing bacterial culture medium in which the bacterial cells have grown for the second period of time are performed in one step, the bacterial cells are disrupted in the second non-protein containing bacterial culture medium, and insoluble cellular debris are removed to collect a whole cell extract and secreted antigens of the bacteria.

170. The immunological composition of embodiment 169, wherein the bacterial cells are disrupted by homogenization, freeze thaw and/or sonication.

171. The immunological composition of embodiment 170, wherein the insoluble bacterial cellular debris are removed by centrifugation or filtration.

172. The immunological composition of any of embodiments 162-171, wherein the antigenic preparation comprising a whole cell extract and a secreted antigen for a different bacterium is prepared separately.

173. A vaccine, which vaccine comprises an effective amount of an immunological composition of any of embodiments 1-172.

174. The vaccine of embodiment 173, which further comprises an adjuvant, an excipient or an immune response potentiator.

175. The vaccine of embodiment 174, wherein the adjuvant comprises an aluminum salt or gel.

176. The vaccine of embodiment 174, wherein the excipient is an antibiotic, an egg protein, a stabilizer or a preservative.

177. The vaccine of embodiment 176, wherein the stabilizer is monosodium glutamate (MSG) or 2-phenoxyethanol.

178. The vaccine of embodiment 176, wherein the preservative is formaldehyde, phenoxyethanol, Thimerosal or a mercury-containing preservative.

179. The vaccine of embodiment 174, wherein the immune response potentiator is selected from the group consisting of Bacille Calmette-Guerin (BCG), Corynebacterium parvum, Brucella abortus extract, glucan, levamisole, tilorone, an enzyme and a non-virulent virus.

180. The vaccine of embodiment 173, which comprises the immunological composition of any of embodiments 161-172.

181. The vaccine of any of embodiments 173-180, which is formulated for intravenous, intraperitoneal, intracorporeal, intra-articular, intraventricular, intrathecal, intramuscular, subcutaneous, intranasal, intravaginal, topical or oral administration.

182. The vaccine of any of embodiments 173-181, which is formulated as a solid, a semi-solid, a gel, a liquid, a semi-liquid, a skin patch or an aerosol.

183. The vaccine of embodiment 182, wherein the solid is a tablet.

184. The vaccine of any of embodiments 173-183, which is formulated for administration with a liposome, an immune stimulating complex (ISCOM), or a micro-needle.

185. A method for immunizing or treating a subject, which method comprises administering to a subject for whom such immunization or treatment is needed or desirable an effective amount of a vaccine of any of embodiments 173-184.

186. The method of embodiment 185, wherein the subject is a human.

187. The method of embodiment 186, which is used to protect or treat the human from a bacterial infection, a tumor or a cancer.

188. The method of embodiment 187, wherein the bacterial infection is caused by a bacterial strain that is resistant to an antibiotic.

189. The method of any of embodiments 186-188, wherein the human is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, a patient in an emergency room, a dialysis patient, a surgery patient, e.g., a patient with elective surgery, especially orthopedic surgery patient for hip, knee, shoulder, or other body part replacement, an athlete, a healthcare worker, or a tumor or cancer patient.

190. The method of embodiment 189, wherein the implanted medical device or part is selected from the group consisting of a catheter, a prosthesis, an artificial hip, knee or limb, a dialysis access graft, a pacemaker and an implantable defibrillator.

191. The method of embodiment 189, wherein the immunocompromised patient is a chemotherapy patient, a patient receiving a steroid treatment or a patient taking an immunosuppressive drug.

192. The method of any of embodiments 186-191, wherein the human suffers, is suspected of suffering, or is at risk of suffering from bacteremia.

193. The method of any of embodiments 186-192, wherein the human suffers, is suspected of suffering or is at risk of suffering from toxic shock syndrome (TSS), Staphylococcal scalded skin syndrome (SSSS, also known as pemphigus neonatorum or Ritter's disease, or localized bullous impetigo), pyaemia (or pyemia), a boil (or furuncle), a carbuncle, staphylococcal endocarditis, staphylococcal pneumonia or atopic dermatitis, and the vaccine comprises an antigenic preparation comprising cellular and secreted antigens from S. aureus.

194. The method of any of embodiments 186-192, wherein the human suffers, is suspected of suffering or is at risk of suffering from bacterial pneumonia, bacterial meningitis, otitis media, streptococcal pharyngitis (strep throat), scarlet fever, acute rheumatic fever, endocarditis, streptococcal toxic shock syndrome, streptococcal bacteremia or perinatal Group B streptococcal disease, and the vaccine comprises an antigenic preparation comprising cellular and secreted antigens from a Streptococcus.

195. The method of embodiment 194, wherein the Streptococcus is Streptococcus pneumoniae (S. pneumoniae).

196. The method of any of embodiments 186-192, wherein the human suffers, is suspected of suffering or is at risk of suffering from gastroenteritis, a urinary tract infection, neonatal meningitis, hemolytic-uremic syndrome (HUS), peritonitis, mastitis, septicemia or Gram-negative pneumonia, and the vaccine comprises an antigenic preparation comprising cellular and secreted antigens from E. coli.

197. The method of any of embodiments 186-192, wherein the human suffers, is suspected of suffering or is at risk of suffering from Klebsiella pneumonia, ankylosing spondylitis (AS, previously known as Bekhterev's disease, Bekhterev syndrome, and Marie-Strümpell disease, a form of Spondyloarthritis), a urinary tract infection, a patient with chronic pulmonary disease, enteric pathogenicity, nasal mucosa atrophy, and rhinoscleroma, and the vaccine comprises an antigenic preparation comprising cellular and secreted antigens from K. pneumoniae.

198. The method of any of embodiments 186-192, wherein the human suffers, is suspected of suffering or is at risk of suffering from neonatal meningitis, and the vaccine comprises an antigenic preparation comprising cellular and secreted antigens from E. faecium.

199. The method of any of embodiments 186-192, wherein the human suffers, is suspected of suffering or is at risk of suffering from bacteremia, pneumonia, acute bacterial meningitis, cellulitis, osteomyelitis, epiglottitis, infectious arthritis, ear infection (otitis media), eye infection (conjunctivitis), sinusitis or pneumonia, and the vaccine comprises an antigenic preparation comprising cellular and secreted antigens from H. influenzae.

200. The method of embodiment 199, wherein the H. influenzae) is H. influenzae type b (Hib) or unencapsulated H. influenzae.

201. The method of any of embodiments 199-200, wherein the human is an infant or a young child.

202. The method of any of embodiments 186-192, wherein the human suffers, is suspected of suffering or is at risk of suffering from pneumonia, septic shock, urinary tract infection, gastrointestinal infection, skin and soft tissue infection, infection of a burn injury, infection of an external ear (otitis externa), hot-tub rash (dermatitis), post-operative infection in a radial keratotomy surgery patient, ecthyma gangrenosum, osteomyelitis involving puncture wound of the foot, and the vaccine comprises an antigenic preparation comprising cellular and secreted antigens from P. aeruginosa.

203. The method of embodiment 202, wherein the human is a cystic fibrosis patient, a neutropenic patient, a premature infant, a neutropaenic cancer patient, a burns victim or a patient with wound infection.

204. The method of any of embodiments 186-192, wherein the human suffers, is suspected of suffering or is at risk of suffering from pneumonia, infection of the urinary tract, bloodstream or other part of the body, wound, necrotizing fasciitis, or nosocomial A. baumannii bacteremia, and the vaccine comprises an antigenic preparation comprising cellular and secreted antigens from A. baumannii.

205. The method of embodiment 204, wherein the A. baumannii is a multidrug-resistant strain.

206. The method of embodiment 204, wherein the human is a hospital patient or a patient with immunodeficiency.

207. The method of any of embodiments 186-206, wherein the vaccine is administered via intravenous, intraperitoneal, intracorporeal, intra-articular, intraventricular, intrathecal, intramuscular, subcutaneous, intranasal, intravaginal, topical or oral administration.

208. The method of any of embodiments 186-207, wherein the vaccine is administered as a solid, a semi-solid, a gel, a liquid, a semi-liquid, a skin patch or an aerosol.

209. The method of embodiment 208, wherein the solid is a tablet.

210. The method of any of embodiments 186-206, wherein the vaccine is administered with a liposome, an immune stimulating complex (ISCOM), or a micro-needle.

211. The method of any of embodiments 186-210, further comprising administering a pharmaceutically acceptable carrier or excipient to the human.

212. The method of any of embodiments 186-211, further comprising administering an additional therapeutic or preventive agent.

213. The method of embodiment 212, wherein the additional therapeutic or preventive agent is an antibiotic, an antimicrobial agent, a bactericidal agent, a bacteriostatic agent, or an immunostimulatory compound.

214. The method of embodiment 213, wherein the antibiotic is penicillin, a penicillinase-resistant penicillin, a glycopeptide or an aminoglycoside.

215. The method of embodiment 214, wherein the penicillinase-resistant penicillin is selected from the group consisting of methicillin, oxacillin, cloxacillin, dicloxacillin and flucloxacillin.

216. The method of embodiment 214, wherein the glycopeptide is vancomycin.

217. The method of embodiment 214, wherein the aminoglycoside is selected from the group consisting of kanamycin, gentamicin and streptomycin.

218. The method of embodiment 213, wherein the immunostimulatory compound is a beta-glucan or GM-CSF.

219. The method of embodiment 213, wherein the antimicrobial agent is lysostaphin.

220. The method of any of embodiments 186-219, wherein the vaccine comprises the immunological composition of any of embodiments 161-172.

221. The method of embodiment 193, which further comprises assaying S. aureus infection in the human.

222. The method of any of embodiments 194-195, which further comprises assaying a Streptococcus infection in the human.

223. The method of embodiment 196, which further comprises assaying E. coli infection in the human.

224. The method of embodiment 197, which further comprises assaying K. pneumoniae infection in the human.

225. The method of embodiment 198, which further comprises assaying E. faecium infection in the human.

226. The method of any of embodiments 199-201, which further comprises assaying H. influenzae infection in the human.

227. The method of any of embodiments 202-203, which further comprises assaying P. aeruginosa infection in the human.

228. The method of any of embodiments 204-206, which further comprises assaying A. baumannii infection in the human.

229. The method of embodiment 187, wherein the bacterial infection is caused by at least two different bacteria selected from the group consisting of Staphylococcus aureus (S. aureus), Escherichia coli (E. coli), a Streptococcus, e.g., S. pneumoniae, Klebsiella pneumoniae (K. pneumoniae), Enterococcus, e.g., E. faecium, Haemophilus influenzae (H. influenzae), Pseudomonas aeruginosa (P. aeruginosa), and Acinetobacter baumannii (A. baumannii).

230. The method of embodiment 187, wherein the vaccine is administered to the tumor or cancer site.

231. The method of embodiment 187, wherein the cancer is bladder cancer or colorectal cancer.

232. The method of embodiment 231, wherein the bladder cancer is a superficial form of bladder cancer.

233. An immunological composition for treating cystic fibrosis, which immunological composition comprises an antigenic composition comprising cellular and secreted antigens from Pseudomonas aeruginosa (P. aeruginosa).

234. The immunological composition of embodiment 233, wherein the antigenic preparation comprises a whole cell extract and a secreted antigen of P. aeruginosa.

235. An immunological composition for treating cystic fibrosis, which immunological composition comprises an antigenic composition comprising cellular and secreted antigens from Burkholderia cepacia complex (BCC).

236. The immunological composition of embodiment 235, wherein the antigenic composition comprises cellular and secreted antigens from B. cepacia, B. multivorans, B. cenocepacia, B. vietnamiensis, B. stabilis, B. ambifaria, B. dolosa, B. anthina, and B. pyrrocinia.

237. The immunological composition of embodiment 235, wherein the antigenic preparation comprises a whole cell extract and a secreted antigen of BCC.

238. The immunological composition of embodiment 237, wherein the antigenic composition comprises a whole cell extract and a secreted antigen of B. cepacia, B. multivorans, B. cenocepacia, B. vietnamiensis, B. stabilis, B. ambifaria, B. dolosa, B. anthina, and B. pyrrocinia.

239. A vaccine for treating cystic fibrosis, which vaccine comprises an effective amount of an immunological composition of any of embodiments 233-238.

240. A method for treating cystic fibrosis, which method comprises administering to a subject in need of such treatment an effective amount of an immunological composition of any of embodiments 233-238.

241. A method for treating cystic fibrosis, which method comprises administering to a subject in need of such treatment an effective amount of the vaccine of embodiment 239.

The numbering of the above Exemplary Embodiments 1-241 applies to the Exemplary Embodiments in the Exemplary Embodiments Section C.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the claims.

1. A pharmaceutical composition for treating or preventing a Haemophilus influenzae (formerly called Pfeiffer's bacillus or Bacillus influenzae) infection, which composition comprises an effective amount of human polyclonal antibodies affinity purified from a human blood sample with an antigenic preparation comprising a cellular antigen and/or a secreted antigen of Haemophilus influenzae cells.

2. The pharmaceutical composition of claim 1, wherein the affinity purified human polyclonal antibodies are purified relative to the same human polyclonal antibodies in the unpurified or non-affinity-purified human blood sample.

3. The pharmaceutical composition of claim 1, wherein the affinity purified human polyclonal antibodies are specific for the Haemophilus influenzae antigen(s) used in the affinity purification.

4. The pharmaceutical composition of claim 1, wherein the affinity purified human polyclonal antibodies are substantially free of human antibodies that specifically bind to non-Haemophilus influenzae antigens in the human blood sample.

5. The pharmaceutical composition of claim 1, wherein the affinity purified human polyclonal antibodies specific to the Haemophilus influenzae antigen(s) have a concentration ranging from about 10 μg/ml to about 10 mg/ml.

6. The pharmaceutical composition of claim 1, wherein the affinity purified human polyclonal antibodies are purified from about 2 fold to about 50,000 fold relative to the same human polyclonal antibodies in the unpurified or non-affinity-purified human blood sample.

7. The pharmaceutical composition of claim 1, wherein the human blood sample is from a normal human.

8. The pharmaceutical composition of claim 1, wherein the human blood sample is from a human infected with Haemophilus influenzae.

9. The pharmaceutical composition of claim 1, wherein the human blood sample is pooled from at least 2 humans.

10. The pharmaceutical composition of claim 1, wherein the Haemophilus influenzae is an unencapsulated strain or an encapsulated strain.

11. The pharmaceutical composition of claim 1, wherein the antigenic preparation comprises a Haemophilus influenzae antigen that confers antibiotic resistance.

12. The pharmaceutical composition of claim 1, wherein the antigenic preparation comprises a whole cell extract and a secreted antigen of Haemophilus influenzae.

13. The pharmaceutical composition of claim 1, wherein the antigenic preparation is prepared by the following steps:
a) growing Haemophilus influenzae cells in a first protein containing culture medium;
b) collecting and resuspending the Haemophilus influenzae cells in a second non-protein containing culture medium;
c) growing the Haemophilus influenzae cells in the second non-protein containing culture medium; and
d) disrupting the bacterial cells and collecting a whole cell extract from the disrupted Haemophilus influenzae cells.

14. The pharmaceutical composition of claim 1, which further comprises an effective amount of human polyclonal antibodies affinity purified from a human blood sample with an antigenic preparation comprising a cellular antigen and/or a secreted antigen of Staphylococcus aureus (S. aureus), a Streptococcus, and/or Pseudomonas aeruginosa (P. aeruginosa) cells.

15. The pharmaceutical composition of claim 14, which comprises an effective amount of human polyclonal antibodies affinity purified from a human blood sample with an antigenic preparation comprising a cellular antigen and/or a secreted antigen of:
a) Haemophilus influenzae and at least one of S. aureus, Streptococcus, and P. aeruginosa; or
b) Haemophilus influenzae and at least two of S. aureus, Streptococcus, and P. aeruginosa; or
c) Haemophilus influenzae and each of S. aureus, Streptococcus, and P. aeruginosa.

16. A method for treating or preventing a Haemophilus influenzae infection, which method comprises administering to a human suffering, suspected of suffering or at risk of suffering from a Haemophilus influenzae infection, an effective amount of a pharmaceutical composition comprising an effective amount of human polyclonal antibodies affinity purified from a human blood sample with an antigenic preparation comprising a cellular antigen and/or a secreted antigen of Haemophilus influenzae cells.

17. The method of claim 16, wherein the human for treatment is selected from the group consisting of a healthy individual, an infant, a child, a teenager, a young adult, an adult, a senior, a nursing mother, a surgical patient, an individual with a foreign implanted medical device or part, a patient with a fistula, an immunocompromised patient, a patient with a chronic illness, a patient being cared for in a health care facility, a patient with an indwelling catheter, and/or a patient who has previously suffered from the Haemophilus influenzae infection.

18. The method of claim 16, wherein the human for treatment has a weakened immune system, bacteremia, pneumonia, acute bacterial meningitis, cellulitis, osteomyelitis, epiglottitis, infectious arthritis, ear infections (otitis media), eye infections (conjunctivitis), and/or sinusitis.

19. The method of claim 16, wherein the Haemophilus influenzae infection is caused by a Haemophilus influenzae strain that is resistant to an anti-bacterial drug or treatment.

20. The method of claim 16, further comprising, prior to administering the affinity purified human polyclonal antibodies to the human, conducting an immunotest to determine the presence, absence and/or amount of a Haemophilus influenzae antigen in a blood sample of the human using the same affinity purified human polyclonal antibodies, to assess the suitability of the human for the therapeutic, removal or preventive treatment, wherein a positive immunotest result indicates that the human is suitable for therapy, removal or prevention of the Haemophilus influenzae infection using the affinity purified human polyclonal antibodies.

21. The method of claim 16, further comprising, before and after administering the affinity purified human polyclonal antibodies to the human, conducting an immunotest to determine the presence, absence and/or amount of a Haemophilus influenzae antigen in a blood sample of the human using the same affinity purified human polyclonal antibodies, to monitor the efficacy of the therapeutic, removal or preventive treatment, wherein the absence or reduction in the Haemophilus influenzae antigen after administering the affinity purified human polyclonal antibodies to the human relative to the amount of Haemophilus influenzae antigen before the administration indicates efficacy of the therapeutic, removal or preventive treatment.

22. The method of claim 16, further comprising, before and after administering the affinity purified human polyclonal antibodies to the human, conducting an immunotest to determine the presence, absence and/or amount of a Haemophilus influenzae antigen in a blood sample of the human using the same affinity purified human polyclonal antibodies, to determine an optimal therapeutic or preventive dose of the affinity purified human polyclonal antibodies, wherein the optimal therapeutic, removal or preventive dose is determined based on the amount of the Haemophilus influenzae antigen remaining after administering the affinity purified human polyclonal antibodies to the human and the extent of reduction in the Haemophilus influenzae antigen after administering the affinity purified human polyclonal antibodies to the human relative to the amount of Haemophilus influenzae antigen before the administration.

23. A method for treating or preventing a bacterial infection, which method comprises administering to a human suffering, suspected of suffering or at risk of suffering from a bacterial infection, an effective amount of the pharmaceutical composition of claim 14 to the human.

24. An antigenic composition comprising at least one, preferably two, Influenza A virus polypeptides, wherein each of said polypeptides comprises an amino acid sequence selected from the group consisting of:
a) polymerase B1 (PB1) sequence, from N-terminus to C-terminus, DAVATTHSWIPKRNRSIL (SEQ ID NO:1),
b) PB1 sequence, from N-terminus to C-terminus, FLKDVMESM (SEQ ID NO:2),
c) PB1 sequence, from N-terminus to C-terminus, FNMLSTVLGV (SEQ ID NO:3),
d) PB1 sequence, from N-terminus to C-terminus, FSMELPSFGV (SEQ ID NO:4),
e) PB1 sequence, from N-terminus to C-terminus, GPATAQMAL (SEQ ID NO:5),
f) PB1 sequence, from N-terminus to C-terminus, DTVNRTHQY (SEQ ID NO:6),
g) polymerase B2 (PB2) sequence, from N-terminus to C-terminus, YMLERELVRKTRFLPVA (SEQ ID NO:7),
h) PB2 sequence, from N-terminus to C-terminus, NFVNRANQRLNPMHQLLR (SEQ ID NO:8),
i) polymerase A (PA) sequence, from N-terminus to C-terminus, FMYSDFHFI (SEQ ID NO:9),
j) PA sequence, from N-terminus to C-terminus, RSKFLLMDALKLSIE (SEQ ID NO:10),
k) PA sequence, from N-terminus to C-terminus, SVKEKDMTK (SEQ ID NO:11),
l) PA sequence, from N-terminus to C-terminus, MRRNYFTAEVSHCRATEY (SEQ ID NO:12),
m) PA sequence, from N-terminus to C-terminus, AESRKLLLI (SEQ ID NO:13),
n) hemagglutinin (HA) sequence, from N-terminus to C-terminus, GLFGAIAGFC (SEQ ID NO:14),
o) HA sequence, from N-terminus to C-terminus, GLFGAIAGFI (SEQ ID NO:15),
p) HA sequence, from N-terminus to C-terminus, TGMVDGWYGYHHQNEQGS (SEQ ID NO:16),
q) HA sequence, from N-terminus to C-terminus, WTYNAELLVLLENERTLD (SEQ ID NO:17),
r) HA sequence, from N-terminus to C-terminus, NKVNSVIEKMNTQFTAVG (SEQ ID NO:18),
s) HA sequence, from N-terminus to C-terminus, GLFGAIAGFIE (SEQ ID NO:19),
t) HA sequence, from N-terminus to C-terminus, YPYDVPDYA (SEQ ID NO:20),
u) HA sequence, from N-terminus to C-terminus, VTGLRNIPSIQCR (SEQ ID NO:21),
v) HA sequence, from N-terminus to C-terminus, SVSSFERFEIFPK (SEQ ID NO:22),
w) nucleoprotein (NP) sequence, from N-terminus to C-terminus, RRSGAAGAAVK (SEQ ID NO:23),
x) NP sequence, from N-terminus to C-terminus, QLVWMACHSAA (SEQ ID NO:24),
y) NP sequence, from N-terminus to C-terminus, YERMCNILKG (SEQ ID NO:25),
z) NP sequence, from N-terminus to C-terminus, TYQRTRALV (SEQ ID NO:26),
aa) NP sequence, from N-terminus to C-terminus, RMVLSAFDER (SEQ ID NO:27),
bb) NP sequence, from N-terminus to C-terminus, LELRSRYWAI (SEQ ID NO:28),
cc) NP sequence, from N-terminus to C-terminus, KLSTRGVQIASNEN (SEQ ID NO:29),
dd) neuraminidase (NA) sequence, from N-terminus to C-terminus, SWPDGAELPF (SEQ ID NO:30),
ee) NA sequence, from N-terminus to C-terminus, PIRGWAI (SEQ ID NO: 31),
ff) NA sequence, from N-terminus to C-terminus, SGSFVQHPELTGL (SEQ ID NO:32),
gg) NA sequence, from N-terminus to C-terminus, VGLISLILQI (SEQ ID NO:33),
hh) matrix protein 1 (M1) sequence, from N-terminus to C-terminus, KTRPILSPLTK (SEQ ID NO:34),
ii) M1 sequence, from N-terminus to C-terminus, QKRMGVQMQRFK (SEQ ID NO:35),
jj) M1 sequence, from N-terminus to C-terminus, AGKNTDLEALMEWLKTR (SEQ ID NO:36),
kk) M1 sequence, from N-terminus to C-terminus, IRHENRMVL (SEQ ID NO:37),
ll) M1 sequence, from N-terminus to C-terminus, GILGFVFTL (SEQ ID NO:38),
mm) M1 sequence, from N-terminus to C-terminus, SLLTEVETYVL (SEQ ID NO:39),
nn) M1 sequence, from N-terminus to C-terminus, KGILGFVFTLTVPSE (SEQ ID NO:40),
oo) M1 sequence, from N-terminus to C-terminus, ILSPLTKGIL (SEQ ID NO:41),
pp) M1 sequence, from N-terminus to C-terminus, RMVLASTTAKAMEQM (SEQ ID NO:42),
qq) matrix protein 2 (M2) sequence, from N-terminus to C-terminus, SLLTEVET (SEQ ID NO:43),
rr) M2 sequence, from N-terminus to C-terminus, EVETPIRN (SEQ ID NO:44),
ss) non-structural protein 1 (NS1) sequence, from N-terminus to C-terminus, GEISPLPSL (SEQ ID NO:45),
tt) NS1 sequence, from N-terminus to C-terminus, DRLRRDQKS (SEQ ID NO:46),
uu) NS1 sequence, from N-terminus to C-terminus, AIMDKNIIL (SEQ ID NO:47),
vv) non-structural protein 2 (NS2) sequence, from N-terminus to C-terminus, ITFMQALQLL (SEQ ID NO:48), and
ww) NS2 sequence, from N-terminus to C-terminus, RTFSFQLI (SEQ ID NO:49),
wherein each of said polypeptides does not comprise any additional amino acid sequence of a naturally occurring Influenza A virus protein besides the amino acid sequences recited in SEQ ID NO:1 to SEQ ID NO:49.

25. A pharmaceutical composition for treating or preventing an Influenza A virus infection, which composition comprises an effective amount of human polyclonal antibodies affinity purified from a human blood sample with the composition of claim 24.

26. A method for treating or preventing an Influenza A virus infection, which method comprises administering to a human suffering, suspected of suffering or at risk of suffering from an Influenza A virus infection, an effective amount of the pharmaceutical composition of claim 25.

27. A pharmaceutical composition for treating or preventing a bacterial and/or viral infection, which composition comprises an effective amount of human polyclonal antibodies affinity purified from a human blood sample with an antigenic preparation selected from the group consisting of an antigenic preparation comprising cellular and/or secreted antigens of Staphylococcus aureus cells, Streptococcus cells, Pseudomonas aeruginosa cells, Haemophilus influenzae cells, K. pneumoniae cells, Enterococcus faecalis (E. faecalis) cells, Enterobacter aerogenes (E. aerogenes) cells, Enterobacter cloacae (E. cloacae) cells, Salmonella cells, TB-causing Mycobacterium cells, Bacillus anthracis cells, Listeria monocytogenes cells, Chlamydophila pneumoniae cells, Ureaplasma urealyticum cells, Mycoplasma hominis cells, Mycoplasma pneumoniae cells, malaria-causing Plasmodium cells, Pneumocystis jirovecii cells, Histoplasma capsulatum cells, Blastomyces dermatitidis cells, Coccidioides cells, Aspergillus cells, Haemophilus influenzae cells, Campylobacter jejuni cells, an antigenic preparation comprising an antigen of Variola virus, an antigen of respiratory syncytial virus (RSV), and/or an antigen of Cytomegalovirus (CMV).

28. A method for treating or preventing a bacterial and/or viral infection, which method comprises administering to a human suffering, suspected of suffering or at risk of suffering from a bacterial and/or viral infection, an effective amount of the pharmaceutical composition of claim 27.

29. A pharmaceutical composition for treating or preventing a bacterial and a viral infection, which composition comprises:
a) an effective amount of human polyclonal antibodies affinity purified from a human blood sample with the composition of claim 24; and
b) an effective amount of human polyclonal antibodies affinity purified from a human blood sample with an antigenic preparation selected from the group consisting of an antigenic preparation comprising cellular and/or secreted antigens of Staphylococcus aureus cells, Streptococcus cells, Pseudomonas aeruginosa cells, Haemophilus influenzae cells, K. pneumoniae cells, Enterococcus faecalis (E. faecalis) cells, Enterobacter aerogenes (E. aerogenes) cells, Enterobacter cloacae (E. cloacae) cells, Salmonella cells, TB-causing Mycobacterium cells, Bacillus anthracis cells, Listeria monocytogenes cells, Chlamydophila pneumoniae cells, Ureaplasma urealyticum cells, Mycoplasma hominis cells, Mycoplasma pneumoniae cells, malaria-causing Plasmodium cells, Pneumocystis jirovecii cells, Histoplasma capsulatum cells, Blastomyces dermatitidis cells, Coccidioides cells, Aspergillus cells, Campylobacter jejuni cells, an antigenic preparation comprising an antigen of Variola virus, an antigen of respiratory syncytial virus (RSV), and/or an antigen of Cytomegalovirus (CMV).

30. The pharmaceutical composition of claim 29, further comprising an effective amount of human polyclonal antibodies to an antigen from human tumor necrosis factor alpha (TNF-α).

31. A method for treating or preventing a bacterial and/or viral infection, which method comprises administering to a human suffering, suspected of suffering or at risk of suffering from a bacterial and/or viral infection, an effective amount of the pharmaceutical composition of claim 29.

32. A method for treating or preventing a bacterial and/or viral infection, which method comprises administering to a human suffering, suspected of suffering or at risk of suffering from a bacterial and/or viral infection, an effective amount of the pharmaceutical composition of claim 30.

33. An immunological composition, which composition comprises an effective amount of an antigenic preparation comprising cellular and secreted antigens from at least two different bacteria selected from the group consisting of Staphylococcus aureus (S. aureus), Escherichia coli (E. coli), a Streptococcus, Klebsiella pneumoniae (K. pneumoniae), Enterococcus, e.g., Enterococcus faecium (E. faecium), Haemophilus influenzae (H. influenzae), Pseudomonas aeruginosa (P. aeruginosa), Acinetobacter baumannii (A. baumannii), Enterococcus faecalis (E. faecalis), Enterobacter aerogenes (E. aerogenes), Enterobacter cloacae (E. cloacae), Clostridium difficile (C. difficile), a Salmonella, a TB-causing Mycobacterium, Bacillus anthracis (B. anthracis), Listeria monocytogenes (L. monocytogenes), Chlamydophila pneumoniae (C. pneumoniae), Ureaplasma urealyticum (U. urealyticum), Mycoplasma hominis (M. hominis), Mycoplasma pneumoniae (M. pneumoniae), and Campylobacter jejuni (C. jejuni).

34. A vaccine, which vaccine comprises an effective amount of an immunological composition of any of claim 33.

35. A method for immunizing or treating a subject, which method comprises administering to a subject for whom such immunization or treatment is needed or desirable an effective amount of a vaccine of claim 34.

36. An immunological composition for treating cystic fibrosis, which immunological composition comprises an antigenic composition comprising cellular and secreted antigens from Pseudomonas aeruginosa (P. aeruginosa) and/or Burkholderia cepacia complex (BCC).

37. A vaccine, which vaccine comprises an effective amount of an immunological composition of any of claim 36.

38. A method for treating cystic fibrosis, which method comprises administering to a subject in need of such treatment an effective amount of an immunological composition of claim 36.


SEQUENCE LISTING (49 sequences)

SEQ ID NO: 1 - protein, 18 aa, Artificial Sequence
  synthetically constucted polypeptide
DAVATTHSWIPKRNRSIL

SEQ ID NO: 2 - protein, 9 aa, Artificial Sequence
  synthetically constucted polypeptide
FLKDVMESM

SEQ ID NO: 3 - protein, 10 aa, Artificial Sequence
  synthetically constucted polypeptide
FNMLSTVLGV

SEQ ID NO: 4 - protein, 10 aa, Artificial Sequence
  synthetically constucted polypeptide
FSMELPSFGV

SEQ ID NO: 5 - protein, 9 aa, Artificial Sequence
  synthetically constucted polypeptide
GPATAQMAL

SEQ ID NO: 6 - protein, 9 aa, Artificial Sequence
  synthetically constucted polypeptide
DTVNRTHQY

SEQ ID NO: 7 - protein, 17 aa, Artificial Sequence
  synthetically constucted polypeptide
YMLERELVRKTRFLPVA

SEQ ID NO: 8 - protein, 18 aa, Artificial Sequence
  synthetically constucted polypeptide
NFVNRANQRLNPMHQLLR

SEQ ID NO: 9 - protein, 9 aa, Artificial Sequence
  synthetically constucted polypeptide
FMYSDFHFI

SEQ ID NO: 10 - protein, 15 aa, Artificial Sequence
  synthetically constucted polypeptide
RSKFLLMDALKLSIE

SEQ ID NO: 11 - protein, 9 aa, Artificial Sequence
  synthetically constucted polypeptide
SVKEKDMTK

SEQ ID NO: 12 - protein, 18 aa, Artificial Sequence
  synthetically constucted polypeptide
MRRNYFTAEVSHCRATEY

SEQ ID NO: 13 - protein, 9 aa, Artificial Sequence
  synthetically constucted polypeptide
AESRKLLLI

SEQ ID NO: 14 - protein, 10 aa, Artificial Sequence
  synthetically constucted polypeptide
GLFGAIAGFC

SEQ ID NO: 15 - protein, 10 aa, Artificial Sequence
  synthetically constucted polypeptide
GLFGAIAGFI

SEQ ID NO: 16 - protein, 18 aa, Artificial Sequence
  synthetically constucted polypeptide
TGMVDGWYGYHHQNEQGS

SEQ ID NO: 17 - protein, 18 aa, Artificial Sequence
  synthetically constucted polypeptide
WTYNAELLVLLENERTLD

SEQ ID NO: 18 - protein, 18 aa, Artificial Sequence
  synthetically constucted polypeptide
NKVNSVIEKMNTQFTAVG

SEQ ID NO: 19 - protein, 11 aa, Artificial Sequence
  synthetically constucted polypeptide
GLFGAIAGFIE

SEQ ID NO: 20 - protein, 9 aa, Artificial Sequence
  synthetically constucted polypeptide
YPYDVPDYA

SEQ ID NO: 21 - protein, 13 aa, Artificial Sequence
  synthetically constucted polypeptide
VTGLRNIPSIQCR

SEQ ID NO: 22 - protein, 13 aa, Artificial Sequence
  synthetically constucted polypeptide
SVSSFERFEIFPK

SEQ ID NO: 23 - protein, 11 aa, Artificial Sequence
  synthetically constucted polypeptide
RRSGAAGAAVK

SEQ ID NO: 24 - protein, 11 aa, Artificial Sequence
  synthetically constucted polypeptide
QLVWMACHSAA

SEQ ID NO: 25 - protein, 10 aa, Artificial Sequence
  synthetically constucted polypeptide
YERMCNILKG

SEQ ID NO: 26 - protein, 9 aa, Artificial Sequence
  synthetically constucted polypeptide
TYQRTRALV

SEQ ID NO: 27 - protein, 10 aa, Artificial Sequence
  synthetically constucted polypeptide
RMVLSAFDER

SEQ ID NO: 28 - protein, 10 aa, Artificial Sequence
  synthetically constucted polypeptide
LELRSRYWAI

SEQ ID NO: 29 - protein, 14 aa, Artificial Sequence
  synthetically constucted polypeptide
KLSTRGVQIASNEN

SEQ ID NO: 30 - protein, 10 aa, Artificial Sequence
  synthetically constucted polypeptide
SWPDGAELPF

SEQ ID NO: 31 - protein, 7 aa, Artificial Sequence
  synthetically constucted polypeptide
PIRGWAI

SEQ ID NO: 32 - protein, 13 aa, Artificial Sequence
  synthetically constucted polypeptide
SGSFVQHPELTGL

SEQ ID NO: 33 - protein, 10 aa, Artificial Sequence
  synthetically constucted polypeptide
VGLISLILQI

SEQ ID NO: 34 - protein, 11 aa, Artificial Sequence
  synthetically constucted polypeptide
KTRPILSPLTK

SEQ ID NO: 35 - protein, 12 aa, Artificial Sequence
  synthetically constucted polypeptide
QKRMGVQMQRFK

SEQ ID NO: 36 - protein, 17 aa, Artificial Sequence
  synthetically constucted polypeptide
AGKNTDLEALMEWLKTR

SEQ ID NO: 37 - protein, 9 aa, Artificial Sequence
  synthetically constucted polypeptide
IRHENRMVL

SEQ ID NO: 38 - protein, 9 aa, Artificial Sequence
  synthetically constucted polypeptide
GILGFVFTL

SEQ ID NO: 39 - protein, 11 aa, Artificial Sequence
  synthetically constucted polypeptide
SLLTEVETYVL

SEQ ID NO: 40 - protein, 15 aa, Artificial Sequence
  synthetically constucted polypeptide
KGILGFVFTLTVPSE

SEQ ID NO: 41 - protein, 10 aa, Artificial Sequence
  synthetically constucted polypeptide
ILSPLTKGIL

SEQ ID NO: 42 - protein, 15 aa, Artificial Sequence
  synthetically constucted polypeptide
RMVLASTTAKAMEQM

SEQ ID NO: 43 - protein, 8 aa, Artificial Sequence
  synthetically constucted polypeptide
SLLTEVET

SEQ ID NO: 44 - protein, 8 aa, Artificial Sequence
  synthetically constucted polypeptide
EVETPIRN

SEQ ID NO: 45 - protein, 9 aa, Artificial Sequence
  synthetically constucted polypeptide
GEISPLPSL

SEQ ID NO: 46 - protein, 9 aa, Artificial Sequence
  synthetically constucted polypeptide
DRLRRDQKS

SEQ ID NO: 47 - protein, 9 aa, Artificial Sequence
  synthetically constucted polypeptide
AIMDKNIIL

SEQ ID NO: 48 - protein, 10 aa, Artificial Sequence
  synthetically constucted polypeptide
ITFMQALQLL

SEQ ID NO: 49 - protein, 8 aa, Artificial Sequence
  synthetically constucted polypeptide
RTFSFQLI